(19) United States
(12) Patent Application Publication
(10) Pub. No.: US 2022/0162244 A1
(43) Pub. Date: May 26, 2022

(54) ORGANIC ELECTROLUMINESCENT MATERIAL AND DEVICE THEREOF

(71) Applicant: BEIJING SUMMER SPROUT TECHNOLOGY CO., LTD., Beijing (CN)

(72) Inventors: Ming Sang, Beijing (CN); Zhen Wang, Beijing (CN); Hongbo Li, Beijing (CN); Wei Cai, Beijing (CN); Yanhua Liu, Beijing (CN); Bin Tang, Beijing (CN); Xuan Zhang, Beijing (CN); Chi Yuen Raymond Kwong, Beijing (CN); Chuanjun Xia, Beijing (CN)

(73) Assignee: BEIJING SUMMER SPROUT TECHNOLOGY CO., LTD., Beijing (CN)

(21) Appl. No.: 17/526,358

(22) Filed: Nov. 15, 2021

(30) Foreign Application Priority Data
Nov. 18, 2020 (CN) 202011291606.7
Sep. 2, 2021 (CN) 202111011390.9

(51) Int. Cl. C07F 15/00; H01L 51/00; C07D 519/00

(52) U.S. Cl. CPC C07F 15/0033; H01L 51/0085; C07D 519/00; H01L 51/5004; H01L 51/0072; H01L 51/0073; H01L 2251/552; H01L 51/0067

(57) ABSTRACT

Provided are an organic electroluminescent material and a device thereof. The organic electroluminescent material is a metal complex containing a ligand Lₐ having a structure of Formula 1A and a ligand L_{b}, having a structure of Formula 1B. Such metal complexes are applicable to electroluminescent devices and can obtain a higher sublimation yield during sublimation and have a lower evaporation temperature and can provide better device performance such as an increased device lifetime and a narrower full width at half maximum. Further provided are an electroluminescent device containing the metal complex and a compound composition containing the metal complex.

### CROSS-REFERENCE TO RELATED APPLICATION(S)

This application claims priority to Chinese Patent Application No. CN202011291606.7 filed on Nov. 18, 2020 and Chinese Patent Application No. CN202111011390.9 filed on Sep. 2, 2021, the disclosure of which are incorporated herein by reference in their entireties.

### TECHNICAL FIELD

The present disclosure relates to compounds for organic electronic devices such as organic light-emitting devices. In particular, the present disclosure relates to a metal complex containing a ligand Lₐ having a structure of Formula 1A and a ligand L_{b} having a structure of Formula 1B, and an organic electroluminescent device and compound composition containing the metal complex.

### BACKGROUND

Organic electronic devices include, but are not limited to, the following types: organic light-emitting diodes (OLEDs), organic field-effect transistors (O-FETs), organic light-emitting transistors (OLETs), organic photovoltaic devices (OPVs), dye-sensitized solar cells (DSSCs), organic optical detectors, organic photoreceptors, organic field-quench devices (OFQDs), light-emitting electrochemical cells (LECs), organic laser diodes and organic plasmon emitting devices.

In 1987, Tang and Van Slyke of Eastman Kodak reported a bilayer organic electroluminescent device, which comprises an arylamine hole transporting layer and a tris-8-hydroxyquinolato-aluminum layer as the electron and emitting layer (Applied Physics Letters, 1987, 51 (12): 913-915). Once a bias is applied to the device, green light was emitted from the device. This device laid the foundation for the development of modern organic light-emitting diodes (OLEDs). State-of-the-art OLEDs may comprise multiple layers such as charge injection and transporting layers, charge and exciton blocking layers, and one or multiple emissive layers between the cathode and anode. Since the OLED is a self-emitting solid state device, it offers tremendous potential for display and lighting applications. In addition, the inherent properties of organic materials, such as their flexibility, may make them well suited for particular applications such as fabrication on flexible substrates.

The OLED can be categorized as three different types according to its emitting mechanism. The OLED invented by Tang and van Slyke is a fluorescent OLED. It only utilizes singlet emission. The triplets generated in the device are wasted through nonradiative decay channels. Therefore, the internal quantum efficiency (IQE) of the fluorescent OLED is only 25%. This limitation hindered the commercialization of OLED. In 1997, Forrest and Thompson reported phosphorescent OLED, which uses triplet emission from heavy metal containing complexes as the emitter. As a result, both singlet and triplets can be harvested, achieving 100% IQE. The discovery and development of phosphorescent OLED contributed directly to the commercialization of active-matrix OLED (AMOLED) due to its high efficiency. Recently, Adachi achieved high efficiency through thermally activated delayed fluorescence (TADF) of organic compounds. These emitters have small singlet-triplet gap that makes the transition from triplet back to singlet possible. In the TADF device, the triplet excitons can go through reverse intersystem crossing to generate singlet excitons, resulting in high IQE.

OLEDs can also be classified as small molecule and polymer OLEDs according to the forms of the materials used. A small molecule refers to any organic or organometallic material that is not a polymer. The molecular weight of the small molecule can be large as long as it has well defined structure. Dendrimers with well-defined structures are considered as small molecules. Polymer OLEDs include conjugated polymers and non-conjugated polymers with pendant emitting groups. Small molecule OLED can become the polymer OLED if post polymerization occurred during the fabrication process.

There are various methods for OLED fabrication. Small molecule OLEDs are generally fabricated by vacuum thermal evaporation. Polymer OLEDs are fabricated by solution process such as spin-coating, inkjet printing, and slit printing. If the material can be dissolved or dispersed in a solvent, the small molecule OLED can also be produced by solution process.

The emitting color of the OLED can be achieved by emitter structural design. An OLED may comprise one emitting layer or a plurality of emitting layers to achieve desired spectrum. In the case of green, yellow, and red OLEDs, phosphorescent emitters have successfully reached commercialization. Blue phosphorescent device still suffers from non-saturated blue color, short device lifetime, and high operating voltage. Commercial full-color OLED displays normally adopt a hybrid strategy, using fluorescent blue and phosphorescent yellow, or red and green. At present, efficiency roll-off of phosphorescent OLEDs at high brightness remains a problem. In addition, it is desirable to have more saturated emitting color, higher efficiency, and longer device lifetime.

Cyano substitutions are not generally introduced into phosphorescent metal complexes such as iridium complexes. US20140252333A1 has disclosed a series of iridium complexes with cyano and phenyl substitutions and has not clearly showed an effect of cyano groups. In addition, since cyano is a very electron-withdrawing substituent, cyano is also used to blue-shift the emission spectrum of a phosphorescent metal complex, as disclosed in US20040121184A1. A previous application US20200251666A1 of the applicant for the present application has disclosed a metal complex having a cyano-substituted ligand. The metal complex is applicable to an organic electroluminescent device and can improve device performance and color saturation to a relatively high level in the industry, but it is still to be improved.

Alkyl substitutions are generally introduced into phosphorescent metal complexes such as iridium complexes for emission of red light. It is found in US2014231755A1 that deuterated methyl at position 5 of 2-phenylpyridine can improve the lifetime of a device.

### SUMMARY

The present disclosure aims to provide a series of metal complexes each containing a ligand Lₐ having a structure of Formula 1A and a ligand L_{b} having a structure of Formula 1B to solve at least part of the preceding problems. These metal complexes may be used as light-emitting materials in electroluminescent devices. These new compounds can obtain a higher sublimation yield during sublimation and have a lower evaporation temperature. These metal complexes are applicable to electroluminescent devices and can provide better device performance such as an improved device lifetime and a narrower full width at half maximum (FWHM).

According to an embodiment of the present disclosure, provided is a metal complex having a general formula of M(Lₐ)ₘ(L_{b})ₙ(L_{c})_{q},

wherein

Lₐ, L_{b} and L_{c} are a first ligand, a second ligand and a third ligand coordinated to the metal M, respectively, and L_{c} is identical to or different from Lₐ or L_{b}; wherein Lₐ, L_{b} and L_{c} can be optionally joined to form a multidentate ligand;

the metal M is selected from a metal with a relative atomic mass greater than 40; preferably, the metal M is, at each occurrence identically or differently, selected from the group consisting of Cu, Ag, Au, Ru, Rh, Pd, Os, Ir and Pt; more preferably, M is, at each occurrence identically or differently, selected from Pt or Ir;

m is 1 or 2, n is 1 or 2, q is 0 or 1, and m+n+q equals to the oxidation state of M; when m is 2, two Lₐ are identical or different; when n is 2, two L_{b} are identical or different;

Lₐ has, at each occurrence identically or differently, a structure represented by Formula 1A and L_{b} has, at each occurrence identically or differently, a structure represented by Formula 1B:

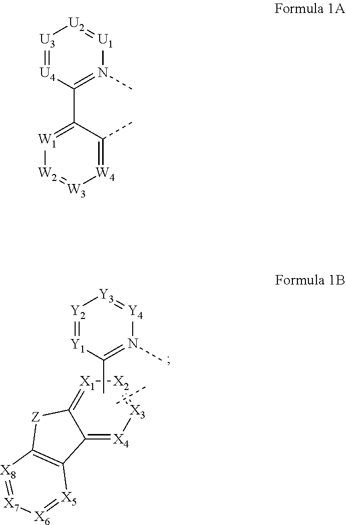

wherein

Z is selected from the group consisting of O, S, Se, NR, CRR and SiRR, wherein when two R are present, the two R are identical or different;

X₁ to X₈ are, at each occurrence identically or differently, selected from C or CRₓ;

Y₁ to Y₄ are, at each occurrence identically or differently, selected from CR_{y} or N;

U₁ to U₄ are, at each occurrence identically or differently, selected from CRᵤ or N;

W₁ to W₄ are, at each occurrence identically or differently, selected from CR_{w} or N;

R, Rₓ, R_{y}, Rᵤ and R_{w} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

at least one or more of U₁ to U₄ are selected from CRᵤ, and the Rᵤ is substituted or unsubstituted alkyl having 1 to 20 carbon atoms, substituted or unsubstituted cycloalkyl having 3 to 20 ring carbon atoms or a combination thereof, and the total number of carbon atoms in all of the Rᵤ is at least 4;

at least one of Rₓ is cyano; and

adjacent substituents R, Rₓ, R_{y}, Rᵤ, R_{w} can be optionally joined to form a ring;

L_{c} is, at each occurrence identically or differently, selected from a structure represented by any one of the group consisting of the following:

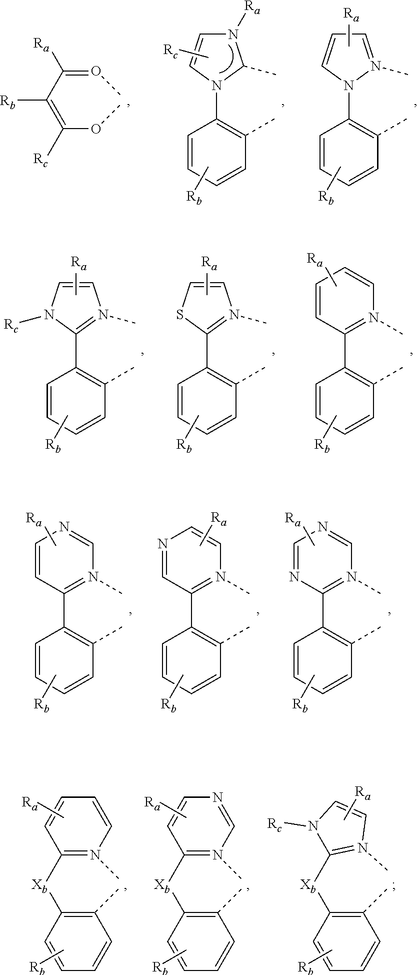

wherein

Rₐ, R_{b} and R_{c} represent, at each occurrence identically or differently, mono-substitution, multiple substitutions or non-substitution;

X_{b} is, at each occurrence identically or differently, selected from the group consisting of: O, S, Se, NR_{N1} and CR_{C1}R_{C2};

Rₐ, R_{b}, R_{c}, R_{N1}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and

adjacent substituents Rₐ, R_{b}, R_{c}, R_{N1}, R_{C1} and R_{C2} can be optionally joined to form a ring.

According to another embodiment of the present disclosure, further provided is an electroluminescent device, comprising:

an anode,

a cathode, and

an organic layer disposed between the anode and the cathode, wherein at least one layer of the organic layer contains the metal complex in the preceding embodiment.

According to another embodiment of the present disclosure, further provided is a compound composition, comprising the metal complex in the preceding embodiment.

The present disclosure provides a series of metal complexes each containing a ligand Lₐ having a structure of Formula 1A and a ligand L_{b} having a structure of Formula 1B, where a particular substituent is introduced into the ligand Lₐ and cyano is introduced into the ligand L_{b} so that these new compounds can obtain the higher sublimation yield during sublimation and have the lower evaporation temperature. These metal complexes may be used as light-emitting materials in electroluminescent devices. These metal complexes are applicable to electroluminescent devices and can provide the better device performance such as the improved device lifetime and the narrower FWHM.

### BRIEF DESCRIPTION OF DRAWINGS

FIG. 1 is a schematic diagram of an organic light-emitting device that may contain a metal complex and a compound composition disclosed herein.

FIG. 2 is a schematic diagram of another organic light-emitting device that may contain a metal complex and a compound composition disclosed herein.

### DETAILED DESCRIPTION

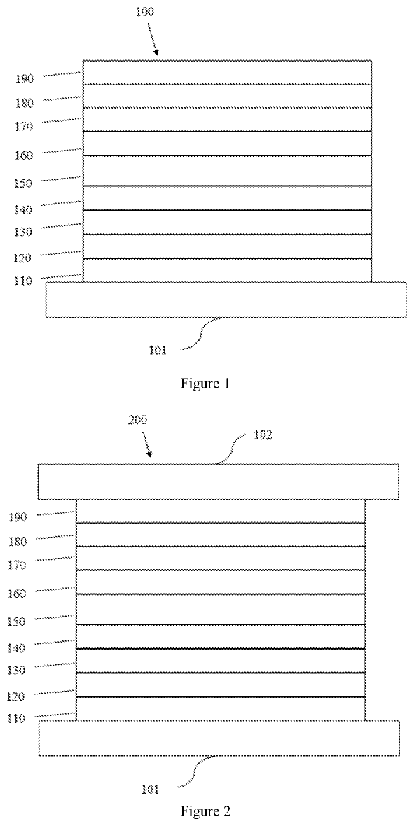

OLEDs can be fabricated on various types of substrates such as glass, plastic, and metal foil. FIG. 1 schematically shows an organic light emitting device 100 without limitation. The figures are not necessarily drawn to scale. Some of the layers in the figures can also be omitted as needed. Device 100 may include a substrate 101, an anode 110, a hole injection layer 120, a hole transport layer 130, an electron blocking layer 140, an emissive layer 150, a hole blocking layer 160, an electron transport layer 170, an electron injection layer 180 and a cathode 190. Device 100 may be fabricated by depositing the layers described in order. The properties and functions of these various layers, as well as example materials, are described in more detail in U.S. Pat. No. 7,279,704 at cols. 6-10, the contents of which are incorporated by reference herein in its entirety.

More examples for each of these layers are available. For example, a flexible and transparent substrate-anode combination is disclosed in U.S. Pat. No. 5,844,363, which is incorporated by reference herein in its entirety. An example of a p-doped hole transport layer is m-MTDATA doped with F4-TCNQ at a molar ratio of 50:1, as disclosed in U.S. Patent Application Publication No. 2003/0230980, which is incorporated by reference herein in its entirety. Examples of host materials are disclosed in U.S. Pat. No. 6,303,238 to Thompson et al., which is incorporated by reference herein in its entirety. An example of an n-doped electron transport layer is BPhen doped with Li at a molar ratio of 1:1, as disclosed in U.S. Patent Application Publication No. 2003/0230980, which is incorporated by reference herein in its entirety. U.S. Pat. Nos. 5,703,436 and 5,707,745, which are incorporated by reference herein in their entireties, disclose examples of cathodes including composite cathodes having a thin layer of metal such as Mg:Ag with an overlying transparent, electrically-conductive, sputter-deposited ITO layer. The theory and use of blocking layers are described in more detail in U.S. Pat. No. 6,097,147 and U.S. Patent Application Publication No. 2003/0230980, which are incorporated by reference herein in their entireties. Examples of injection layers are provided in U.S. Patent Application Publication No. 2004/0174116, which is incorporated by reference herein in its entirety. A description of protective layers may be found in U.S. Patent Application Publication No. 2004/0174116, which is incorporated by reference herein in its entirety.

The layered structure described above is provided by way of non-limiting examples. Functional OLEDs may be achieved by combining the various layers described in different ways, or layers may be omitted entirely. It may also include other layers not specifically described. Within each layer, a single material or a mixture of multiple materials can be used to achieve optimum performance. Any functional layer may include several sublayers. For example, the emissive layer may have two layers of different emitting materials to achieve desired emission spectrum.

In one embodiment, an OLED may be described as having an “organic layer” disposed between a cathode and an anode. This organic layer may comprise a single layer or multiple layers.

An OLED can be encapsulated by a barrier layer. FIG. 2 schematically shows an organic light emitting device 200 without limitation. FIG. 2 differs from FIG. 1 in that the organic light emitting device include a barrier layer 102, which is above the cathode 190, to protect it from harmful species from the environment such as moisture and oxygen. Any material that can provide the barrier function can be used as the barrier layer such as glass or organic-inorganic hybrid layers. The barrier layer should be placed directly or indirectly outside of the OLED device. Multilayer thin film encapsulation was described in U.S. Pat. No. 7,968,146, which is incorporated by reference herein in its entirety.

Devices fabricated in accordance with embodiments of the present disclosure can be incorporated into a wide variety of consumer products that have one or more of the electronic component modules (or units) incorporated therein. Some examples of such consumer products include flat panel displays, monitors, medical monitors, televisions, billboards, lights for interior or exterior illumination and/or signaling, heads-up displays, fully or partially transparent displays, flexible displays, smart phones, tablets, phablets, wearable devices, smart watches, laptop computers, digital cameras, camcorders, viewfinders, micro-displays, 3-D displays, vehicles displays, and vehicle tail lights.

The materials and structures described herein may be used in other organic electronic devices listed above.

As used herein, “top” means furthest away from the substrate, while “bottom” means closest to the substrate. Where a first layer is described as “disposed over” a second layer, the first layer is disposed further away from the substrate. There may be other layers between the first and second layers, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

It is believed that the internal quantum efficiency (IQE) of fluorescent OLEDs can exceed the 25% spin statistics limit through delayed fluorescence. As used herein, there are two types of delayed fluorescence, i.e. P-type delayed fluorescence and E-type delayed fluorescence. P-type delayed fluorescence is generated from triplet-triplet annihilation (TTA).

On the other hand, E-type delayed fluorescence does not rely on the collision of two triplets, but rather on the transition between the triplet states and the singlet excited states. Compounds that are capable of generating E-type delayed fluorescence are required to have very small singlet-triplet gaps to convert between energy states. Thermal energy can activate the transition from the triplet state back to the singlet state. This type of delayed fluorescence is also known as thermally activated delayed fluorescence (TADF). A distinctive feature of TADF is that the delayed component increases as temperature rises. If the reverse intersystem crossing (RISC) rate is fast enough to minimize the non-radiative decay from the triplet state, the fraction of back populated singlet excited states can potentially reach 75%. The total singlet fraction can be 100%, far exceeding 25% of the spin statistics limit for electrically generated excitons.

E-type delayed fluorescence characteristics can be found in an exciplex system or in a single compound. Without being bound by theory, it is believed that E-type delayed fluorescence requires the luminescent material to have a small singlet-triplet energy gap (ΔES-T). Organic, non-metal containing, donor-acceptor luminescent materials may be able to achieve this. The emission in these materials is generally characterized as a donor-acceptor charge-transfer (CT) type emission. The spatial separation of the HOMO and LUMO in these donor-acceptor type compounds generally results in small ΔES-T. These states may involve CT states. Generally, donor-acceptor luminescent materials are constructed by connecting an electron donor moiety such as amino- or carbazole-derivatives and an electron acceptor moiety such as N-containing six-membered aromatic rings.

### Definition of Terms of Substituents

Halogen or halide—as used herein includes fluorine, chlorine, bromine, and iodine.

Alkyl—as used herein includes both straight and branched chain alkyl groups. Alkyl may be alkyl having 1 to 20 carbon atoms, preferably alkyl having 1 to 12 carbon atoms, and more preferably alkyl having 1 to 6 carbon atoms. Examples of alkyl groups include a methyl group, an ethyl group, a propyl group, an isopropyl group, a n-butyl group, an s-butyl group, an isobutyl group, a t-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, a neopentyl group, a 1-methylpentyl group, a 2-methylpentyl group, a 1-pentylhexyl group, a 1-butylpentyl group, a 1-heptyloctyl group, and a 3-methylpentyl group. Of the above, preferred are a methyl group, an ethyl group, a propyl group, an isopropyl group, a n-butyl group, an s-butyl group, an isobutyl group, a t-butyl group, an n-pentyl group, a neopentyl group, and an n-hexyl group. Additionally, the alkyl group may be optionally substituted.

Cycloalkyl—as used herein includes cyclic alkyl groups. The cycloalkyl groups may be those having 3 to 20 ring carbon atoms, preferably those having 4 to 10 carbon atoms. Examples of cycloalkyl include cyclobutyl, cyclopentyl, cyclohexyl, 4-methylcyclohexyl, 4,4-dimethylcylcohexyl, 1-adamantyl, 2-adamantyl, 1-norbornyl, 2-norbornyl, and the like. Of the above, preferred are cyclopentyl, cyclohexyl, 4-methylcyclohexyl, and 4,4-dimethylcylcohexyl. Additionally, the cycloalkyl group may be optionally substituted.

Heteroalkyl—as used herein, includes a group formed by replacing one or more carbons in an alkyl chain with a hetero-atom(s) selected from the group consisting of a nitrogen atom, an oxygen atom, a sulfur atom, a selenium atom, a phosphorus atom, a silicon atom, a germanium atom, and a boron atom. Heteroalkyl may be those having 1 to 20 carbon atoms, preferably those having 1 to 10 carbon atoms, and more preferably those having 1 to 6 carbon atoms. Examples of heteroalkyl include methoxymethyl, ethoxymethyl, ethoxyethyl, methylthiomethyl, ethylthiomethyl, ethylthioethyl, methoxymethoxymethyl, ethoxymethoxymethyl, ethoxyethoxyethyl, hydroxymethyl, hydroxyethyl, hydroxypropyl, mercaptomethyl, mercaptoethyl, mercaptopropyl, aminomethyl, aminoethyl, aminopropyl, dimethylaminomethyl, trimethylsilyl, dimethylethylsilyl, dimethylisopropylsilyl, t-butyldimethylsilyl, triethylsilyl, triisopropylsilyl, trimethylsilylmethyl, trimethylsilylethyl, and trimethylsilylisopropyl. Additionally, the heteroalkyl group may be optionally substituted.

Alkenyl—as used herein includes straight chain, branched chain, and cyclic alkene groups. Alkenyl may be those having 2 to 20 carbon atoms, preferably those having 2 to 10 carbon atoms. Examples of alkenyl include vinyl, 1-propenyl group, 1-butenyl, 2-butenyl, 3-butenyl, 1,3-butandienyl, 1-methylvinyl, styryl, 2,2-diphenylvinyl, 1,2-diphenylvinyl, 1-methylallyl, 1,1-dimethylallyl, 2-methylallyl, 1-phenylallyl, 2-phenylallyl, 3-phenylallyl, 3,3-diphenylallyl, 1,2-dimethylallyl, 1-phenyl-1-butenyl, 3-phenyl-1-butenyl, cyclopentenyl, cyclopentadienyl, cyclohexenyl, cycloheptenyl, cycloheptatrienyl, cyclooctenyl, cyclooctatetraenyl, and norbornenyl. Additionally, the alkenyl group may be optionally substituted.

Alkynyl—as used herein includes straight chain alkynyl groups. Alkynyl may be those having 2 to 20 carbon atoms, preferably those having 2 to 10 carbon atoms. Examples of alkynyl groups include ethynyl, propynyl, propargyl, 1-butynyl, 2-butynyl, 3-butynyl, 1-pentynyl, 2-pentynyl, 3,3-dimethyl-1-butynyl, 3-ethyl-3-methyl-1-pentynyl, 3,3-diisopropyl-1-pentynyl, phenylethynyl, phenylpropynyl, etc. Of the above, preferred are ethynyl, propynyl, propargyl, 1-butynyl, 2-butynyl, 3-butynyl, 1-pentynyl, and phenylethynyl. Additionally, the alkynyl group may be optionally substituted.

Aryl or an aromatic group—as used herein includes non-condensed and condensed systems. Aryl may be those having 6 to 30 carbon atoms, preferably those having 6 to 20 carbon atoms, and more preferably those having 6 to 12 carbon atoms. Examples of aryl groups include phenyl, biphenyl, terphenyl, triphenylene, tetraphenylene, naphthalene, anthracene, phenalene, phenanthrene, fluorene, pyrene, chrysene, perylene, and azulene, preferably phenyl, biphenyl, terphenyl, triphenylene, fluorene, and naphthalene. Examples of non-condensed aryl groups include phenyl, biphenyl-2-yl, biphenyl-3-yl, biphenyl-4-yl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-(2-phenylpropyl)phenyl, 4′-methylbiphenylyl, 4″-t-butyl-p-terphenyl-4-yl, o-cumenyl, m-cumenyl, p-cumenyl, 2,3-xylyl, 3,4-xylyl, 2,5-xylyl, mesityl, and m-quarterphenyl. Additionally, the aryl group may be optionally substituted.

Heterocyclic groups or heterocycle—as used herein include non-aromatic cyclic groups. Non-aromatic heterocyclic groups includes saturated heterocyclic groups having 3 to 20 ring atoms and unsaturated non-aromatic heterocyclic groups having 3 to 20 ring atoms, where at least one ring atom is selected from the group consisting of a nitrogen atom, an oxygen atom, a sulfur atom, a selenium atom, a silicon atom, a phosphorus atom, a germanium atom, and a boron atom. Preferred non-aromatic heterocyclic groups are those having 3 to 7 ring atoms, each of which includes at least one hetero-atom such as nitrogen, oxygen, silicon, or sulfur. Examples of non-aromatic heterocyclic groups include oxiranyl, oxetanyl, tetrahydrofuranyl, tetrahydropyranyl, dioxolanyl, dioxanyl, aziridinyl, dihydropyrrolyl, tetrahydropyrrolyl, piperidinyl, oxazolidinyl, morpholinyl, piperazinyl, oxepinyl, thiepinyl, azepinyl, and tetrahydrosilolyl. Additionally, the heterocyclic group may be optionally substituted.

Heteroaryl—as used herein, includes non-condensed and condensed hetero-aromatic groups having 1 to 5 hetero-atoms, where at least one hetero-atom is selected from the group consisting of a nitrogen atom, an oxygen atom, a sulfur atom, a selenium atom, a silicon atom, a phosphorus atom, a germanium atom, and a boron atom. A hetero-aromatic group is also referred to as heteroaryl. Heteroaryl may be those having 3 to 30 carbon atoms, preferably those having 3 to 20 carbon atoms, and more preferably those having 3 to 12 carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridoindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Alkoxy—as used herein, is represented by —O-alkyl, —O-cycloalkyl, —O-heteroalkyl, or —O-heterocyclic group. Examples and preferred examples of alkyl, cycloalkyl, heteroalkyl, and heterocyclic groups are the same as those described above. Alkoxy groups may be those having 1 to 20 carbon atoms, preferably those having 1 to 6 carbon atoms. Examples of alkoxy groups include methoxy, ethoxy, propoxy, butoxy, pentyloxy, hexyloxy, cyclopropyloxy, cyclobutyloxy, cyclopentyloxy, cyclohexyloxy, tetrahydrofuranyloxy, tetrahydropyranyloxy, methoxypropyloxy, ethoxyethyloxy, methoxymethyloxy, and ethoxymethyloxy. Additionally, the alkoxy group may be optionally substituted.

Aryloxy—as used herein, is represented by —O-aryl or —O-heteroaryl. Examples and preferred examples of aryl and heteroaryl are the same as those described above. Aryloxy groups may be those having 6 to 30 carbon atoms, preferably those having 6 to 20 carbon atoms. Examples of aryloxy groups include phenoxy and biphenyloxy. Additionally, the aryloxy group may be optionally substituted.

Arylalkyl—as used herein, contemplates alkyl substituted with an aryl group. Arylalkyl may be those having 7 to 30 carbon atoms, preferably those having 7 to 20 carbon atoms, and more preferably those having 7 to 13 carbon atoms. Examples of arylalkyl groups include benzyl, 1-phenylethyl, 2-phenylethyl, 1-phenylisopropyl, 2-phenylisopropyl, phenyl-t-butyl, alpha-naphthylmethyl, 1-alpha-naphthylethyl, 2-alpha-naphthylethyl, 1-alpha-naphthylisopropyl, 2-alpha-naphthylisopropyl, beta-naphthylmethyl, 1-beta-naphthylethyl, 2-beta-naphthylethyl, 1-beta-naphthylisopropyl, 2-beta-naphthylisopropyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, and 1-chloro-2-phenylisopropyl. Of the above, preferred are benzyl, p-cyanobenzyl, m-cyanobenzyl, o-cyanobenzyl, 1-phenylethyl, 2-phenylethyl, 1-phenylisopropyl, and 2-phenylisopropyl. Additionally, the arylalkyl group may be optionally substituted.

Alkylsilyl—as used herein, contemplates a silyl group substituted with an alkyl group. Alkylsilyl groups may be those having 3 to 20 carbon atoms, preferably those having 3 to 10 carbon atoms. Examples of alkylsilyl groups include trimethylsilyl, triethylsilyl, methyldiethylsilyl, ethyldimethylsilyl, tripropylsilyl, tributylsilyl, triisopropylsilyl, methyldiisopropylsilyl, dimethylisopropylsilyl, tri-t-butylsilyl, triisobutylsilyl, dimethyl t-butylsilyl, and methyldi-t-butylsilyl. Additionally, the alkylsilyl group may be optionally substituted.

Arylsilyl—as used herein, contemplates a silyl group substituted with an aryl group. Arylsilyl groups may be those having 6 to 30 carbon atoms, preferably those having 8 to 20 carbon atoms. Examples of arylsilyl groups include triphenylsilyl, phenyldibiphenylylsilyl, diphenylbiphenylsilyl, phenyldiethylsilyl, diphenylethylsilyl, phenyldimethylsilyl, diphenylmethylsilyl, phenyldiisopropylsilyl, diphenylisopropylsilyl, diphenylbutylsilyl, diphenylisobutylsilyl, diphenyl t-butylsilyl. Additionally, the arylsilyl group may be optionally substituted.

The term “aza” in azadibenzofuran, azadibenzothiophene, etc. means that one or more of C—H groups in the respective aromatic fragment are replaced by a nitrogen atom. For example, azatriphenylene encompasses dibenzo[f,h]quinoxaline, dibenzo[f,h]quinoline and other analogs with two or more nitrogens in the ring system. One of ordinary skill in the art can readily envision other nitrogen analogs of the aza-derivatives described above, and all such analogs are intended to be encompassed by the terms as set forth herein.

In the present disclosure, unless otherwise defined, when any term of the group consisting of substituted alkyl, substituted cycloalkyl, substituted heteroalkyl, substituted heterocyclic group, substituted arylalkyl, substituted alkoxy, substituted aryloxy, substituted alkenyl, substituted alkynyl, substituted aryl, substituted heteroaryl, substituted alkylsilyl, substituted arylsilyl, substituted amino, substituted acyl, substituted carbonyl, a substituted carboxylic acid group, a substituted ester group, substituted sulfinyl, substituted sulfonyl, and substituted phosphino is used, it means that any group of alkyl, cycloalkyl, heteroalkyl, heterocyclic group, arylalkyl, alkoxy, aryloxy, alkenyl, alkynyl, aryl, heteroaryl, alkylsilyl, arylsilyl, amino, acyl, carbonyl, a carboxylic acid group, an ester group, sulfinyl, sulfonyl, and phosphino may be substituted with one or more moieties selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted heteroaryl having 3 to 30 carbon atoms, unsubstituted alkylsilyl having 3 to 20 carbon atoms, unsubstituted arylsilyl group having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or an attached fragment are considered to be equivalent.

In the compounds mentioned in the present disclosure, hydrogen atoms may be partially or fully replaced by deuterium. Other atoms such as carbon and nitrogen may also be replaced by their other stable isotopes. The replacement by other stable isotopes in the compounds may be preferred due to its enhancements of device efficiency and stability.

In the compounds mentioned in the present disclosure, multiple substitution refers to a range that includes a di-substitution, up to the maximum available substitution. When substitution in the compounds mentioned in the present disclosure represents multiple substitution (including di-, tri-, and tetra-substitutions etc.), that means the substituent may exist at a plurality of available substitution positions on its linking structure, the substituents present at a plurality of available substitution positions may have the same structure or different structures.

In the compounds mentioned in the present disclosure, adjacent substituents in the compounds cannot be joined to form a ring unless otherwise explicitly defined, for example, adjacent substituents can be optionally joined to form a ring. In the compounds mentioned in the present disclosure, the expression that adjacent substituents can be optionally joined to form a ring includes a case where adjacent substituents may be joined to form a ring and a case where adjacent substituents are not joined to form a ring. When adjacent substituents can be optionally joined to form a ring, the ring formed may be monocyclic or polycyclic, as well as alicyclic, heteroalicyclic, aromatic, or heteroaromatic. In such expression, adjacent substituents may refer to substituents bonded to the same atom, substituents bonded to carbon atoms which are directly bonded to each other, or substituents bonded to carbon atoms which are more distant from each other. Preferably, adjacent substituents refer to substituents bonded to the same carbon atom and substituents bonded to carbon atoms which are directly bonded to each other.

The expression that adjacent substituents can be optionally joined to form a ring is also intended to mean that two substituents bonded to the same carbon atom are joined to each other via a chemical bond to form a ring, which can be exemplified by the following formula:

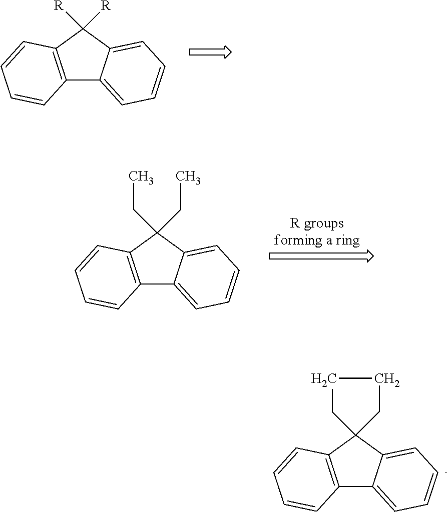

The expression that adjacent substituents can be optionally joined to form a ring is also intended to mean that two substituents bonded to carbon atoms which are directly bonded to each other are joined to each other via a chemical bond to form a ring, which can be exemplified by the following formula:

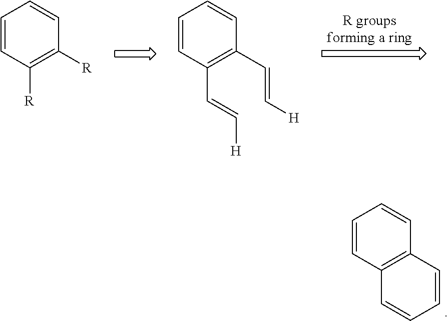

Furthermore, the expression that adjacent substituents can be optionally joined to form a ring is also intended to mean that, in the case where one of the two substituents bonded to carbon atoms which are directly bonded to each other represents hydrogen, the second substituent is bonded at a position at which the hydrogen atom is bonded, thereby forming a ring. This is exemplified by the following formula:

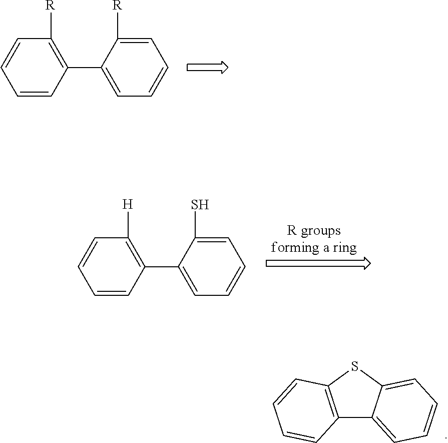

According to an embodiment of the present disclosure, provided is a metal complex having a general formula of M(Lₐ)ₘ(L_{b})ₙ(L_{c})_{q},

wherein

Lₐ, L_{b} and L_{c} are a first ligand, a second ligand and a third ligand coordinated to the metal M, respectively, and L_{c} is identical to or different from Lₐ or L_{b}; wherein Lₐ, L_{b} and L_{c} can be optionally joined to form a multidentate ligand; for example, any two of Lₐ, L_{b} and L_{c} may be joined to form a tetradentate ligand; in another example, Lₐ, L_{b} and L_{c} may be joined to each other to form a hexadentate ligand; in another example, none of Lₐ, L_{b} and L_{c} are joined so that no multidentate ligand is formed;

the metal M is selected from a metal with a relative atomic mass greater than 40; preferably, the metal M is, at each occurrence identically or differently, selected from the group consisting of Cu, Ag, Au, Ru, Rh, Pd, Os, Ir and Pt; more preferably, M is, at each occurrence identically or differently, selected from Pt or Ir;

m is 1 or 2, n is 1 or 2, q is 0 or 1, and m+n+q equals to the oxidation state of M; when m is 2, two Lₐ are identical or different; when n is 2, two L_{b} are identical or different;

Lₐ has, at each occurrence identically or differently, a structure represented by Formula 1A and L_{b} has, at each occurrence identically or differently, a structure represented by Formula 1B:

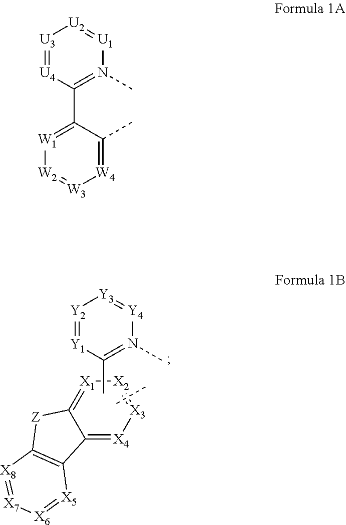

wherein

Z is selected from the group consisting of O, S, Se, NR, CRR and SiRR, wherein when two R are present, the two R are identical or different;

X₁ to X₈ are, at each occurrence identically or differently, selected from C or CRₓ;

Y₁ to Y₄ are, at each occurrence identically or differently, selected from CR_{y} or N;

U₁ to U₄ are, at each occurrence identically or differently, selected from CRᵤ or N;

W₁ to W₄ are, at each occurrence identically or differently, selected from CR_{w} or N;

R, Rₓ, R_{y}, Rᵤ and R_{w} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

at least one or more of U₁ to U₄ are selected from CRᵤ, and the Rᵤ is substituted or unsubstituted alkyl having 1 to 20 carbon atoms, substituted or unsubstituted cycloalkyl having 3 to 20 ring carbon atoms or a combination thereof, and the total number of carbon atoms in all of the Rᵤ is at least 4;

at least one of Rₓ is cyano; and

adjacent substituents R, Rₓ, R_{y}, Rᵤ, R_{w} can be optionally joined to form a ring;

L_{c} is, at each occurrence identically or differently, selected from a structure represented by any one of the group consisting of the following:

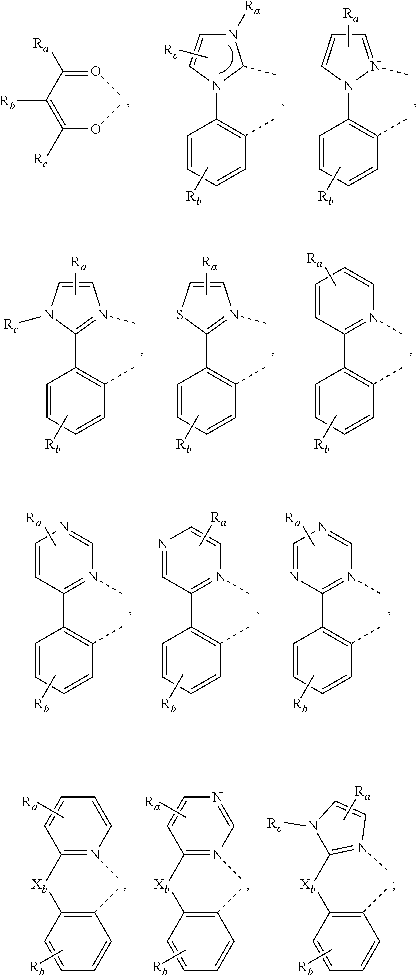

wherein

Rₐ, R_{b} and R_{c} represent, at each occurrence identically or differently, mono-substitution, multiple substitutions or non-substitution;

X_{b} is, at each occurrence identically or differently, selected from the group consisting of: O, S, Se, NR_{N1} and CR_{C1}R_{C2};

Rₐ, R_{b}, R_{c}, R_{N1}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and

adjacent substituents Rₐ, R_{b}, R_{c}, R_{N1}, R_{C1} and R_{C2} can be optionally joined to form a ring.

In the present disclosure, the expression that “the total number of carbon atoms in all of the Rᵤ is at least 4” means that the total number of carbon atoms in all Rᵤ that satisfies the condition that “one or more of U₁ to U₄ are selected from CRᵤ, and the Rᵤ is substituted or unsubstituted alkyl having 1 to 20 carbon atoms, substituted or unsubstituted cycloalkyl having 3 to 20 ring carbon atoms or a combination thereof” is greater than or equal to 4. When one of U₁ to U₄ satisfies the preceding condition, the number of carbon atoms in this substituent is greater than or equal to 4; when two of U₁ to U₄ satisfy the preceding condition, the total number of carbon atoms in these two substituents is greater than or equal to 4; when three of U₁ to U₄ satisfy the preceding condition, the total number of carbon atoms in these three substituents is greater than or equal to 4; when four of U₁ to U₄ satisfy the preceding condition, the total number of carbon atoms in these four substituents is greater than or equal to 4. For example, when U₂ is selected from CRᵤ and satisfies the preceding condition, the number of carbon atoms in the substituent Rᵤ of U₂ is greater than or equal to 4; when U₃ is selected from CRᵤ and satisfies the preceding condition, the number of carbon atoms in the substituent Rᵤ of U₃ is greater than or equal to 4. It is true in other cases.

In this embodiment, the expression that “adjacent substituents R, Rₓ, R_{y}, Rᵤ, R_{w} can be optionally joined to form a ring” is intended to mean that any one or more of groups of adjacent substituents, such as two substituents R, two substituents Rₓ, two substituents R_{y}, two substituents Rᵤ, two substituents R_{w}, two substituents R_{w} and Rᵤ, and two substituents R_{y} and Rₓ can be joined to form a ring. Obviously, it is possible that none of these substituents are joined to form a ring.

In the present disclosure, the expression that “adjacent substituents Rₐ, R_{b}, R_{c}, R_{N1}, R_{C1} and R_{C2} can be optionally joined to form a ring” is intended to mean that any one or more of groups of adjacent substituents, such as two substituents Rₐ, two substituents R_{b}, two substituents R_{c}, substituents Rₐ and R_{b}, substituents Rₐ and R_{c}, substituents R_{b} and R_{c}, substituents Rₐ and R_{N1}, substituents R_{b} and R_{N1}, substituents Rₐ and R_{C1}, substituents Rₐ and R_{C2}, substituents R_{b} and R_{C1}, substituents R_{b} and R_{C2}, and substituents R_{C1} and R_{C2}, can be joined to form a ring. Obviously, it is possible that none of these substituents are joined to form a ring.

According to an embodiment of the present disclosure, wherein, L_{b} has a structure represented by each of Formulas 1Ba to 1Bd:

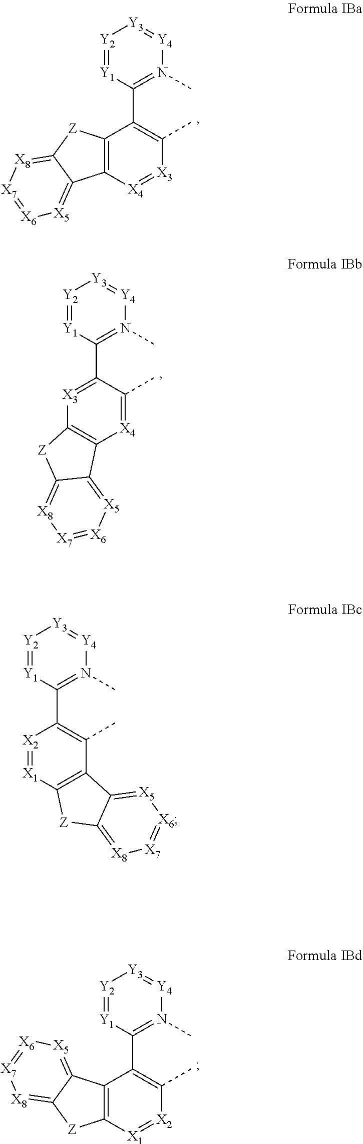

wherein

Z is selected from the group consisting of O, S, Se, NR, CRR and SiRR, wherein when two R are present, the two R are identical or different;

in Formula 1Ba, X₃ to X₈ are, at each occurrence identically or differently, selected from CRₓ;

in Formula 1Bb, X₁ and X₄ to X₈ are, at each occurrence identically or differently, selected from CRₓ;

in Formula 1Bc and Formula 1Bd, X₁, X₂ and X₅ to X₈ are, at each occurrence identically or differently, selected from CRₓ;

Y₁ to Y₄ are, at each occurrence identically or differently, selected from CR_{y} or N;

R, Rₓ and R_{y} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and

adjacent substituents R, Rₓ, R_{y} can be optionally joined to form a ring.

In this embodiment, the expression that “adjacent substituents R, Rₓ, R_{y} can be optionally joined to form a ring” is intended to mean that any one or more of groups of adjacent substituents, such as two substituents R, two substituents Rₓ, two substituents R_{y}, and two substituents R_{y} and Rₓ, can be joined to form a ring. Obviously, it is possible that none of these substituents are joined to form a ring.

According to an embodiment of the present disclosure, wherein, the metal complex has a structure represented by Formula 2:

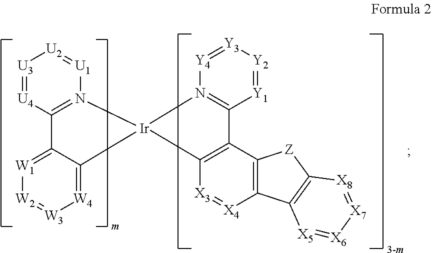

wherein

m is selected from 1 or 2; when m=1, two L_{b} are identical or different; when m=2, two Lₐ are identical or different;

Z is selected from the group consisting of O, S, Se, NR, CRR and SiRR, wherein when two R are present, the two R are identical or different;

X₃ to X₈ are, at each occurrence identically or differently, selected from CRₓ;

Y₁ to Y₄ are, at each occurrence identically or differently, selected from CR_{y} or N;

U₁ to U₄ are, at each occurrence identically or differently, selected from CRᵤ or N;

W₁ to W₄ are, at each occurrence identically or differently, selected from CR_{w} or N;

R, Rₓ, R_{y}, Rᵤ and R_{w} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

at least one or more of U₁ to U₄ are selected from CRᵤ, and the Rᵤ is substituted or unsubstituted alkyl having 1 to 20 carbon atoms, substituted or unsubstituted cycloalkyl having 3 to 20 ring carbon atoms or a combination thereof, and the total number of carbon atoms in all of the Rᵤ is at least 4;

at least one of Rₓ is cyano; and

adjacent substituents R, Rₓ, R_{y}, Rᵤ can be optionally joined to form a ring.

In this embodiment, the expression that “adjacent substituents R, Rₓ, R_{y}, Rᵤ can be optionally joined to form a ring” is intended to mean that any one or more of groups of adjacent substituents, such as two substituents R, two substituents Rₓ, two substituents R_{y}, two substituents Rᵤ, and two substituents R_{y} and Rₓ, can be joined to form a ring. Obviously, it is possible that none of these substituents are joined to form a ring.

According to an embodiment of the present disclosure, wherein, Z is selected from O or S.

According to an embodiment of the present disclosure, wherein, Z is O.

According to an embodiment of the present disclosure, wherein, one of Rₓ is cyano; and at least another one of Rₓ is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof.

According to an embodiment of the present disclosure, wherein, one of Rₓ is cyano; and at least another one of Rₓ is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, a cyano group, a hydroxyl group, a sulfanyl group and combinations thereof.

According to an embodiment of the present disclosure, wherein, one of Rₓ is cyano; and at least another one of Rₓ is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof.

According to an embodiment of the present disclosure, wherein, one of Rₓ is cyano, and at least another one of Rₓ is selected from the group consisting of: substituted or unsubstituted aryl having 6 to 15 carbon atoms, substituted or unsubstituted heteroaryl having 3 to 15 carbon atoms and combinations thereof.

According to an embodiment of the present disclosure, wherein, one of Rₓ is cyano, and at least another one of Rₓ is selected from substituted or unsubstituted aryl having 6 to 12 carbon atoms.

According to an embodiment of the present disclosure, wherein, one of Rₓ is cyano, and at least another one of Rₓ is selected from the group consisting of: fluorine, deuterium, methyl, deuterated methyl, isopropyl, deuterated isopropyl, cyclohexyl, deuterated cyclohexyl, phenyl, deuterated phenyl, methylphenyl and deuterated methylphenyl.

According to an embodiment of the present disclosure, wherein, at least one of X₅ to X₈ is CRₓ and the Rₓ is cyano.

According to an embodiment of the present disclosure, at least one of X₇ and X₈ is CRₓ and the Rₓ is cyano.

According to an embodiment of the present disclosure, X₇ is CRₓ and the Rₓ is cyano.

According to an embodiment of the present disclosure, X₈ is CRₓ and the Rₓ is cyano.

According to an embodiment of the present disclosure, wherein, U₁ to U₄ are, at each occurrence identically or differently, selected from CRᵤ, at least one of Rᵤ is selected from substituted or unsubstituted alkyl having 1 to 20 carbon atoms, substituted or unsubstituted cycloalkyl having 3 to 20 ring carbon atoms or a combination thereof, and the total number of carbon atoms in all of the Rᵤ is at least 4.

According to an embodiment of the present disclosure, wherein, U₁ to U₄ are, at each occurrence identically or differently, selected from N or CRᵤ, at least one of U₁ to U₄ is CRᵤ, and the Rᵤ is substituted or unsubstituted alkyl having 1 to 20 carbon atoms, substituted or unsubstituted cycloalkyl having 3 to 20 ring carbon atoms or a combination thereof, and the total number of carbon atoms in all of the Rᵤ is at least 4.

According to an embodiment of the present disclosure, wherein, at least one of Rᵤ is selected from substituted or unsubstituted alkyl having 4 to 20 carbon atoms, substituted or unsubstituted cycloalkyl having 4 to 20 carbon atoms or a combination thereof.

According to an embodiment of the present disclosure, at least one of Rᵤ is selected from the group consisting of the following substituents that are either substituted or unsubstituted:

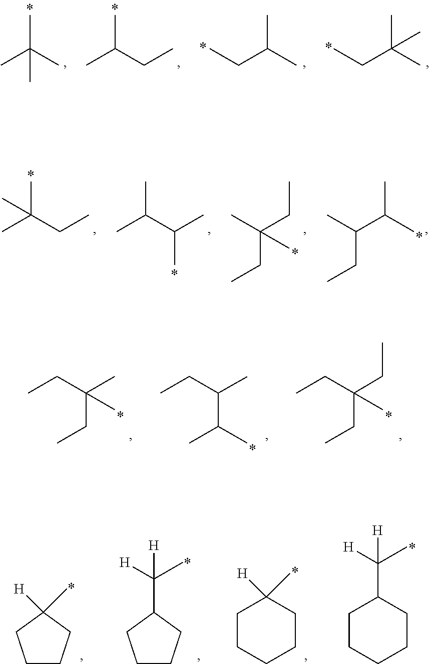

and combinations thereof; optionally, hydrogen in the above groups is partially or fully deuterated;

wherein “*” represents a position where the substituent is joined to carbon.

According to an embodiment of the present disclosure, wherein, at least one of Rᵤ is selected from substituted or unsubstituted alkyl having 4 to 6 carbon atoms, substituted or unsubstituted cycloalkyl having 4 to 6 carbon atoms or a combination thereof.

According to an embodiment of the present disclosure, wherein, U₂ or U₃ is CRᵤ and the Rᵤ is selected from substituted or unsubstituted alkyl having 4 to 20 carbon atoms, substituted or unsubstituted cycloalkyl having 4 to 20 carbon atoms or a combination thereof.

According to an embodiment of the present disclosure, wherein, U₂ or U₃ is CRᵤ, Rᵤ may be, at each occurrence, identical or different, and the Rᵤ is selected from substituted or unsubstituted alkyl having 4 to 6 carbon atoms, substituted or unsubstituted cycloalkyl having 4 to 6 carbon atoms or a combination thereof.

According to an embodiment of the present disclosure, wherein, U₂ and U₃ are CRᵤ, and the Rᵤ is, at each occurrence identically or differently, selected from substituted or unsubstituted alkyl having 1 to 20 carbon atoms, substituted or unsubstituted cycloalkyl having 3 to 20 ring carbon atoms or a combination thereof, and the number of carbon atoms in at least one Rᵤ is greater than or equal to 4.

According to an embodiment of the present disclosure, wherein, U₁ and U₄ are CRᵤ and Rᵤ is selected from hydrogen, deuterium, methyl or deuterated methyl.

According to an embodiment of the present disclosure, wherein, W₁ to W₄ are, at each occurrence identically or differently, selected from CR_{w}, Y₁ to Y₄ are, at each occurrence identically or differently, selected from CR_{y}, and R_{w} and R_{y} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof.

According to an embodiment of the present disclosure, R_{w} and R_{y} are, at each occurrence identically or differently, selected from the group consisting of: hydrogen, deuterium, substituted or unsubstituted alkyl having 1 to 10 carbon atoms, substituted or unsubstituted cycloalkyl having 3 to 10 ring carbon atoms, substituted or unsubstituted aryl having 6 to 10 carbon atoms and combinations thereof.

According to an embodiment of the present disclosure, R_{w} and R_{y} are, at each occurrence identically or differently, selected from the group consisting of: hydrogen, deuterium, substituted or unsubstituted alkyl having 1 to 10 carbon atoms, substituted or unsubstituted cycloalkyl having 3 to 10 ring carbon atoms and combinations thereof.

According to an embodiment of the present disclosure, wherein, W₁, to W₄ are, at each occurrence identically or differently, selected from CR_{w}, and at least one of R_{w} is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof; and/or Y₁ to Y₄ are, at each occurrence identically or differently, selected from CR_{y}, and at least one R_{y} is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof.

According to an embodiment of the present disclosure, wherein, R is substituted or unsubstituted alkyl having 1 to 20 carbon atoms or substituted or unsubstituted cycloalkyl having 3 to 20 ring carbon atoms.

According to an embodiment of the present disclosure, wherein, R is selected from methyl or deuterated methyl.

According to an embodiment of the present disclosure, wherein, Lₐ is, at each occurrence identically or differently, selected from the group consisting of Lₐ₁ to Lₐ₂₀₆, wherein the specific structures of Lₐ₁ to Lₐ₂₀₆ are referred to claim 17.

According to an embodiment of the present disclosure, wherein, L_{b} is, at each occurrence identically or differently, selected from the group consisting of L_{b}, to L_{b972}, wherein the specific structures of L_{b1} to L_{b972} are referred to claim 18.

According to an embodiment of the present disclosure, wherein, the metal complex has a structure of Ir(Lₐ)₂L_{b}, wherein the two Lₐ are identical; Lₐ is selected from the group consisting of Lₐ₁ to Lₐ₂₀₆, wherein the specific structures of Lₐ₁ to Lₐ₂₀₆ are referred to claim 17; and L_{b} is selected from the group consisting of L_{b1} to L_{b972}, wherein the specific structures of L_{b1} to L_{b972} are referred to claim 18.

According to an embodiment of the present disclosure, wherein, the metal complex is selected from the group consisting of Metal Complex 1 to Metal Complex 448, wherein the specific structures of Metal Complex 1 to Metal Complex 448 are referred to claim 19.

According to an embodiment of the present disclosure, further provided is an electroluminescent device. The electroluminescent device includes an anode, a cathode and an organic layer disposed between the anode and the cathode, wherein at least one layer of the organic layer contains the metal complex in any one of the preceding embodiments.

According to an embodiment of the present disclosure, wherein, in the electroluminescent device, the organic layer containing the metal complex is a light-emitting layer.

According to an embodiment of the present disclosure, wherein, in the electroluminescent device, the light-emitting layer emits green light.

According to an embodiment of the present disclosure, wherein, in the electroluminescent device, the light-emitting layer further contains at least one first host compound.

According to an embodiment of the present disclosure, wherein, in the electroluminescent device, the light-emitting layer further contains at least one first host compound and at least one second host compound.

According to an embodiment of the present disclosure, wherein, in the electroluminescent device, at least one of the host compounds comprises at least one chemical group selected from the group consisting of: benzene, pyridine, pyrimidine, triazine, carbazole, azacarbazole, indolocarbazole, dibenzothiophene, aza-dibenzothiophene, dibenzofuran, azadibenzofuran, dibenzoselenophene, triphenylene, azatriphenylene, fluorene, silafluorene, naphthalene, quinoline, isoquinoline, quinazoline, quinoxaline, phenanthrene, azaphenanthrene and combinations thereof.

According to an embodiment of the present disclosure, wherein, the first host compound has a structure represented by Formula 3:

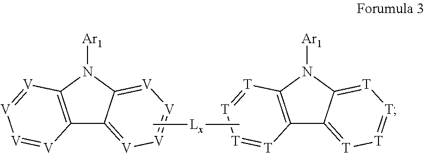

wherein

Lₓ is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or a combination thereof;

V is, at each occurrence identically or differently, selected from C, CRᵥ or N, and at least one of V is C and joined to Lₓ;

T is, at each occurrence identically or differently, selected from C, CRₜ or N, and at least one of T is C and joined to Lₓ;

Rᵥ and Rₜ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

Ar₁ is, at each occurrence identically or differently, selected from substituted or unsubstituted aryl having 6 to 30 carbon atoms, substituted or unsubstituted heteroaryl having 3 to 30 carbon atoms or a combination thereof; and

adjacent substituents Rᵥ and Rₜ can be optionally joined to form a ring.

In this embodiment, the expression that “adjacent substituents Rᵥ and Rₜ can be optionally joined to form a ring” is intended to mean that any one or more of groups of adjacent substituents, such as two substituents Rᵥ, two substituents Rₜ, and two substituents Rᵥ and Rₜ can be joined to form a ring. Obviously, it is possible that none of these substituents are joined to form a ring.

According to an embodiment of the present disclosure, wherein, the first host compound has a structure represented by one of Formulas 3-a to 3-j:

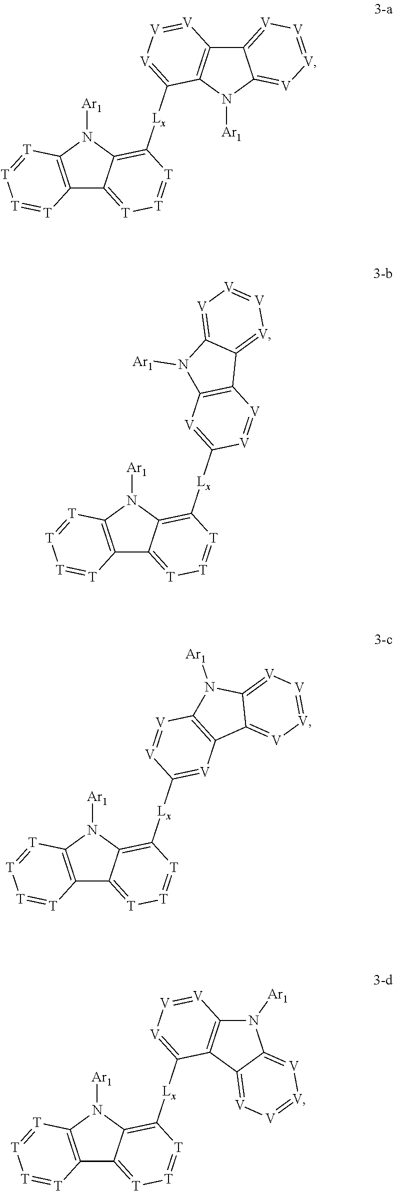

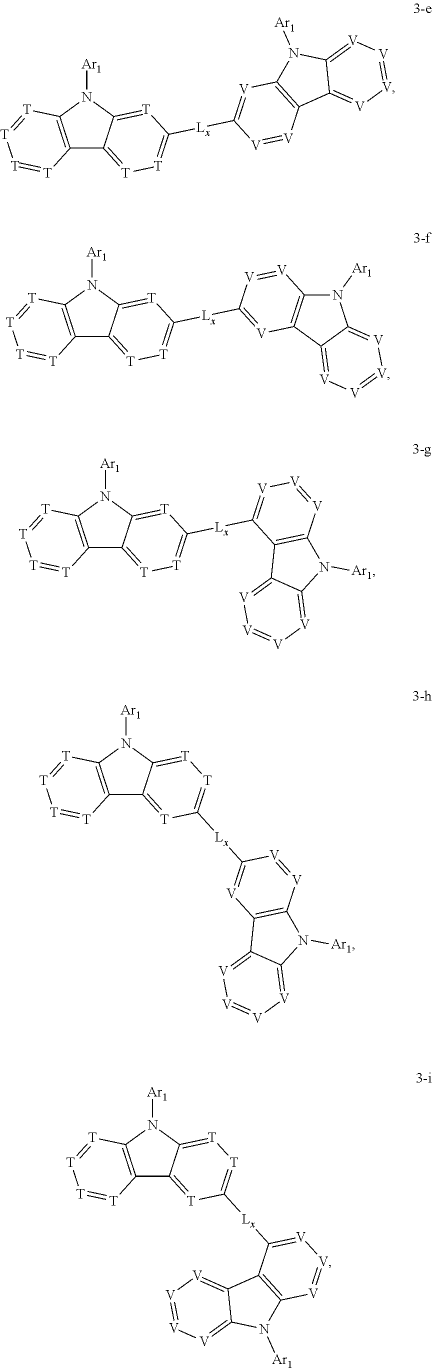

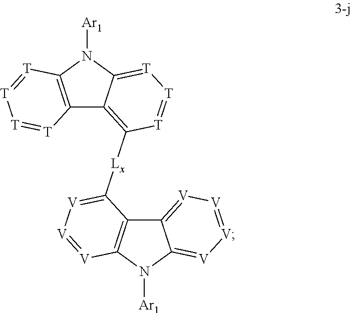

wherein

Lₓ is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or a combination thereof;

V is, at each occurrence identically or differently, selected from CRᵥ or N;

T is, at each occurrence identically or differently, selected from CRₜ or N;

Rᵥ and Rₜ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

Ar₁ is, at each occurrence identically or differently, selected from substituted or unsubstituted aryl having 6 to 30 carbon atoms, substituted or unsubstituted heteroaryl having 3 to 30 carbon atoms or a combination thereof; and

adjacent substituents Rᵥ and Rₜ can be optionally joined to form a ring.

According to an embodiment of the present disclosure, wherein, in the electroluminescent device, the metal complex is doped in the first host compound and the second host compound, and the weight of the metal complex accounts for 1% to 30% of the total weight of the light-emitting layer.

According to an embodiment of the present disclosure, wherein, in the electroluminescent device, the metal complex is doped in the first host compound and the second host compound, and the weight of the metal complex accounts for 3% to 13% of the total weight of the light-emitting layer.

According to another embodiment of the present disclosure, further provided is a compound composition which includes a metal complex whose specific structure is as shown in any one of the preceding embodiments.

Combination with Other Materials

The materials described in the present disclosure for a particular layer in an organic light emitting device can be used in combination with various other materials present in the device. The combinations of these materials are described in more detail in U.S. Pat. App. No. 20160359122 at paragraphs 0132-0161, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

The materials described herein as useful for a particular layer in an organic light emitting device may be used in combination with a variety of other materials present in the device. For example, dopants disclosed herein may be used in combination with a wide variety of hosts, transport layers, blocking layers, injection layers, electrodes and other layers that may be present. The combination of these materials is described in detail in paragraphs 0080-0101 of U.S. Pat. App. No. 20150349273, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

In the embodiments of material synthesis, all reactions were performed under nitrogen protection unless otherwise stated. All reaction solvents were anhydrous and used as received from commercial sources. Synthetic products were structurally confirmed and tested for properties using one or more conventional equipment in the art (including, but not limited to, nuclear magnetic resonance instrument produced by BRUKER, liquid chromatograph produced by SHIMADZU, liquid chromatograph-mass spectrometry produced by SHIMADZU, gas chromatograph-mass spectrometry produced by SHIMADZU, differential Scanning calorimeters produced by SHIMADZU, fluorescence spectrophotometer produced by SHANGHAI LENGGUANG TECH., electrochemical workstation produced by WUHAN CORRTEST, and sublimation apparatus produced by ANHUI BEQ, etc.) by methods well known to the persons skilled in the art. In the embodiments of the device, the characteristics of the device were also tested using conventional equipment in the art (including, but not limited to, evaporator produced by ANGSTROM ENGINEERING, optical testing system produced by SUZHOU FATAR, life testing system produced by SUZHOU FATAR, and ellipsometer produced by BEIJING ELLITOP, etc.) by methods well known to the persons skilled in the art. As the persons skilled in the art are aware of the above-mentioned equipment use, test methods and other related contents, the inherent data of the sample can be obtained with certainty and without influence, so the above related contents are not further described in this patent.

### MATERIAL SYNTHESIS EXAMPLE

The method for preparing a compound in the present disclosure is not limited herein. Typically, the following compounds are used as examples without limitations, and synthesis routes and preparation methods thereof are described below.

### Synthesis Example 1: Synthesis of Metal Complex 13

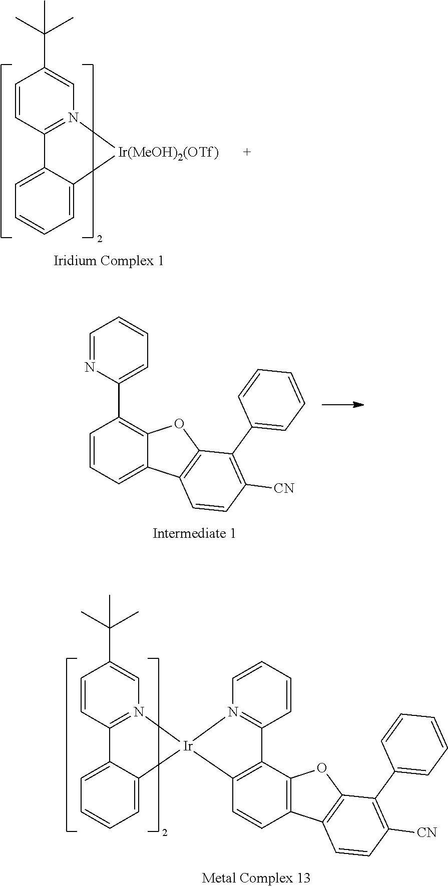

Intermediate 1 (1.6 g, 4.6 mmol), iridium complex 1 (3.18 g, 3.8 mmol), 2-ethoxyethanol (30 mL) and DMF (30 mL) were sequentially added into a dry 250 mL round-bottom flask and heated to 90° C. for 144 h under N₂ protection. The reaction was cooled, filtered through Celite, and washed twice with methanol and n-hexane separately. Yellow solids on the Celite were dissolved with dichloromethane. The organic phases were collected, concentrated under reduced pressure, and purified through column chromatography to obtain Metal Complex 13 as a yellow solid (0.82 g with a yield of 22.3%). The product was confirmed as the target product with a molecular weight of 958.3.

### Synthesis Example 2: Synthesis of Metal Complex 7

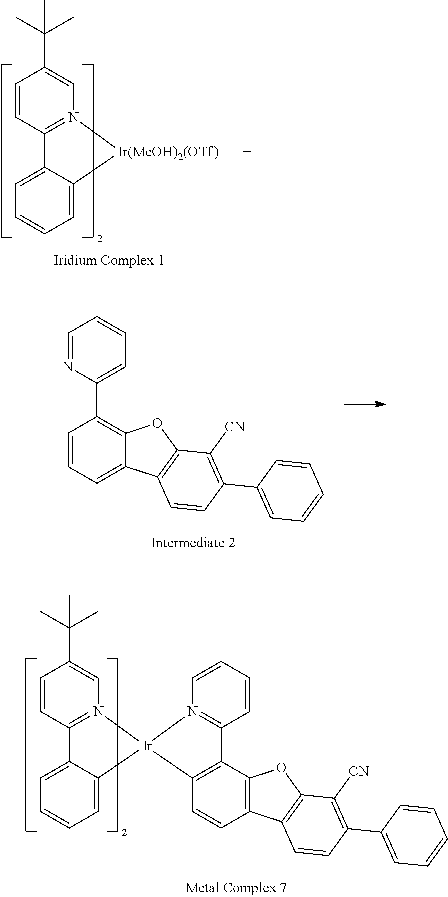

Intermediate 2 (1.0 g, 2.9 mmol), iridium complex 1 (2.2 g, 2.6 mmol), 2-ethoxyethanol (40 mL) and DMF (40 mL) were sequentially added into a dry 250 mL round-bottom flask and heated to 100° C. for 120 h under N₂ protection. The reaction was cooled, filtered through Celite, and washed twice with methanol and n-hexane separately. Yellow solids on the Celite were dissolved with dichloromethane. The organic phases were collected, concentrated under reduced pressure, and purified through column chromatography to obtain Metal Complex 7 as a yellow solid (0.45 g with a yield of 18.1%). The product was confirmed as the target product with a molecular weight of 958.3.

### Synthesis Example 3: Synthesis of Metal Complex 17

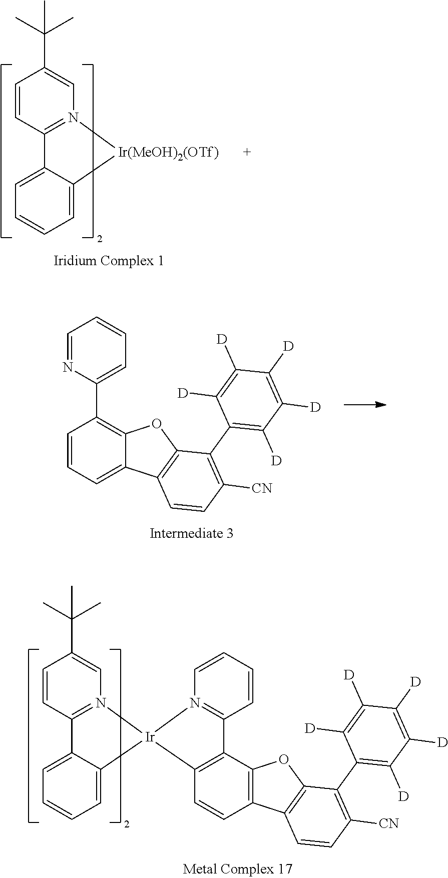

Intermediate 3 (1.2 g, 4.5 mmol), iridium complex 1 (2.5 g, 3.0 mmol), 2-ethoxyethanol (30 mL) and DMF (30 mL) were sequentially added into a dry 250 mL round-bottom flask and heated to 90° C. for 144 h under N₂ protection. The reaction was cooled, filtered through Celite, and washed twice with methanol and n-hexane separately. Yellow solids on the Celite were dissolved with dichloromethane. The organic phases were collected, concentrated under reduced pressure, and purified through column chromatography to obtain Metal Complex 17 as a yellow solid (0.73 g with a yield of 25.3%). The product was confirmed as the target product with a molecular weight of 963.3.

### Synthesis Example 4: Synthesis of Metal Complex 163

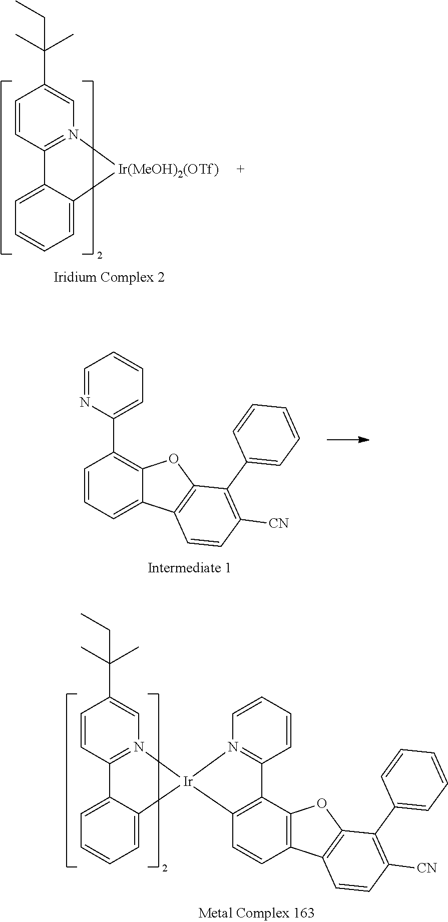

Intermediate 1 (1.3 g, 3.7 mmol), iridium complex 2 (2.2 g, 2.6 mmol), 2-ethoxyethanol (30 mL) and DMF (30 mL) were sequentially added into a dry 250 mL round-bottom flask and heated to 90° C. for 144 h under N₂ protection. The reaction was cooled, filtered through Celite, and washed twice with methanol and n-hexane separately. Yellow solids on the Celite were dissolved with dichloromethane. The organic phases were collected, concentrated under reduced pressure, and purified through column chromatography to obtain Metal Complex 163 as a yellow solid (0.78 g with a yield of 30.4%). The product was confirmed as the target product with a molecular weight of 986.3.

### Synthesis Example 5: Synthesis of Metal Complex 43

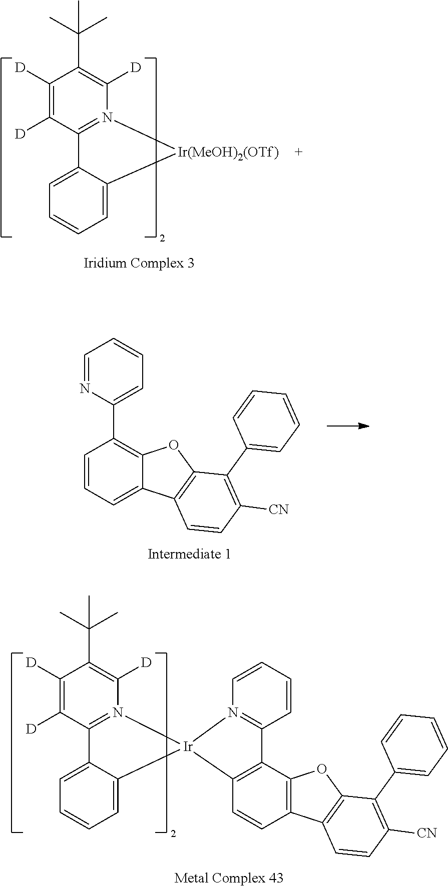

Intermediate 1 (1.5 g, 4.9 mmol), iridium complex 3 (3.0 g, 3.6 mmol), 2-ethoxyethanol (30 mL) and DMF (30 mL) were sequentially added into a dry 250 mL round-bottom flask and heated to 95° C. for 144 h under N₂ protection. The reaction was cooled, filtered through Celite, and washed twice with methanol and n-hexane separately. Yellow solids on the Celite were dissolved with dichloromethane. The organic phases were collected, concentrated under reduced pressure, and purified through column chromatography to obtain Metal Complex 43 as a yellow solid (1.23 g with a yield of 35.4%). The product structure was confirmed as the target product with a molecular weight of 964.4.

Those skilled in the art will appreciate that the above preparation methods are merely exemplary. Those skilled in the art can obtain other compound structures of the present disclosure through the modifications of the preparation methods.

### Device Example 1

First, a glass substrate having an Indium Tin Oxide (ITO) anode with a thickness of 80 nm was cleaned and then treated with oxygen plasma and UV ozone. After the treatment, the substrate was dried in a glovebox to remove moisture. Then, the substrate was mounted on a substrate holder and placed in a vacuum chamber. Organic layers specified below were sequentially deposited through vacuum thermal evaporation on the ITO anode at a rate of 0.2 to 2 Angstroms per second and a vacuum degree of about 10⁻⁸ torr. Compound HI was used as a hole injection layer (HIL). Compound HT was used as a hole transporting layer (HTL). Compound H1 was used as an electron blocking layer (EBL). Metal Complex 13 of the present disclosure was doped in Compound H1 and Compound H2, and the resulting mixture was deposited for use as an emissive layer (EML). On the EML, Compound H2 was used as a hole blocking layer (HBL). On the HBL, Compound ET and 8-hydroxyquinolinolato-lithium (Liq) were co-deposited for use as an electron transporting layer (ETL). Finally, 8-hydroxyquinolinolato-lithium (Liq) was deposited as an electron injection layer, with a thickness of 1 nm and A1 was deposited as a cathode with a thickness of 120 nm. The device was transferred back to the glovebox and encapsulated with a glass lid and a moisture getter to complete the device.

### Device Example 3

The implementation mode in Device Example 3 was the same as that in Device Example 1, except that in the emissive layer (EML), Metal Complex 13 of the present disclosure was replaced with Metal Complex 17.

### Device Comparative Example 1

The implementation mode in Device Comparative Example 1 was the same as that in Device Example 1, except that in the emissive layer (EML), Metal Complex 13 of the present disclosure was replaced with Compound GD1.

### Device Comparative Example 2

The implementation mode in Device Comparative Example 2 was the same as that in Device Example 1, except that in the emissive layer (EML), Metal Complex 13 of the present disclosure was replaced with Compound GD2.

Detailed structures and thicknesses of layers of the devices are shown in the following table. A layer using more than one material is obtained by doping different compounds at their weight ratio as recorded.

TABLE 1
Device structures in Example 1 and 3 and Comparative Examples 1 and 2

| Device ID | HIL | HTL | EBL | EML | HBL | ETL |
|---|---|---|---|---|---|---|
| Example 1 | Compound | Compound | Compound | Compound | Compound | Compound |
| | HI (100 Å) | HT (350 Å) | H1 (50 Å) | H1:Compound | H2 (50 Å) | ET:Liq |
| | | | | H2:Metal | | (40:60) |
| | | | | Complex 13 | | (350 Å) |
| | | | | (46:46:8) (400 Å) | | |
| Example 3 | Compound | Compound | Compound | Compound | Compound | Compound |
| | HI (100 Å) | HT (350 Å) | H1 (50 Å) | H1:Compound | H2 (50 Å) | ET:Liq |
| | | | | H2:Metal | | (40:60) |
| | | | | Complex 17 | | (350 Å) |
| | | | | (46:46:8) (400 Å) | | |
| Comparative | Compound | Compound | Compound | Compound | Compound | Compound |
| Example 1 | HI (100 Å) | HT (350 Å) | H1 (50 Å) | H1:Compound | H2 (50 Å) | ET:Liq |
| | | | | H2:Compound | | (40:60) |
| | | | | GD1 (46:46:8) | | (350 Å) |
| | | | | (400 Å) | | |
| Comparative | Compound | Compound | Compound | Compound | Compound | Compound |
| Example 2 | HI (100 Å) | HT (350 Å) | H1 (50 Å) | H1:Compound | H2 (50 Å) | ET:Liq |
| | | | | H2:Compound | | (40:60) |
| | | | | GD2 (46:46:8) | | (350 Å) |
| | | | | (400 Å) | | |

The structures of the materials used in the devices are shown as follows:

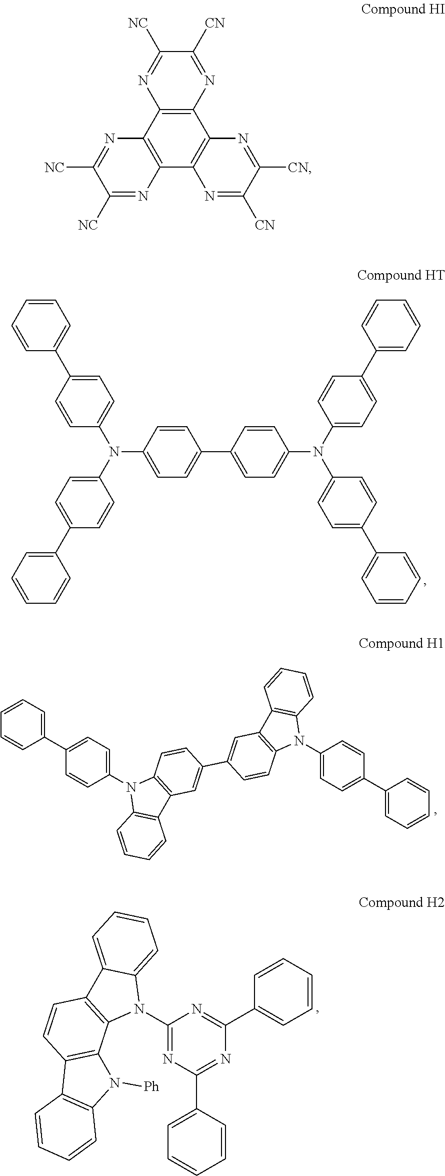 [SMILES: N#Cc1nc2c3nc(C#N)c(C#N)nc3c3nc(C#N)c(C#N)nc3c2nc1C#N ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2nc(-c3ccccc3)nc(-n3c4ccccc4c4ccc5c6ccccc6n(-c6ccccc6)c5c43)n2)cc1]

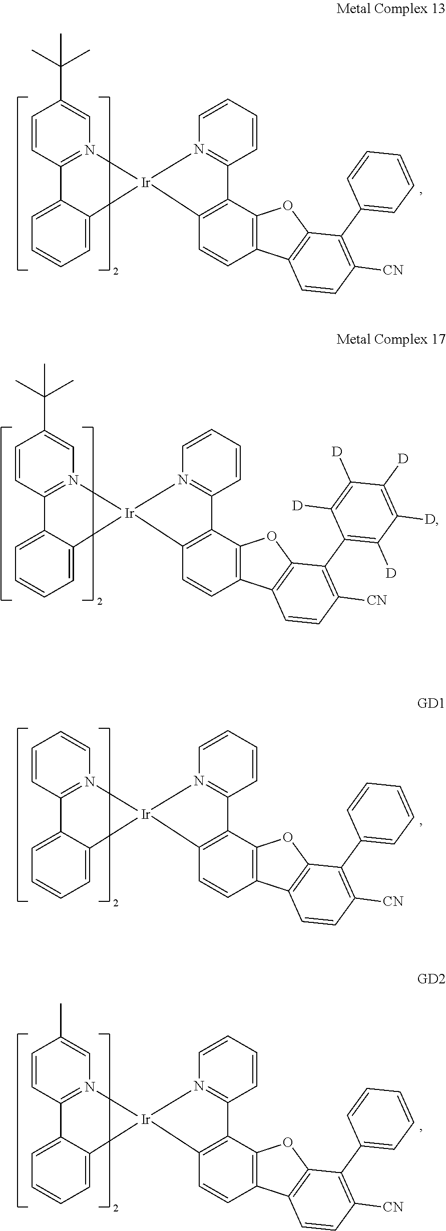

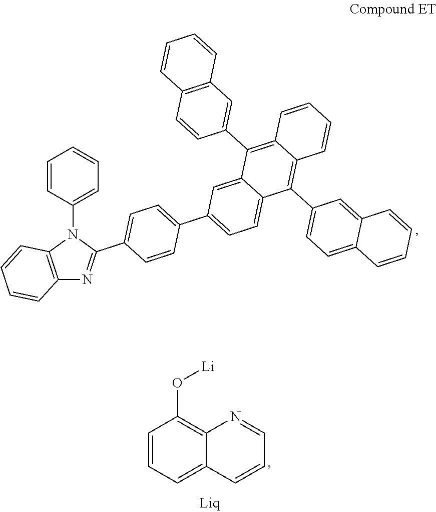

Current-voltage-luminance (IVL) characteristics of the devices were measured. The CIE data, maximum emission wavelengths λₘₐₓ and full width at half maxima (FWHM) of the devices were measured at 1000 cd/m². The evaporation temperature (Sub T) of a material is a temperature tested when the metal complex is subjected to vacuum thermal evaporation at a rate of 0.2 angstroms per second and a vacuum degree of about 10⁻⁸ Torr. Lifetime (LT97) data was tested at a constant current of 80 mA/cm². The data was recorded and shown in Table 2.

TABLE 2
Device data of Examples 1 and 3 and
Comparative Examples 1 and 2

| | Sub T | | λₘₐₓ | FWHM | LT 97 |
|---|---|---|---|---|---|
| Device ID | (° C.) | CIE (x, y) | (nm) | (nm) | (h) |
| Example 1 | 258 | (0.349, 0.630) | 533 | 37.6 | 17.20 |
| Example 3 | 252 | (0.345, 0.633) | 532 | 36.3 | 21.40 |
| Comparative Example 1 | 291 | (0.335, 0.638) | 528 | 40.9 | 11.35 |
| Comparative Example 2 | 287 | (0.346, 0.631) | 531 | 40.6 | 14.90 |

As can be seen from the data in Table 2, the FWHM of Example 1 is 3.3 nm narrower than that of Comparative Example 1 and 3.0 nm narrower than that of Comparative Example 2. Meanwhile, the evaporation temperature of Device Example 1 is nearly 33° C. lower than that of Comparative Example 1 and nearly 29° C. lower than that of Comparative Example 2. The lower evaporation temperature helps the complex of the present disclosure remain stable in an evaporation process and a low evaporation temperature is beneficial to the industrial application of materials and can reduce energy consumption. In addition, the lifetime of Example 1 is as much as 51.5% longer than that of Comparative Example 1 and 15.4% longer than that of Comparative Example 2. Similarly, the FWHM of Example 3 where Metal Complex 17 is applied to the device is 4.6 nm and 4.3 nm narrower than those of Comparative Example 1 and Comparative Example 2, respectively, the evaporation temperature of Example 3 is nearly 40° C. and 37° C. lower, respectively, and the lifetime of Example 3 is 88.5% and 43.6% longer, respectively. That is, Example 3 has a narrower FWHM, a lower evaporation temperature and a greatly improved device lifetime. The overall performance of the device is improved significantly.

Metal Complex 13 used in Example 1 contains the same ligand L_{b} as Metal Complex GD1 used in Comparative Example 1 and Metal Complex GD2 used in Comparative Example 2, but the ligand Lₐ has different substituents. Compared with comparative examples with no substitution or with only a methyl substitution, Example 1 using the ligand Lₐ with a particular substitution has the narrower FWHM, the lower evaporation temperature, and the longer device lifetime. Metal Complex 17 used in Example 3 further contains a deuterium substitution on the ligand L_{b}, which further improves the performance of the device and improves the overall performance of the device.

### Device Example 2

The implementation mode in Device Example 2 was the same as that in Device Example 1, except that in the emissive layer (EML), Metal Complex 13 of the present disclosure was replaced with Metal Complex 7.

### Device Comparative Example 3

The implementation mode in Device Comparative Example 3 was the same as that in Device Example 1, except that in the emissive layer (EML), Metal Complex 13 of the present disclosure was replaced with Compound GD3.

Detailed structures and thicknesses of layers of the devices are shown in the following table. A layer using more than one material is obtained by doping different compounds at their weight ratio as recorded.

TABLE 3
Device structures in Example 2 and Comparative Example 3

| Device ID | HIL | HTL | EBL | EML | HBL | ETL |
|---|---|---|---|---|---|---|
| Example 2 | Compound | Compound | Compound | Compound | Compound | Compound |
| | HI (100 Å) | HT (350 Å) | H1 (50 Å) | H1:Compound | H2 (50 Å) | ET:Liq |
| | | | | H2:Metal | | (40:60) |
| | | | | Complex 7 | | (350 Å) |
| | | | | (46:46:8) (400 Å) | | |
| Comparative | Compound | Compound | Compound | Compound | Compound | Compound |
| Example 3 | HI (100 Å) | HT (350 Å) | H1 (50 Å) | H1:Compound | H2 (50 Å) | ET:Liq |
| | | | | H2:Compound | | (40:60) |
| | | | | GD3 (46:46:8) | | (350 Å) |
| | | | | (400 Å) | | |

The structures of the new materials used in the devices are shown as follows:

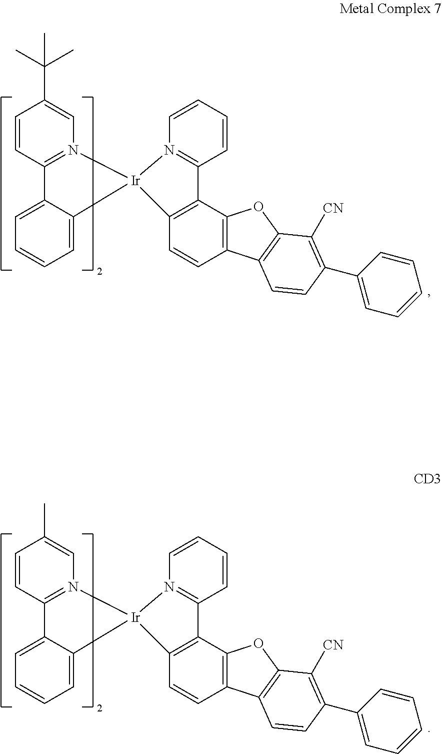

IVL characteristics of the devices were measured. The CIE data, maximum emission wavelengths λₘₐₓ and full width at half maxima (FWHM) of the devices were measured at 1000 cd/m². The evaporation temperature (Sub T) of a material is the temperature tested when the metal complex is subjected to vacuum thermal evaporation at a rate of 0.2 angstroms per second and a vacuum degree of about 10⁻⁸ Torr. Lifetime (LT97) data was tested at a constant current of 80 mA/cm². The data was recorded and shown in Table 4.

TABLE 4
Device data of Example 2 and Comparative Example 3

| | Sub T | | λₘₐₓ | FWHM | LT 97 |
|---|---|---|---|---|---|
| Device ID | (° C.) | CIE (x, y) | (nm) | (nm) | (h) |
| Example 2 | 270 | (0.354, 0.626) | 534 | 43.7 | 17.70 |
| Comparative | 296 | (0.352, 0.627) | 534 | 46.0 | 15.20 |
| Example 3 | | | | | |

As can be seen from the data in Table 4, the FWHM of Device Example 2 is 2.3 nm narrower than that of Device Comparative Example 3 and the evaporation temperature of Device Example 2 is nearly 26° C. lower than that of Comparative Example 3. In addition, the lifetime of Example 2 is 16.4% longer than that of Comparative Example 3. Metal Complex 7 used in Example 2 contains the same ligand L_{b} as Metal Complex GD3 used in Comparative Example 3, but the ligand Lₐ has different substituents. Example 2 has the narrower FWHM, the lower evaporation temperature and the longer device lifetime than Comparative Example 3, which proves the excellent effects of the present disclosure again.

Sublimation Data

Metal complexes and comparative compounds in the present disclosure were sublimated using sublimation equipment with a model number of BOF-A1-3-60 and produced by Anhui BEQ Equipment Technology Co., Ltd. Metal Complex 13, Metal Complex 17, Metal Complex 7 and Reference Complexes GD1, GD2 and GD3 in the present disclosure were separately placed in sublimation tubes of the sublimation equipment and heat to 300° C. to 370° C. to be stably sublimated so that metal complexes were obtained, where the vacuum degree in the sublimation tubes was reduced to be lower than 9.9×10⁻⁴ pa using a molecular pump. Data on the sublimation yields of these materials were recorded and shown in Table 5. The sublimation yield is a ratio of a mass after sublimation to a mass before sublimation.

TABLE 5
Sublimation data

| | Sublimation |
|---|---|
| Compound No. | Yield (%) |
| Metal Complex 13 | 85.3 |
| Metal Complex 17 | 88.8 |
| Metal Complex 7 | 71.1 |
| Compound GD1 | 32.5 |
| Compound GD2 | 58.9 |
| Compound GD3 | 48.8 |

As can be seen from the data in Table 5, Metal Complex 13 and Metal Complex 17 with particular substitutions on the ligand L in the present disclosure exhibit excellent sublimation performance, and the sublimation yields of Metal Complex 13 and Metal Complex 17 reach 85.3% and 88.8%, respectively, which are nearly 1.6 and 1.7 times higher than the sublimation yield (32.8%) of Reference Compound GD1, respectively. Similarly, the sublimation yields of Metal Complex 13 and Metal Complex 17 are 44.8% and 50.7% higher than the sublimation yield (58.9%) of Reference Compound GD2, respectively. In addition, the sublimation yield of Metal Complex 7 reaches 71.1%, which is 45.6% higher than the sublimation yield (48.8%) of Reference Compound GD3. The results show that the metal complex with a particular (cyclo)alkyl substitution introduced into the structure of the ligand Lₐ in the present disclosure has a higher sublimation yield than the metal complex without such a particular substitution. A significant increase of the sublimation yield is unexpected and the increase of the sublimation yield is of great significance to the mass production of metal complexes in the industry.

### Device Example 4

The implementation mode in Device Example 4 was the same as that in Device Example 1, except that in the emissive layer (EML), Compound H2 was replaced with Compound H3 and a ratio of Compound H1, Compound H3 and Metal Complex 13 was 63:31:6.

### Device Comparative Example 4

The implementation mode in Device Comparative Example 4 was the same as that in Device Example 4, except that in the emissive layer (EML), Metal Complex 13 of the present disclosure was replaced with Compound GD2.

### Device Comparative Example 5

The implementation mode in Device Comparative Example 5 was the same as that in Device Example 4, except that in the emissive layer (EML), Metal Complex 13 of the present disclosure was replaced with Compound GD4.

### Device Comparative Example 6

The implementation mode in Device Comparative Example 6 was the same as that in Device Example 4, except that in the emissive layer (EML), Metal Complex 13 of the present disclosure was replaced with Compound GD5.

### Device Comparative Example 7

The implementation mode in Device Comparative Example 7 was the same as that in Device Example 4, except that in the emissive layer (EML), Metal Complex 13 of the present disclosure was replaced with Compound GD6.

Detailed structures and thicknesses of layers of the devices are shown in Table 6. A layer using more than one material is obtained by doping different compounds at their weight ratio as recorded.

TABLE 6
Device structures in Example 4 and Comparative Examples 4 to 7

| Device ID | HIL | HTL | EBL | EML | HBL | ETL |
|---|---|---|---|---|---|---|
| Example 4 | Compound | Compound | Compound | Compound | Compound | Compound |
| | HI (100 Å) | HT (350 Å) | H1 (50 Å) | H1:Compound | H2 (50 Å) | ET:Lig |
| | | | | H3:Metal | | (40:60) |
| | | | | Complex 13 | | (350 Å) |
| | | | | (63:31:6) (400 Å) | | |
| Comparative | Compound | Compound | Compound | Compound | Compound | Compound |
| Example 4 | HI (100 Å) | HT (350 Å) | H1 (50 Å) | H1:Compound | H2 (50 Å) | ET:Lig |
| | | | | H3:GD2 | | (40:60) |
| | | | | (63:31:6) (400 Å) | | (350 Å) |
| Comparative | Compound | Compound | Compound | Compound | Compound | Compound |
| Example 5 | HI (100 Å) | HT (350 Å) | H1 (50 Å) | H1:Compound | H2 (50 Å) | ET:Lig |
| | | | | H3:GD4 | | (40:60) |
| | | | | (63:31:6) (400 Å) | | (350 Å) |
| Comparative | Compound | Compound | Compound | Compound | Compound | Compound |
| Example 6 | HI (100 Å) | HT (350 Å) | H1 (50 Å) | H1:Compound | H2 (50 Å) | ET:Lig |
| | | | | H3:GD5 | | (40:60) |
| | | | | (63:31:6) (400 Å) | | (350 Å) |
| Comparative | Compound | Compound | Compound | Compound | Compound | Compound |
| Example 7 | HI (100 Å) | HT (350 Å) | H1 (50 Å) | H1:Compound | H2 (50 Å) | ET:Lig |
| | | | | H3:GD6 | | (40:60) |
| | | | | (63:31:6) (400 Å) | | (350 Å) |

The structures of the new materials used in the devices are shown as follows:

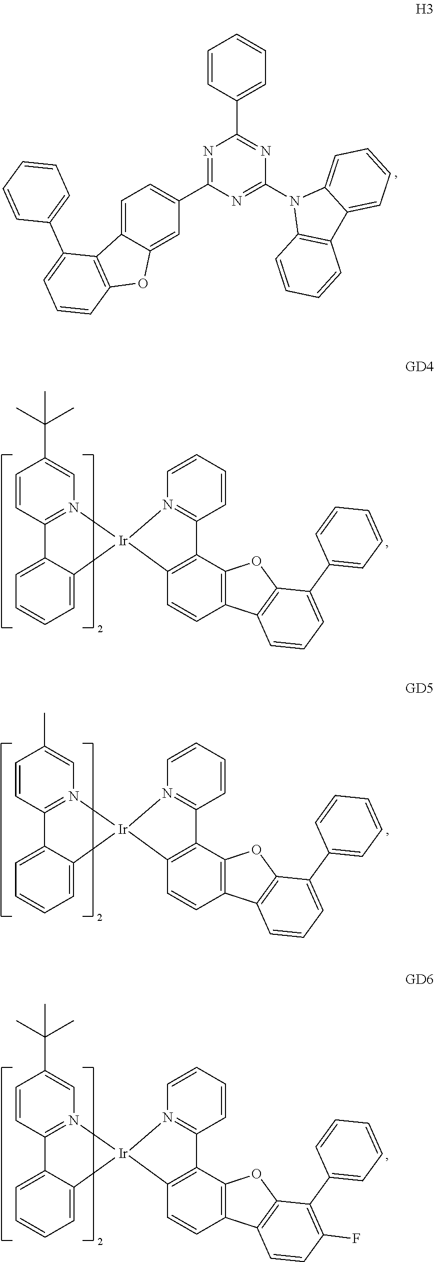

IVL characteristics of the devices were measured. The CIE data, maximum emission wavelengths λₘₐₓ and full width at half maxima (FWHM) of the devices were measured at 1000 cd/m². The lifetime (LT95) is a time taken for an initial luminance of 10000 cd/m² to decay to 95% of the initial luminance. The data was recorded and shown in Table 7.

TABLE 7
Device data of Example 4 and Comparative Examples 4 to 7

| | | λₘₐₓ | FWHM | LT95 |
|---|---|---|---|---|
| Device ID | CIE (x, y) | (nm) | (nm) | (h) |
| Example 4 | (0.346, 0.632) | 531 | 37.5 | 1159 |
| Comparative Example 4 | (0.342, 0.634) | 529 | 37.9 | 829 |
| Comparative Example 5 | (0.353, 0.623) | 531 | 58.9 | 1001 |
| Comparative Example 6 | (0.352, 0.623 | 528 | 60.3 | 910 |
| Comparative Example 7 | (0.355, 0.621) | 531 | 59.5 | 940 |

As can be seen from the data in Table 7, at 10000 cd/m², the lifetime of Example 4 reaches 1159 h, which is greatly improved compared with those of Comparative Examples 4 to 7. The lifetime of Example 4 is 39.8% longer than that of Comparative Example 4 with no particular substituents on the ligand Lₐ, nearly 15.8% and 23.3% higher than those of Comparative Examples 5 and 7 with no cyano substitution on the ligand L_{b}, respectively, and 27.4% longer than that of Comparative Example 6 with no particular substituents on the ligands Lₐ and L_{b}. In addition, the FWHM of Example 4 is only 37.5 nm and much lower than about 59 nm of Comparative Examples 5 and 7, which is very rare among green phosphorescent devices.

When there is no cyano substituent on the ligand L_{b}, the lifetime of Comparative Example 5 with a particular substitution on the ligand Lₐ is only 10% longer than the lifetime of Comparative Example 6 with no particular substitution on the ligand Lₐ, while when there is a cyano substituent on the ligand L_{b}, the lifetime of Example 4 with a particular substitution on the ligand Lₐ is 39.8% longer than that of Comparative Example 4 with no particular substitution on the ligand Lₐ. Similarly, when there is the same ligand Lₐ, the lifetime of Example 4 with a cyano substitution on the ligand L_{b} is 15.8% longer than that of Comparative Example 5 with no cyano substitution on the ligand L_{b}, while the lifetime of Comparative Example 7 with a fluorine substitution on the ligand L_{b} is slightly short than that of Comparative Example 5. All the preceding results indicate that the metal complex containing the ligand Lₐ with the particular substitution and the ligand L_{b} with the cyano substitution in the present disclosure can achieve excellent device performance, especially a greatly improved device lifetime.

In summary, the metal complexes of the present disclosure containing ligands Lₐ and L_{b} with particular substitutions may be used as light-emitting materials in light-emitting layers of electroluminescent devices. When used in combination with host materials with different structures, the metal complexes can all achieve excellent device performance. The metal complexes of the present disclosure containing ligands Lₐ and L_{b} with particular substitutions can maintain the FWHMs of related devices at a high level in the industry and greatly improve the device lifetime. In addition, the metal complexes of the present disclosure can also greatly improve the sublimation yield and the evaporation temperature and has huge advantages and a broad prospect in industrial applications.

It should be understood that various embodiments described herein are merely examples and not intended to limit the scope of the present disclosure. Therefore, it is apparent to those skilled in the art that the present disclosure as claimed may include variations from specific embodiments and preferred embodiments described herein. Many of materials and structures described herein may be substituted with other materials and structures without departing from the spirit of the present disclosure. It should be understood that various theories as to why the present disclosure works are not intended to be limitative.

What is claimed is:

1. A metal complex having a general formula of M(Lₐ)ₘ(L_{b})ₙ(L_{c})_{q},
wherein
Lₐ, L_{b} and L_{c} are a first ligand, a second ligand and a third ligand coordinated to the metal M, respectively, and L_{c} is identical to or different from Lₐ or L_{b}; wherein Lₐ, L_{b} and L_{c} can be optionally joined to form a multidentate ligand;
the metal M is selected from a metal with a relative atomic mass greater than 40; preferably, the metal M is, at each occurrence identically or differently, selected from the group consisting of Cu, Ag, Au, Ru, Rh, Pd, Os, Ir and Pt; more preferably, M is, at each occurrence identically or differently, selected from Pt or Ir;
m is 1 or 2, n is 1 or 2, q is 0 or 1, and m+n+q equals to the oxidation state of M; when m is 2, two Lₐ are identical or different; when n is 2, two L_{b} are identical or different;
Lₐ has, at each occurrence identically or differently, a structure represented by Formula 1A and L_{b} has, at each occurrence identically or differently, a structure represented by Formula 1B: 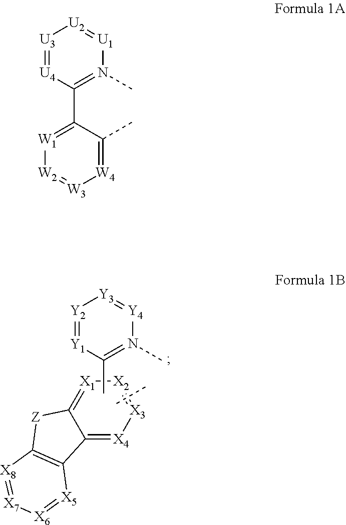
wherein
Z is selected from the group consisting of O, S, Se, NR, CRR and SiRR, wherein when two R are present, the two R are identical or different;
X₁ to X₈ are, at each occurrence identically or differently, selected from C or CRₓ;
Y₁ to Y₄ are, at each occurrence identically or differently, selected from CR_{y} or N;
U₁ to U₄ are, at each occurrence identically or differently, selected from CRᵤ or N;
W₁ to W₄ are, at each occurrence identically or differently, selected from CR_{w} or N;
R, Rₓ, R_{y}, Rᵤ and R_{w} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
at least one or more of U₁ to U₄ are selected from CRᵤ, and the Rᵤ is substituted or unsubstituted alkyl having 1 to 20 carbon atoms, substituted or unsubstituted cycloalkyl having 3 to 20 ring carbon atoms or a combination thereof, and the total number of carbon atoms in all of the Rᵤ is at least 4;
at least one of Rₓ is cyano; and
adjacent substituents R, Rₓ, R_{y}, Rᵤ, R_{w} can be optionally joined to form a ring;
L_{c} is, at each occurrence identically or differently, selected from a structure represented by any one of the group consisting of the following: 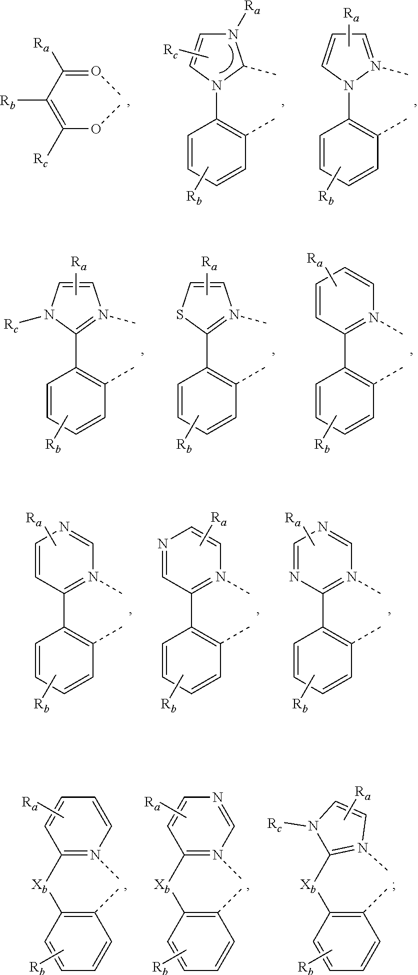
wherein
Rₐ, R_{b} and R_{c} represent, at each occurrence identically or differently, mono-substitution, multiple substitutions or non-substitution;
X_{b} is, at each occurrence identically or differently, selected from the group consisting of: O, S, Se, NR_{N1} and CR_{C1}R_{C2};
Rₐ, R_{b}, R_{c}, R_{N1}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents Rₐ, R_{b}, R_{c}, R_{N1}, R_{C1} and R_{C2} can be optionally joined to form a ring.

2. The metal complex of claim 1, wherein L_{b} has a structure represented by each of Formulas 1Ba to 1Bd: 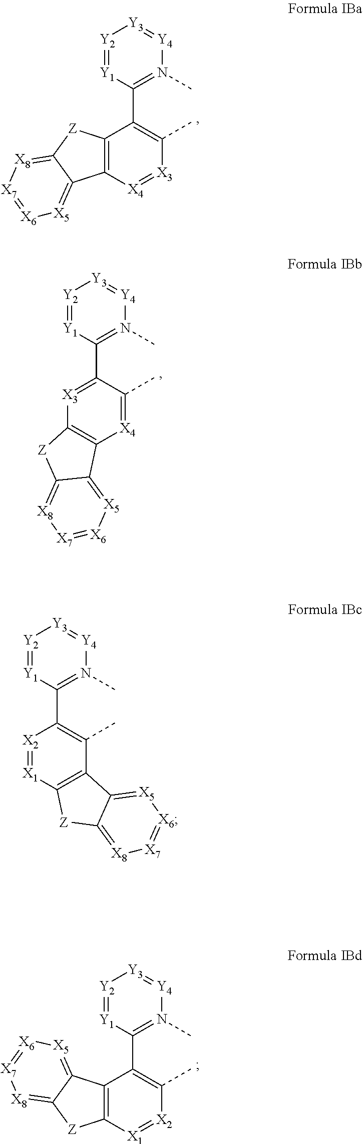
wherein
Z is selected from the group consisting of O, S, Se, NR, CRR and SiRR, wherein when two R are present, the two R are identical or different;
X₁ to X₈ are, at each occurrence identically or differently, selected from CRₓ;
Y₁ to Y₄ are, at each occurrence identically or differently, selected from CR_{y} or N;
R, Rₓ and R_{y} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R, Rₓ, R_{y} can be optionally joined to form a ring.

3. The metal complex of claim 1, wherein the metal complex has a structure represented by Formula 2: 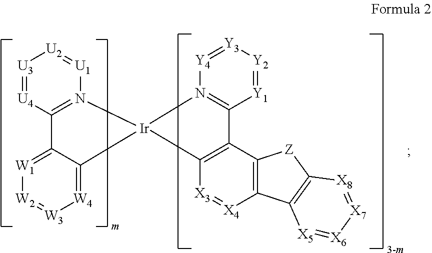
wherein
m is selected from 1 or 2; when m=1, two L_{b} are identical or different; when m=2, two Lₐ are identical or different;
Z is selected from the group consisting of O, S, Se, NR, CRR and SiRR, wherein when two R are present, the two R are identical or different;
X₃ to X₈ are, at each occurrence identically or differently, selected from CRₓ;
Y₁ to Y₄ are, at each occurrence identically or differently, selected from CR_{y} or N;
U₁ to U₄ are, at each occurrence identically or differently, selected from CRᵤ or N;
W₁ to W₄ are, at each occurrence identically or differently, selected from CR_{w} or N;
R, Rₓ, R_{y}, Rᵤ and R_{w} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
at least one or more of U₁ to U₄ are selected from CRᵤ, and the Rᵤ is substituted or unsubstituted alkyl having 1 to 20 carbon atoms, substituted or unsubstituted cycloalkyl having 3 to 20 ring carbon atoms or a combination thereof, and the total number of carbon atoms in all of the Rᵤ is at least 4;
at least one of Rₓ is cyano; and
adjacent substituents R, Rₓ, R_{y}, Rᵤ can be optionally joined to form a ring.

4. The metal complex of claim 1, wherein Z is selected from O or S;
preferably, Z is O.

5. The metal complex of claim 1, wherein one of Rₓ is cyano; and at least another one of Rₓ is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
preferably, one of Rₓ is cyano; and at least another one of Rₓ is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, a cyano group, a hydroxyl group, a sulfanyl group and combinations thereof;
more preferably, one of Rₓ is cyano; and at least another one of Rₓ is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof.

6. The metal complex of claim 1, wherein one of Rₓ is cyano, and at least another one of Rₓ is selected from the group consisting of: substituted or unsubstituted aryl having 6 to 15 carbon atoms, substituted or unsubstituted heteroaryl having 3 to 15 carbon atoms and combinations thereof.

7. The metal complex of claim 1, wherein one of Rₓ is cyano, and at least another one of Rₓ is selected from the group consisting of: fluorine, deuterium, methyl, deuterated methyl, deuterated isopropyl, cyclohexyl, deuterated cyclohexyl, phenyl, deuterated phenyl, methylphenyl and deuterated methylphenyl.

8. The metal complex of claim 1, wherein at least one of X₅ to X₈ is CRₓ and the Rₓ is cyano;
preferably, X₇ is CRₓ and the Rₓ is cyano; or X₈ is CRₓ and the Rₓ is cyano.

9. The metal complex of claim 1, wherein U₁ to U₄ are, at each occurrence identically or differently, selected from CRᵤ, at least one of Rᵤ is selected from substituted or unsubstituted alkyl having 1 to 20 carbon atoms, substituted or unsubstituted cycloalkyl having 3 to 20 ring carbon atoms or a combination thereof, and the total number of carbon atoms in all of the Rᵤ is at least 4.

10. The metal complex of claim 1, wherein U₁ to U₄ are, at each occurrence identically or differently, selected from N or CRᵤ, and at least one of U₁ to U₄ is CRᵤ, and Rᵤ is selected from substituted or unsubstituted alkyl having 1 to 20 carbon atoms, substituted or unsubstituted cycloalkyl having 3 to 20 ring carbon atoms or a combination thereof, and the total number of carbon atoms in all of the Rᵤ is at least 4.

11. The metal complex of claim 1, wherein at least one of Rᵤ is selected from substituted or unsubstituted alkyl having 4 to 20 carbon atoms, substituted or unsubstituted cycloalkyl having 4 to 20 carbon atoms or a combination thereof;
preferably, at least one of Rᵤ is selected from the group consisting of the following substituents that are either substituted or unsubstituted: 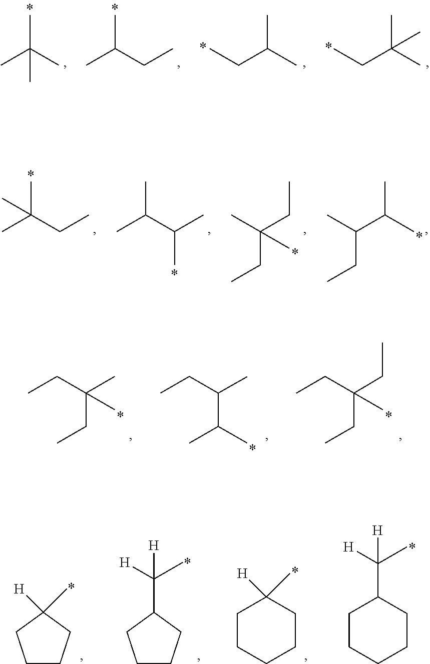
and combinations thereof; optionally, hydrogen in the above groups is partially or fully deuterated;
wherein “*” represents a position where the substituent is joined to carbon.

12. The metal complex of claim 1, wherein U₂ or U₃ is CRᵤ, and the Rᵤ is selected from substituted or unsubstituted alkyl having 4 to 20 carbon atoms, substituted or unsubstituted cycloalkyl having 4 to 20 carbon atoms or a combination thereof;
preferably, Rᵤ is selected from substituted or unsubstituted alkyl having 4 to 6 carbon atoms, substituted or unsubstituted cycloalkyl having 4 to 6 carbon atoms or a combination thereof.

13. The metal complex of claim 1, wherein U₂ and U₃ are CRᵤ, and the Rᵤ is, at each occurrence identically or differently, selected from substituted or unsubstituted alkyl having 1 to 20 carbon atoms, substituted or unsubstituted cycloalkyl having 3 to 20 ring carbon atoms or a combination thereof, and the number of carbon atoms in at least one Rᵤ is greater than or equal to 4.

14. The metal complex of claim 13, wherein U₁ and U₄ are CRᵤ and Rᵤ is selected from hydrogen, deuterium, methyl or deuterated methyl.

15. The metal complex of claim 1, wherein W₁ to W₄ are, at each occurrence identically or differently, selected from CR_{w}, Y₁ to Y₄ are, at each occurrence identically or differently, selected from CR_{y}, and R_{w} and R_{y} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof;
preferably, R_{w} and R_{y} are, at each occurrence identically or differently, selected from the group consisting of: hydrogen, deuterium, substituted or unsubstituted alkyl having 1 to 10 carbon atoms, substituted or unsubstituted cycloalkyl having 3 to 10 ring carbon atoms, substituted or unsubstituted aryl having 6 to 10 carbon atoms and combinations thereof;
more preferably, R_{w} and R_{y} are, at each occurrence identically or differently, selected from the group consisting of: hydrogen, deuterium, substituted or unsubstituted alkyl having 1 to 10 carbon atoms, substituted or unsubstituted cycloalkyl having 3 to 10 ring carbon atoms and combinations thereof.

16. The metal complex of claim 1, wherein W₁ to W₄ are, at each occurrence identically or differently, selected from CR_{w}, and at least one R_{w} is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof; and/or Y₁ to Y₄ are, at each occurrence identically or differently, selected from CR_{y}, and at least one R_{y} is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof.

17. The metal complex of claim 1, wherein Lₐ is, at each occurrence identically or differently, selected from the group consisting of the following:

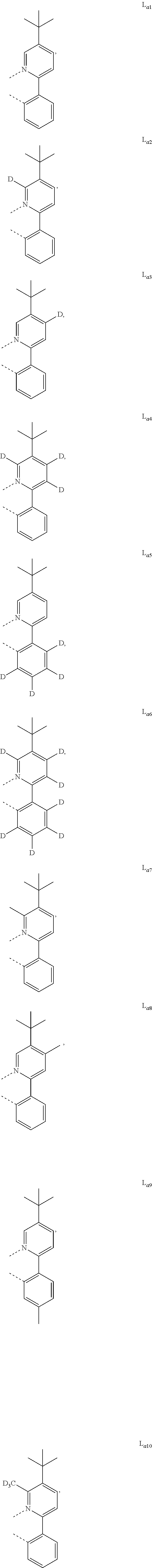

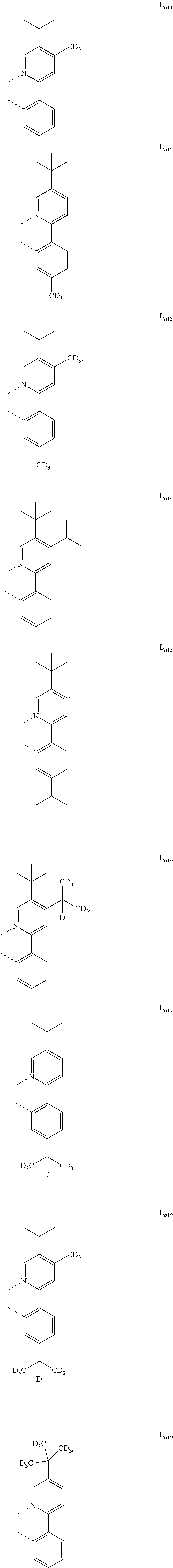

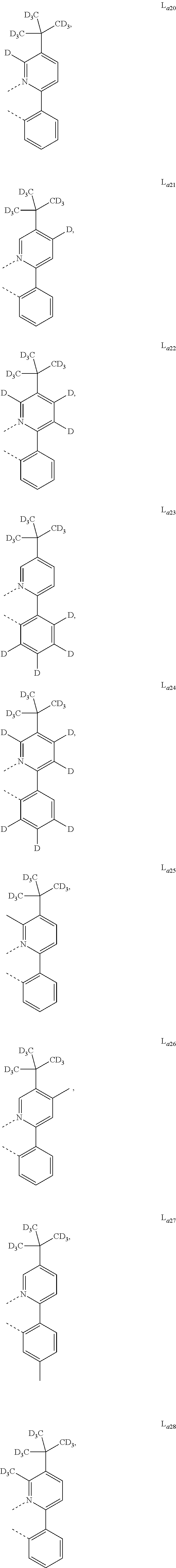

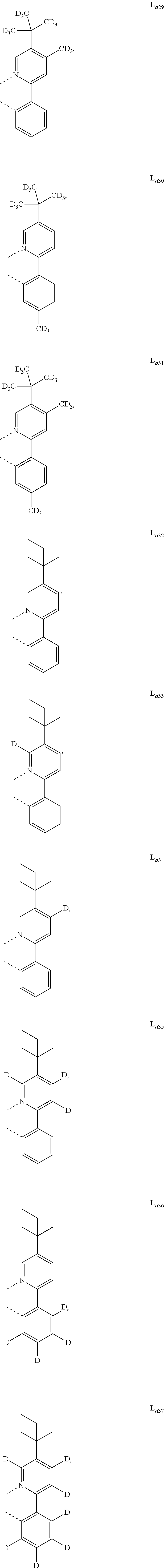

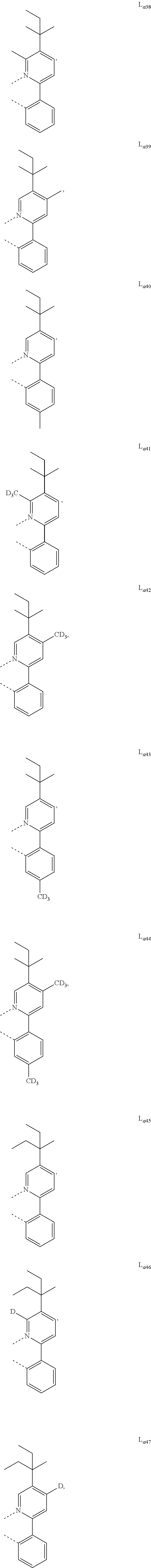

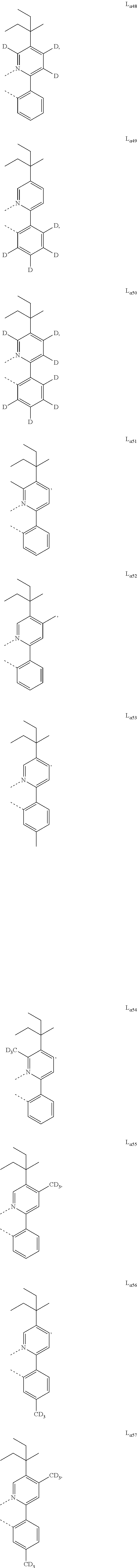

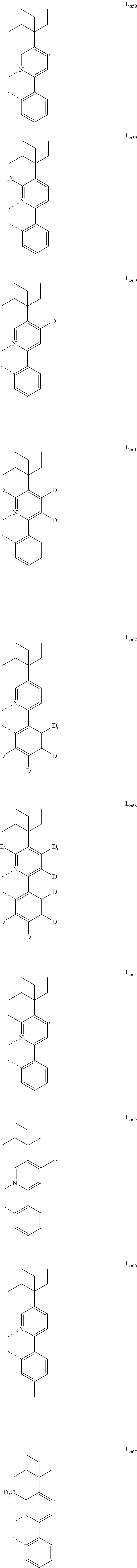

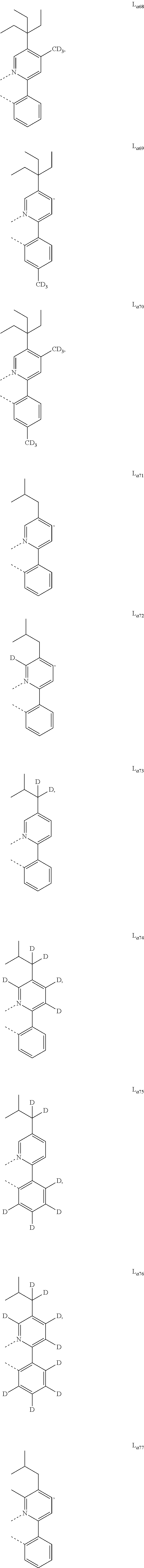

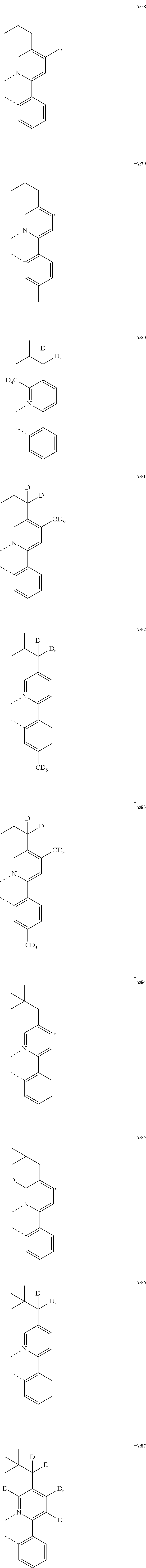

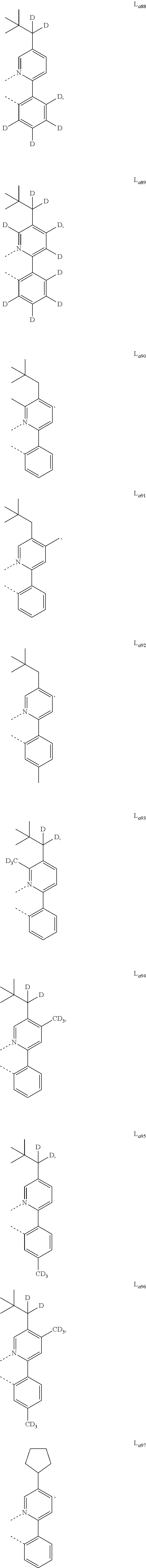

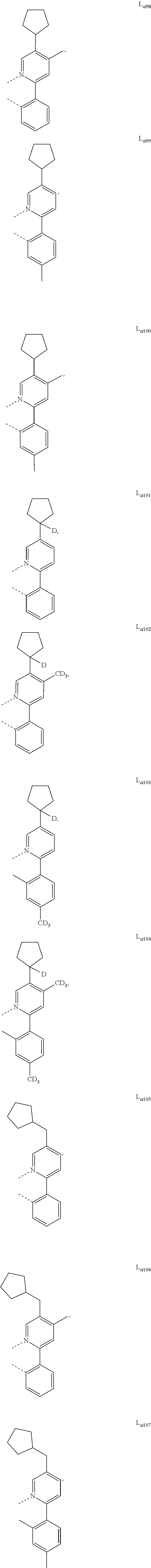

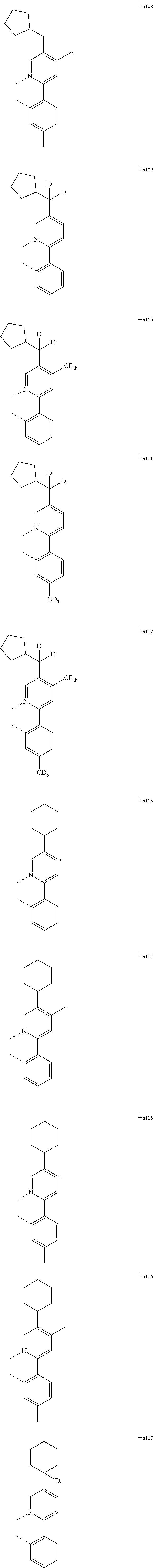

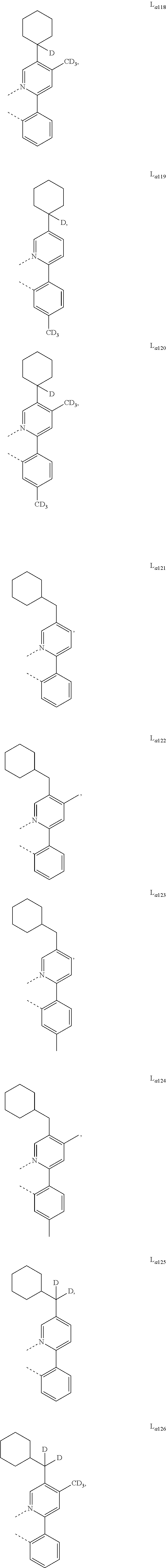

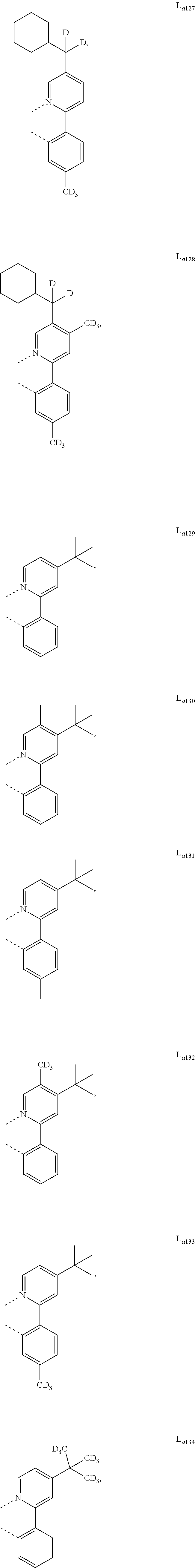

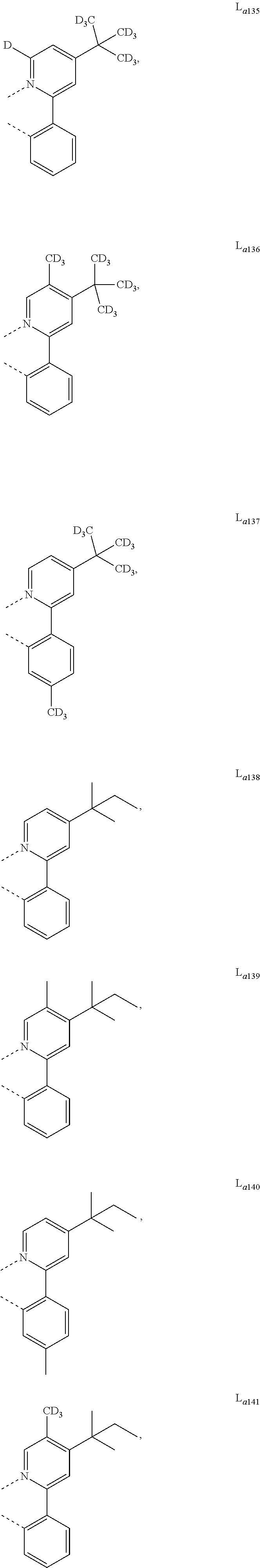

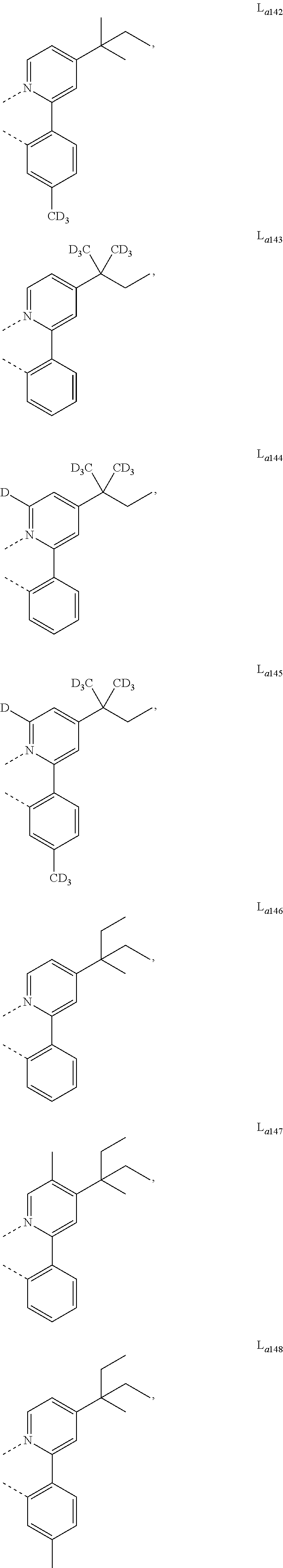

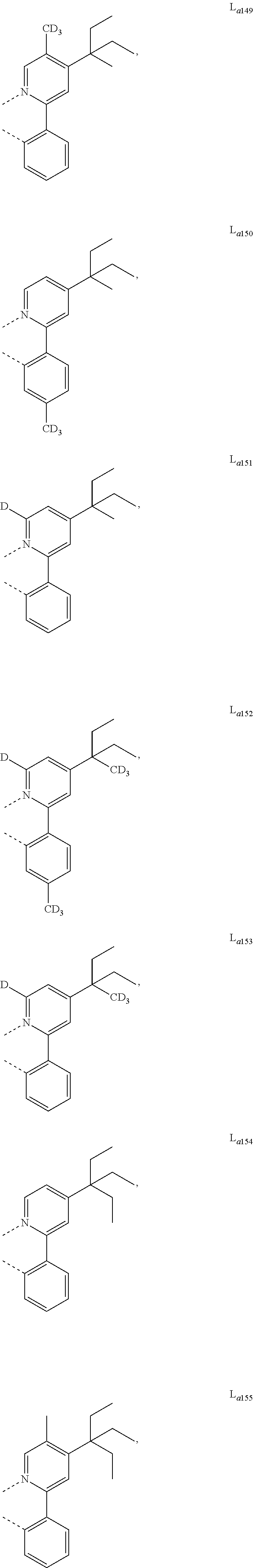

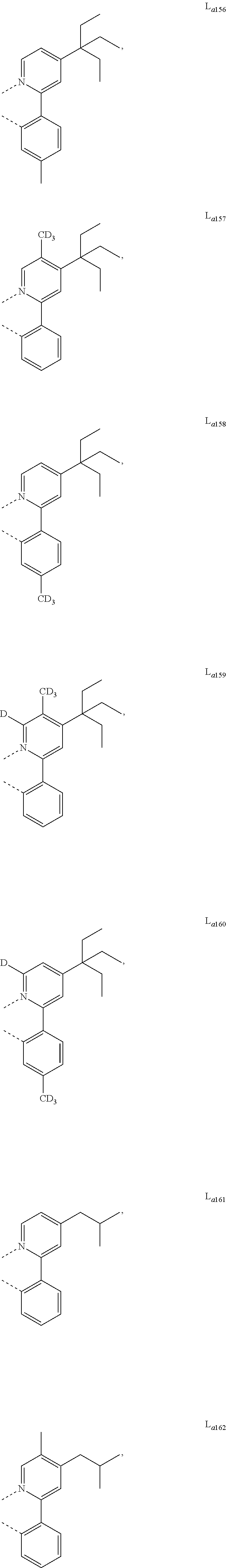

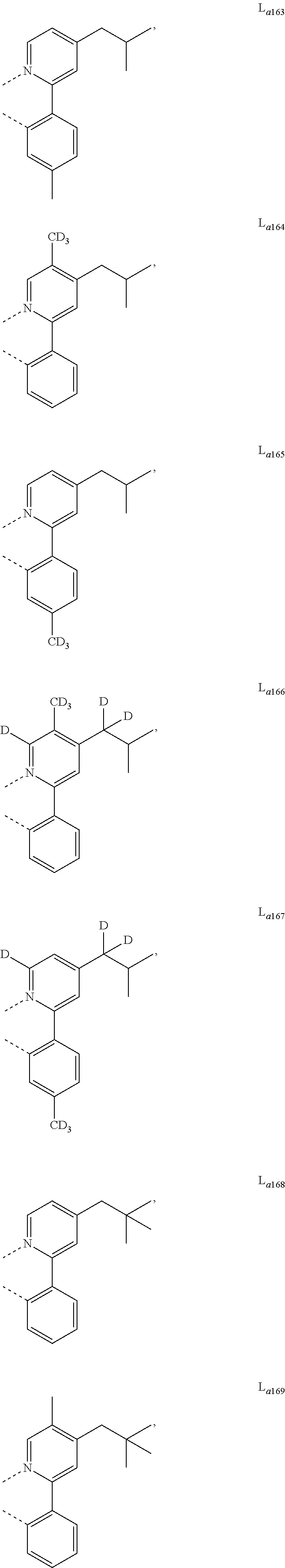

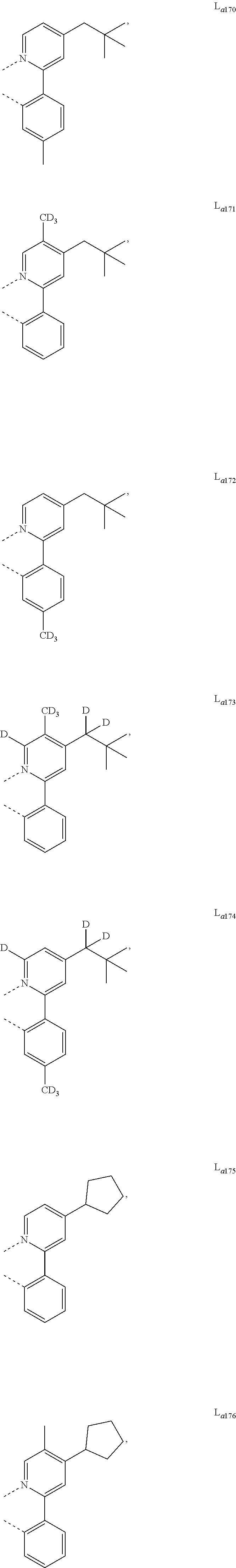

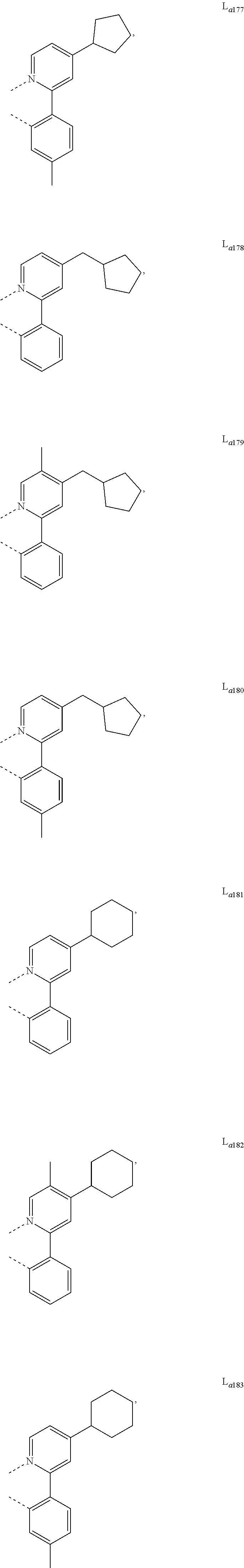

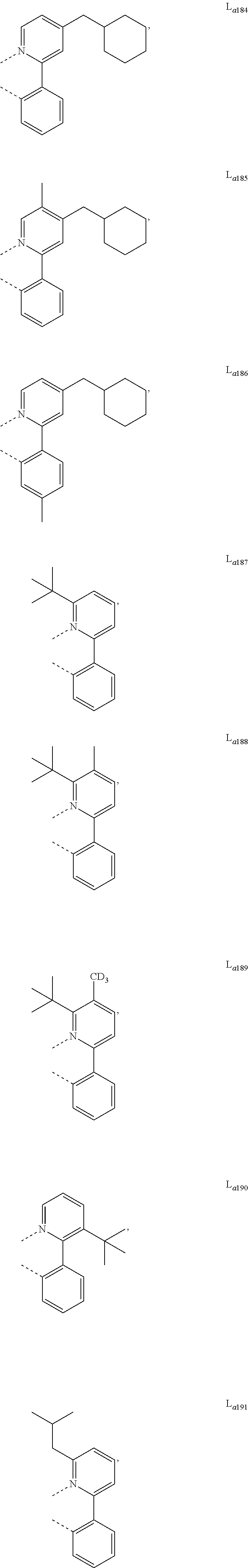

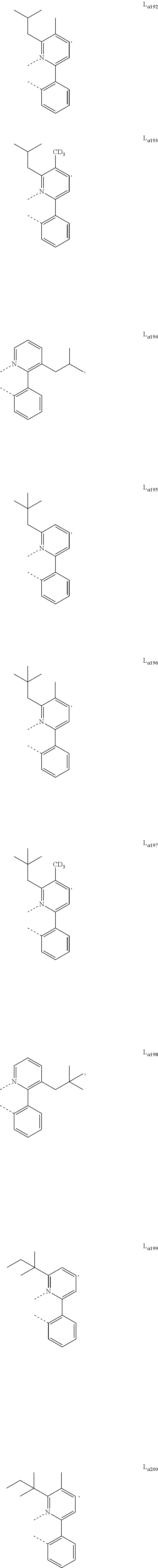

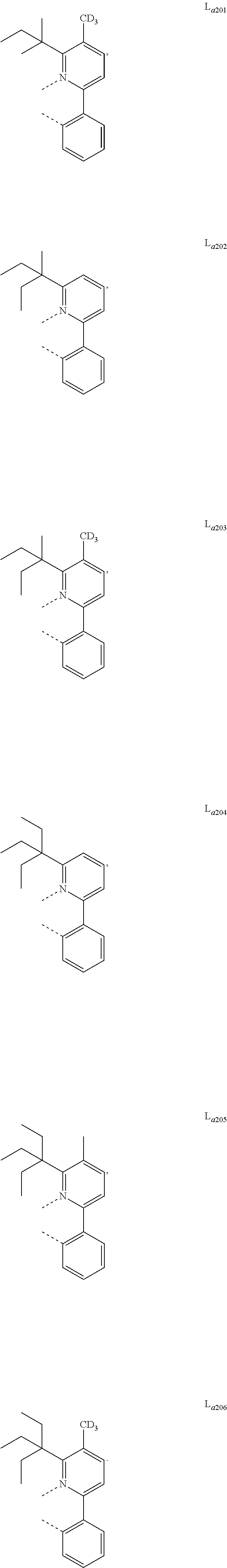

18. The metal complex of claim 1, wherein L_{b} is, at each occurrence identically or differently, selected from the group consisting of the following:

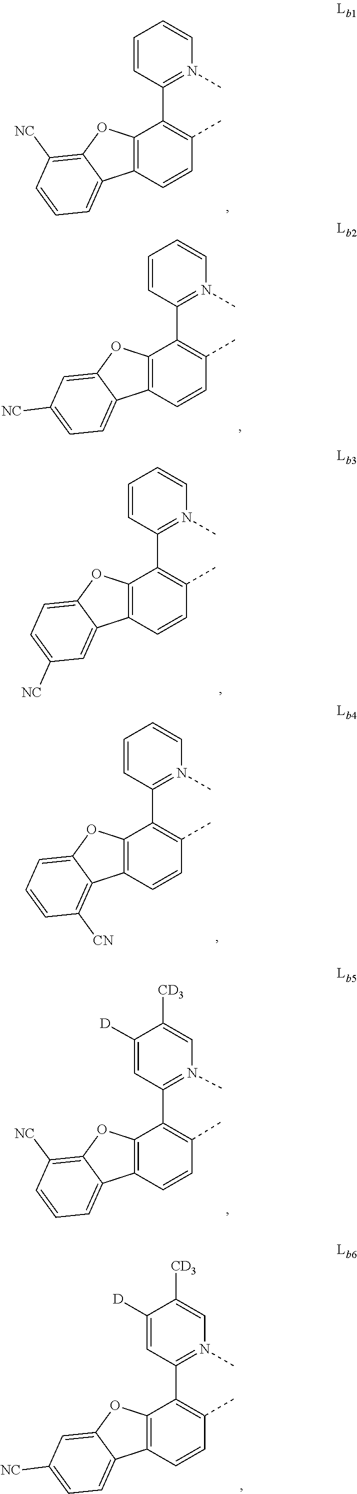

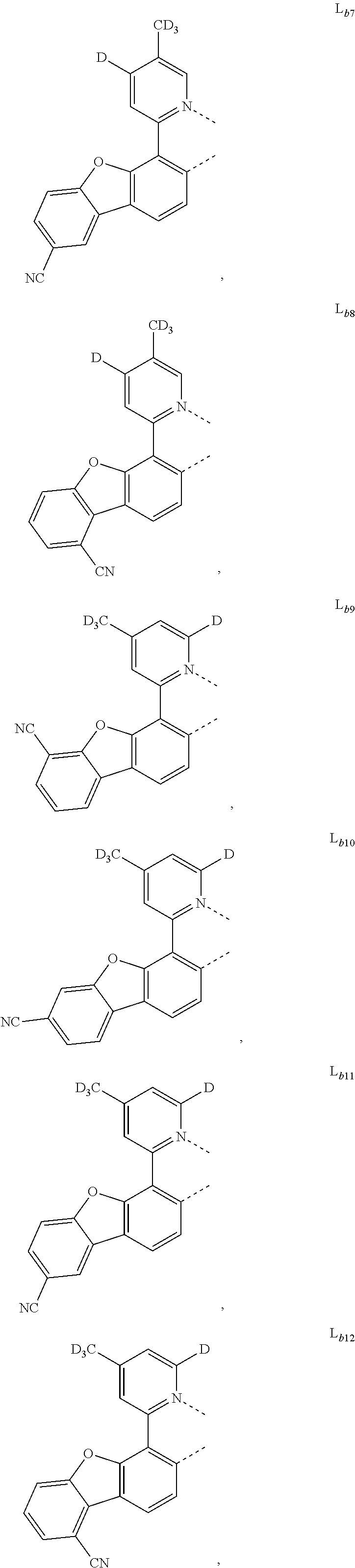

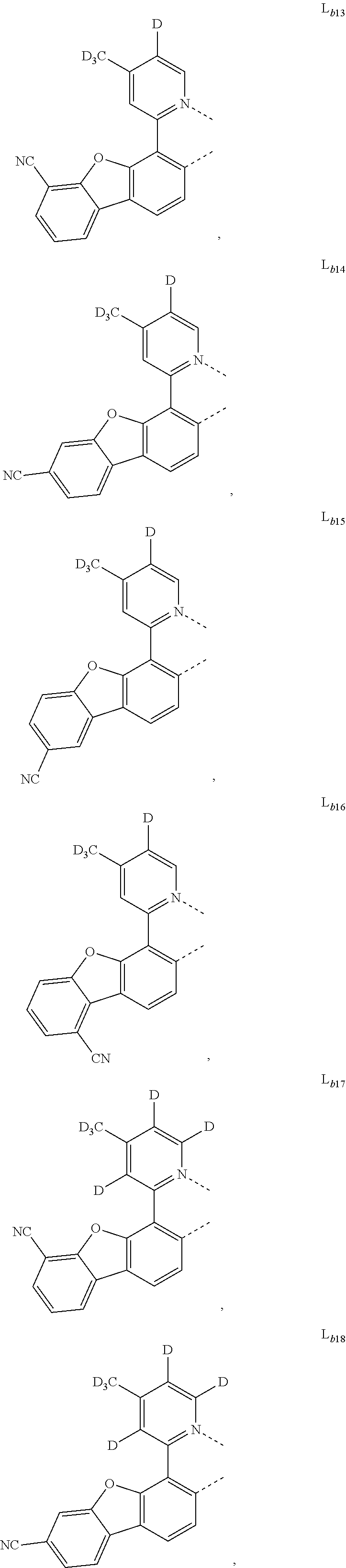

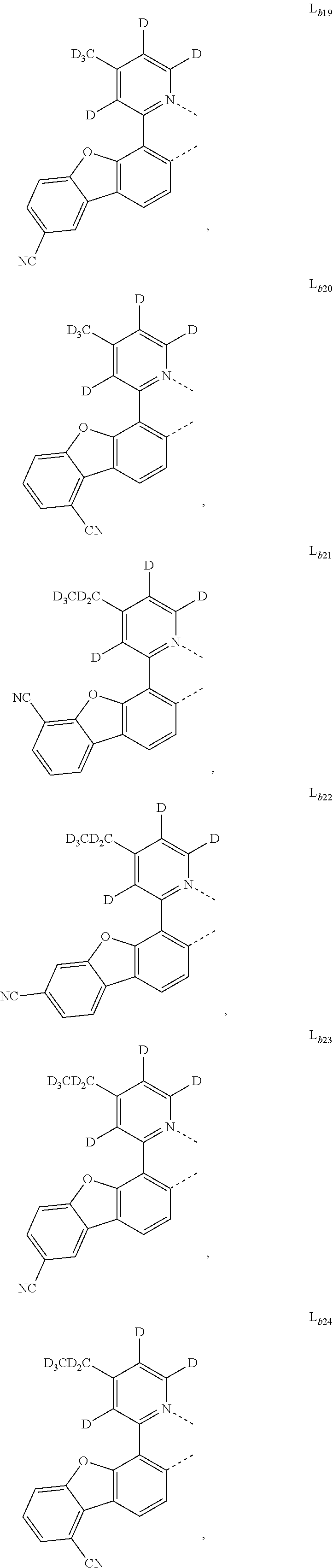

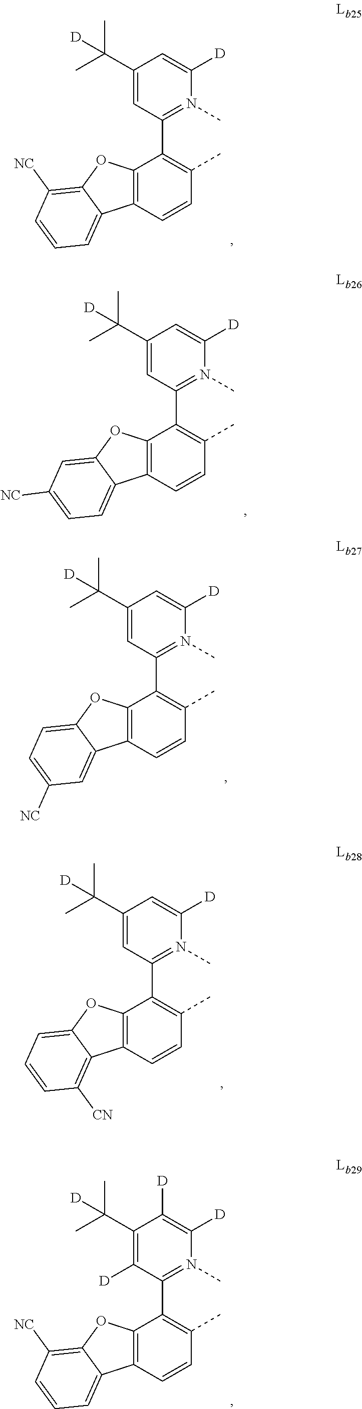

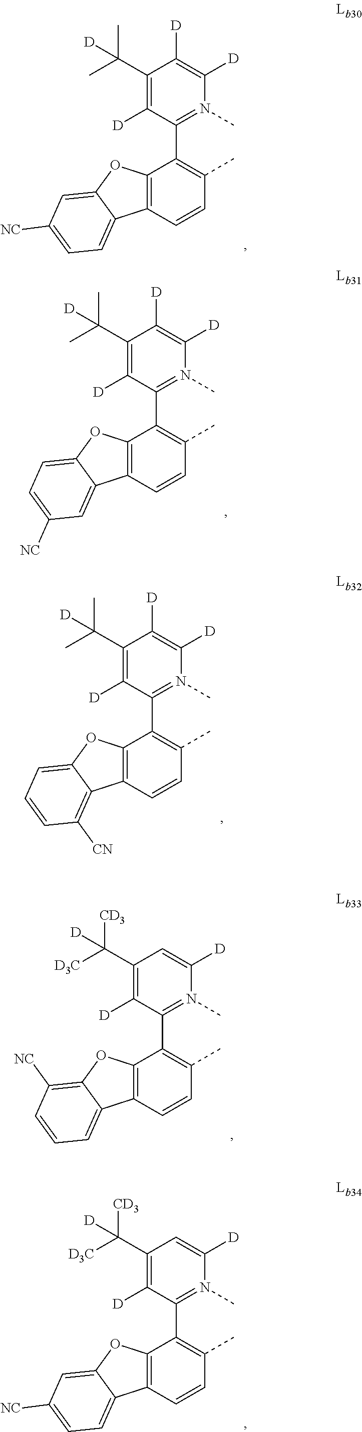

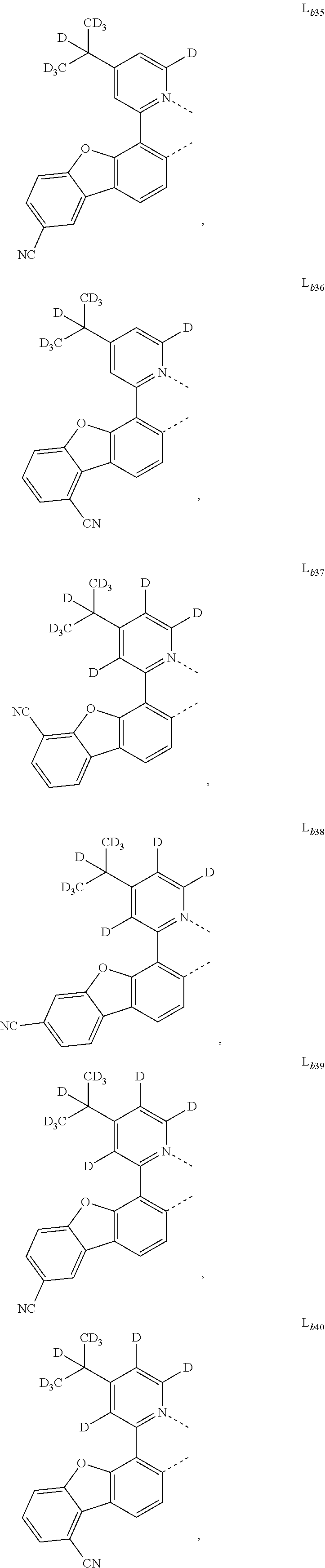

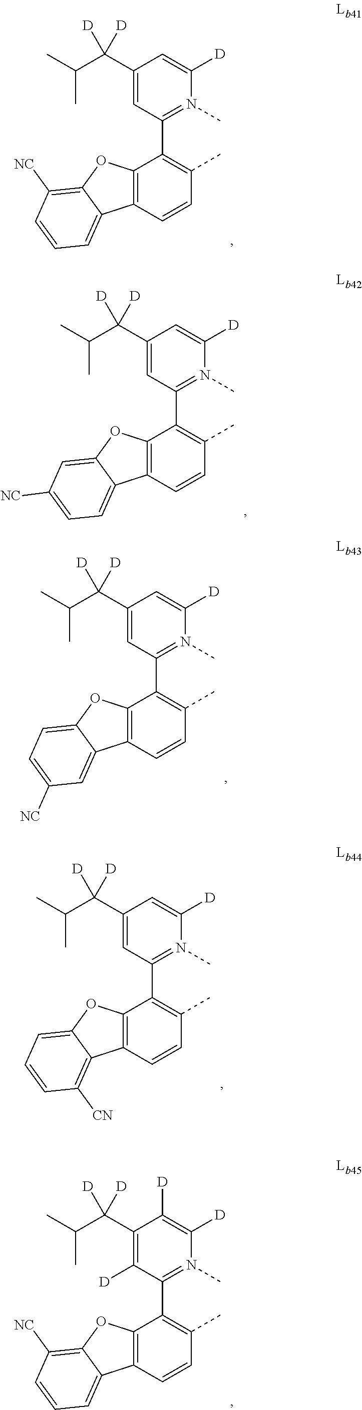

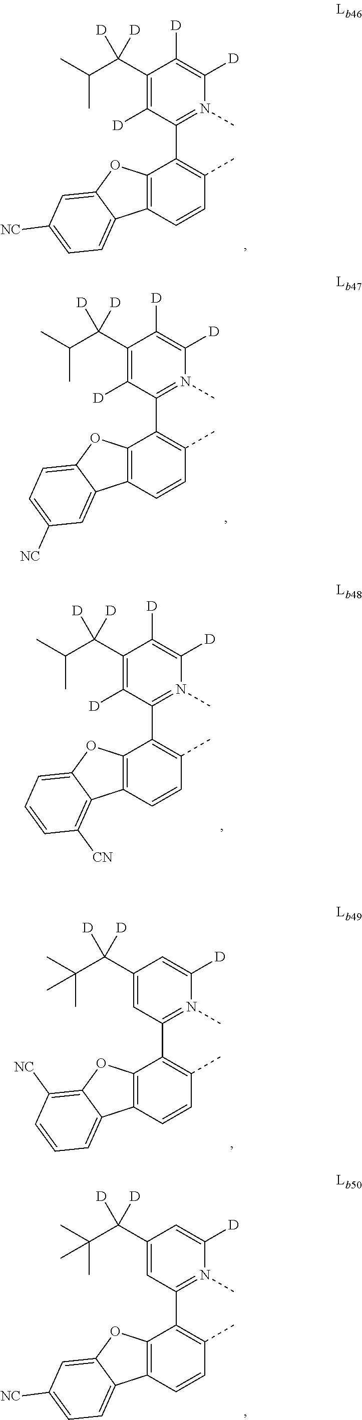

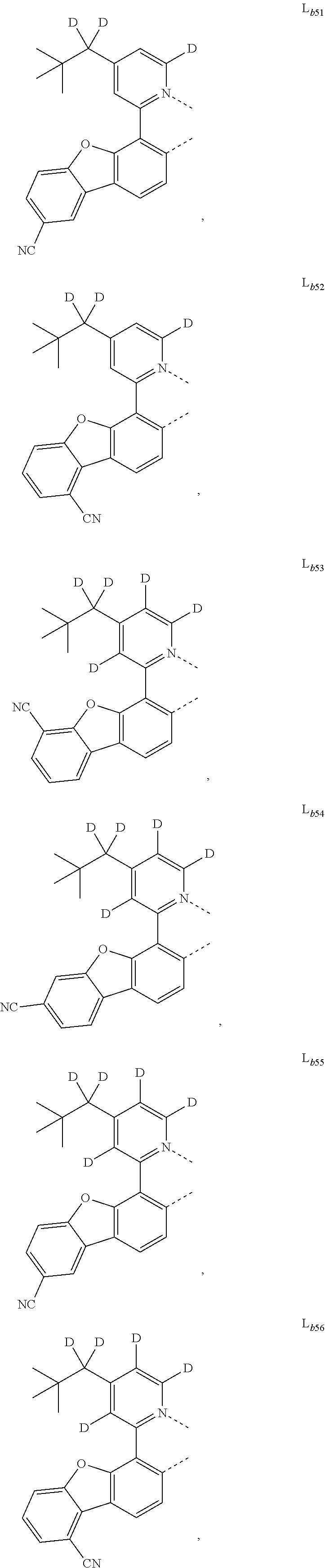

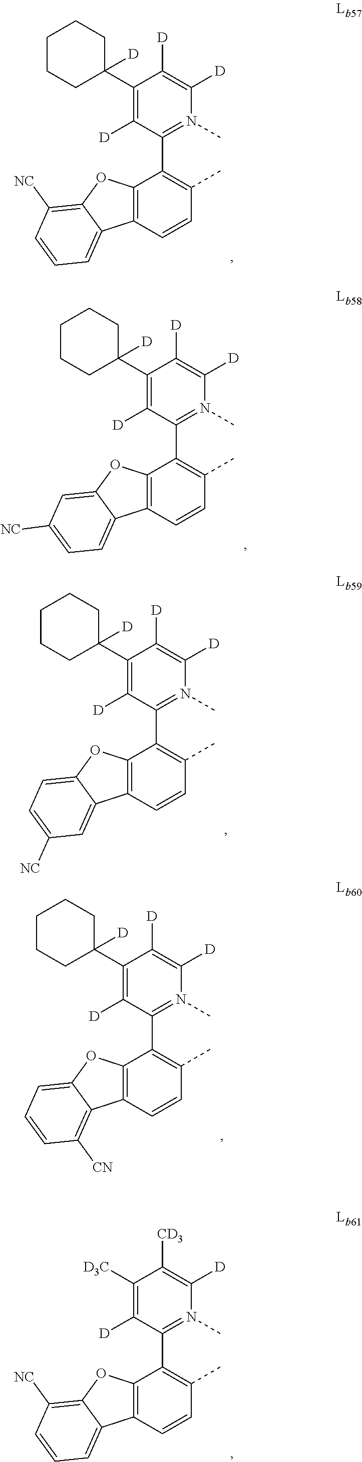

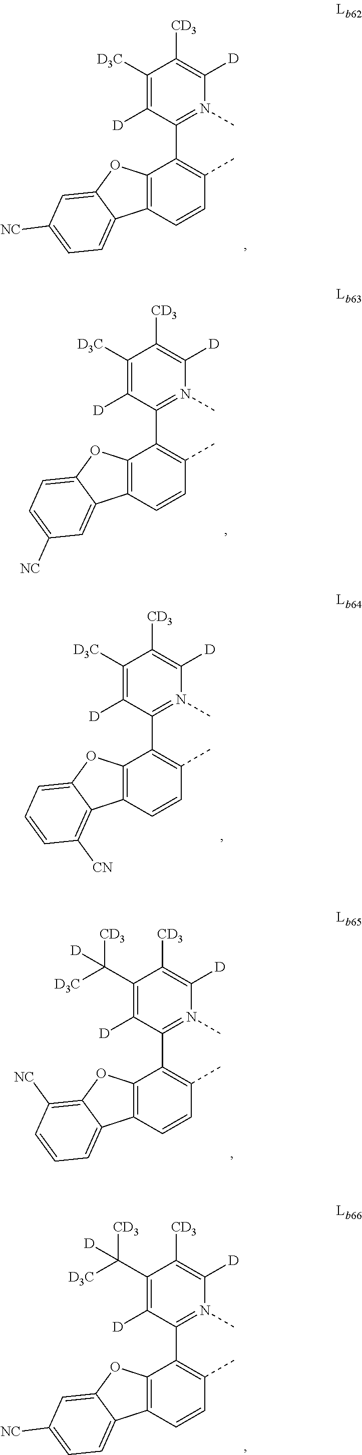

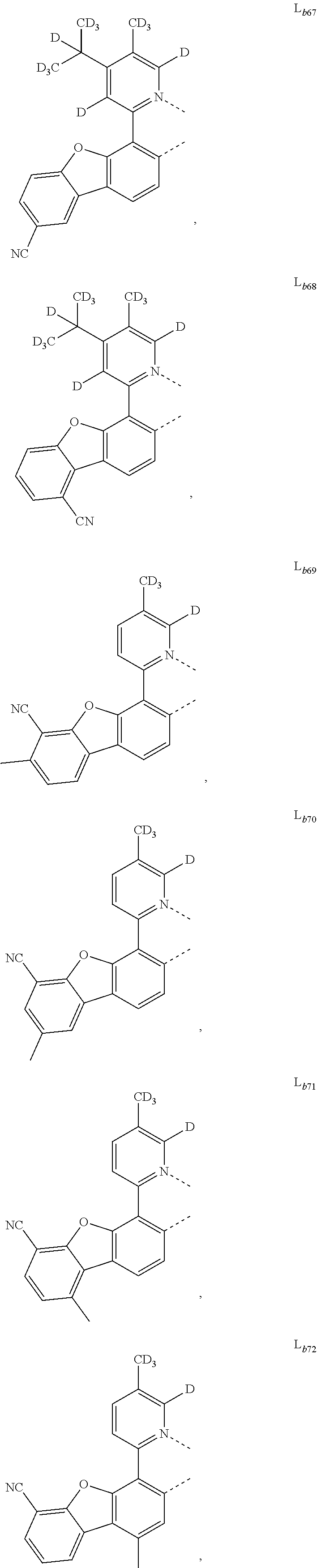

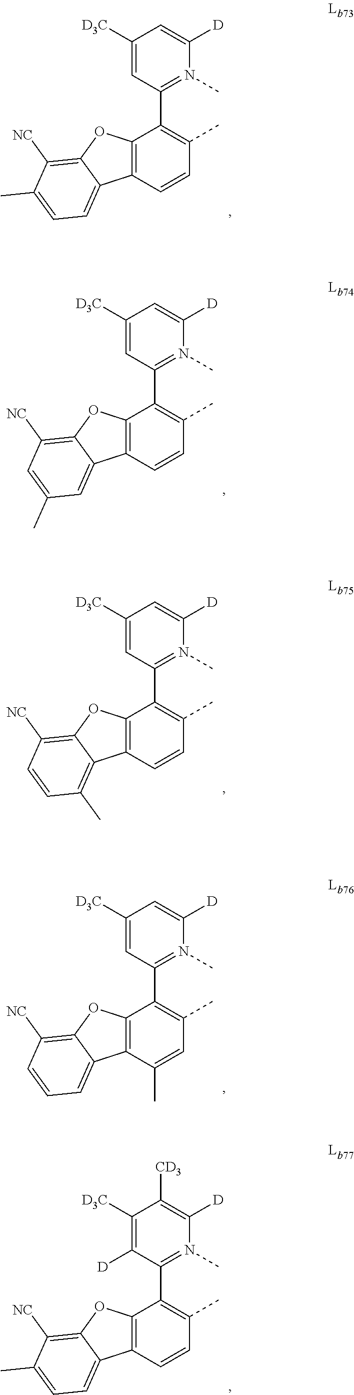

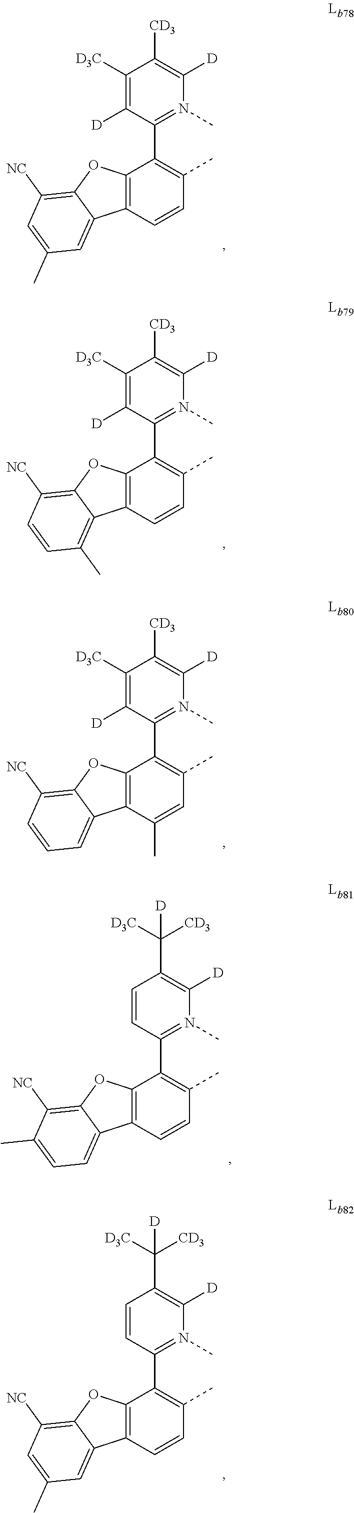

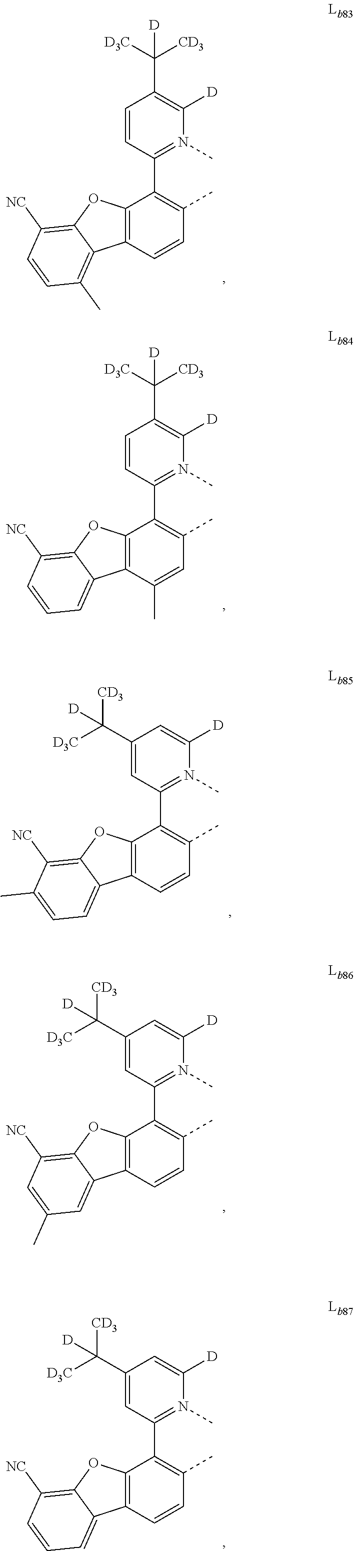

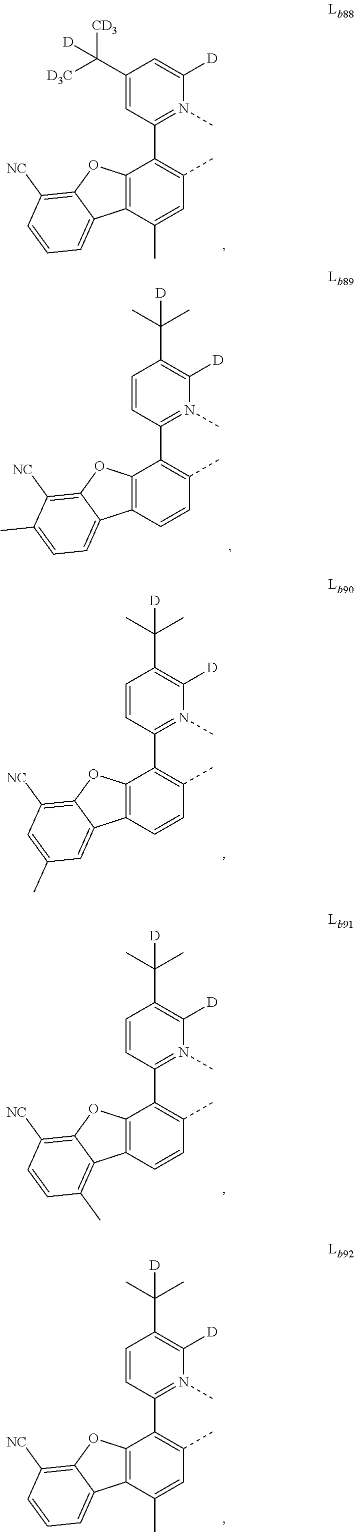

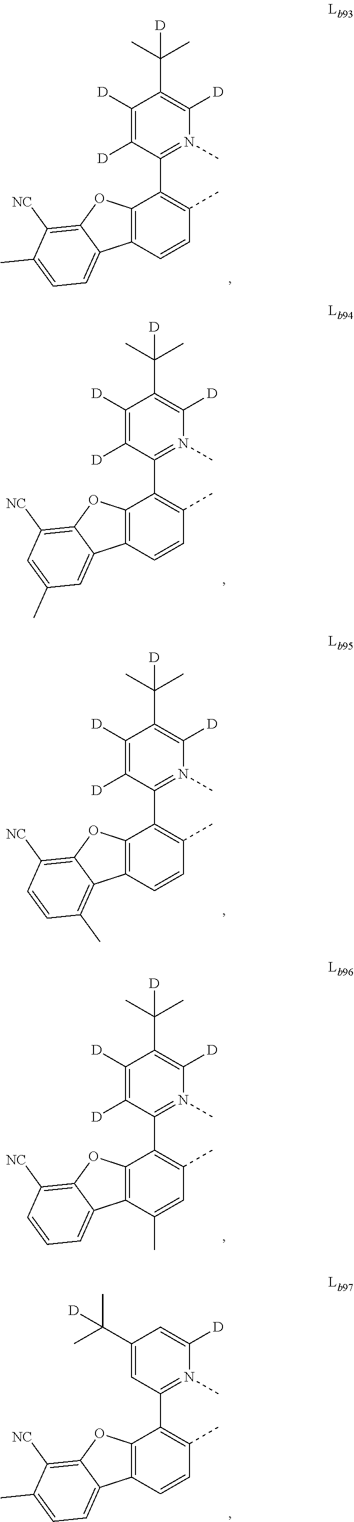

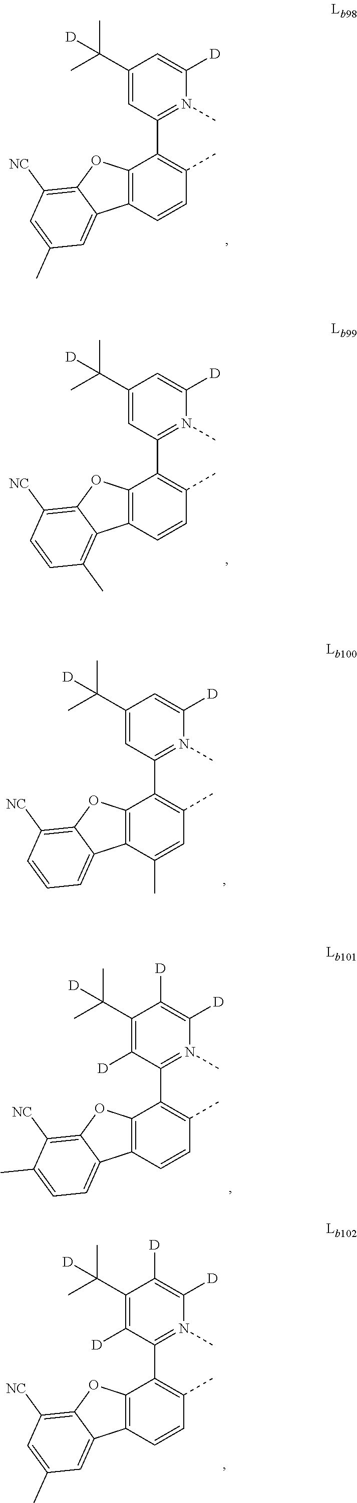

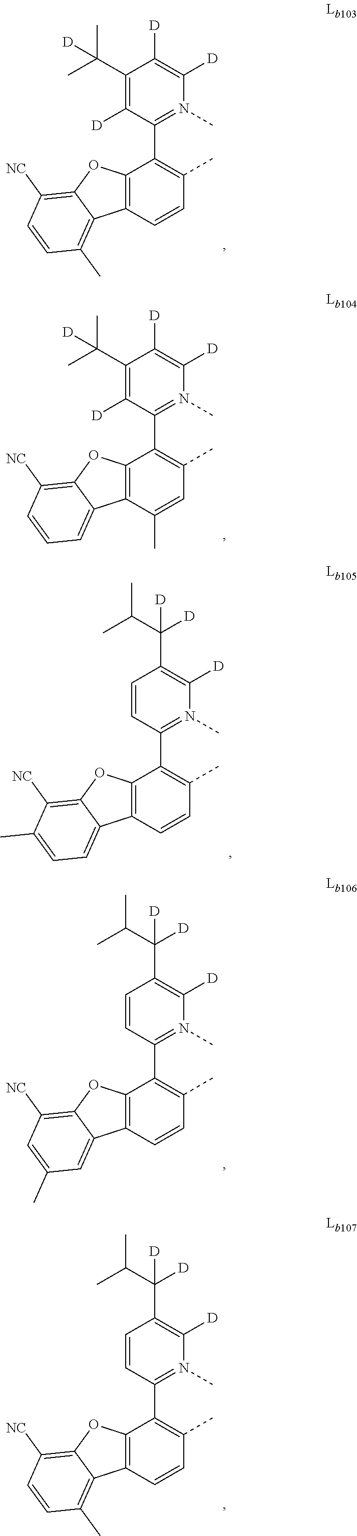

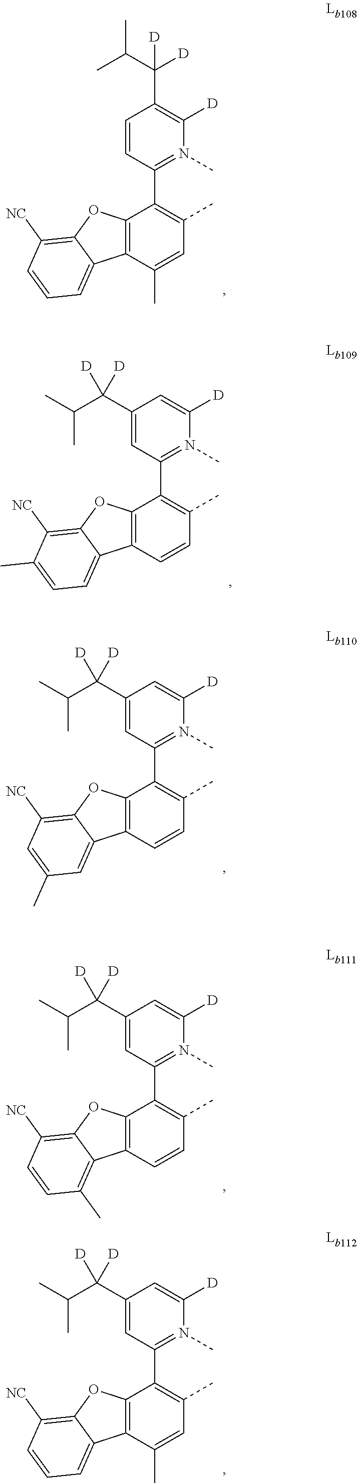

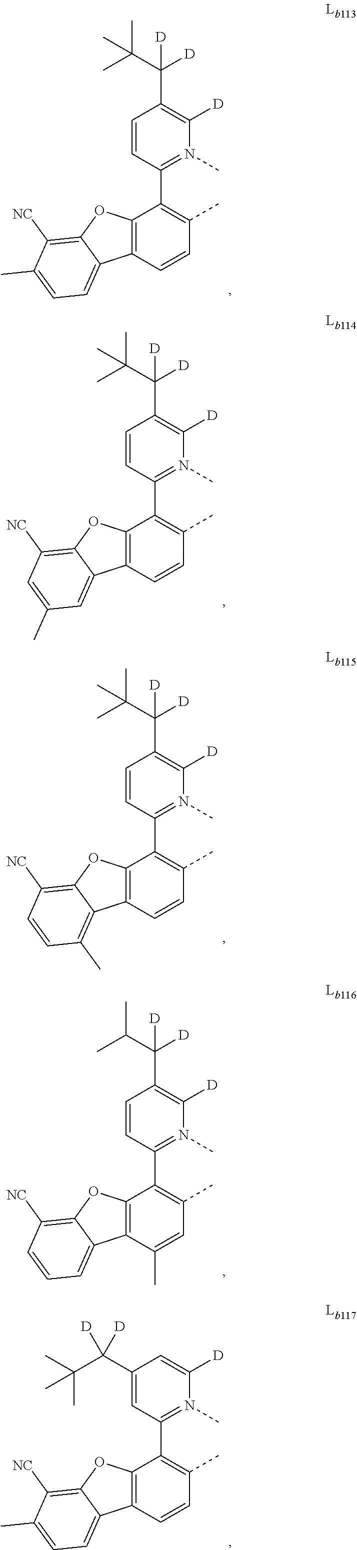

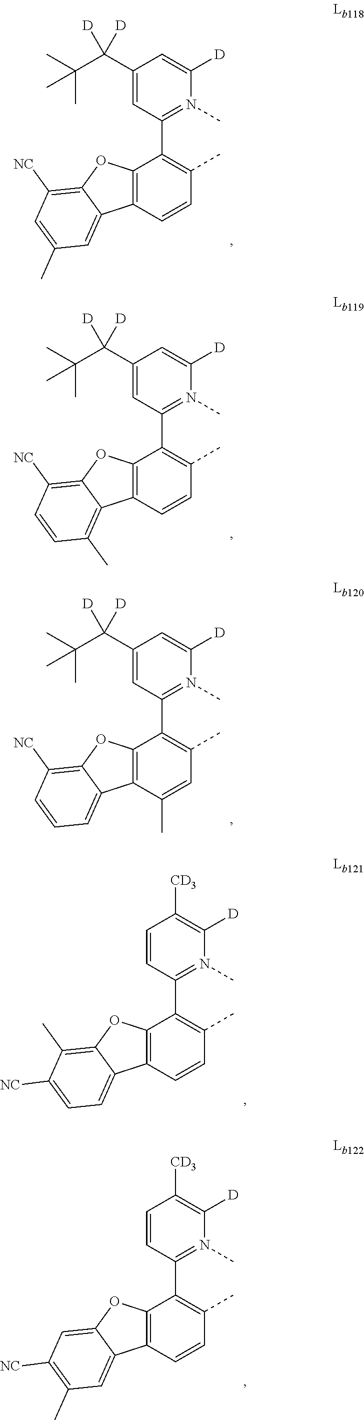

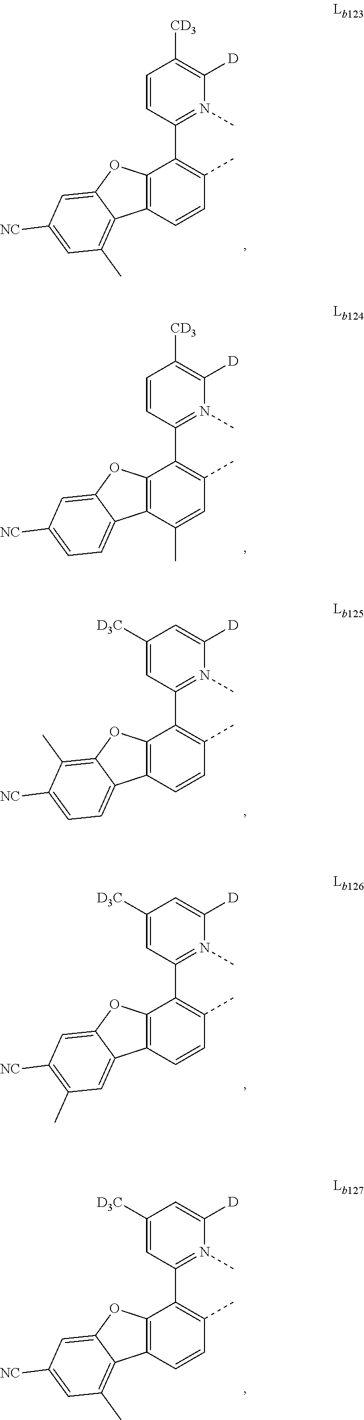

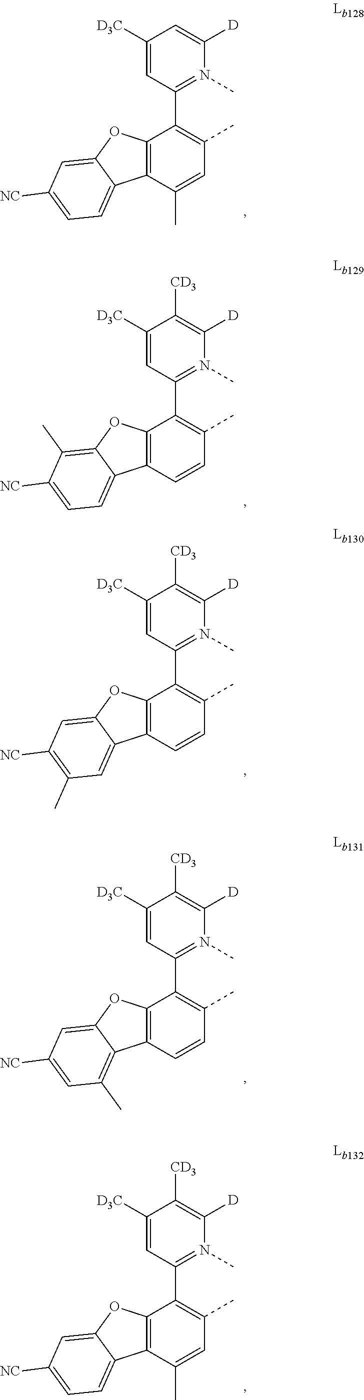

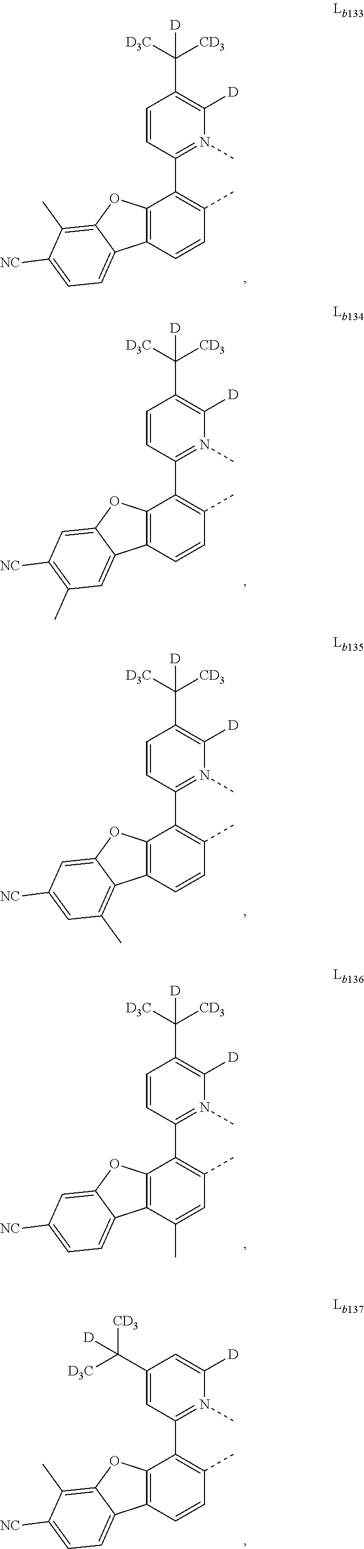

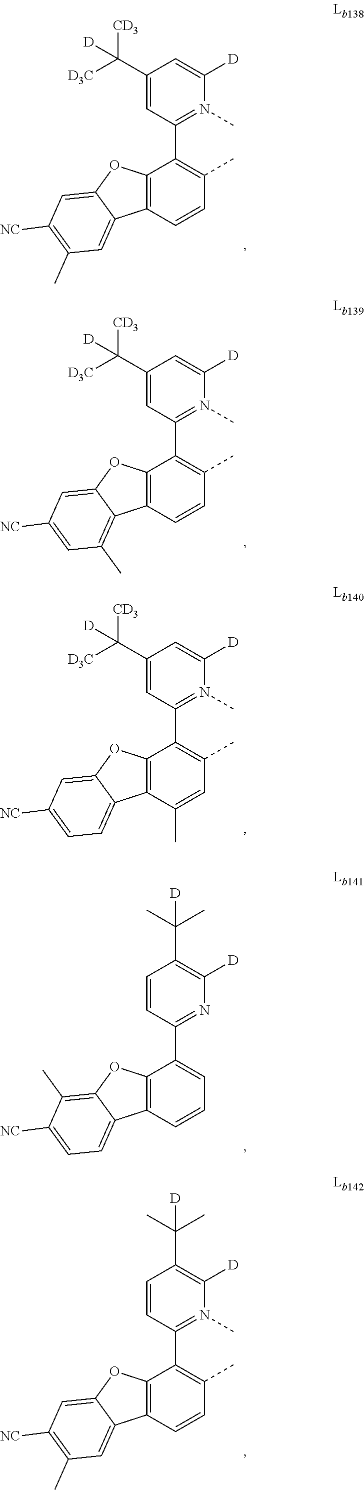

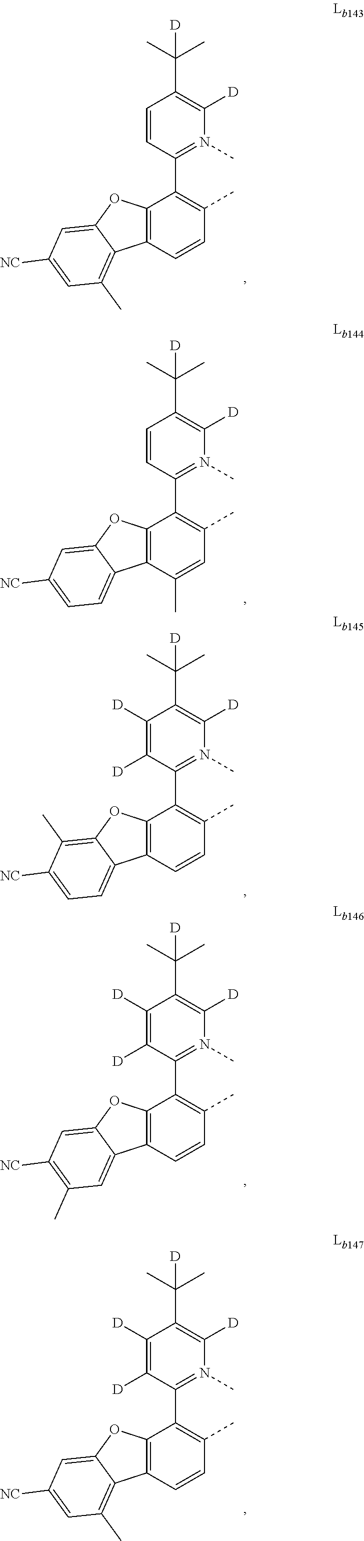

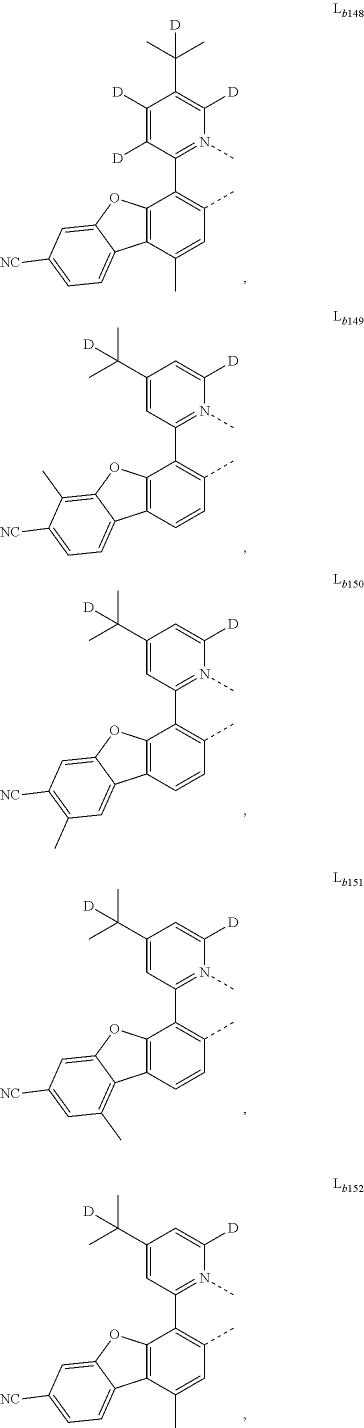

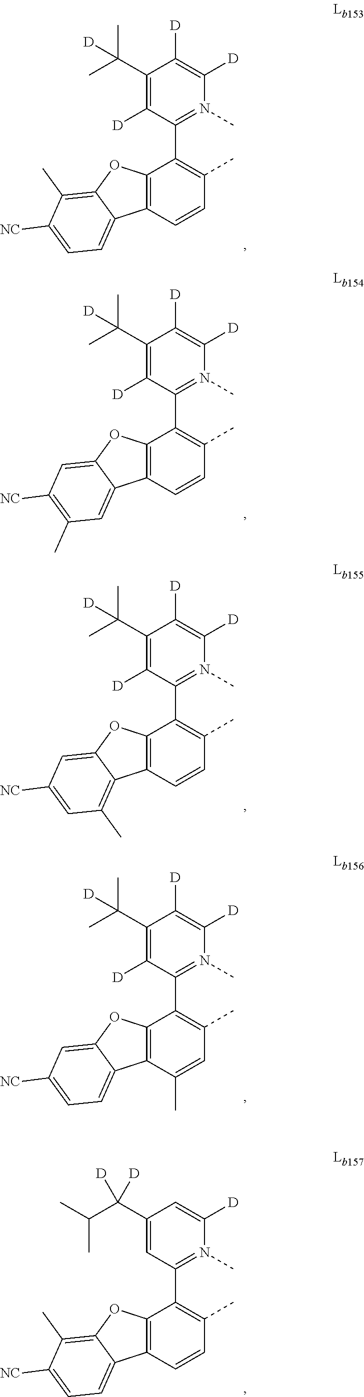

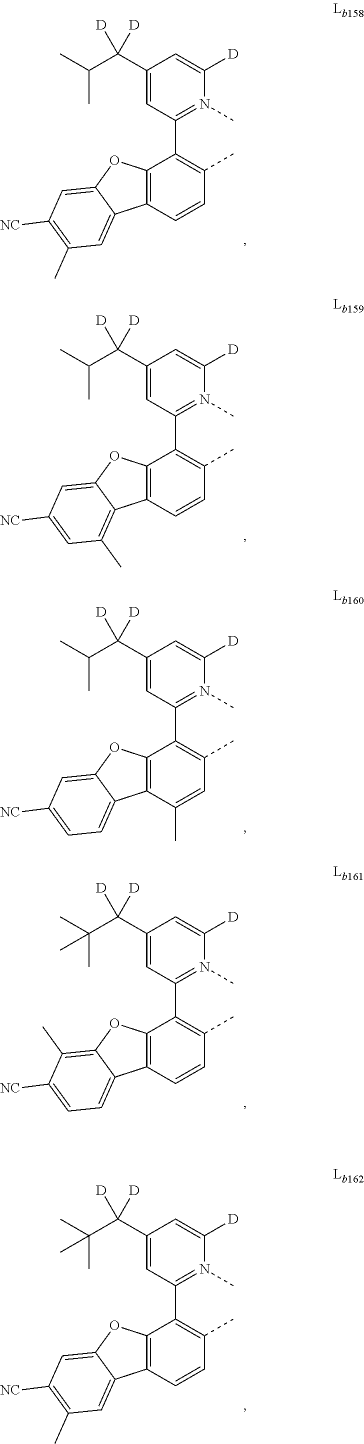

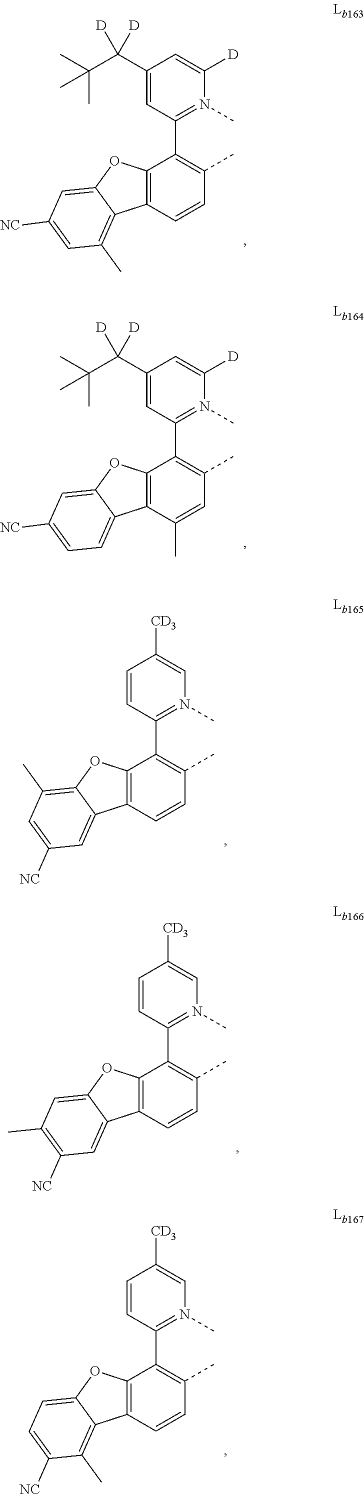

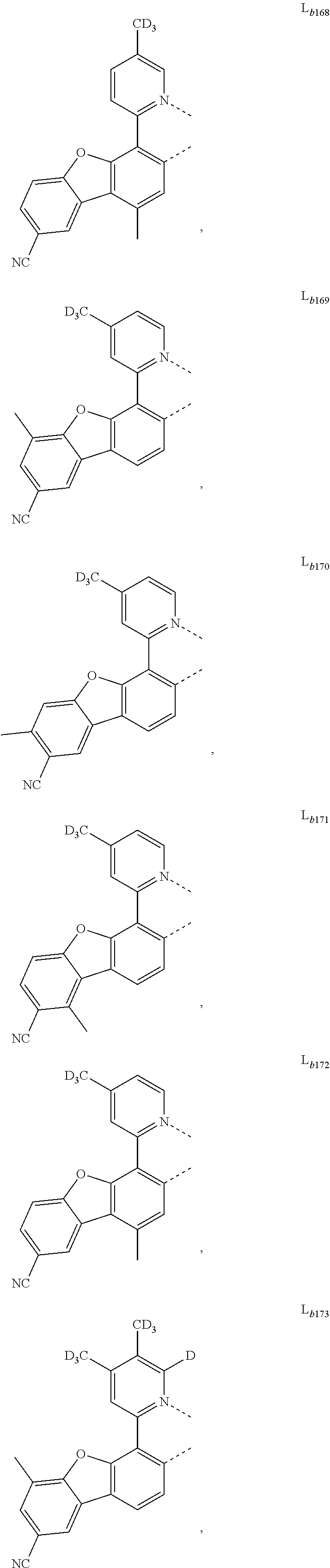

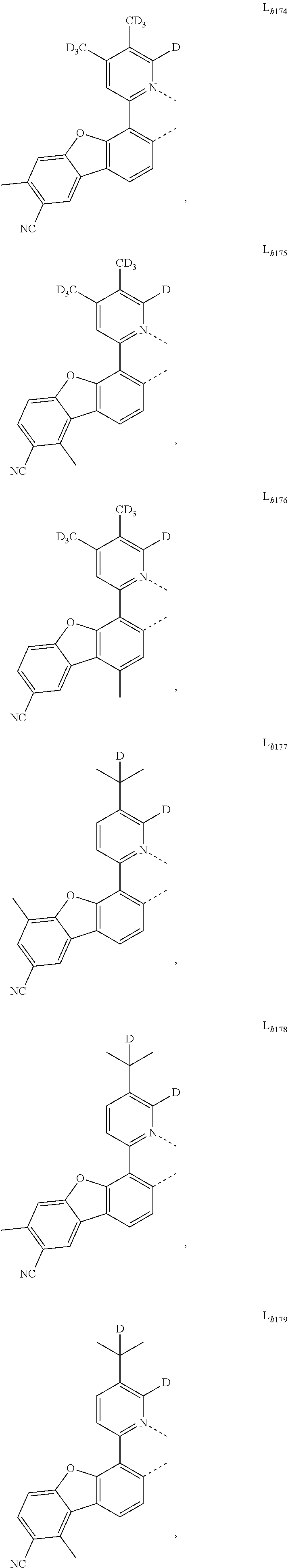

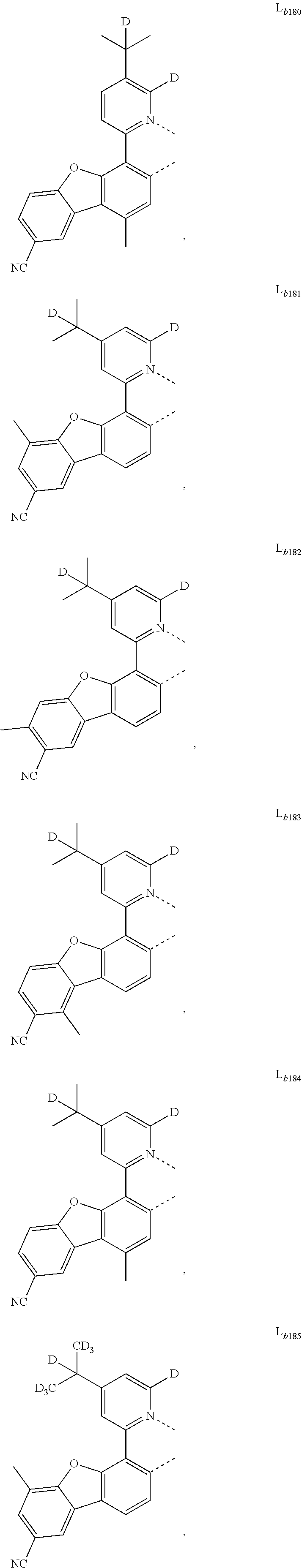

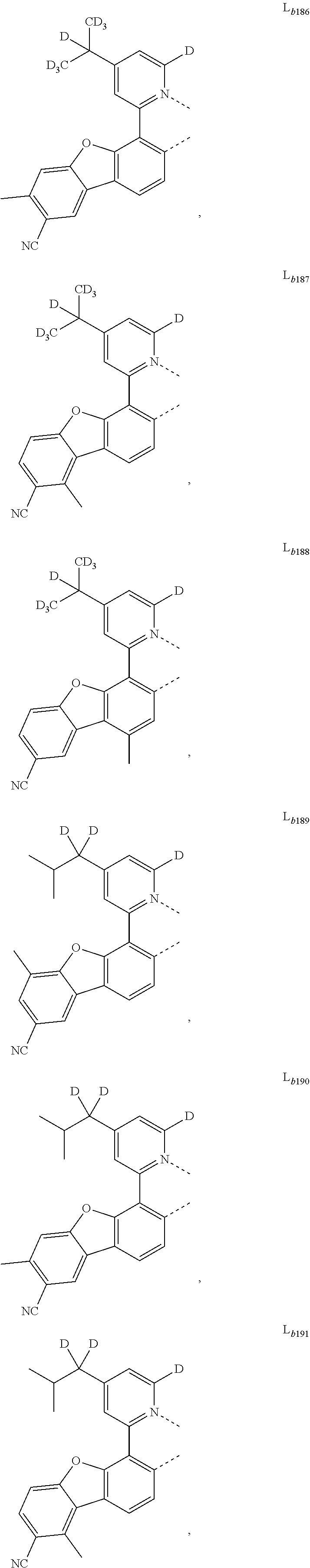

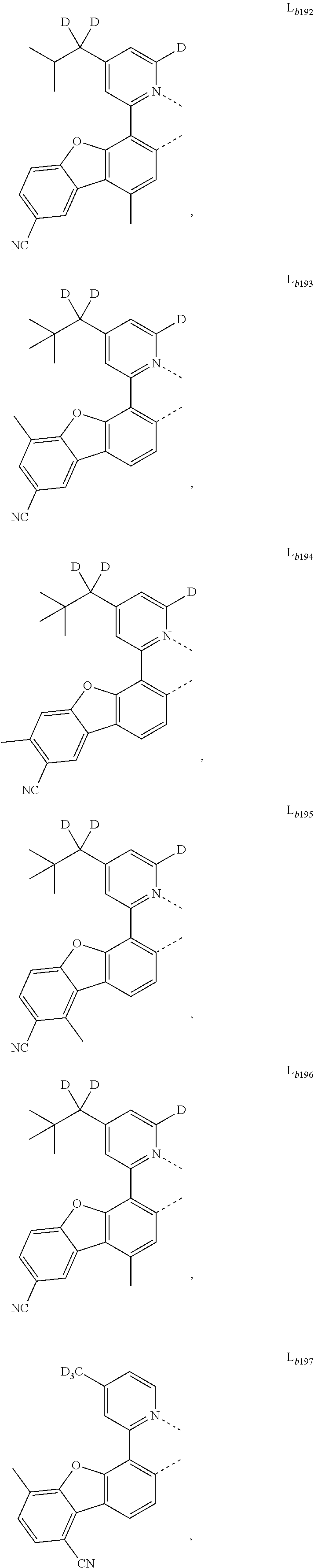

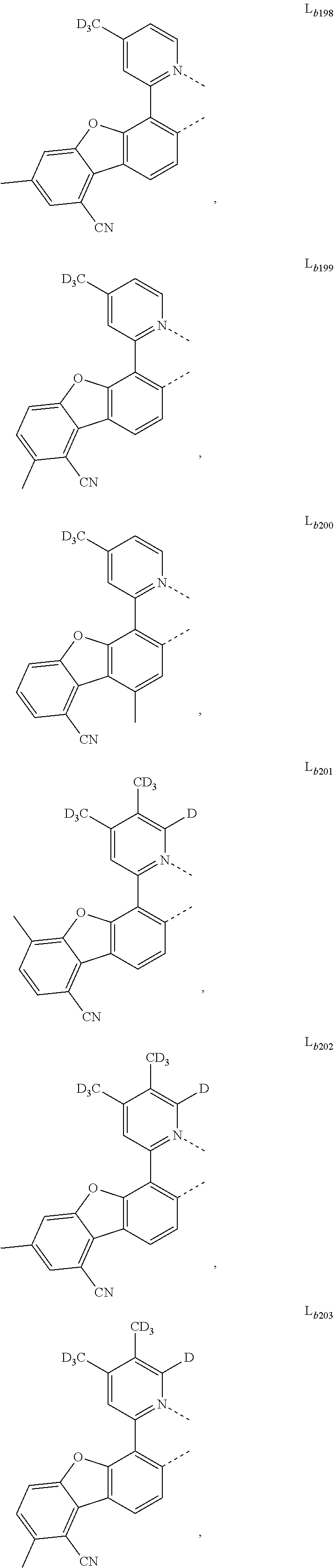

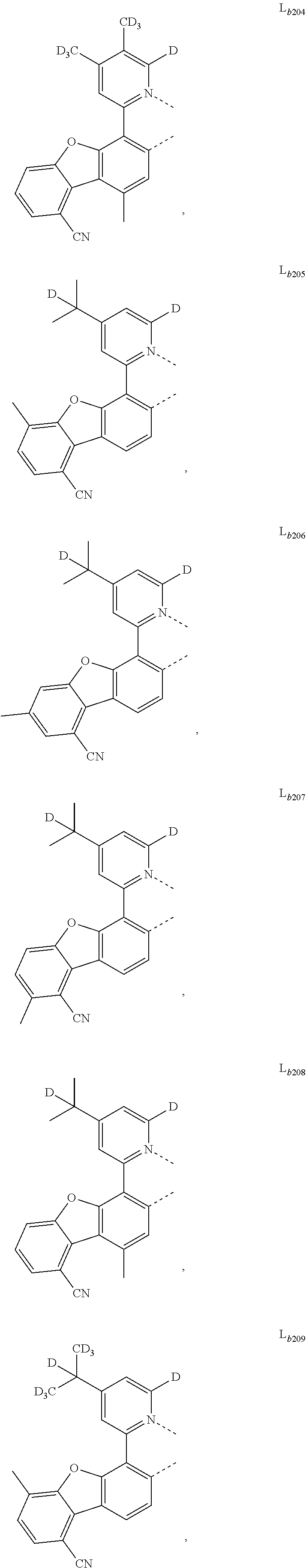

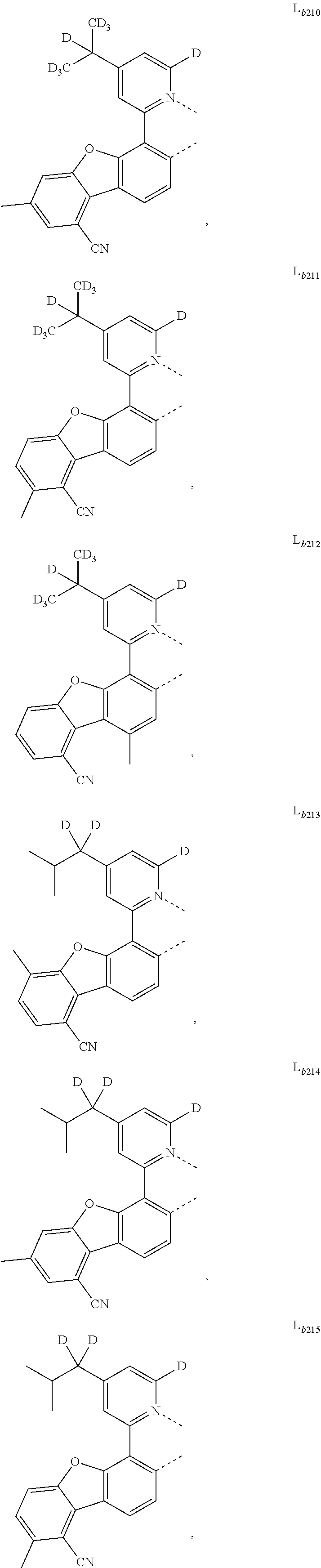

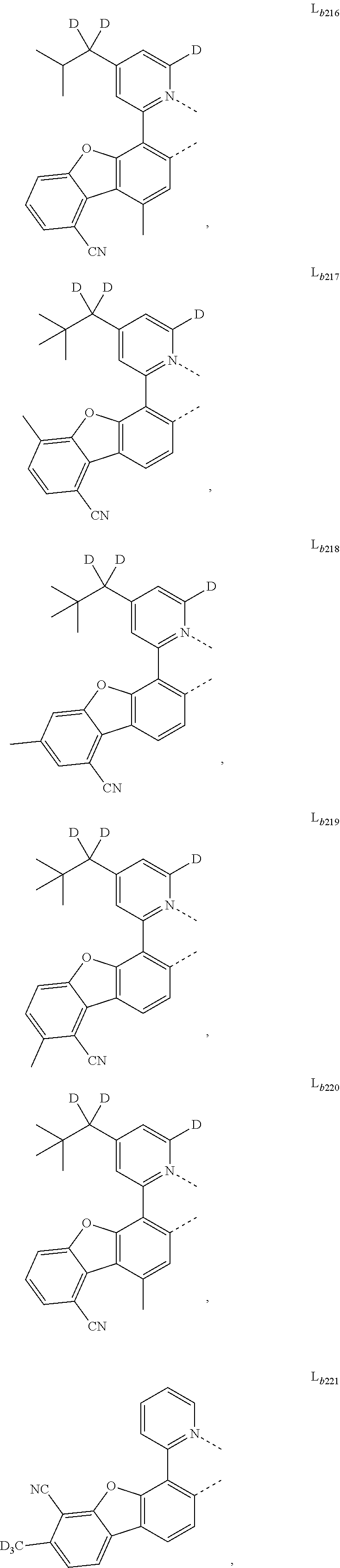

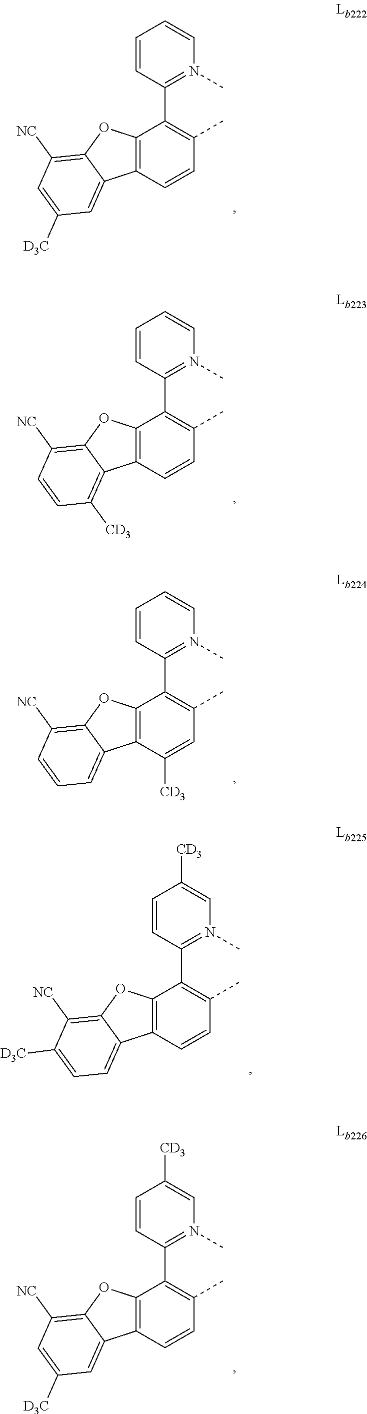

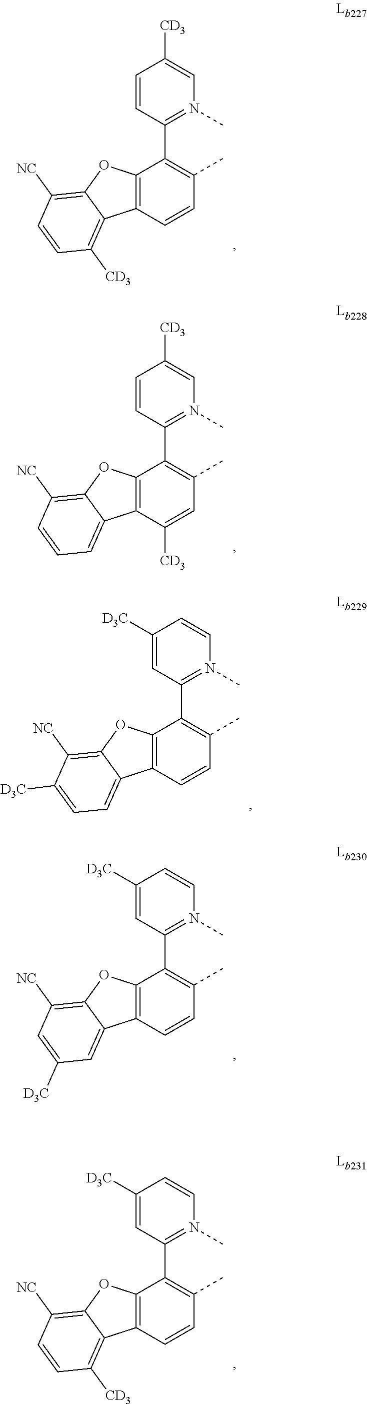

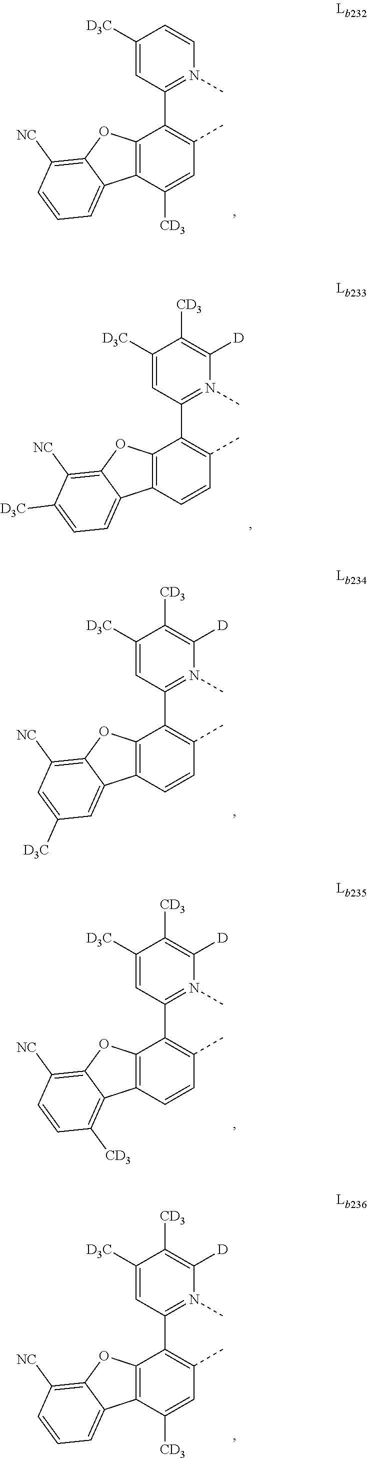

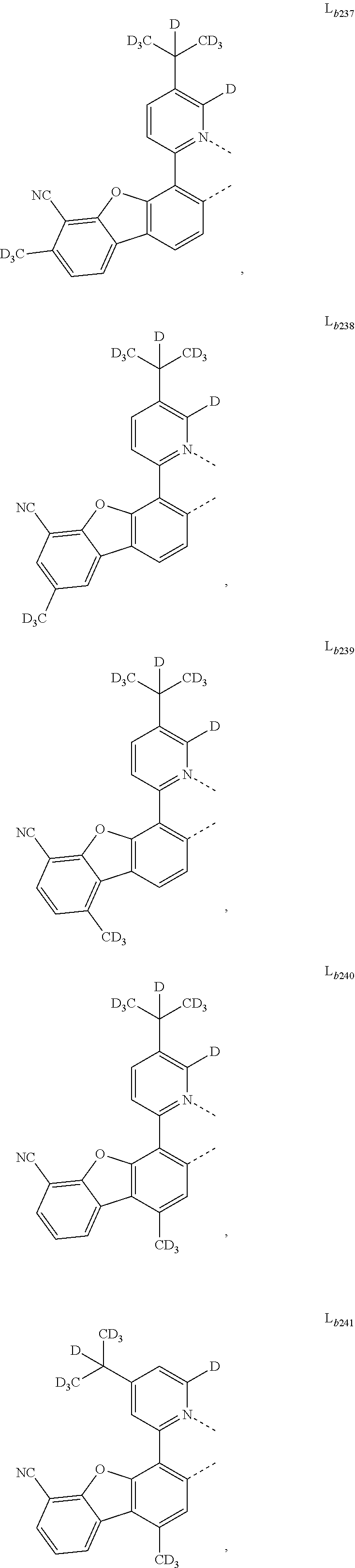

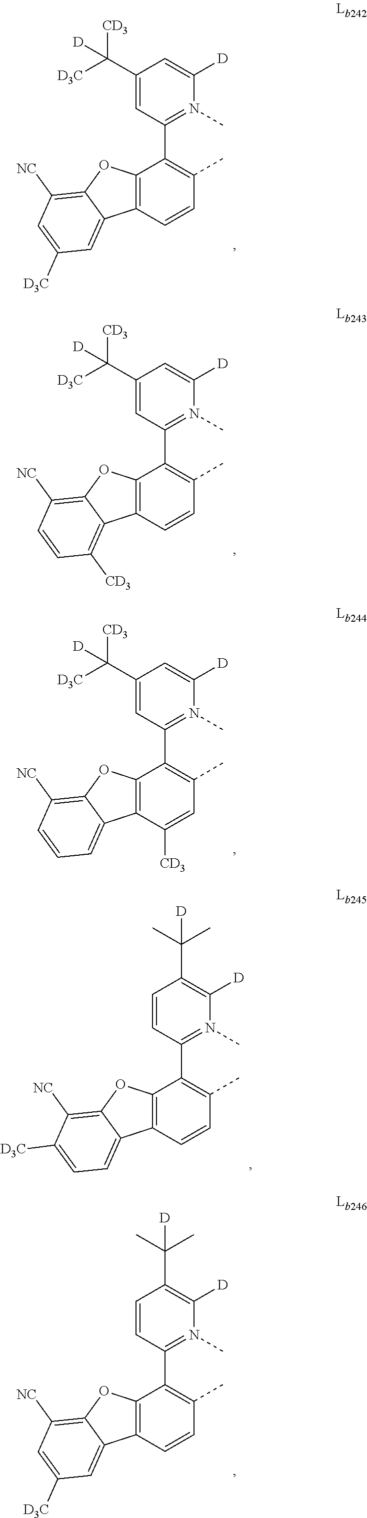

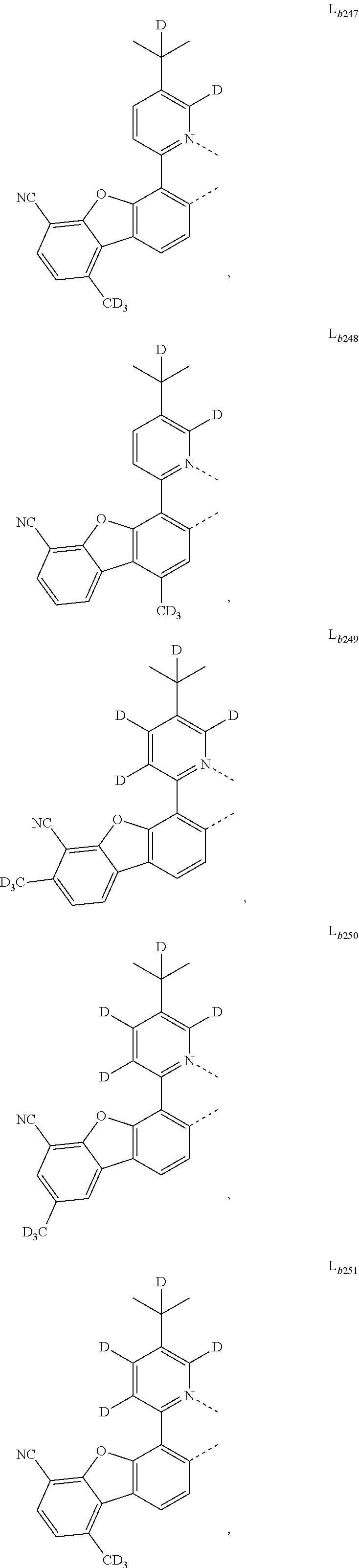

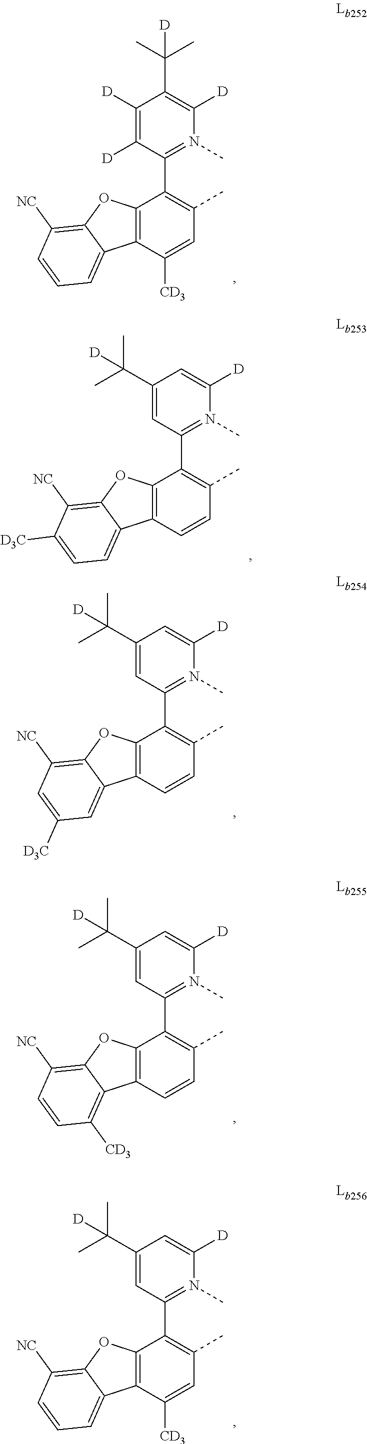

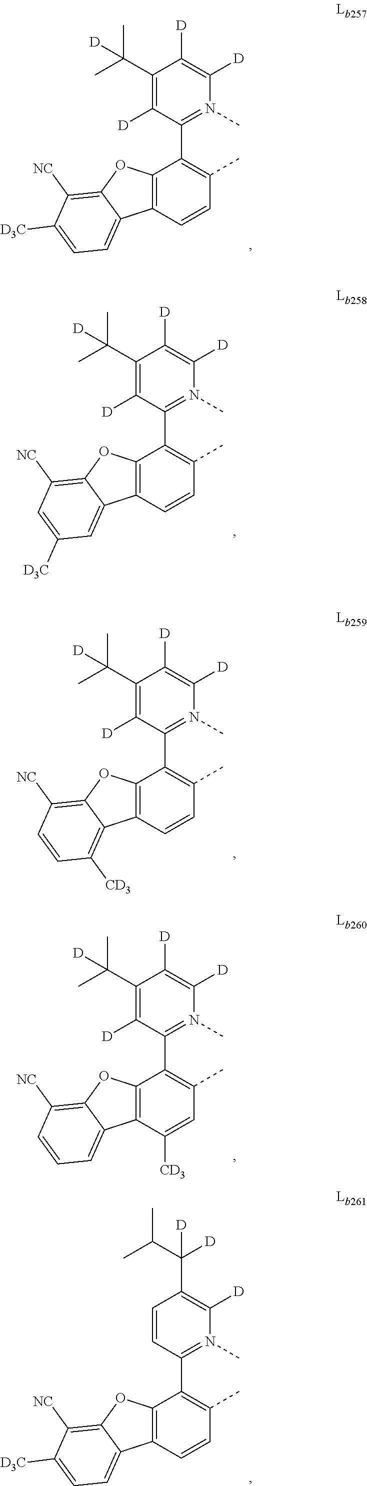

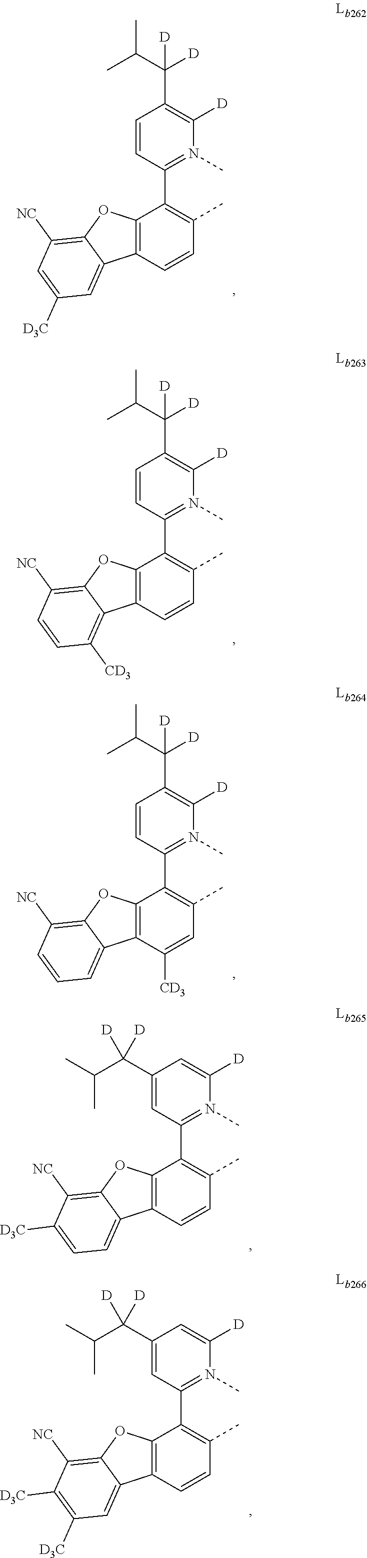

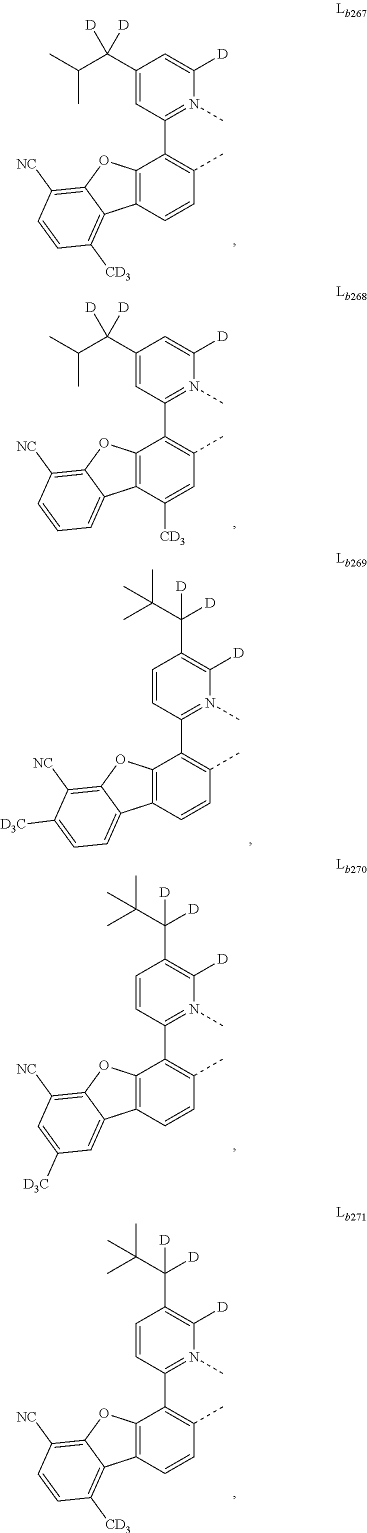

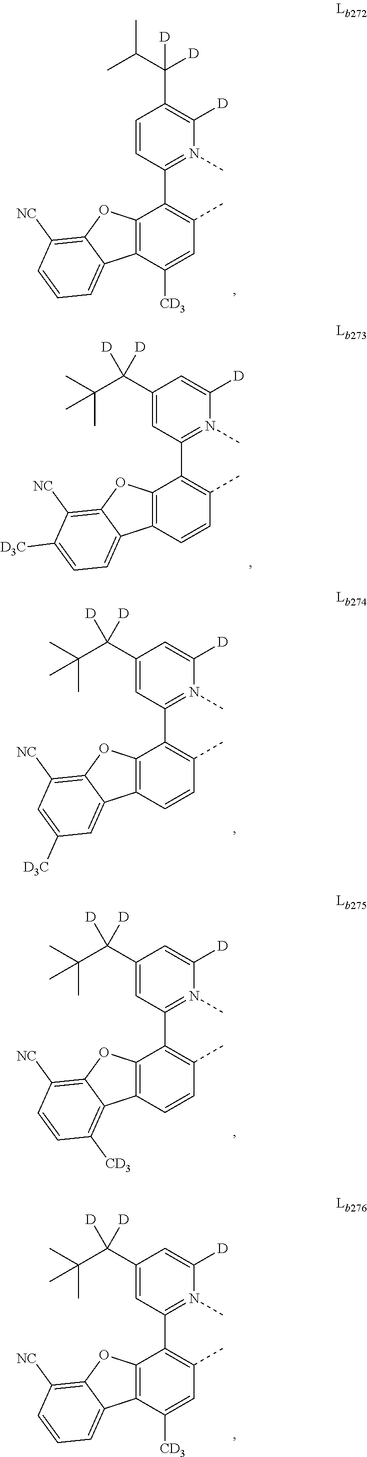

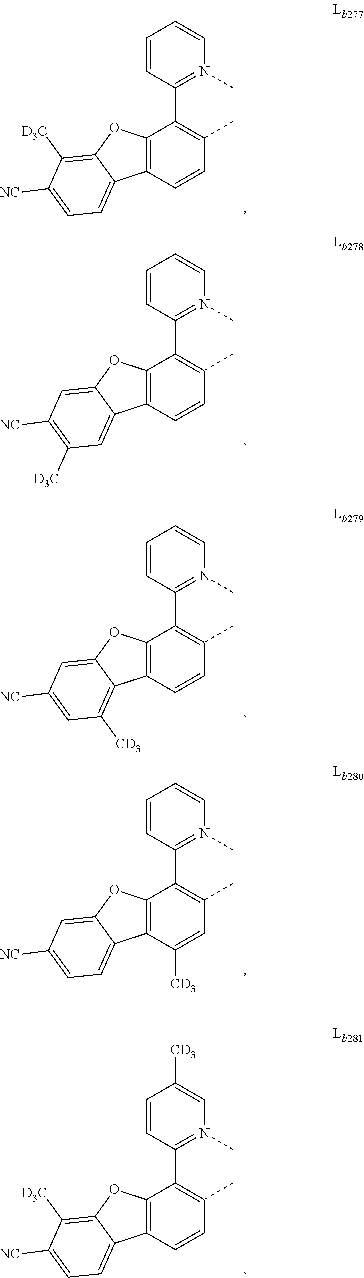

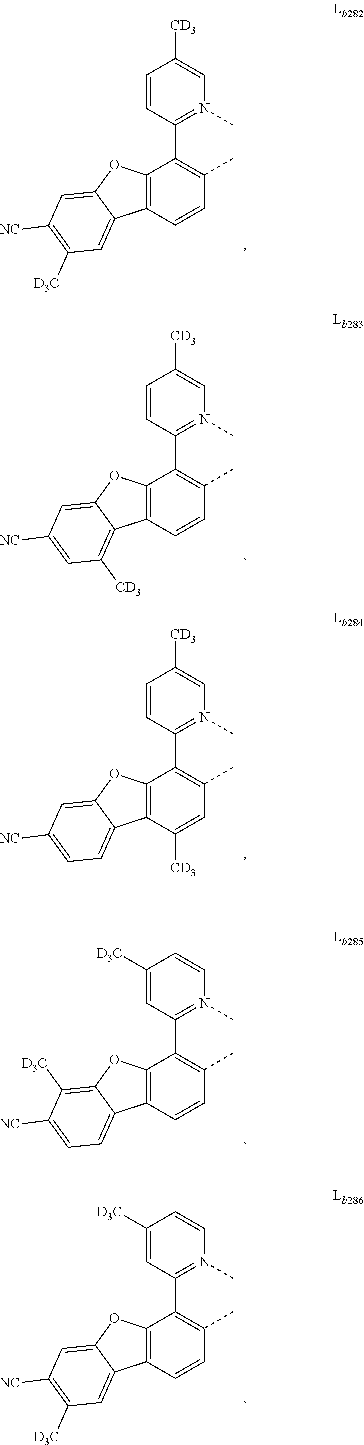

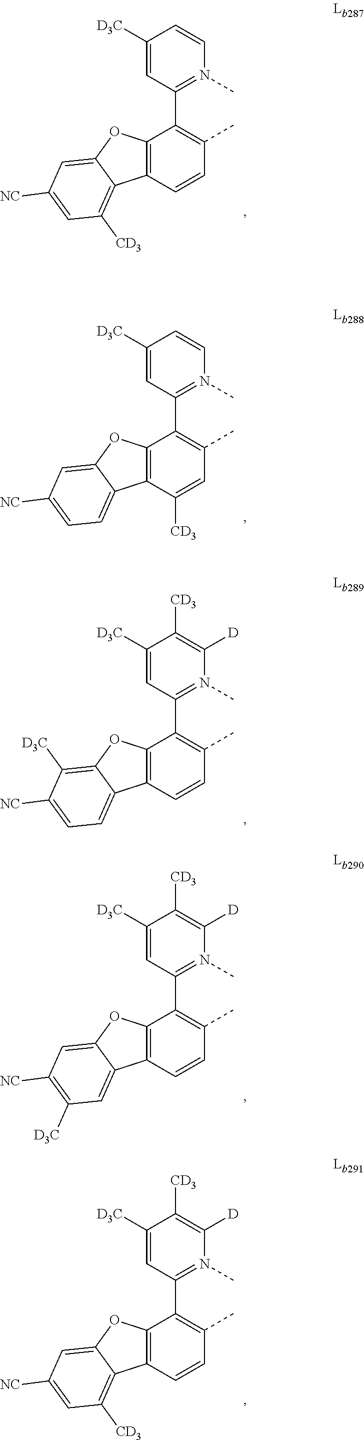

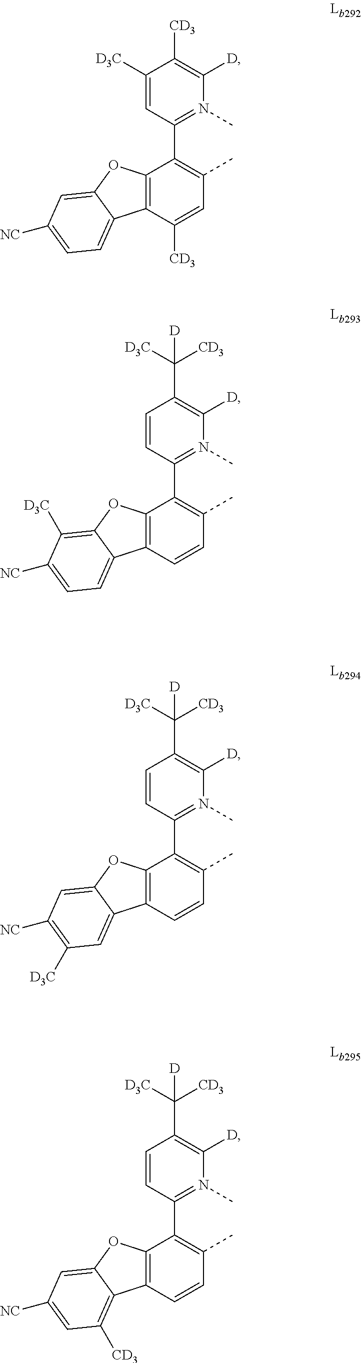

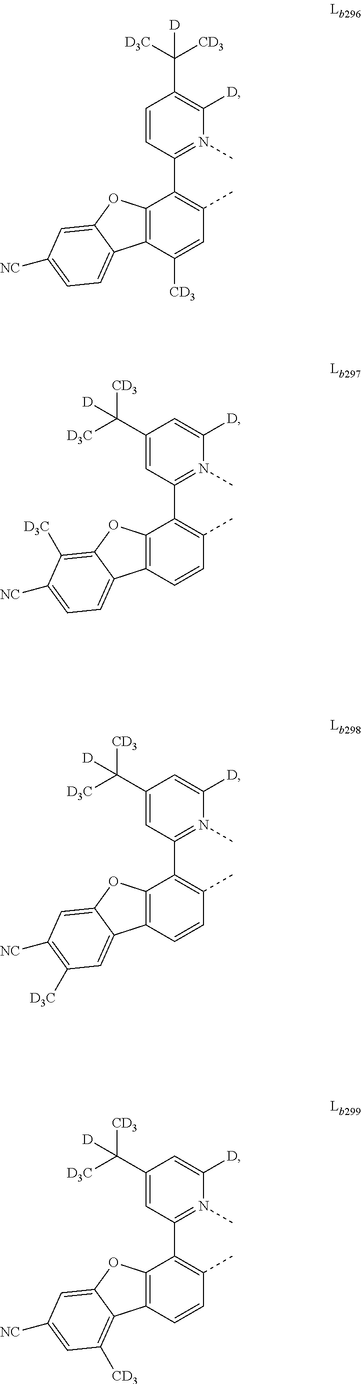

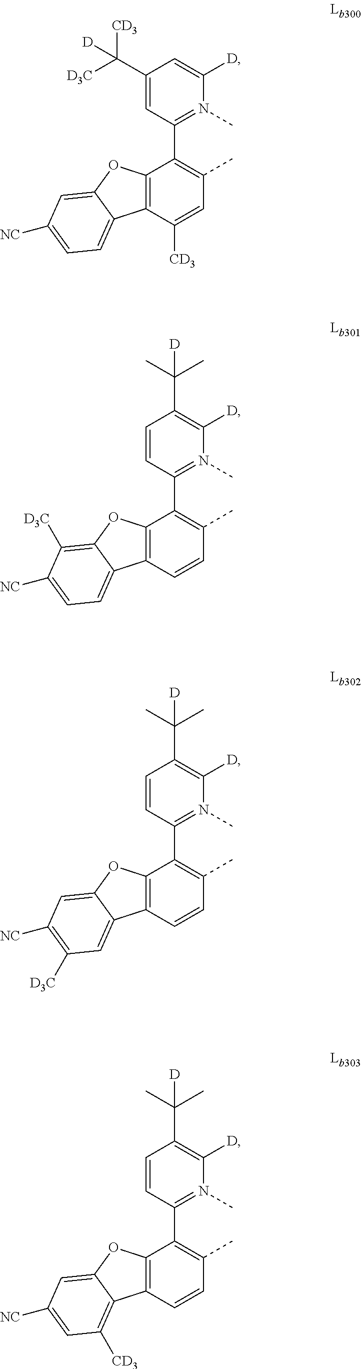

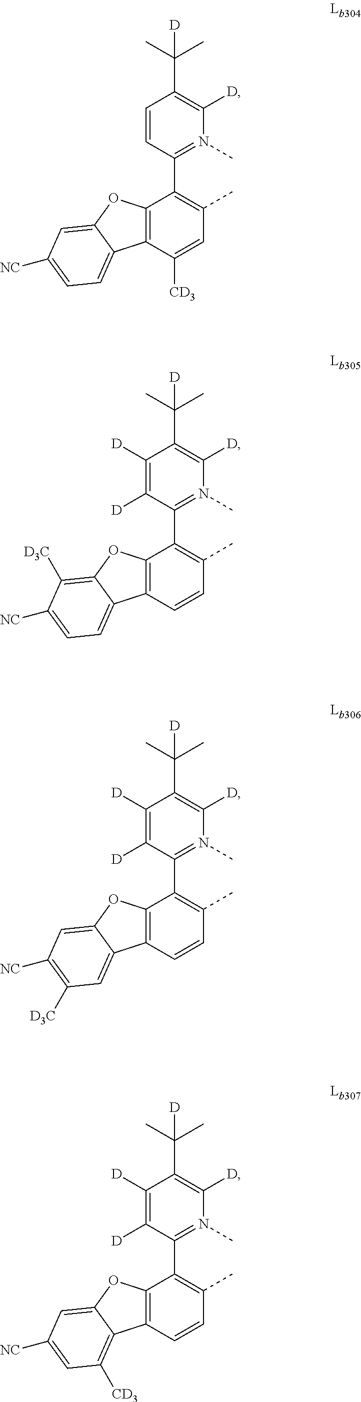

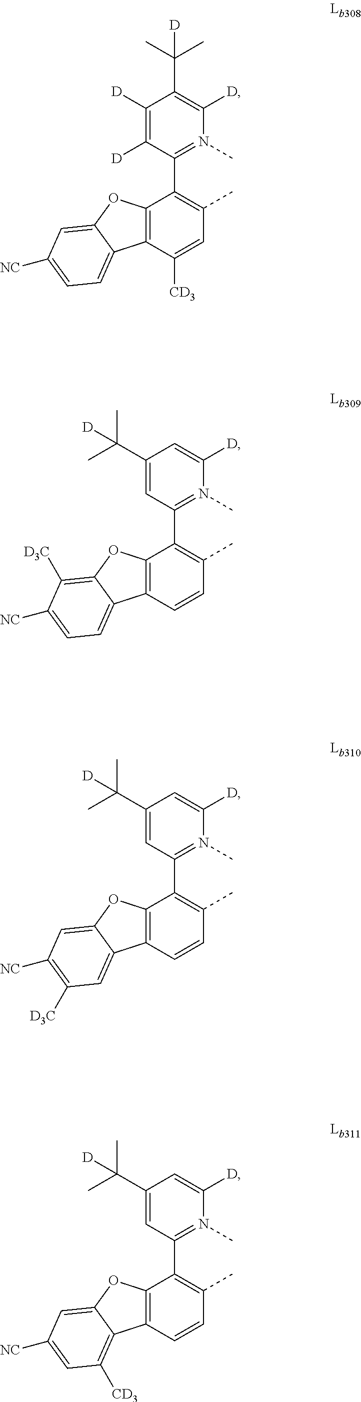

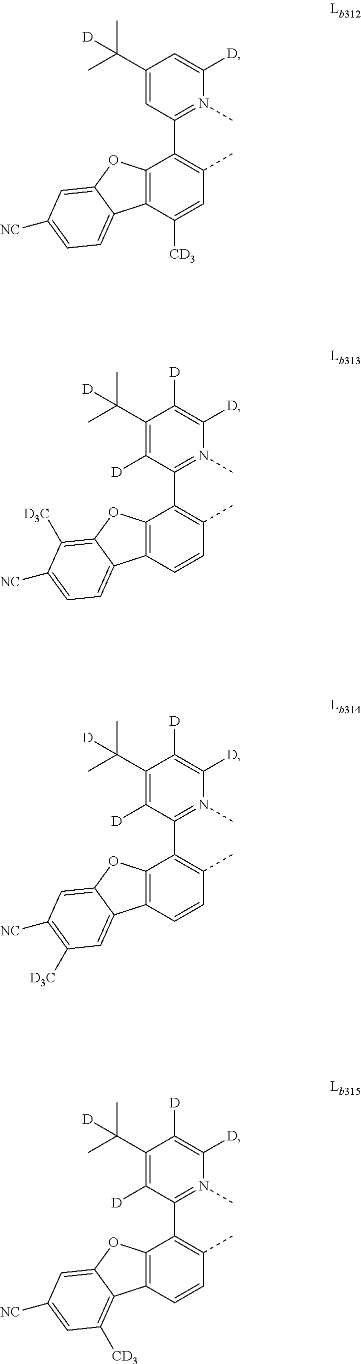

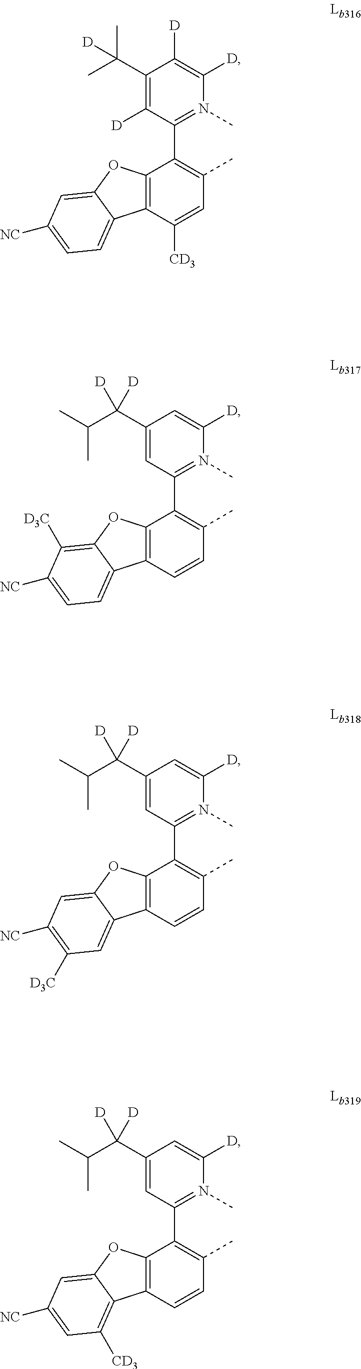

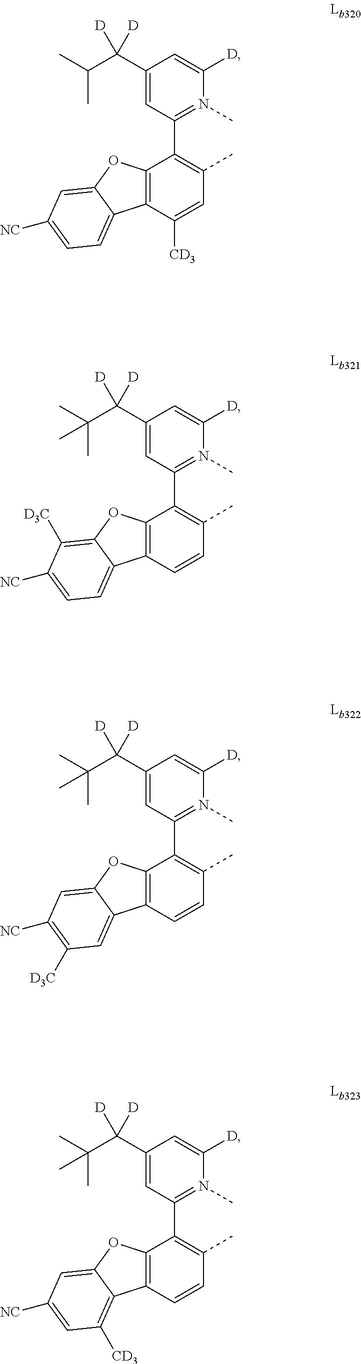

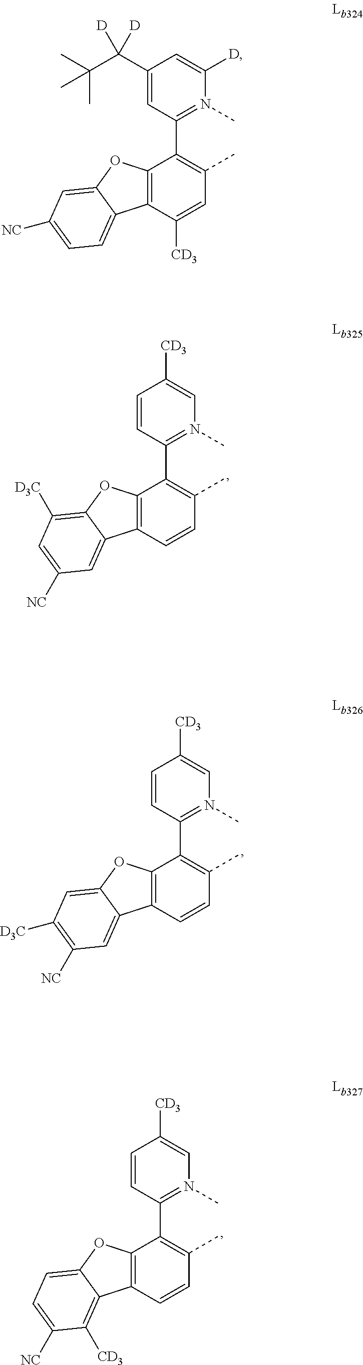

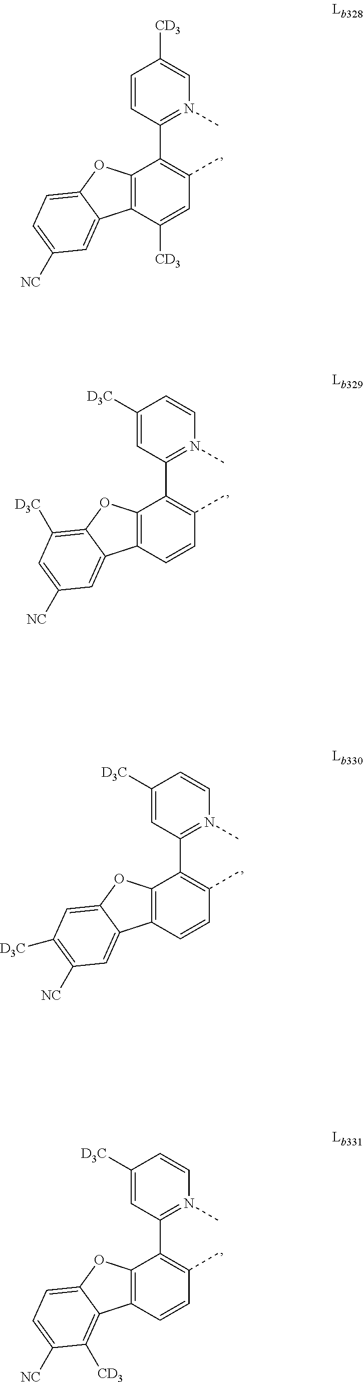

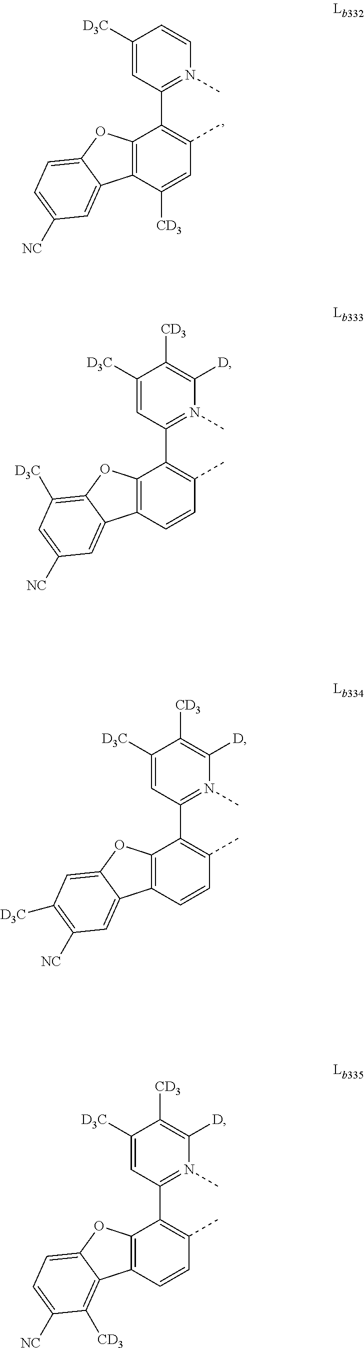

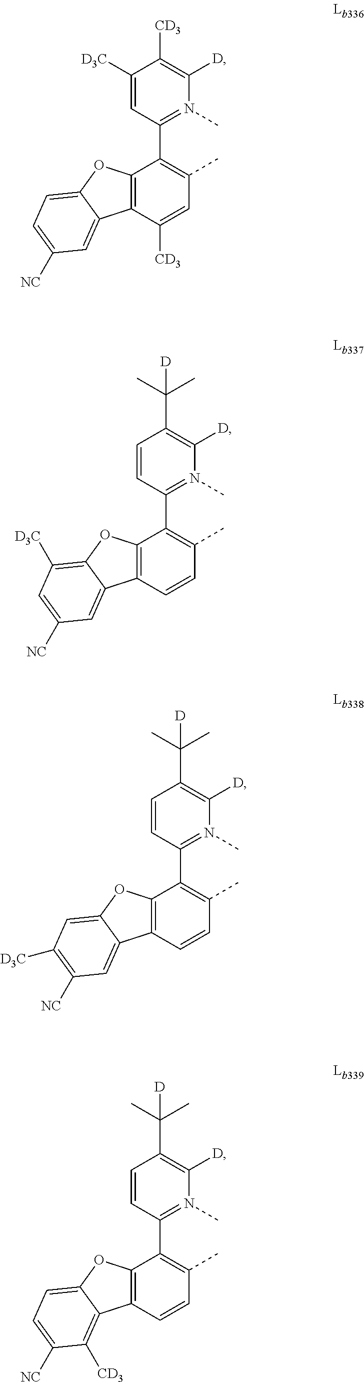

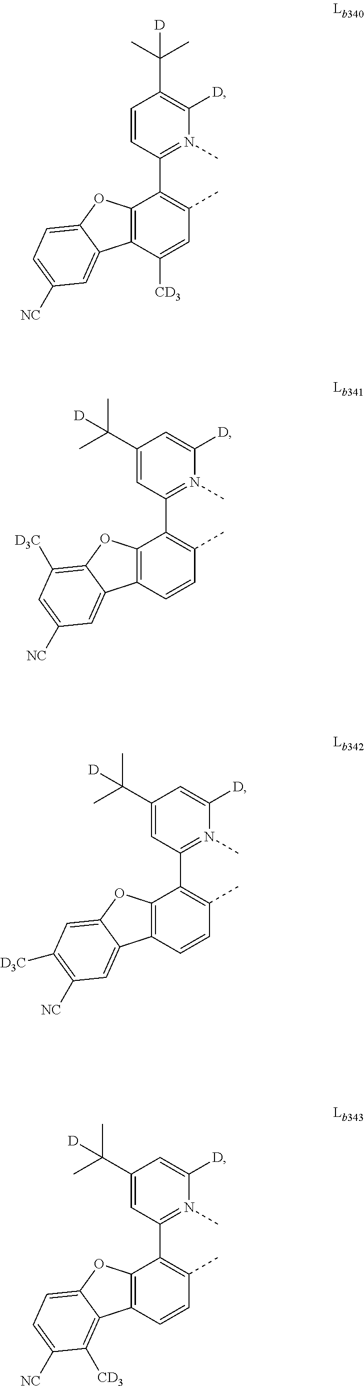

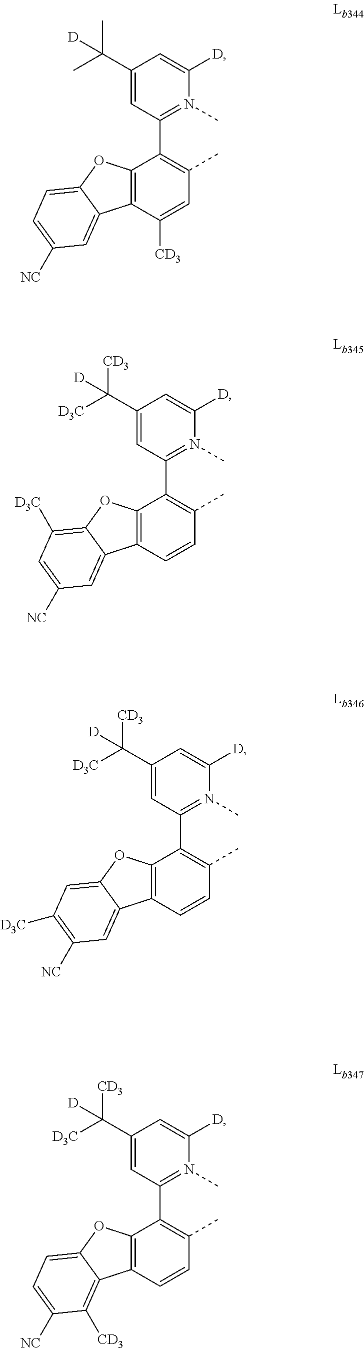

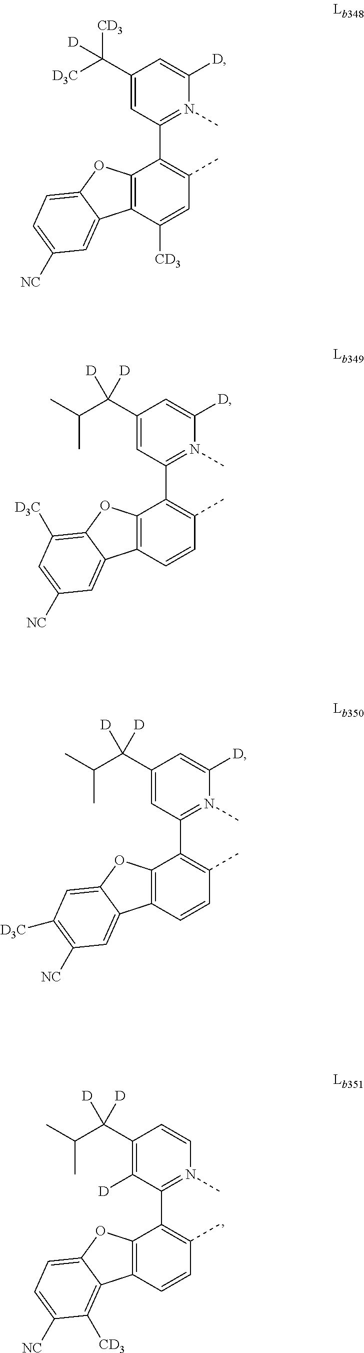

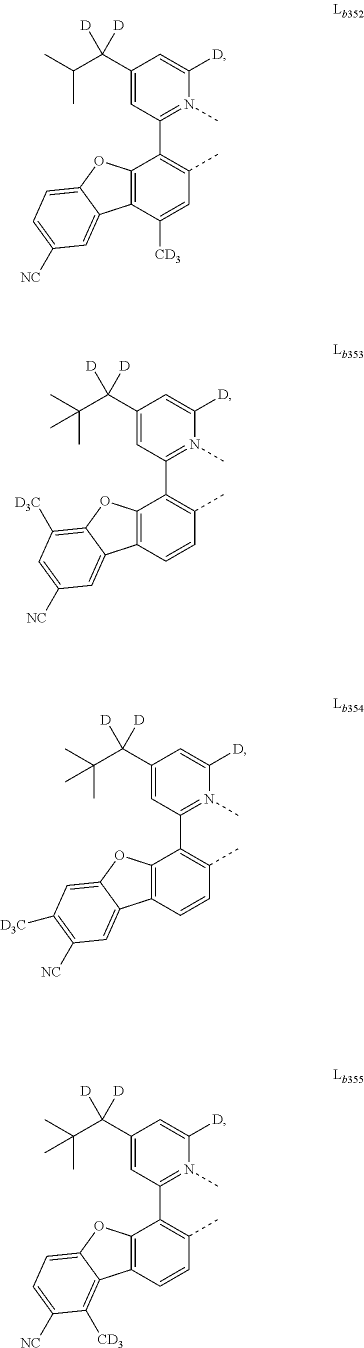

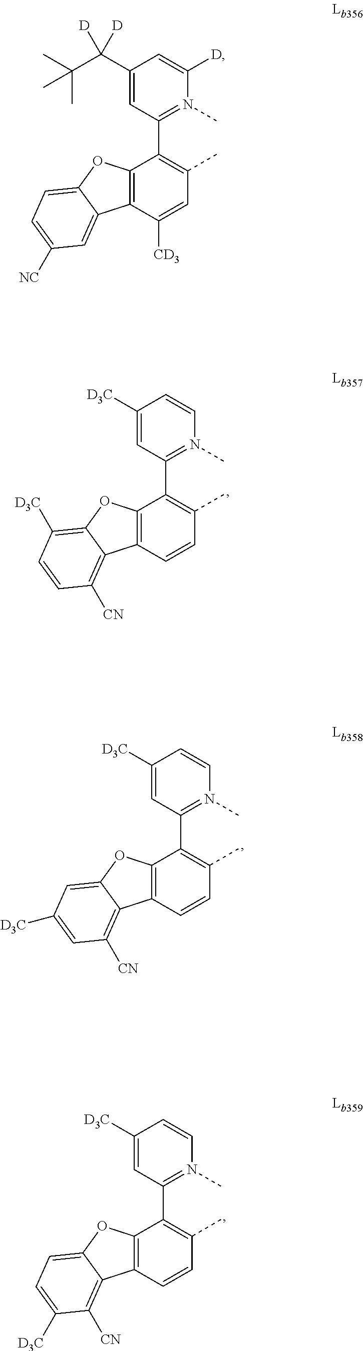

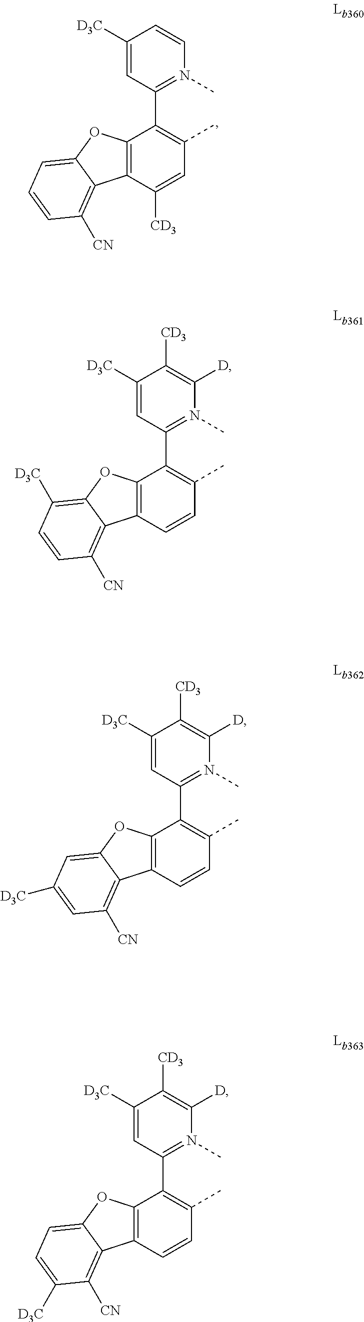

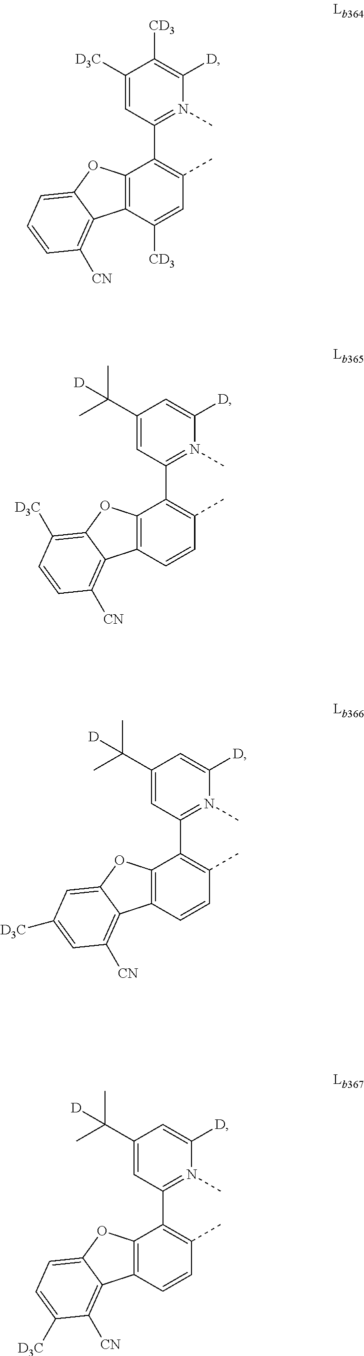

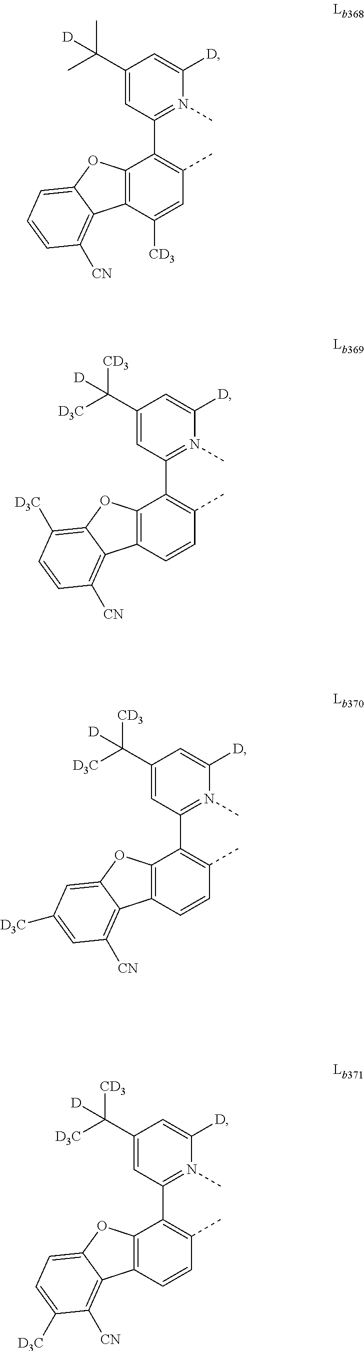

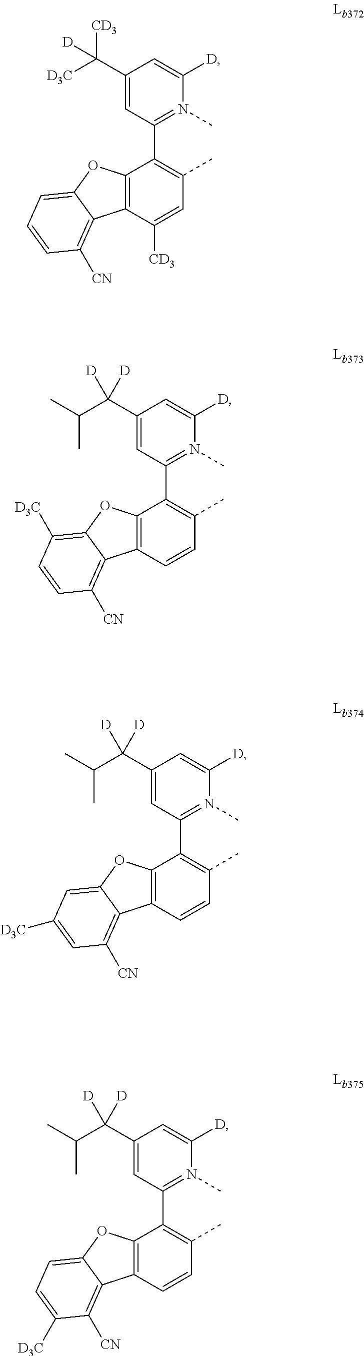

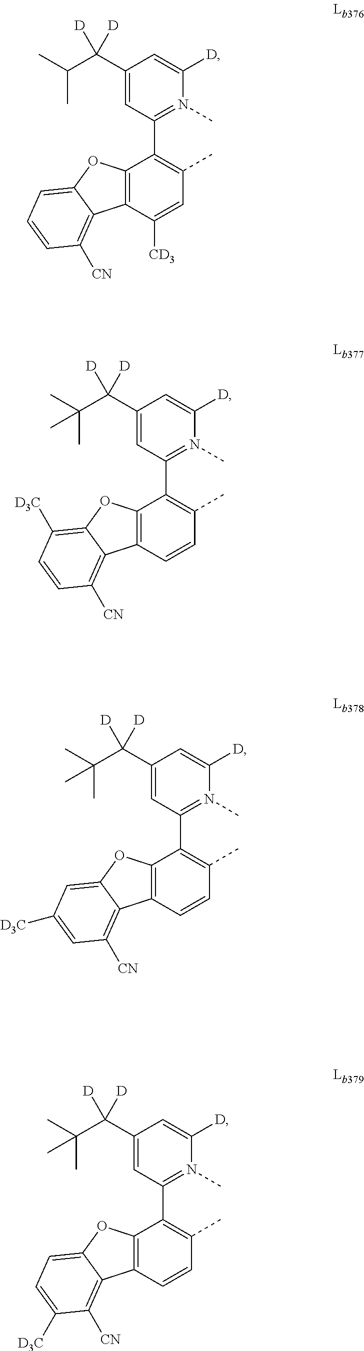

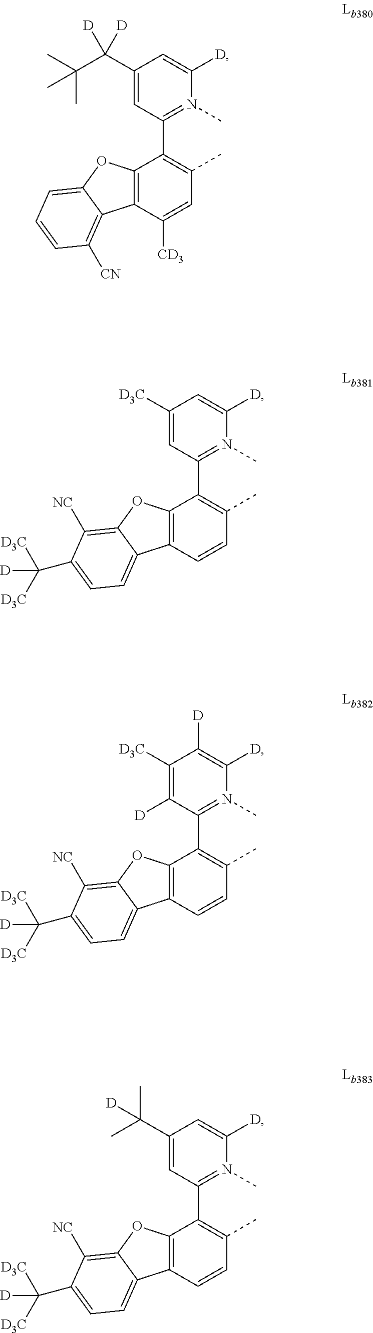

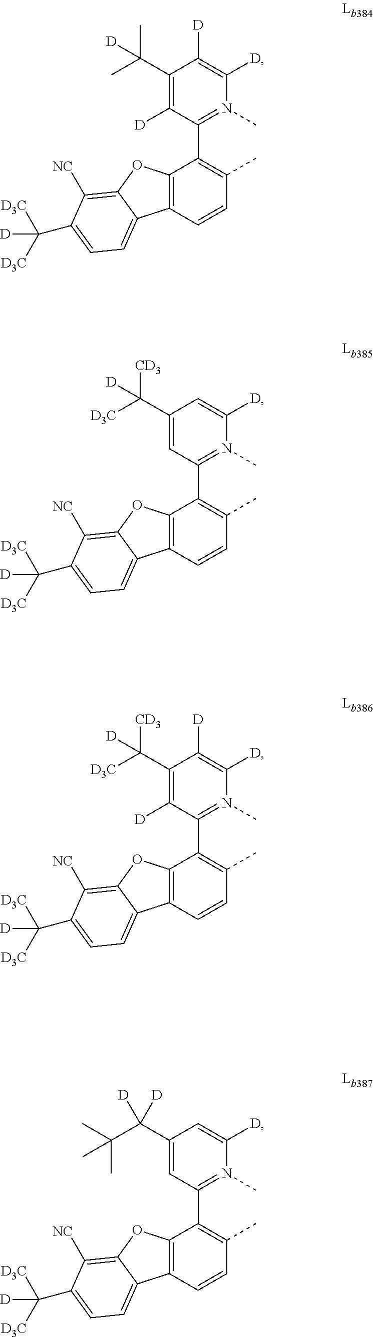

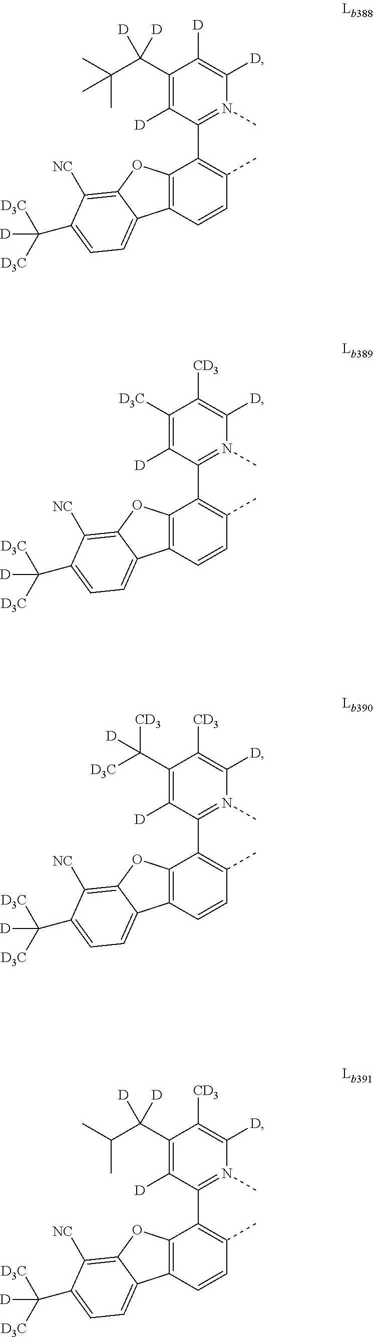

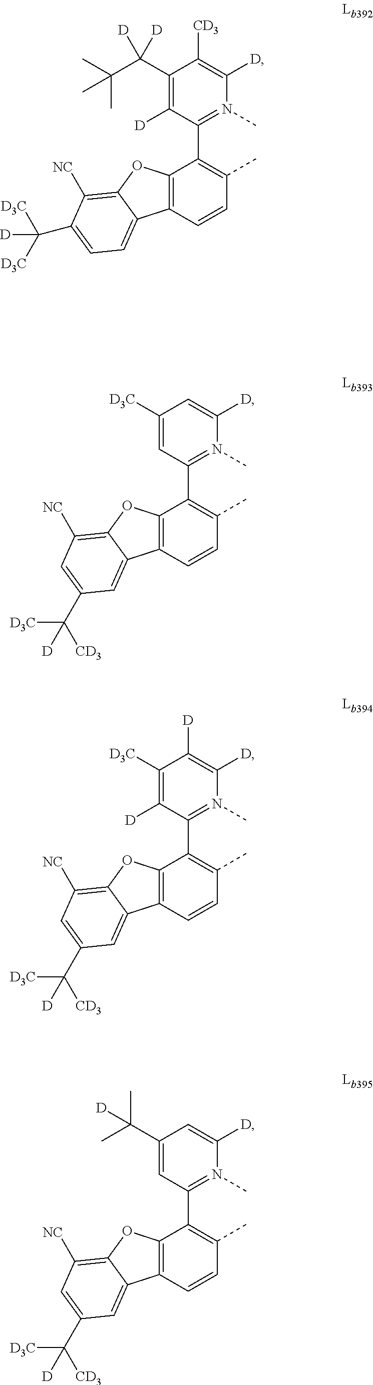

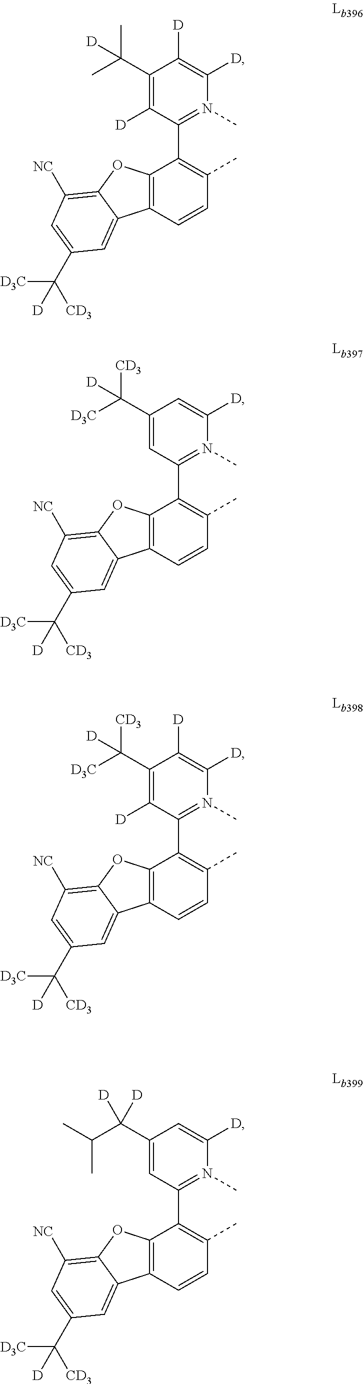

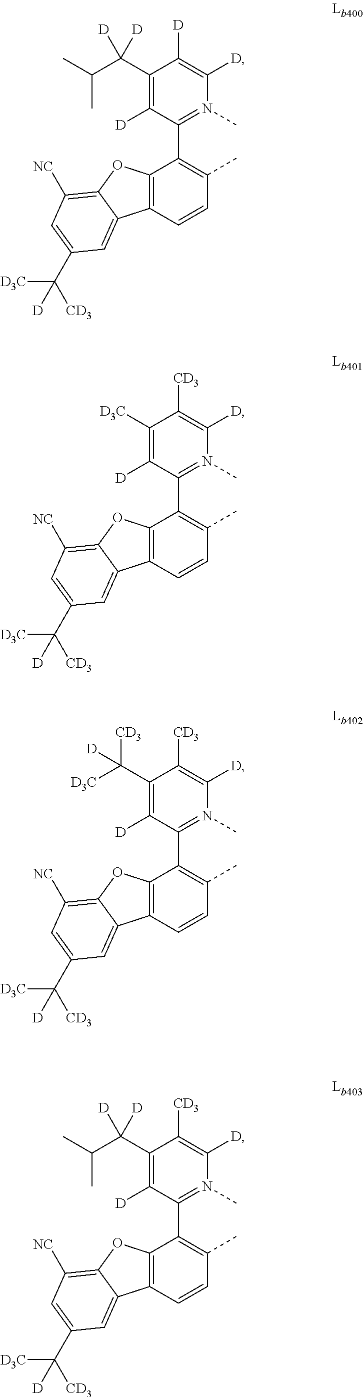

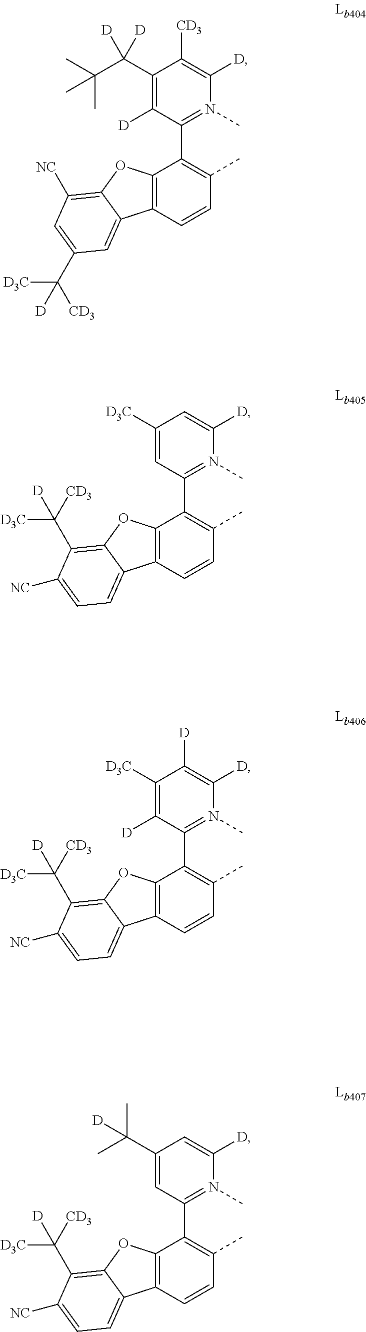

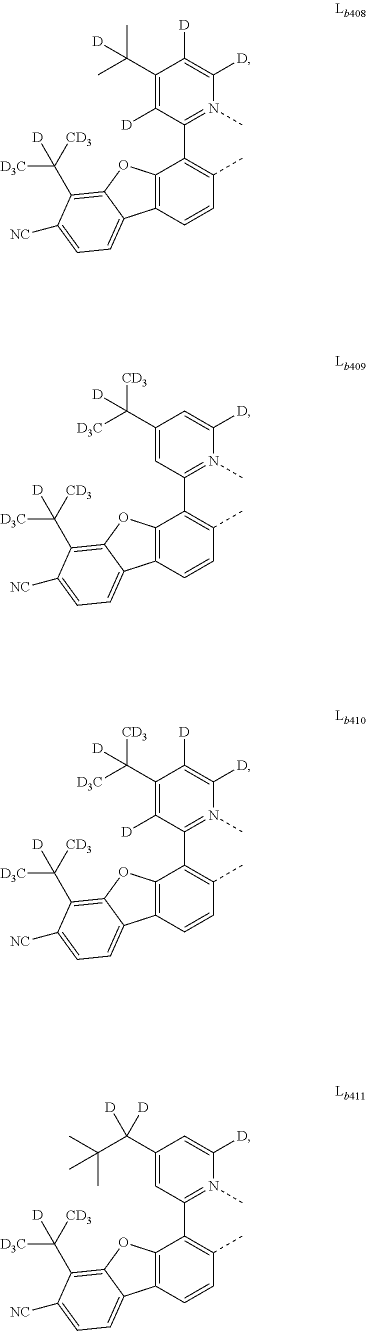

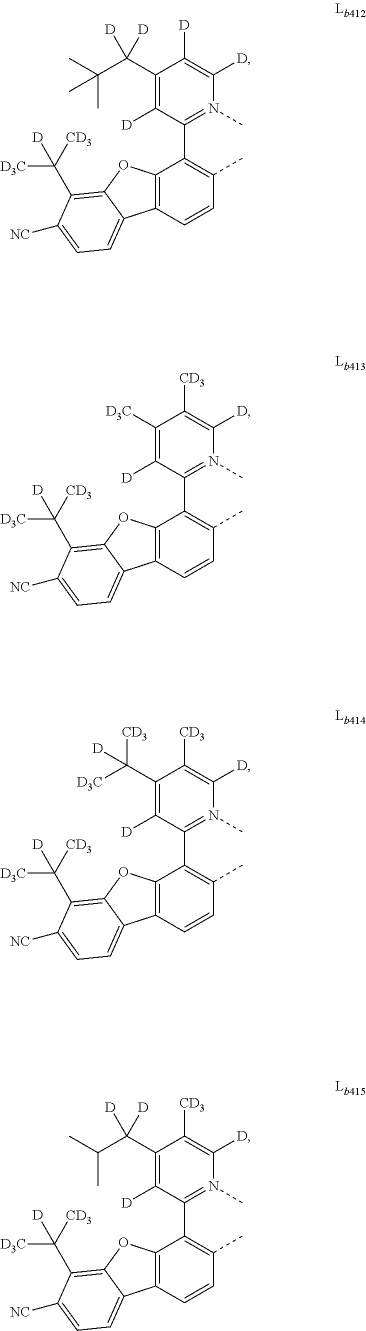

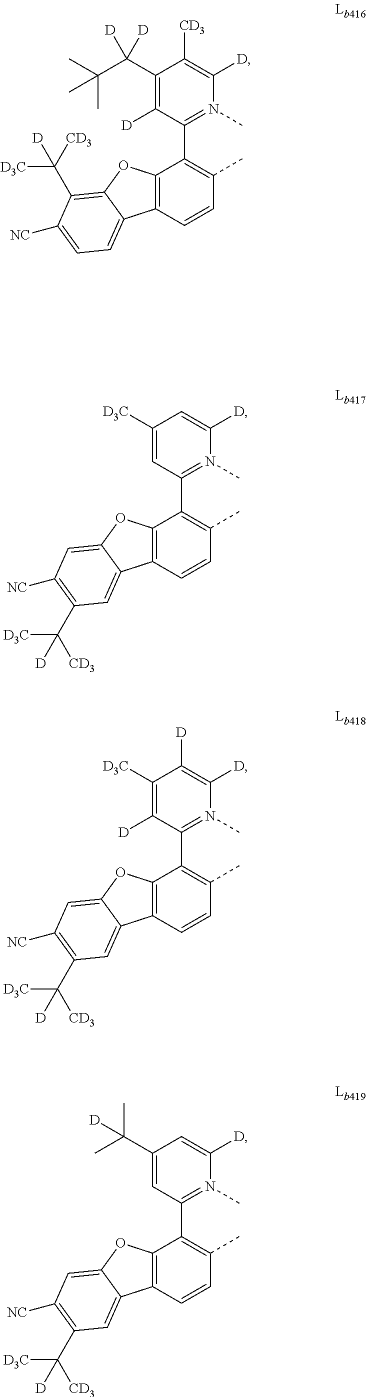

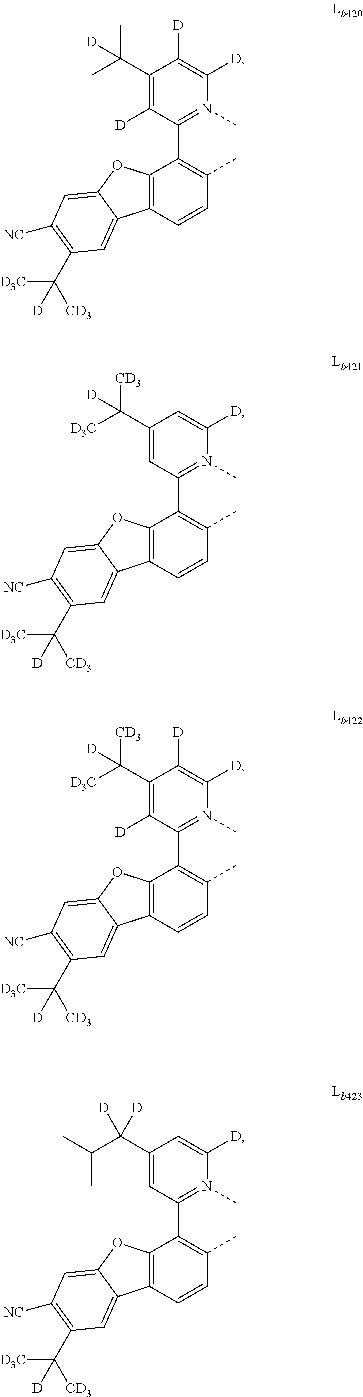

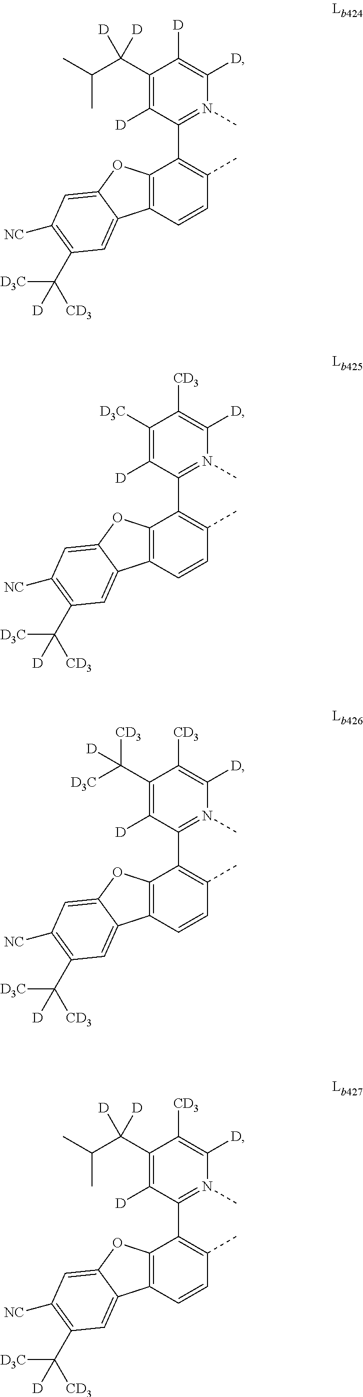

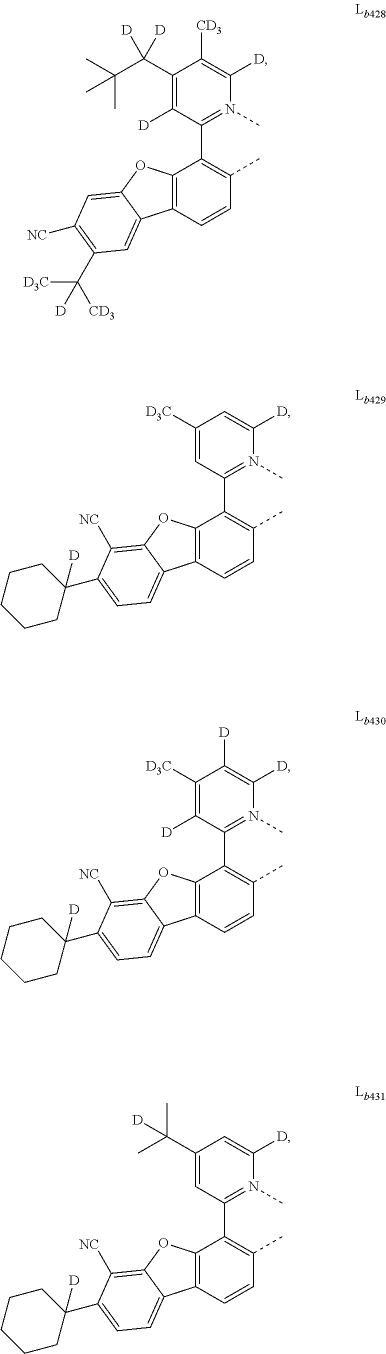

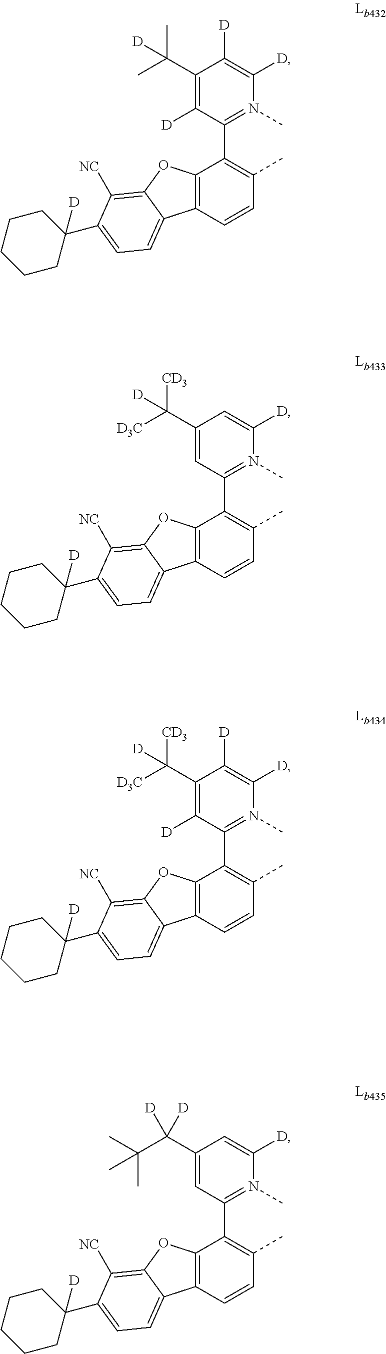

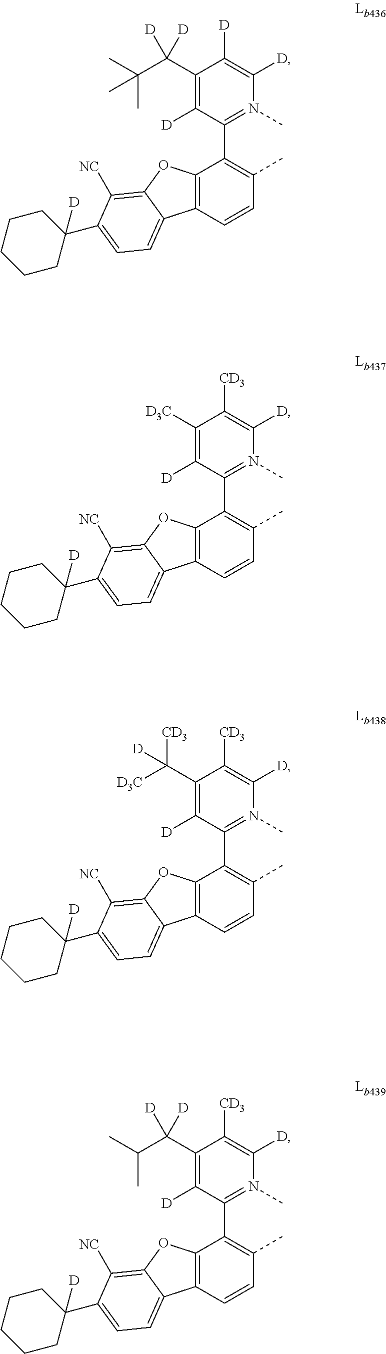

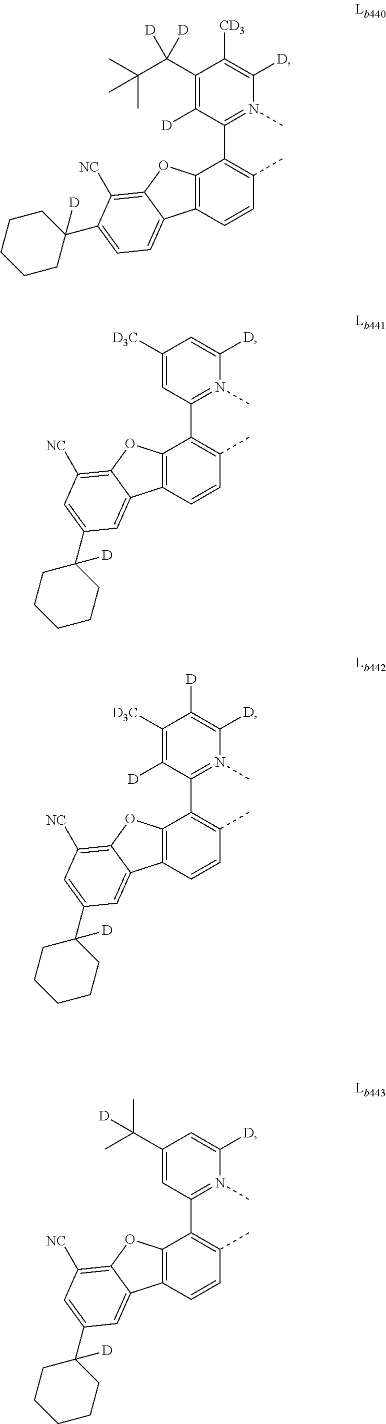

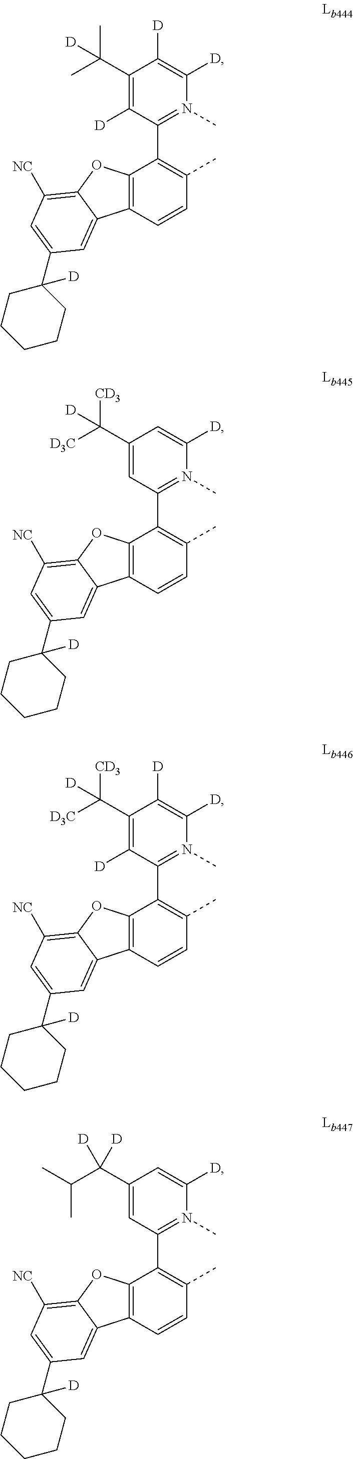

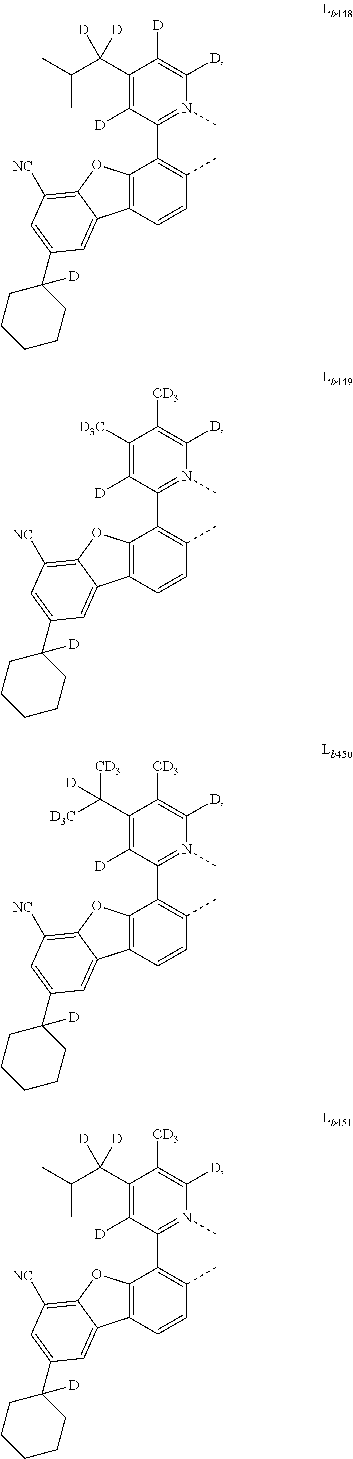

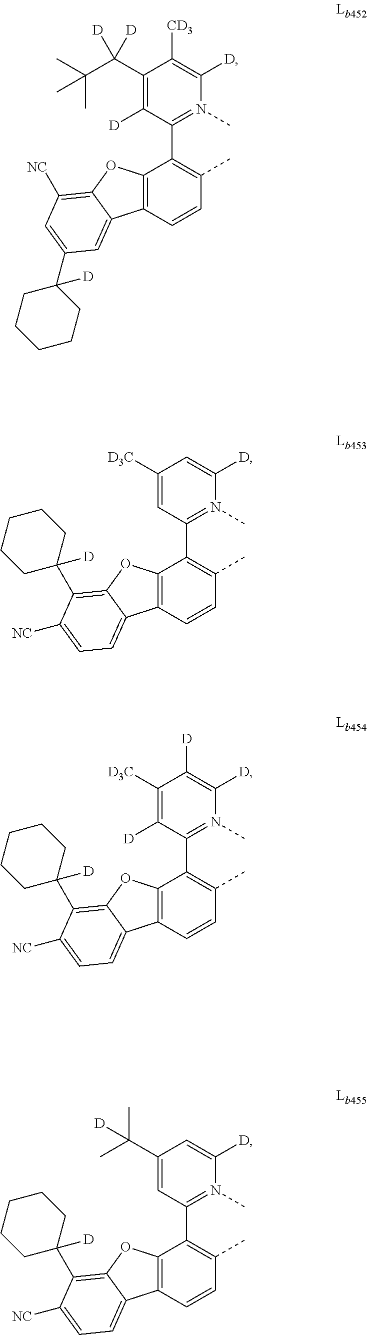

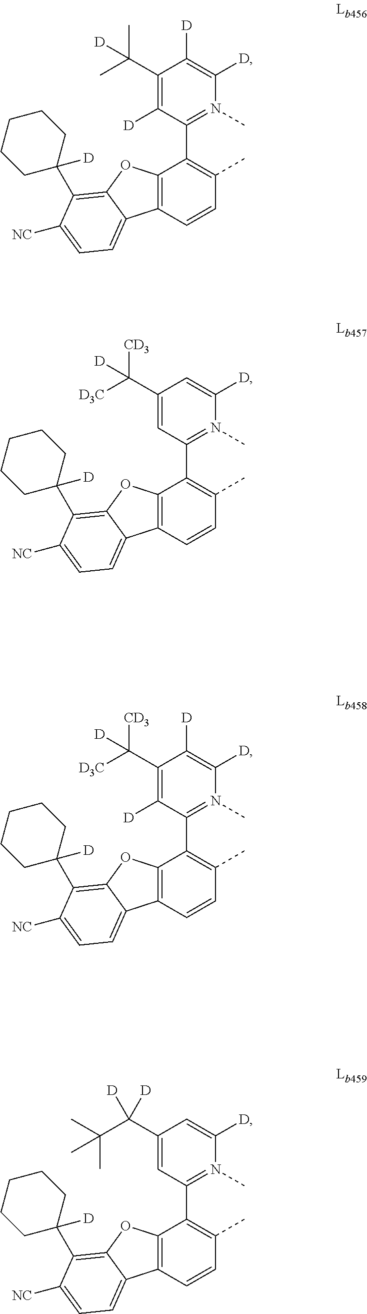

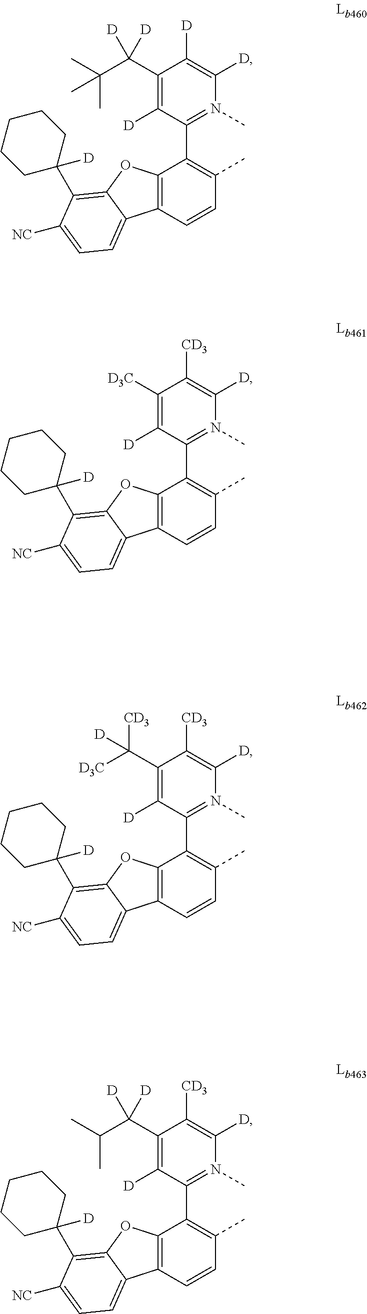

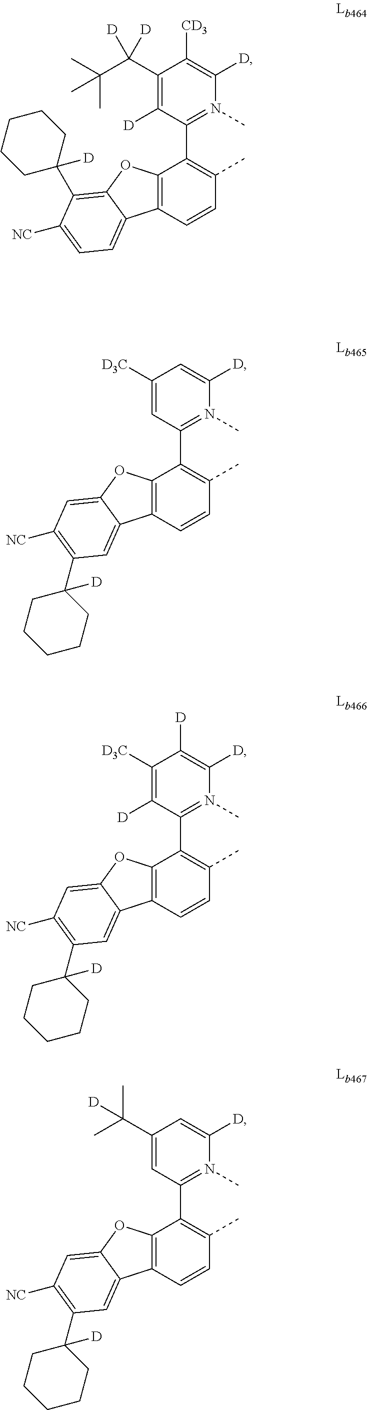

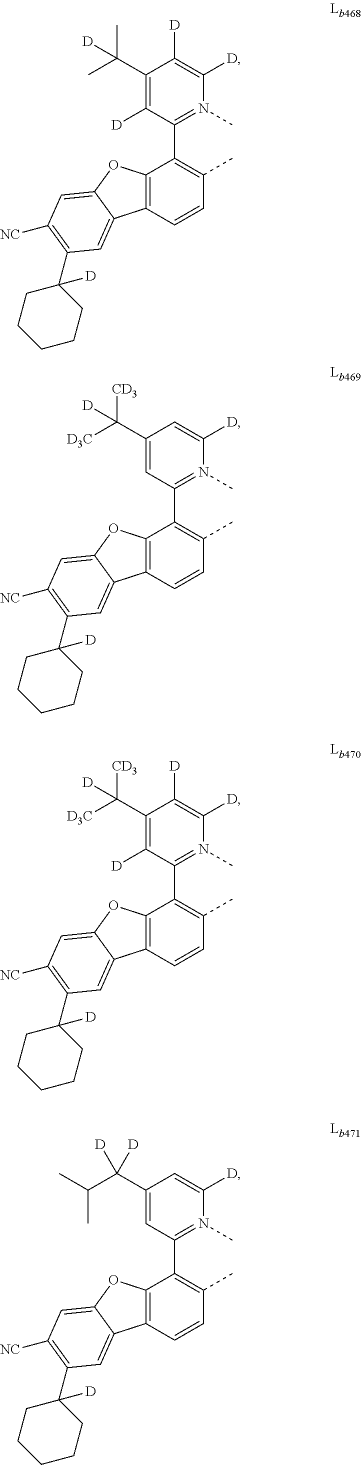

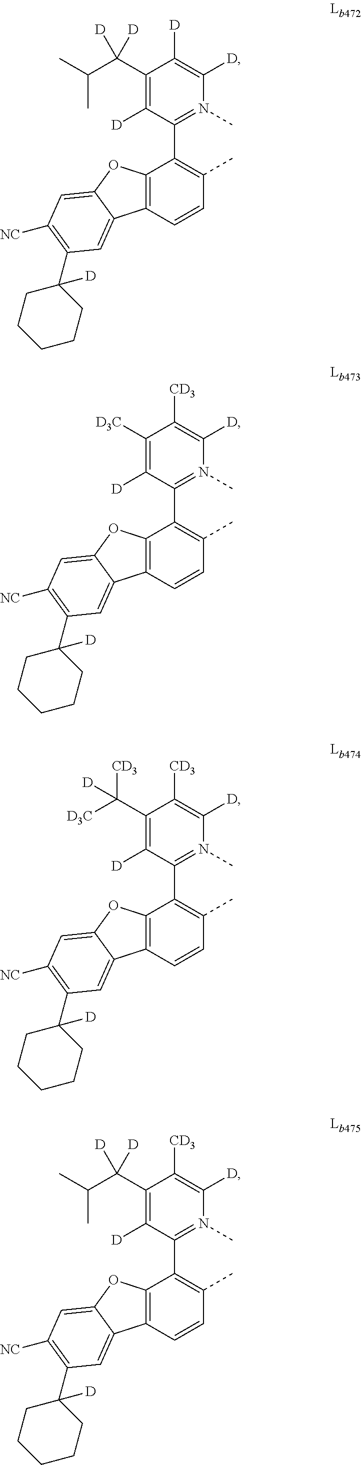

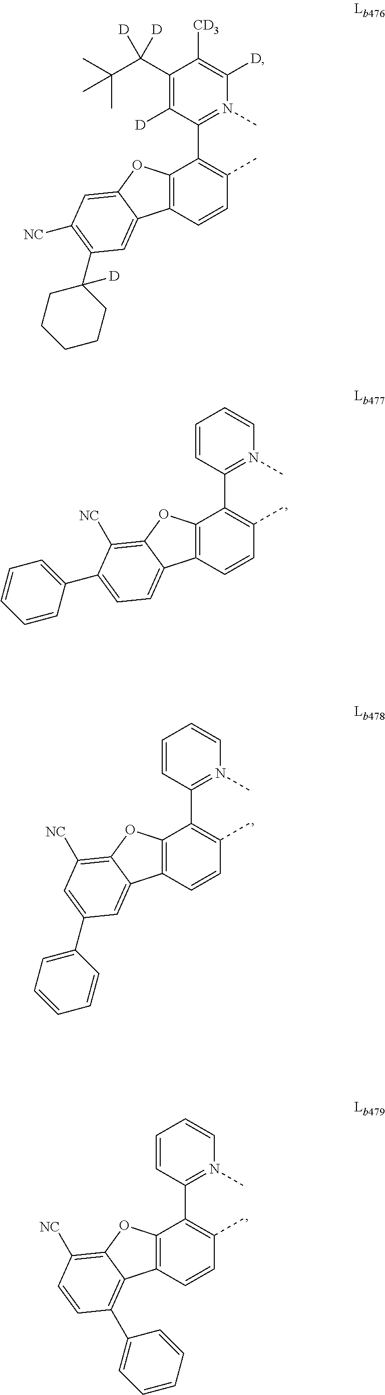

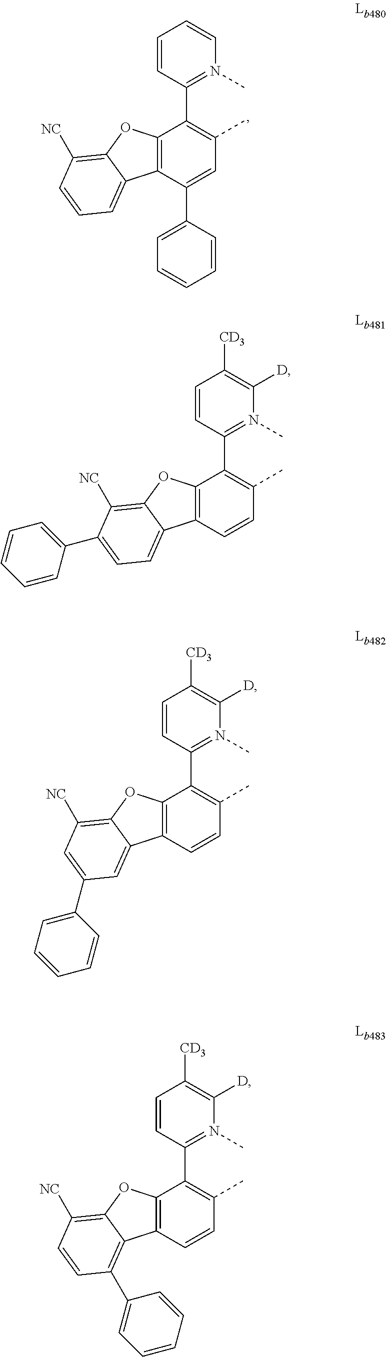

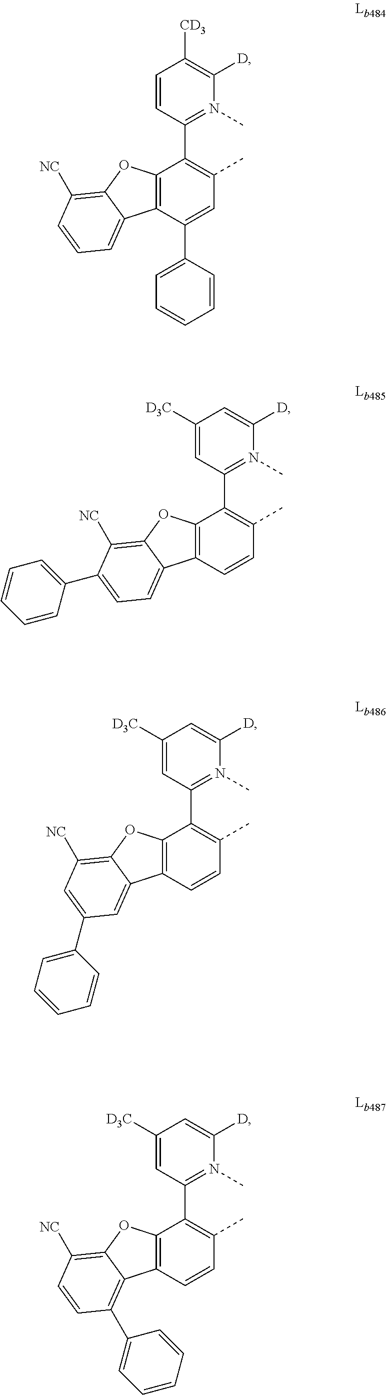

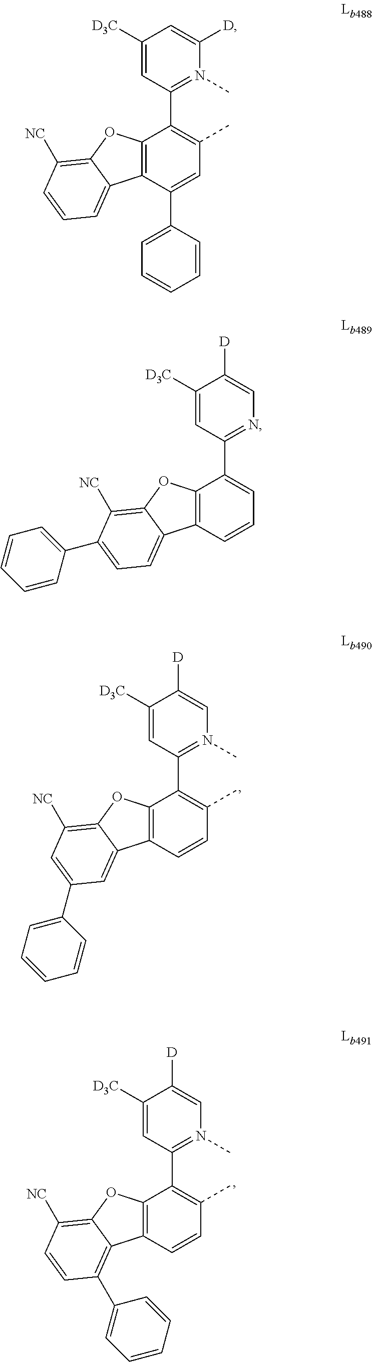

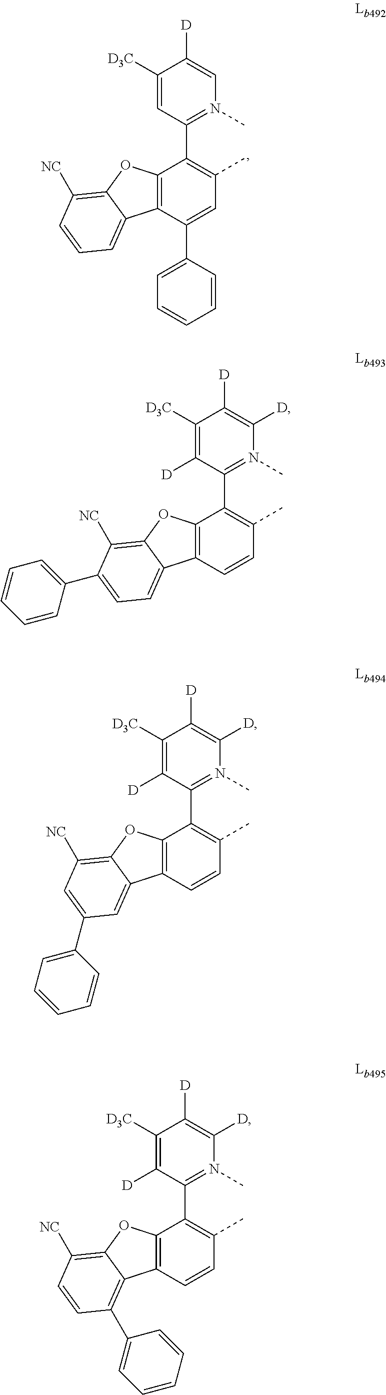

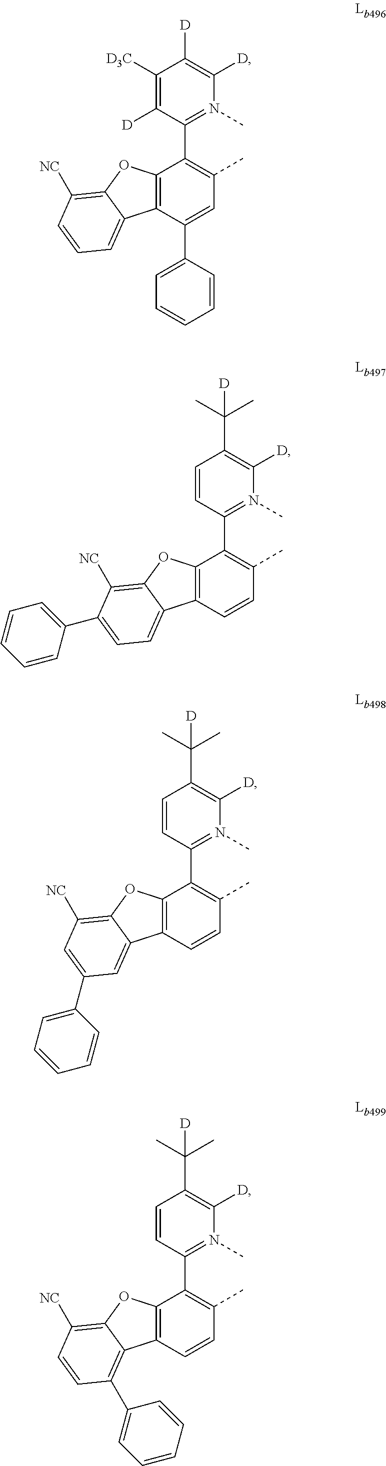

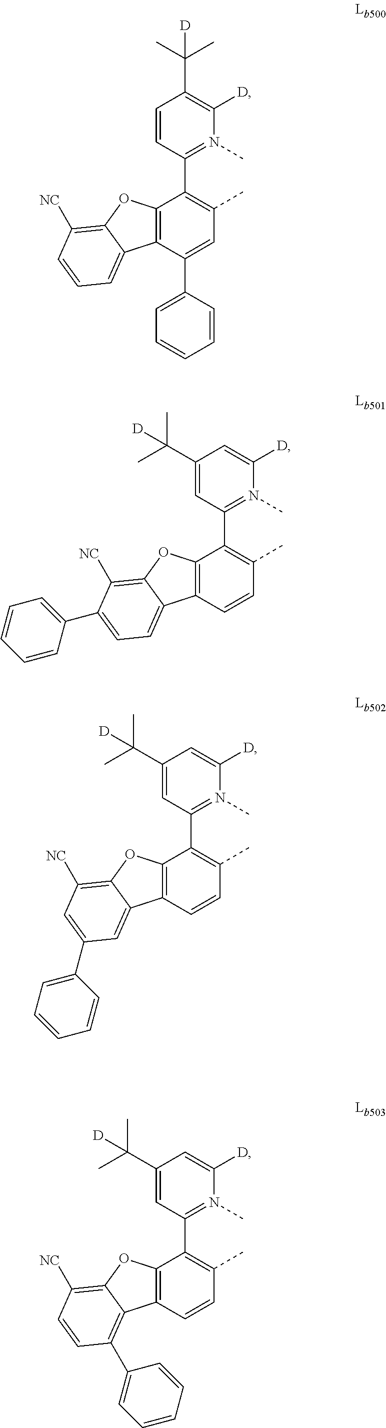

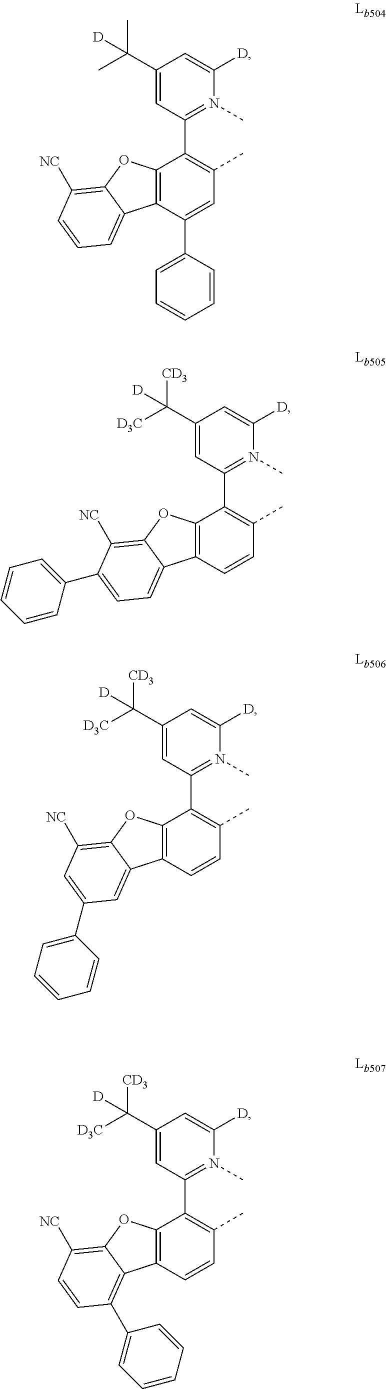

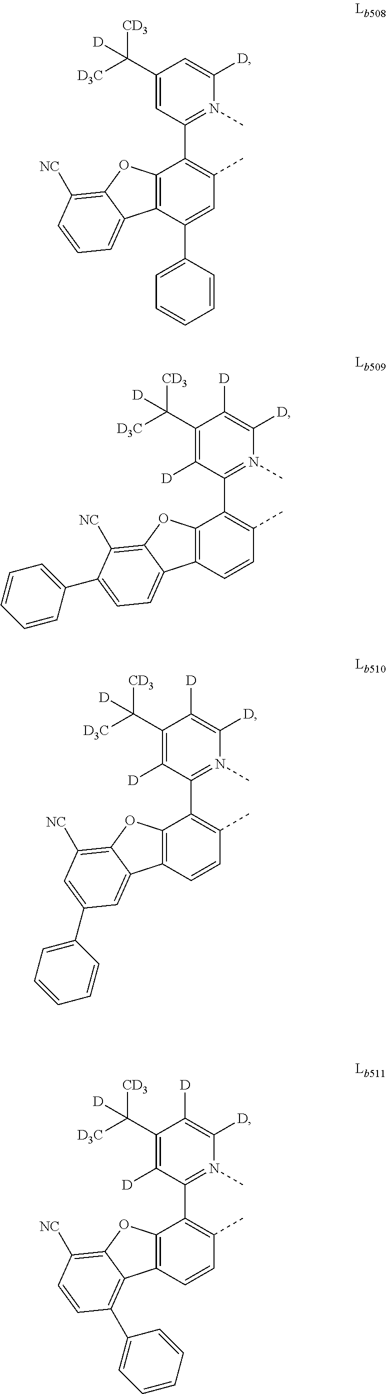

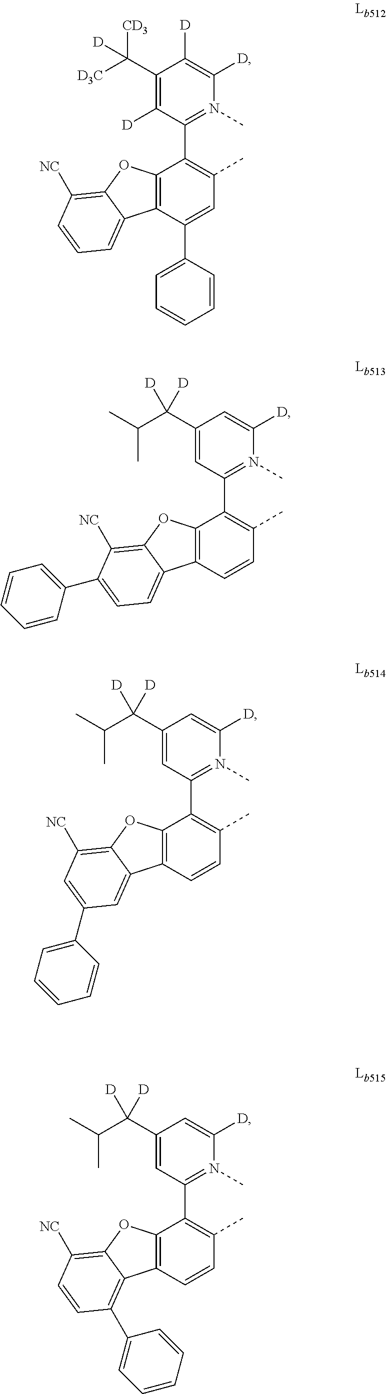

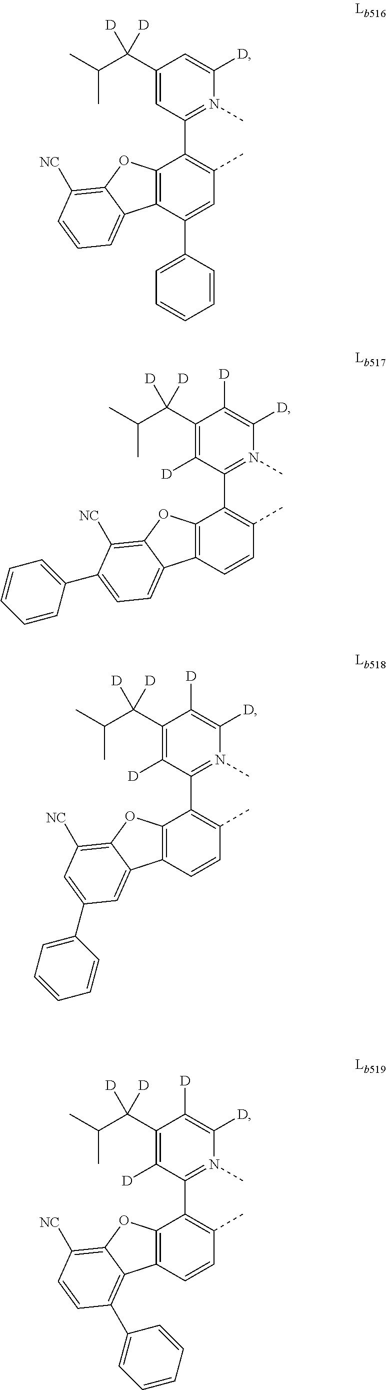

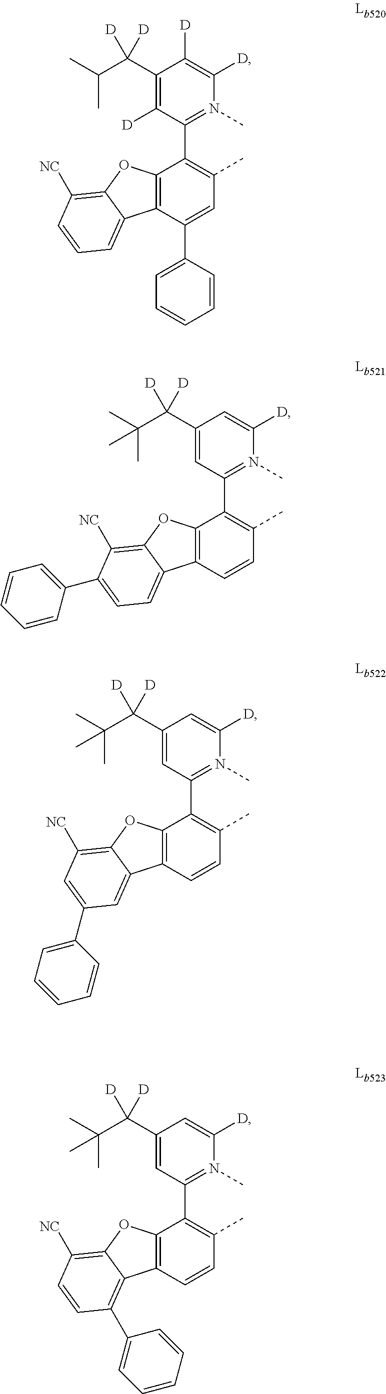

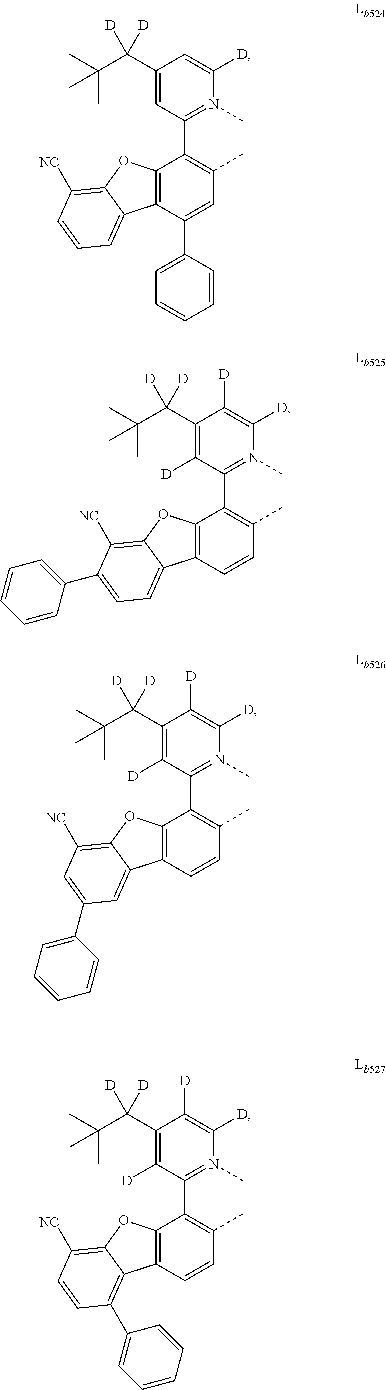

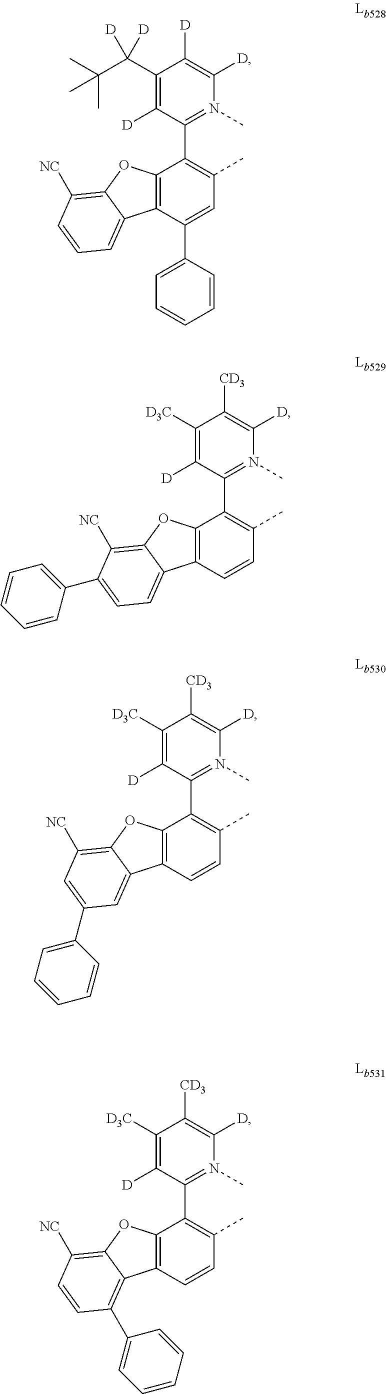

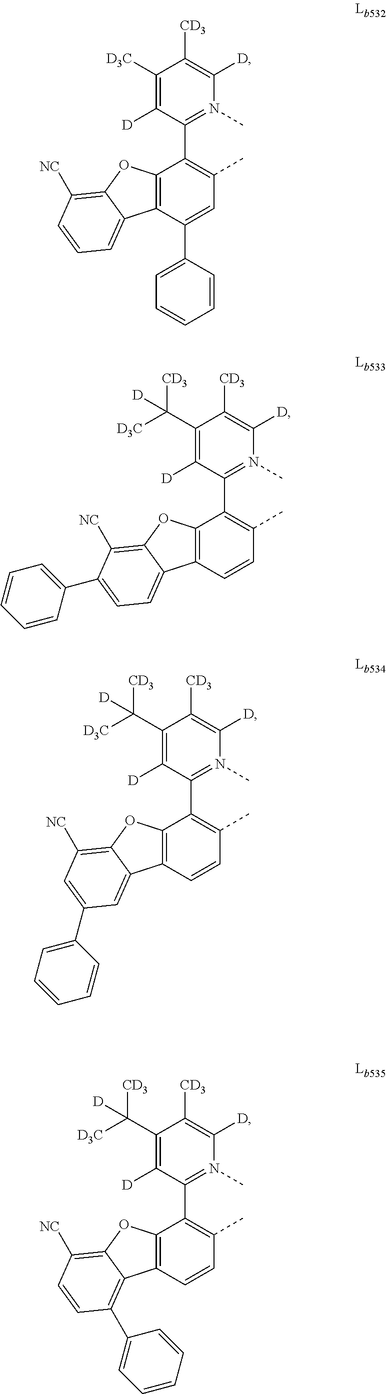

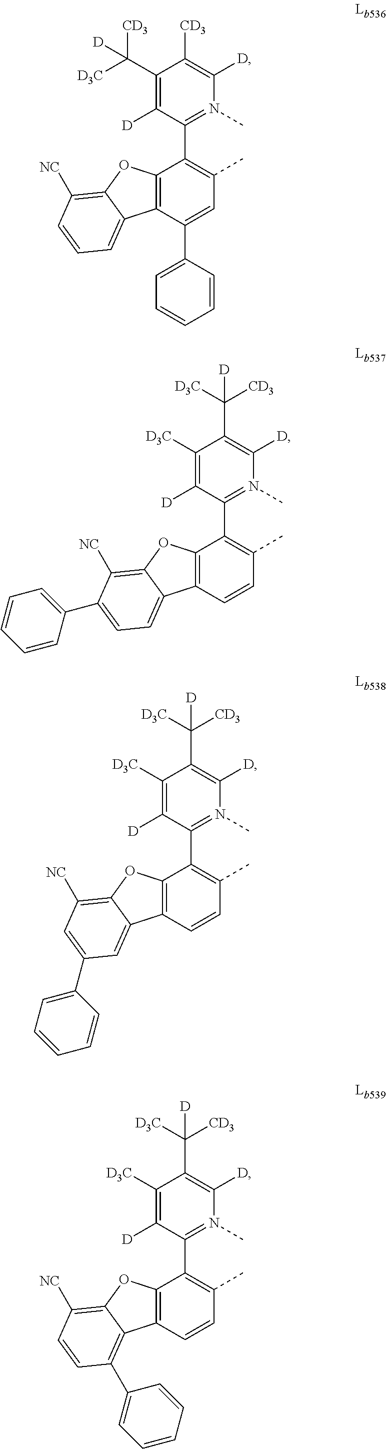

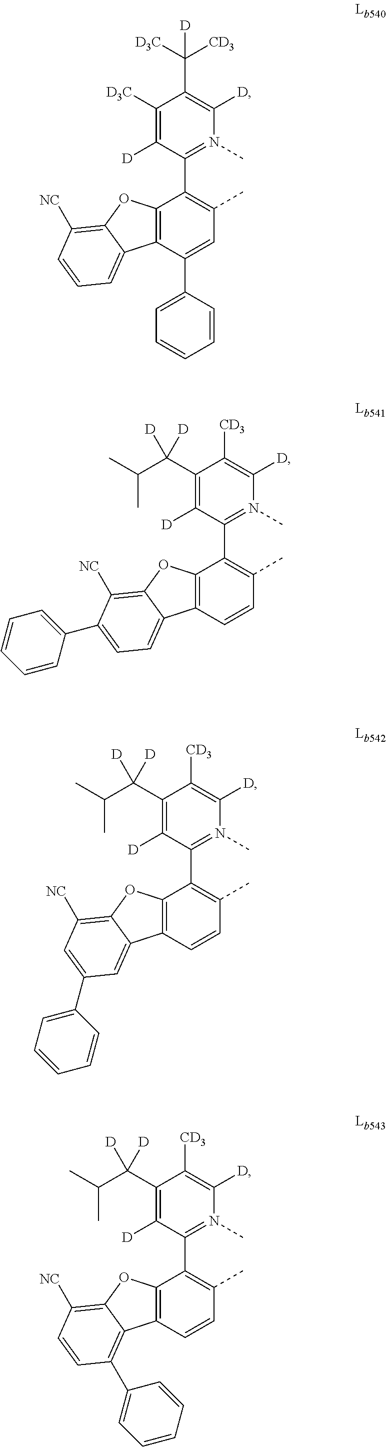

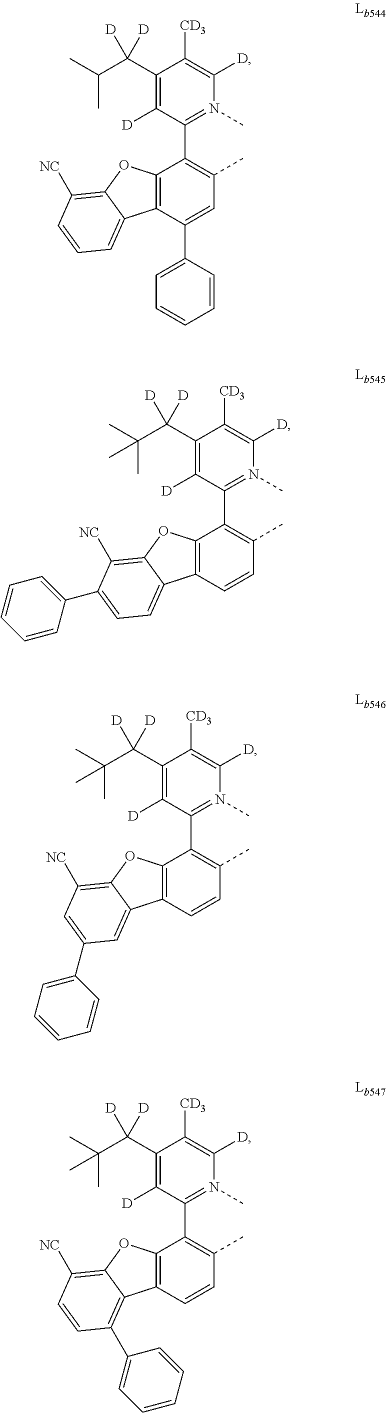

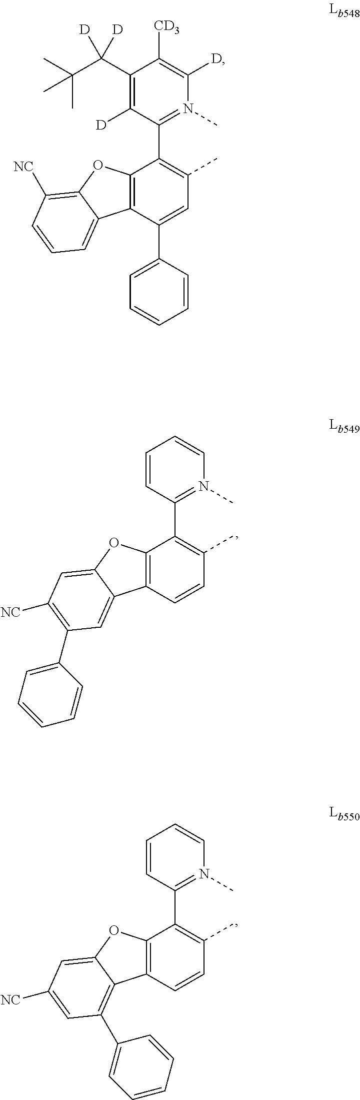

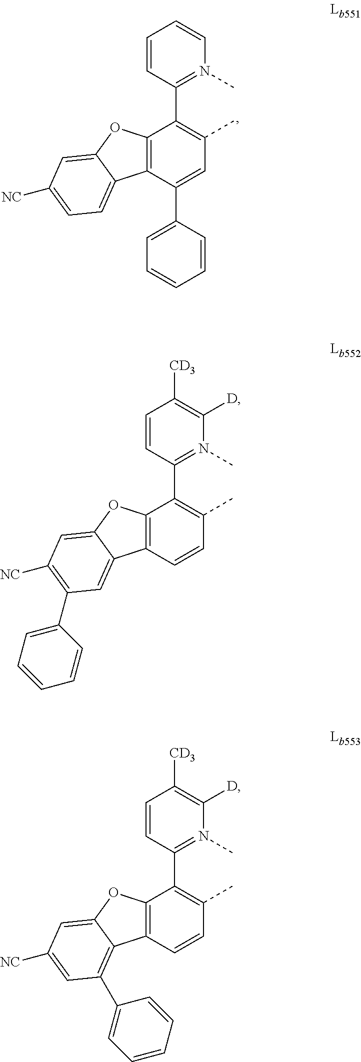

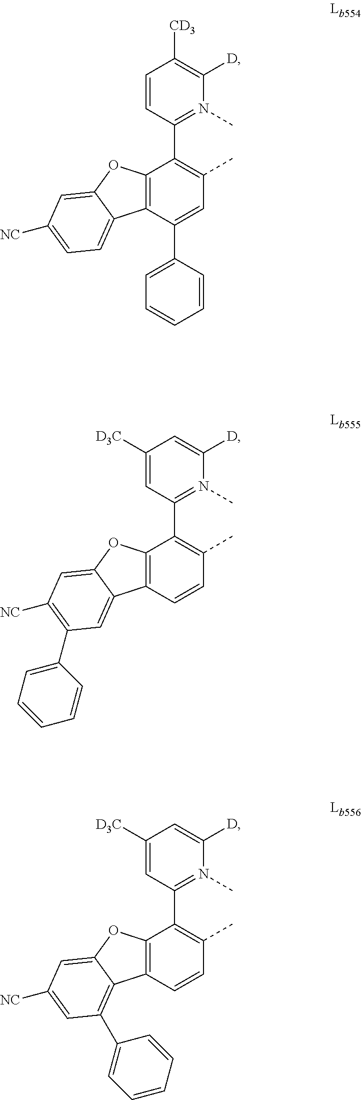

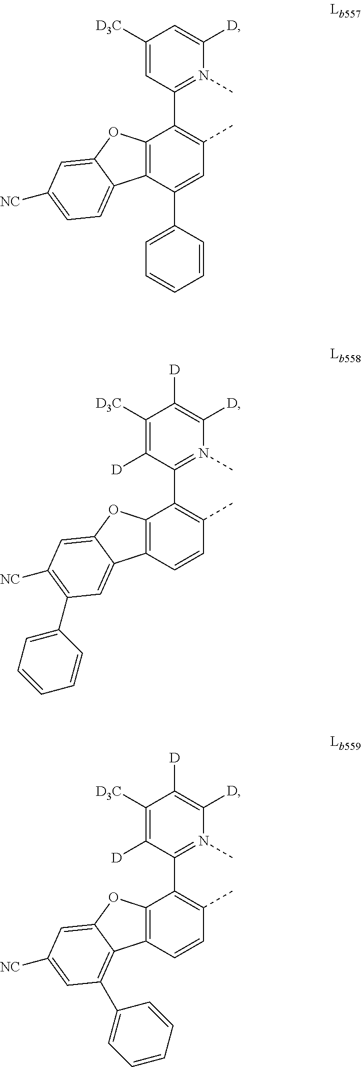

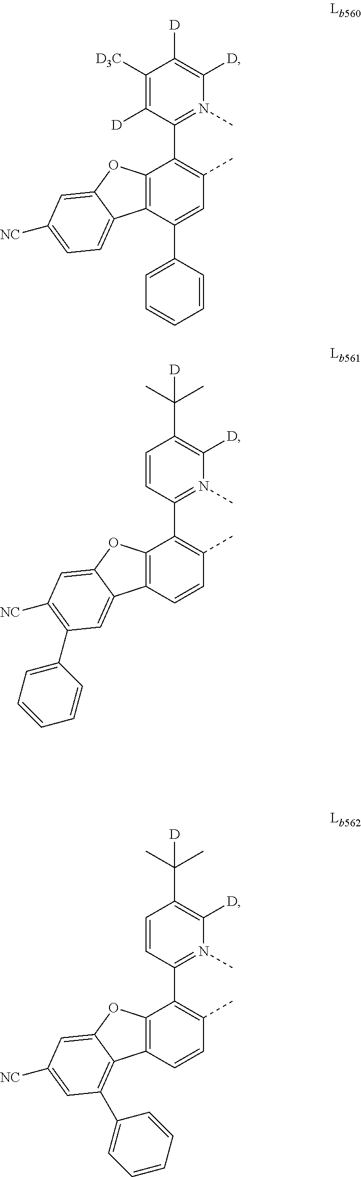

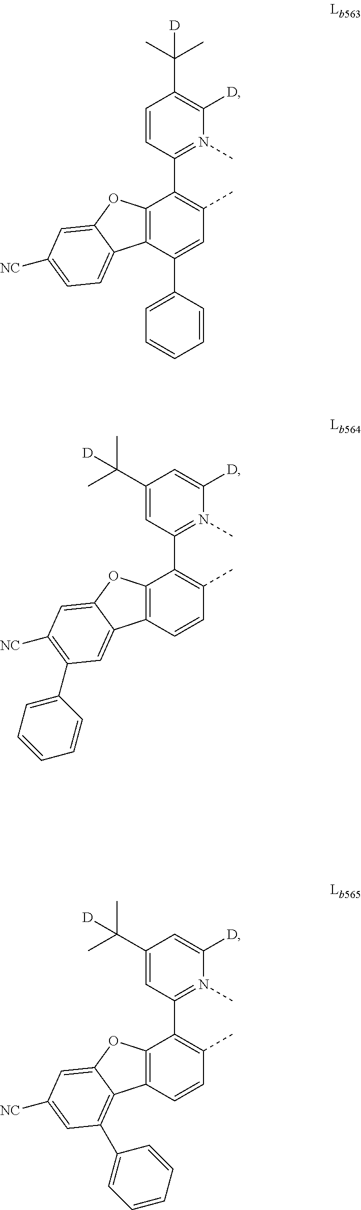

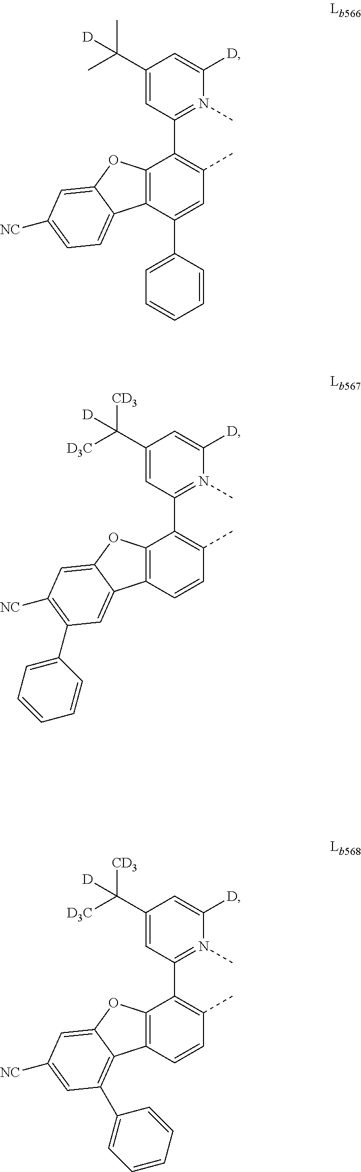

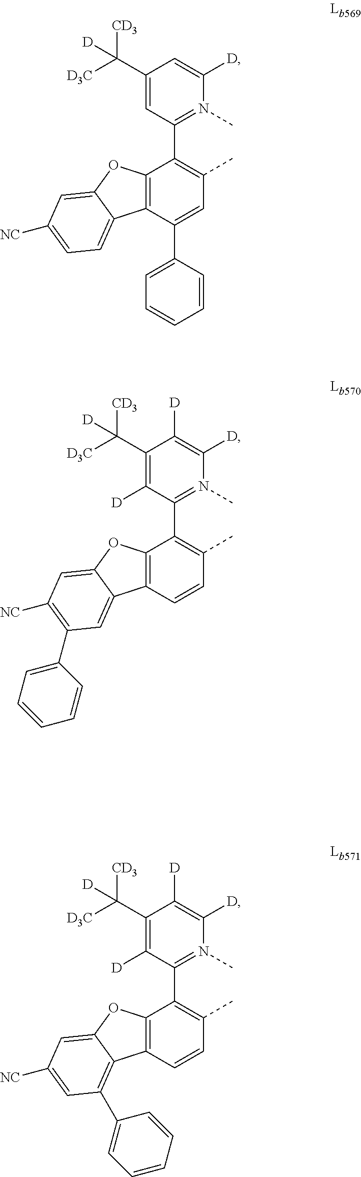

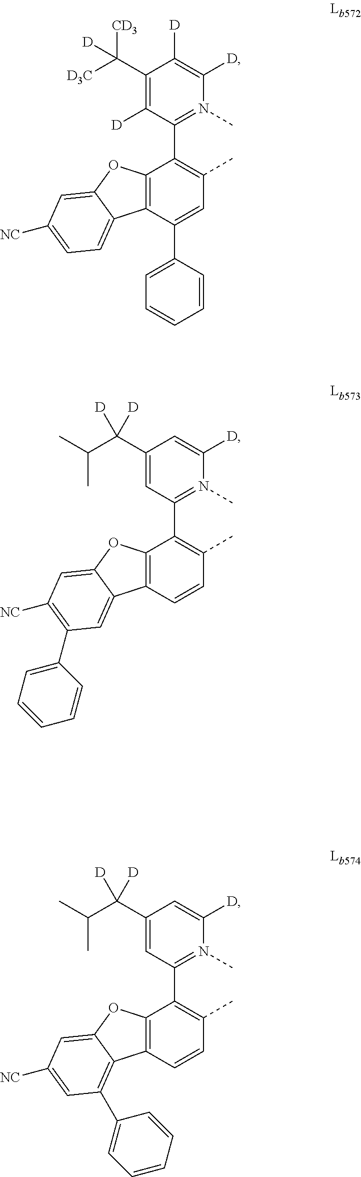

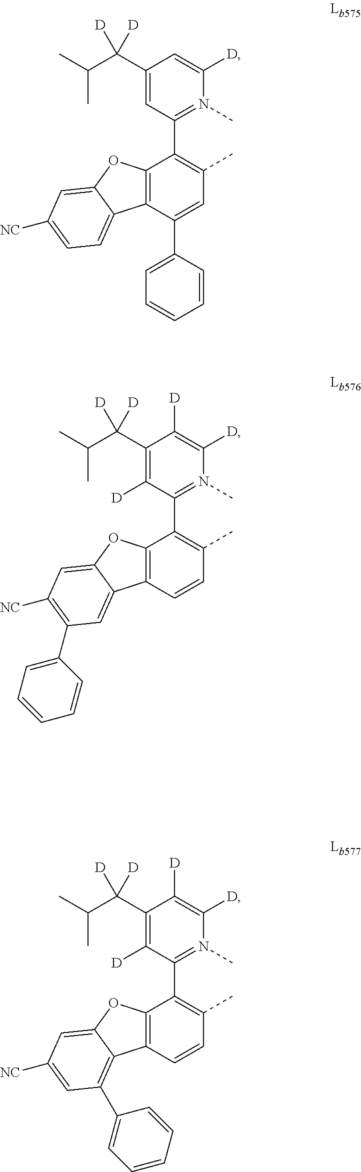

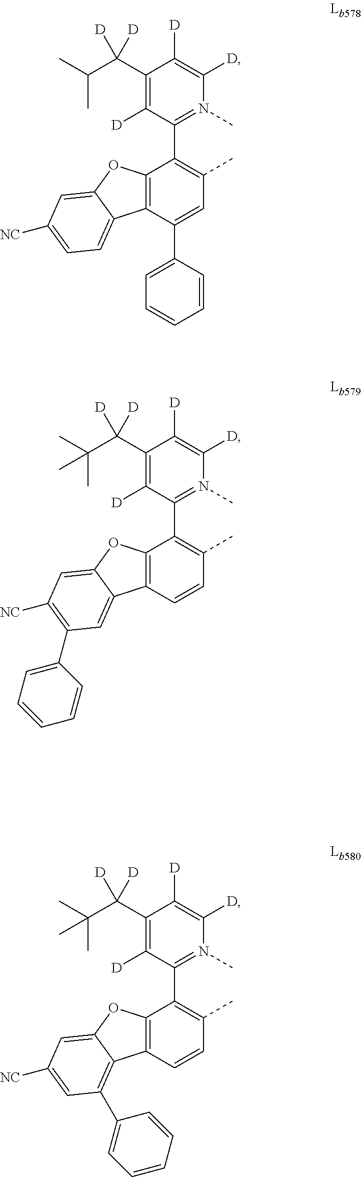

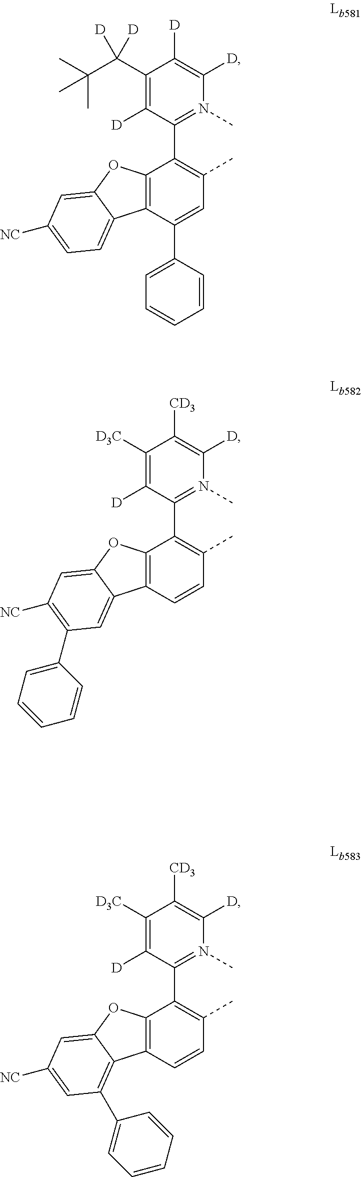

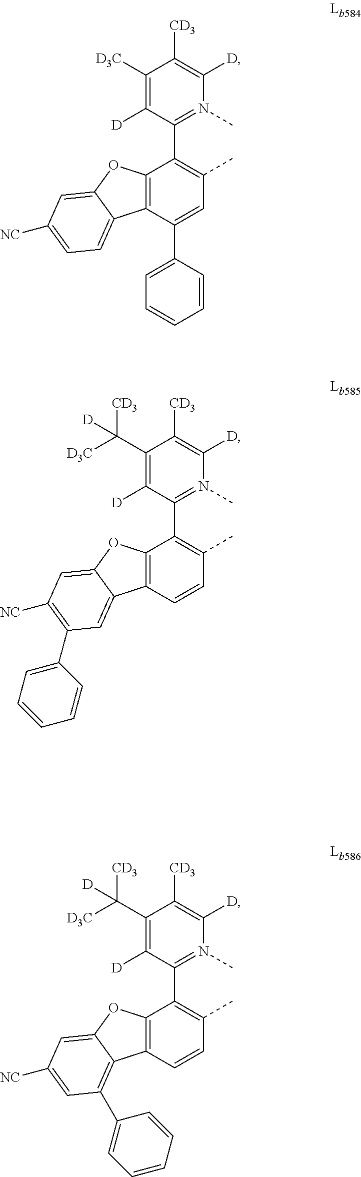

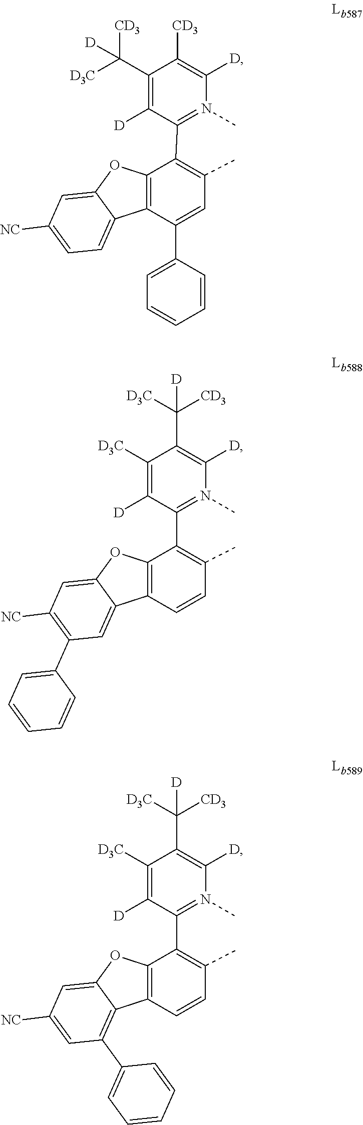

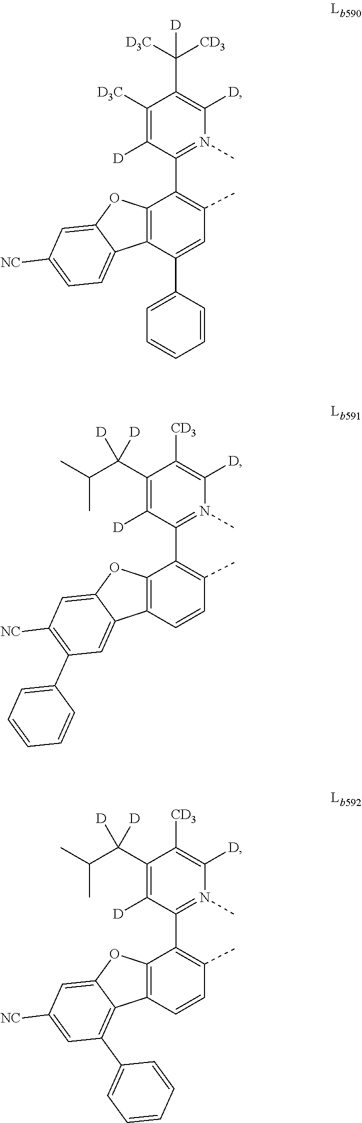

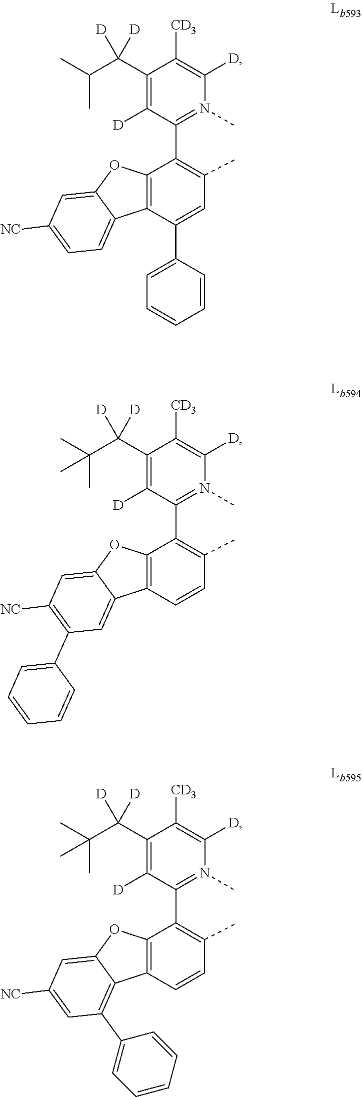

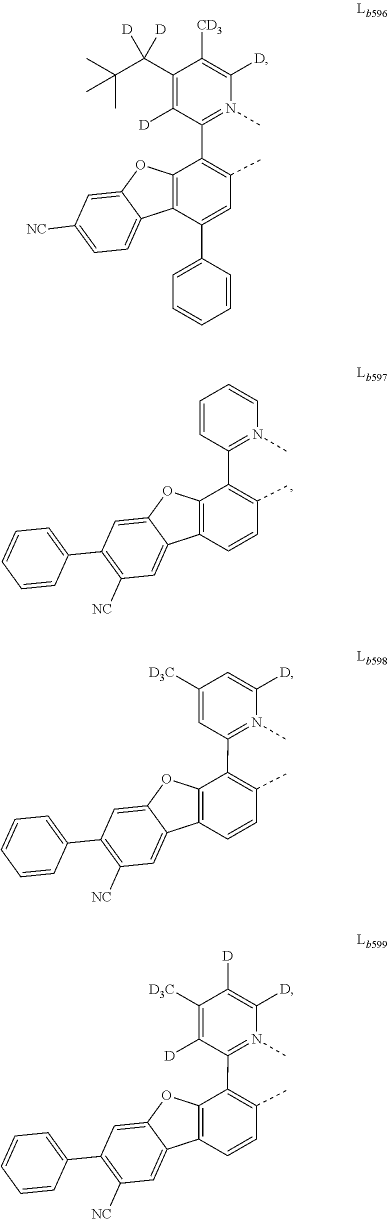

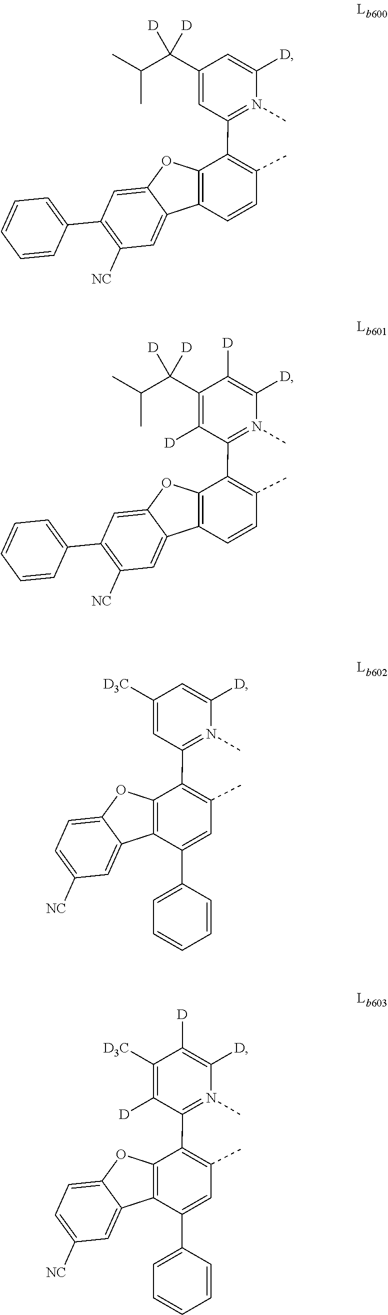

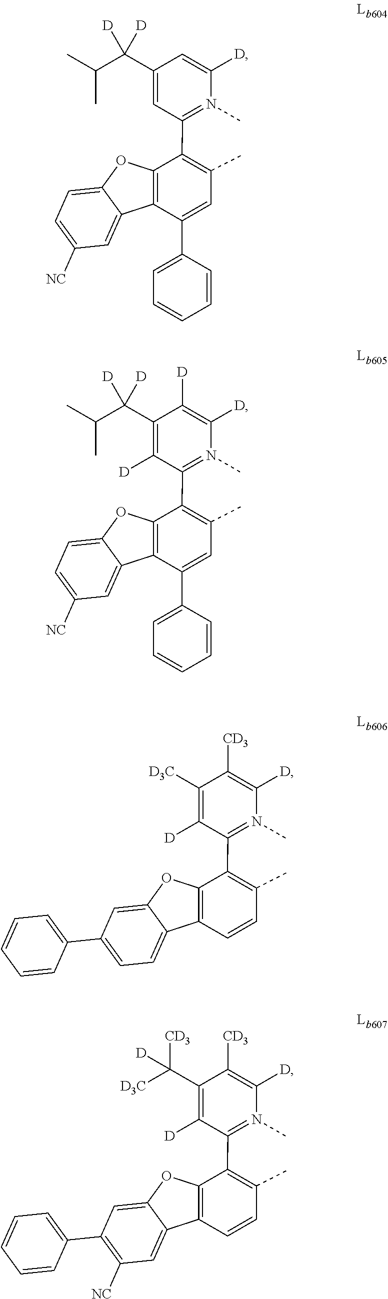

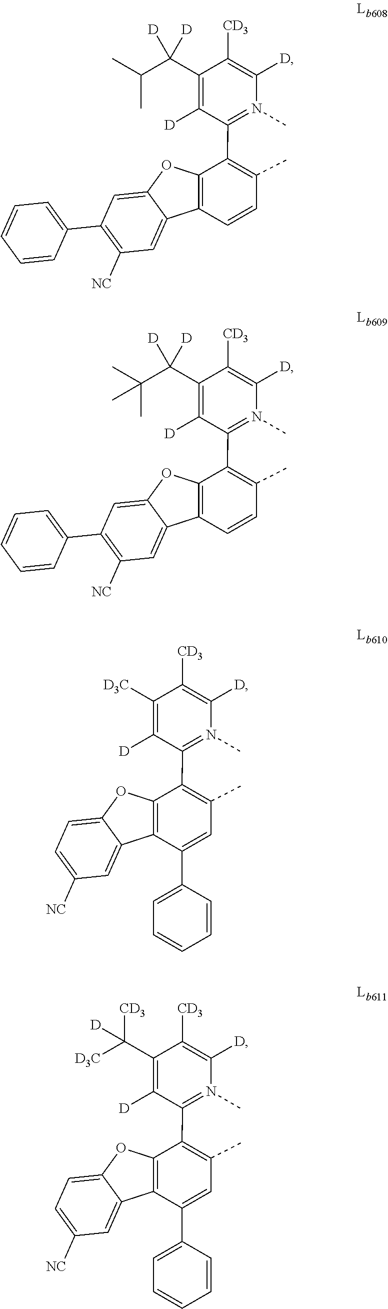

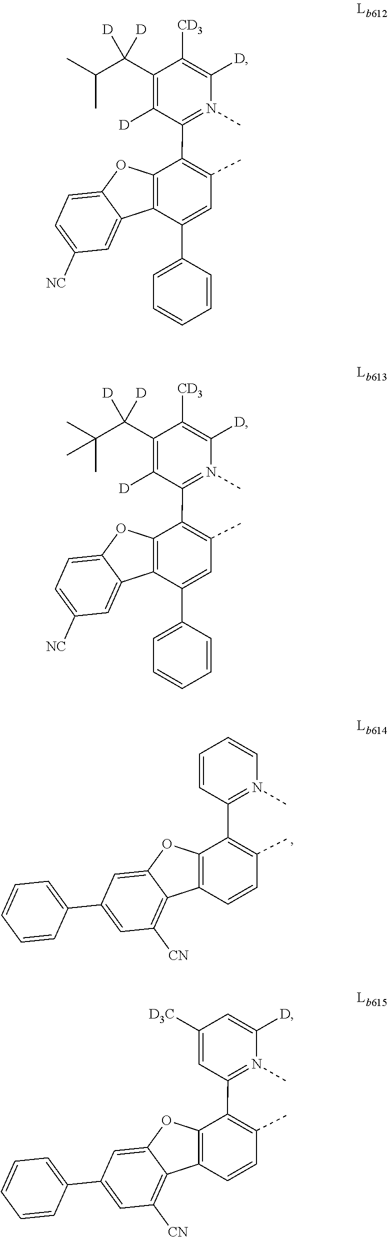

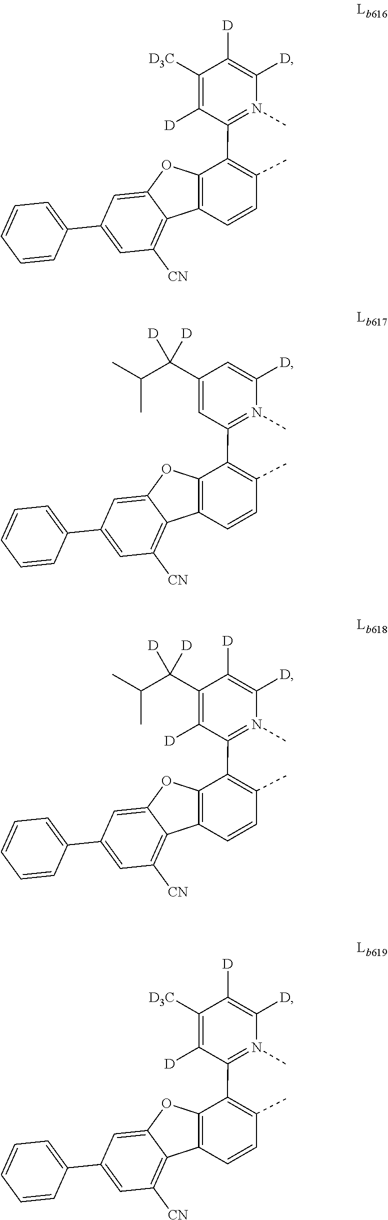

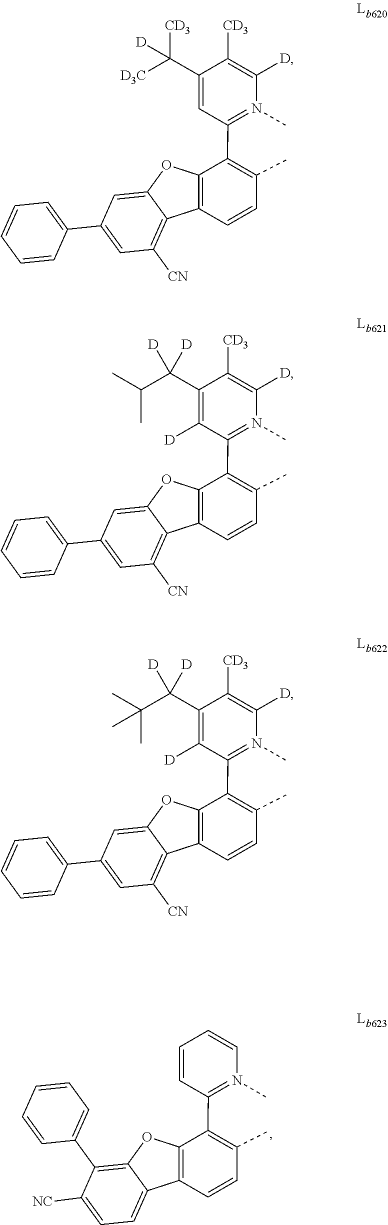

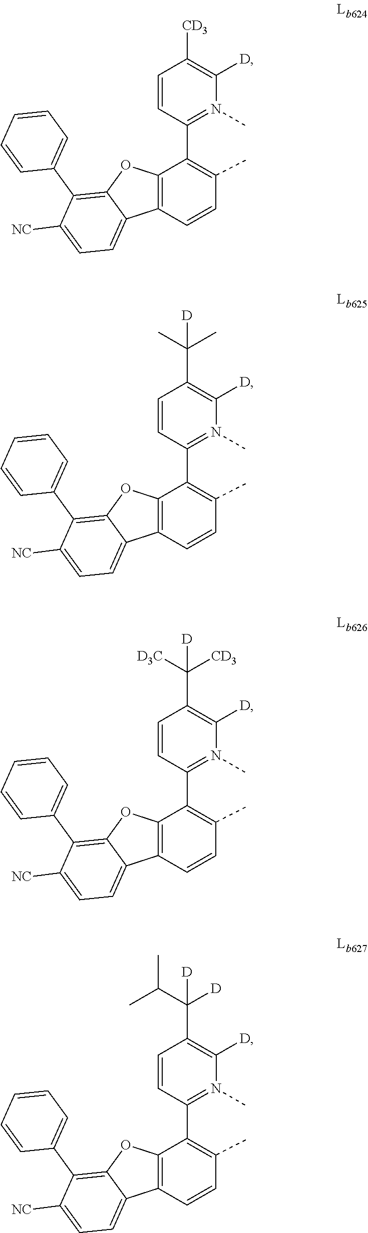

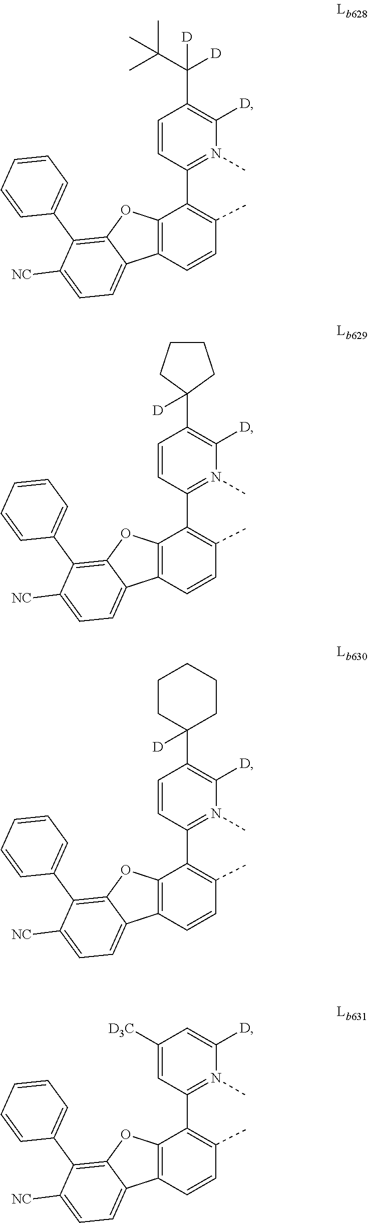

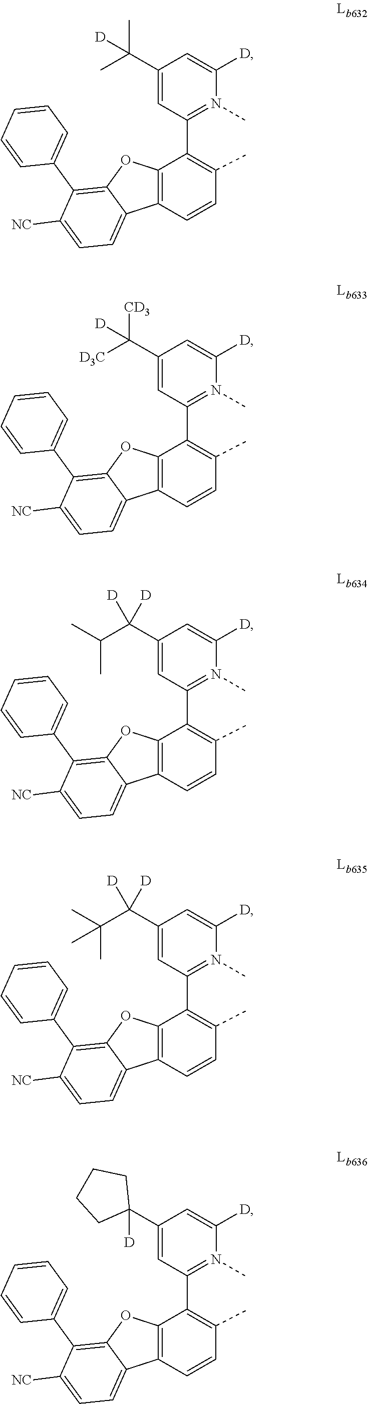

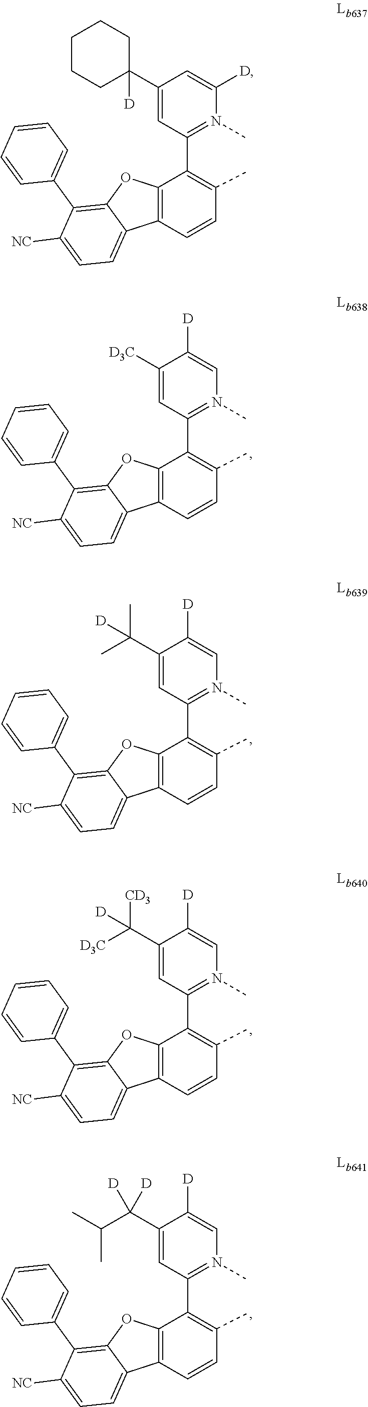

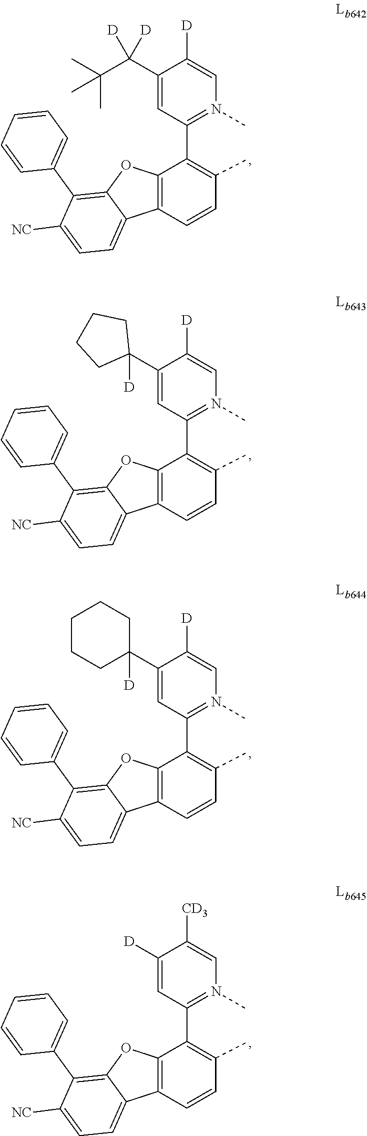

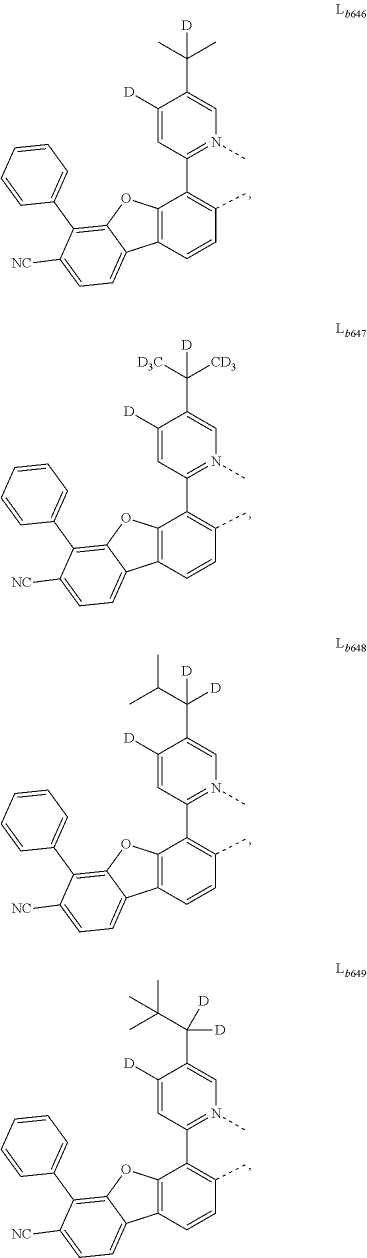

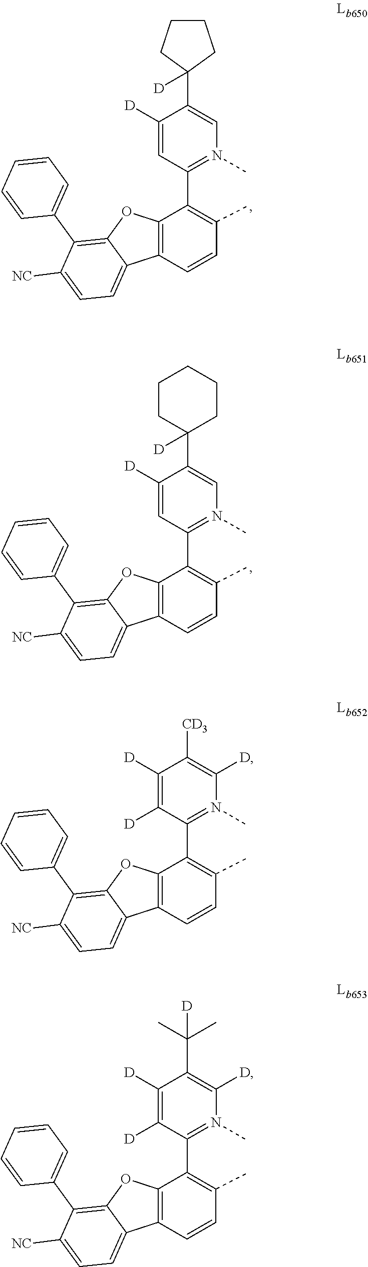

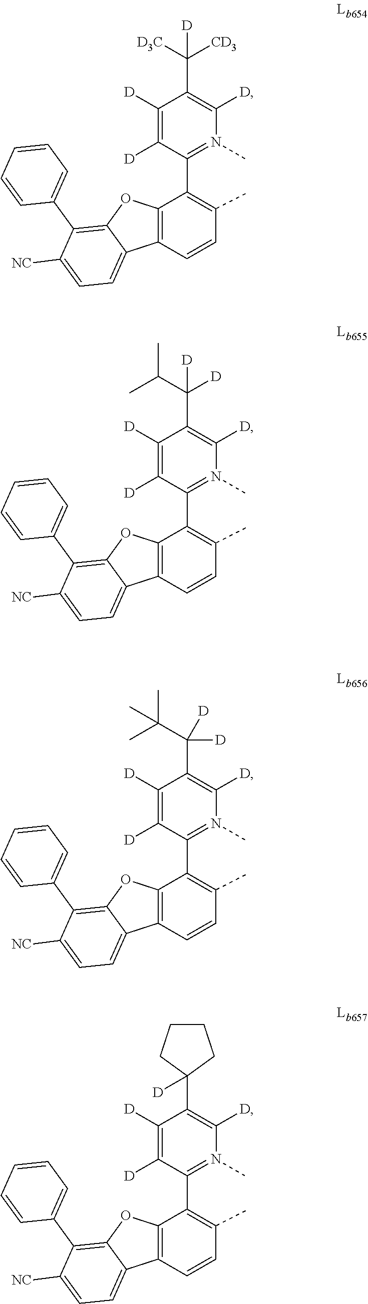

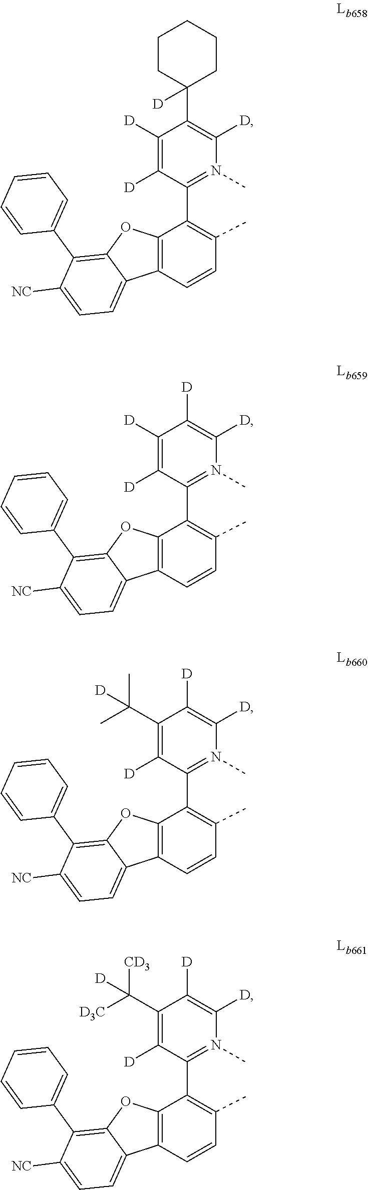

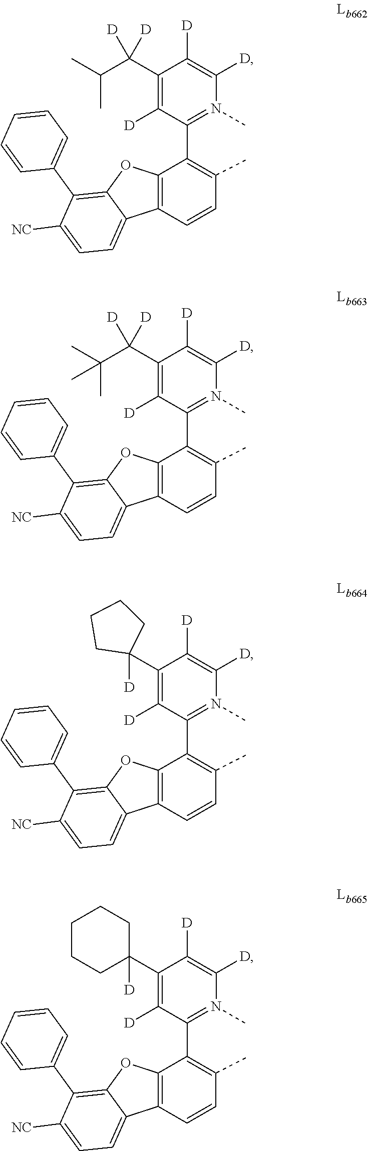

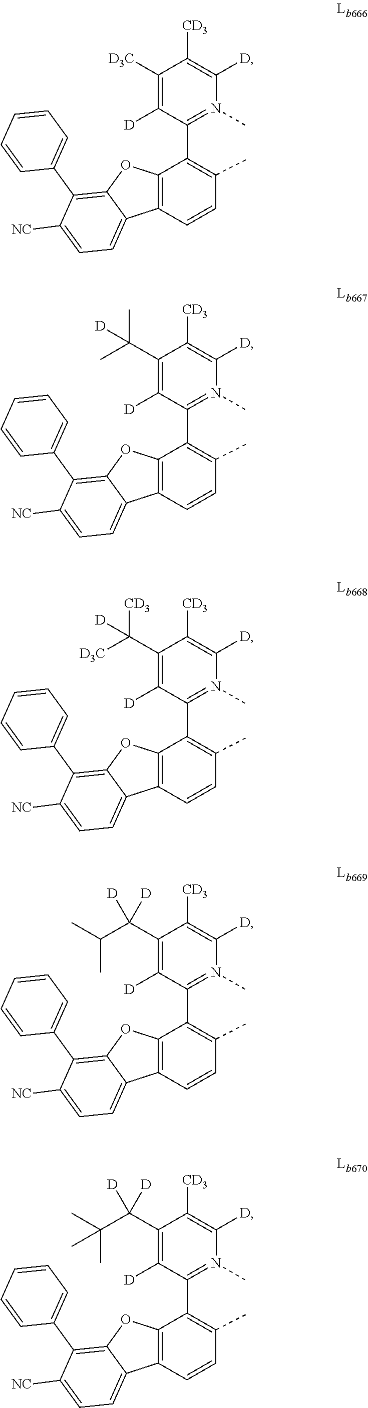

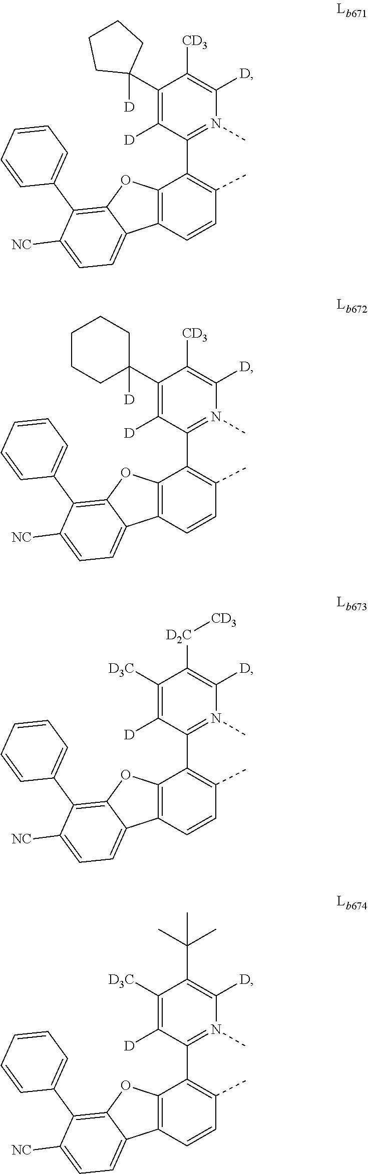

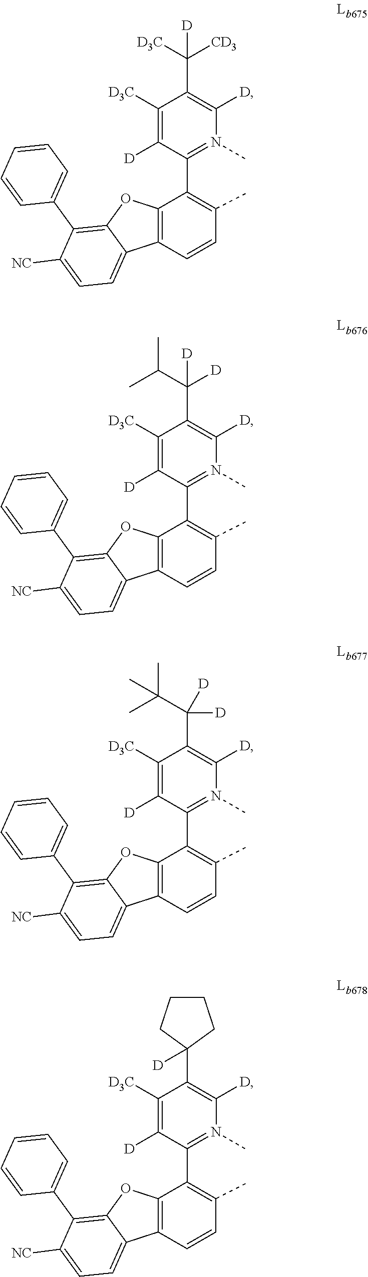

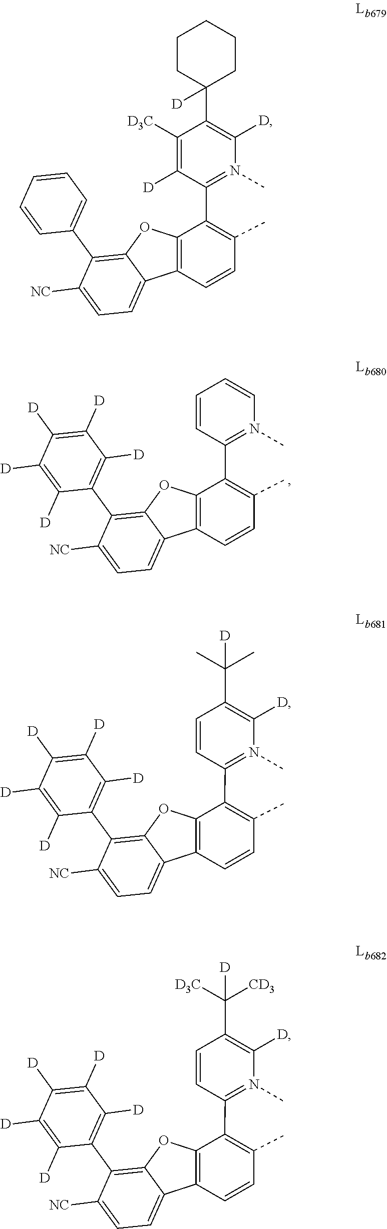

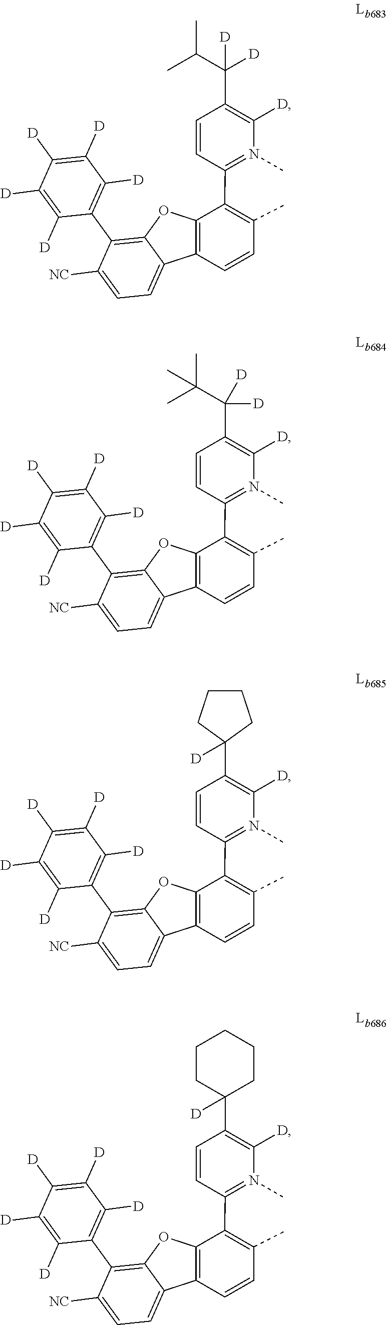

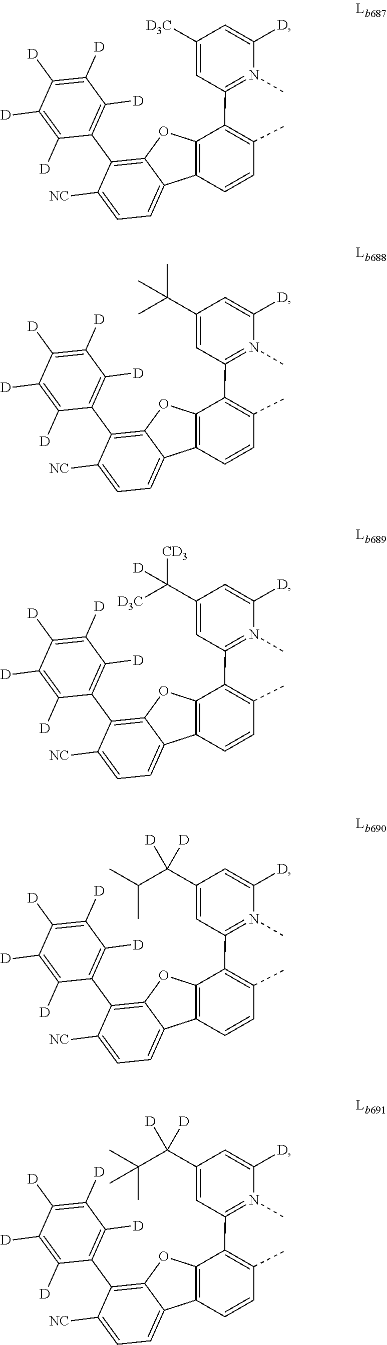

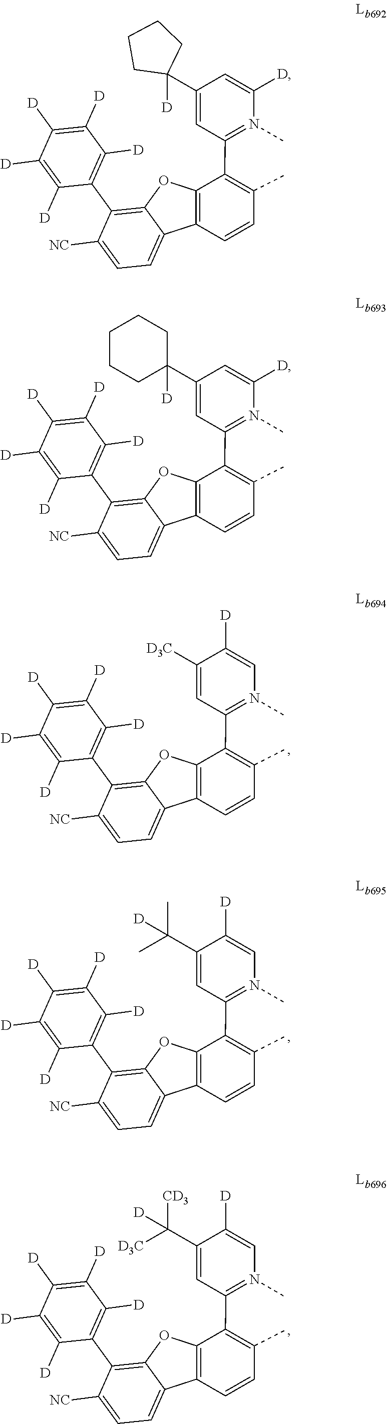

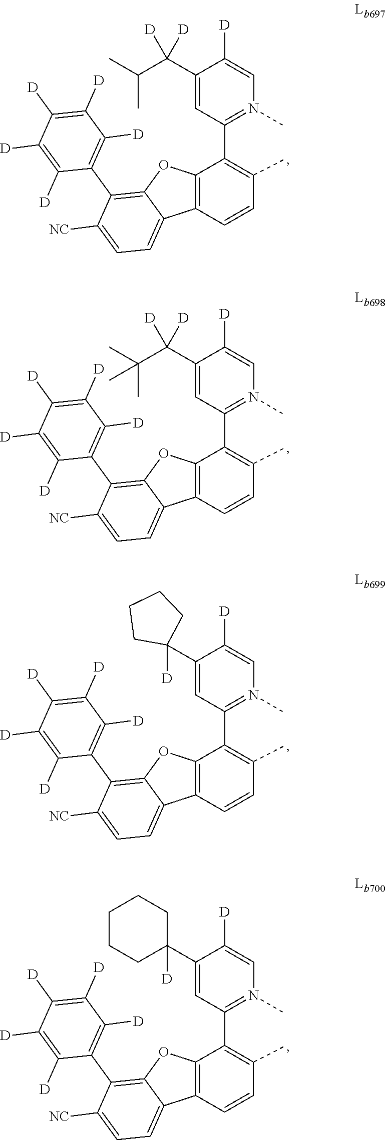

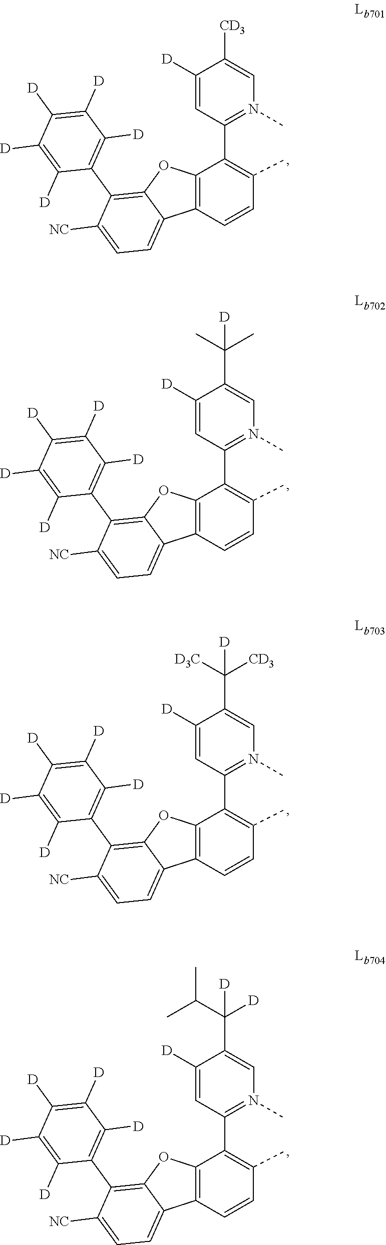

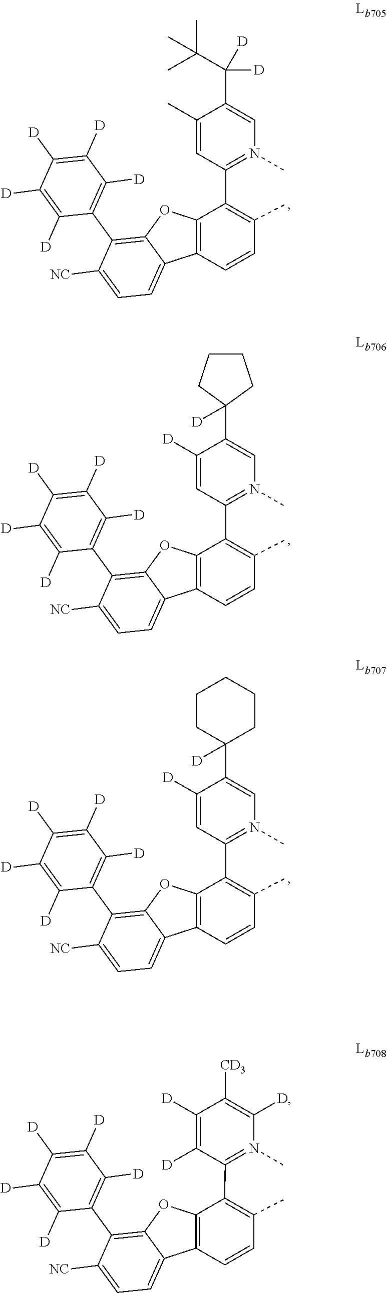

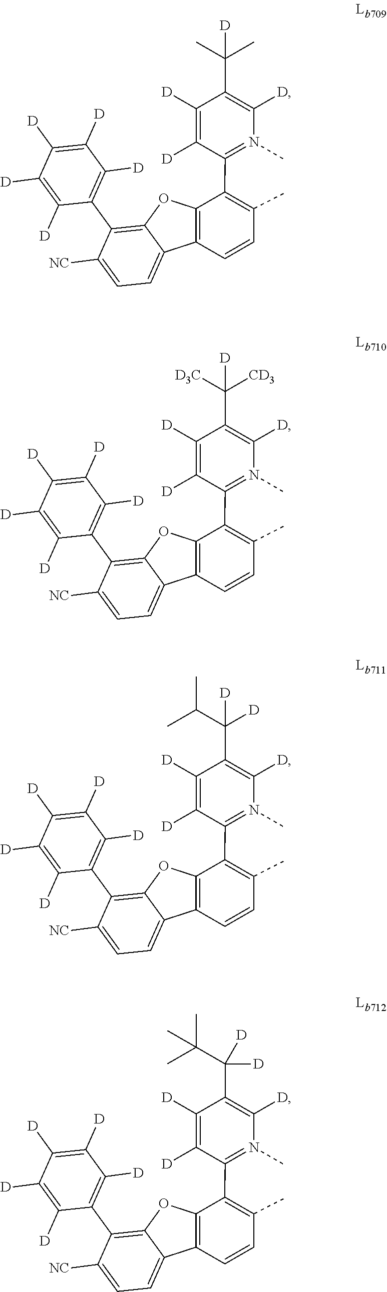

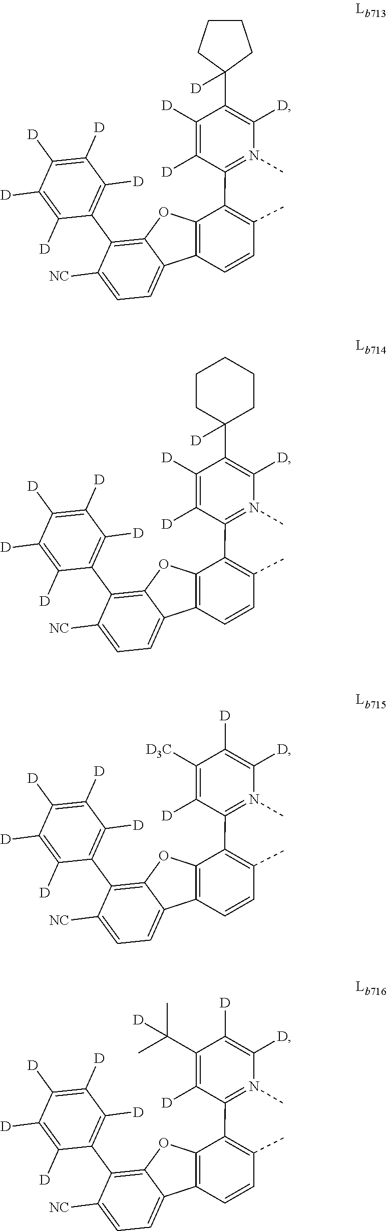

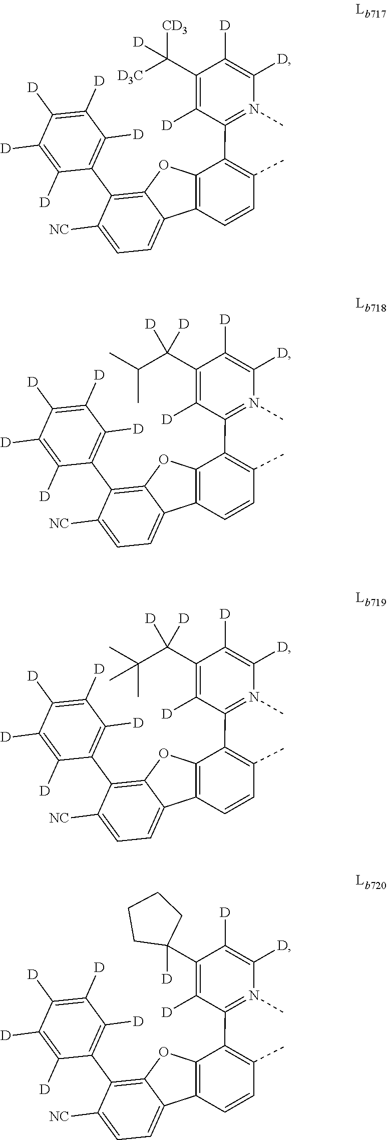

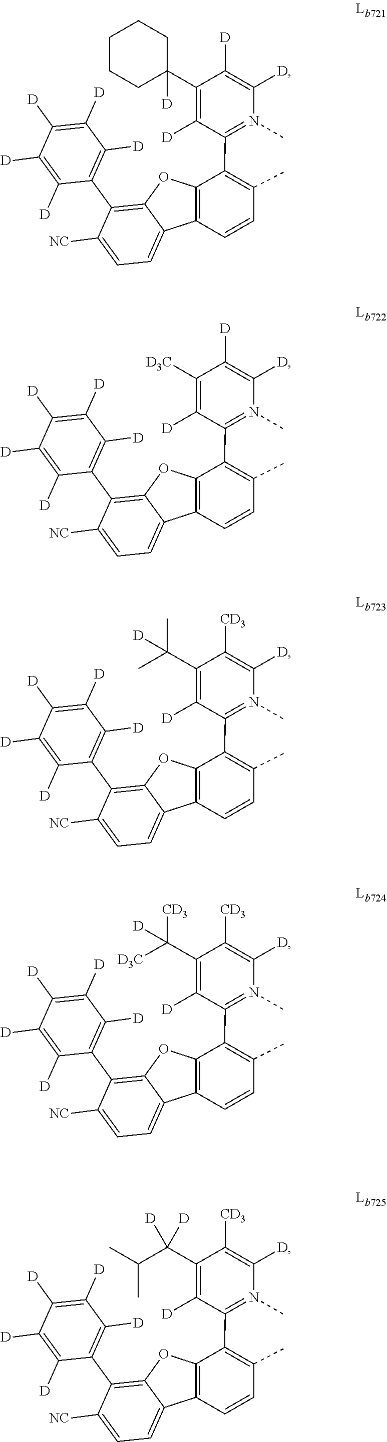

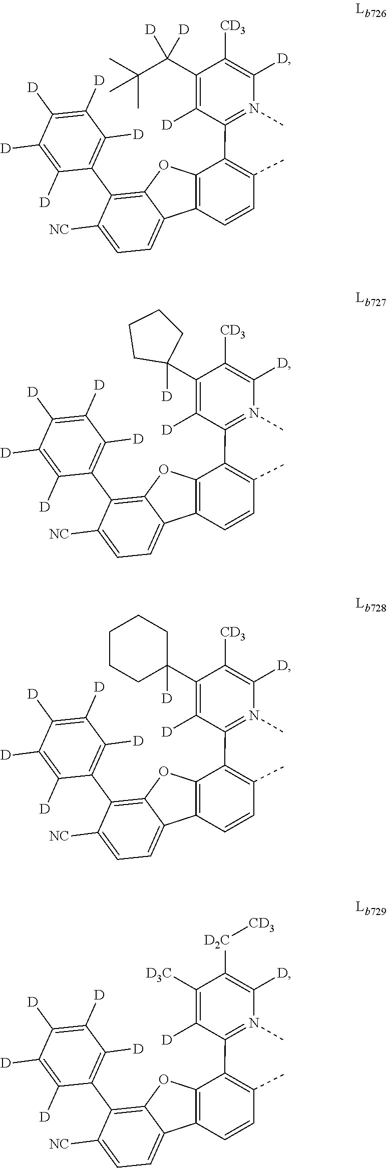

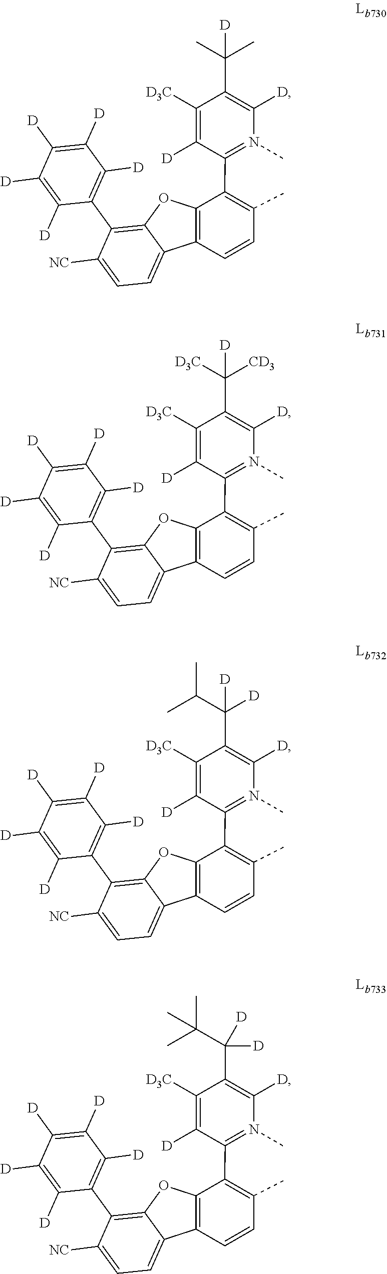

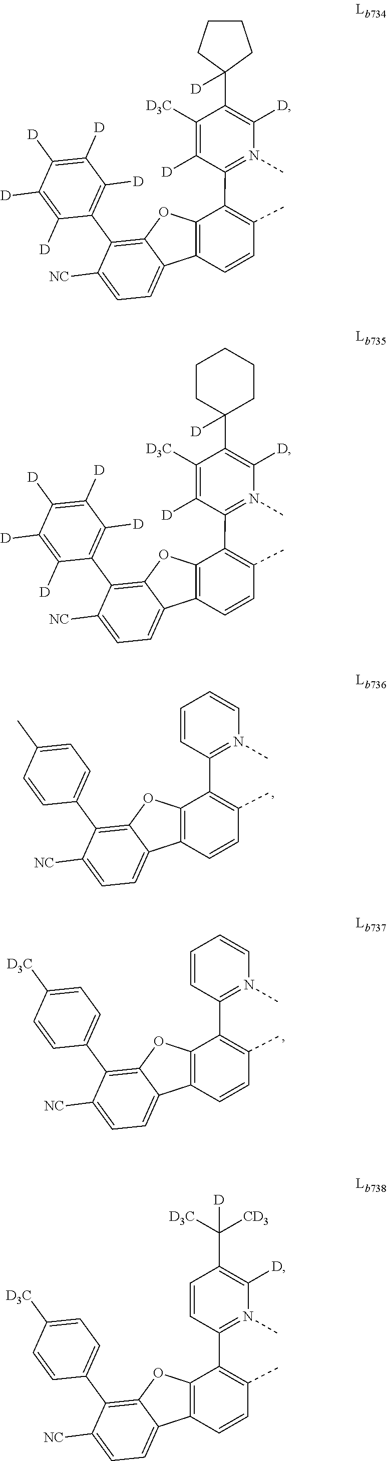

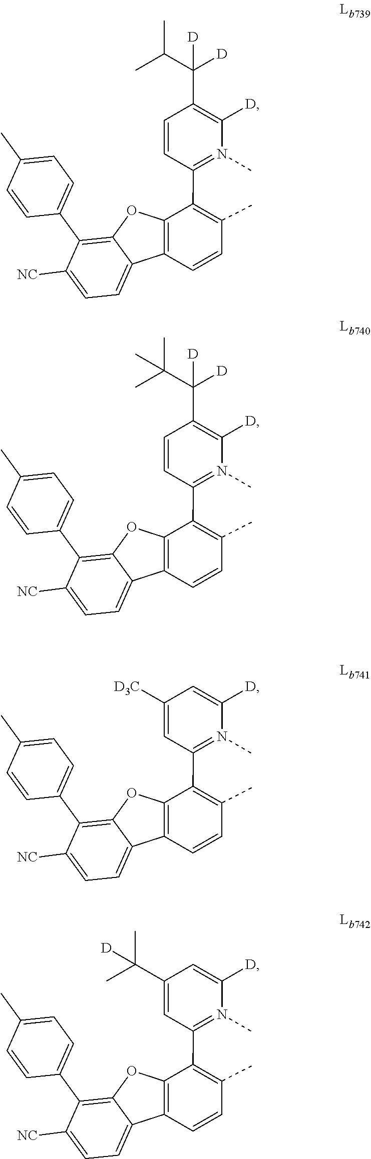

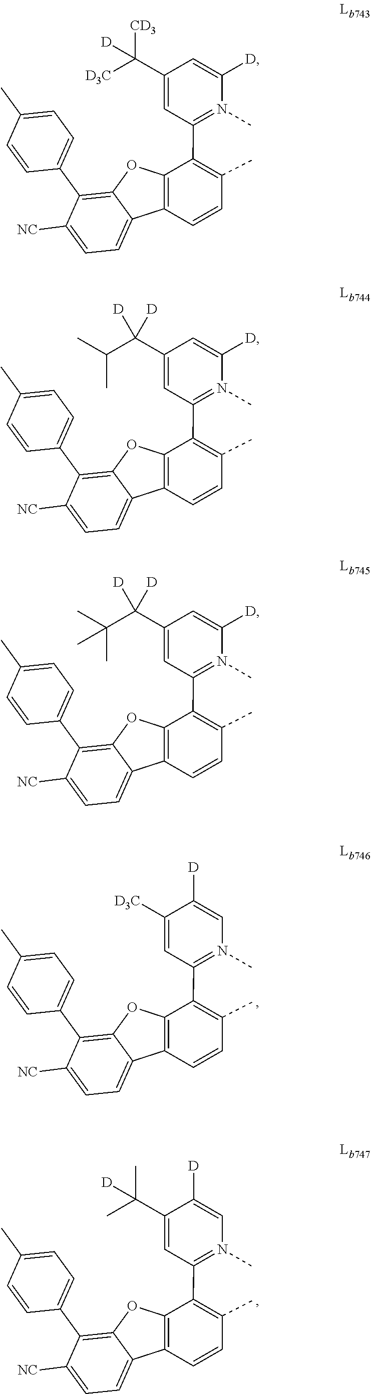

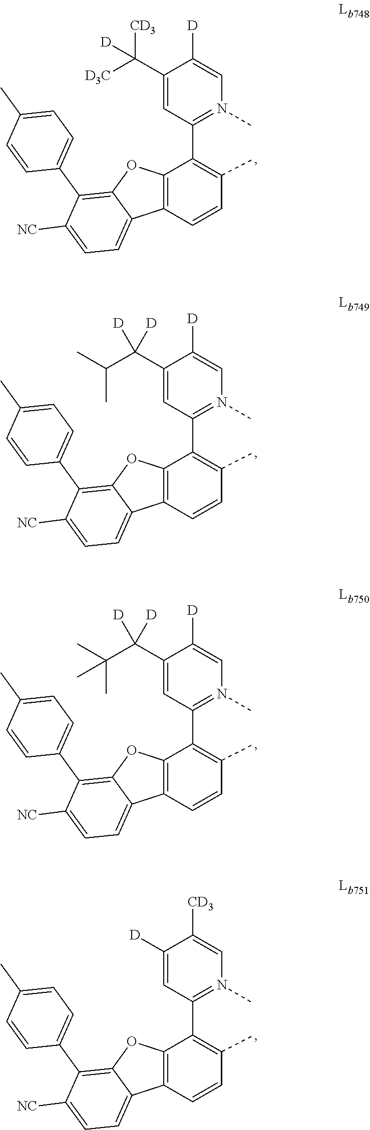

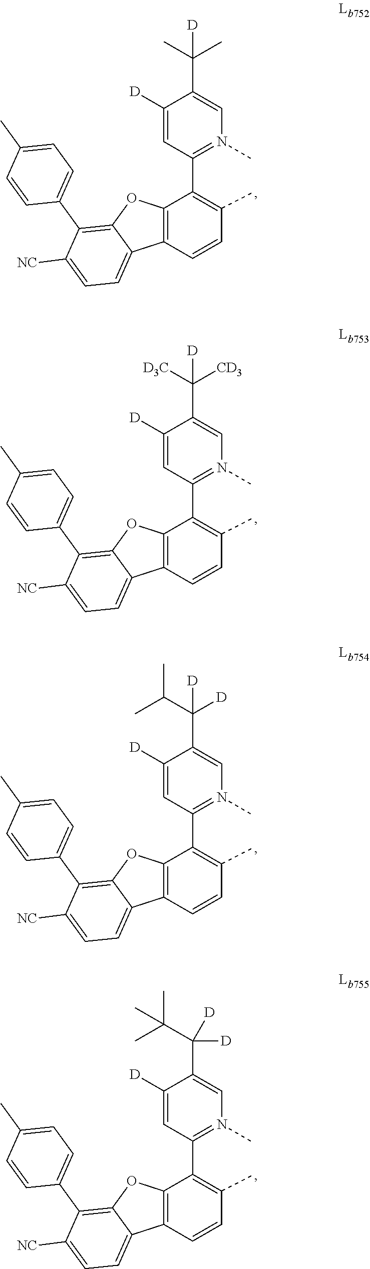

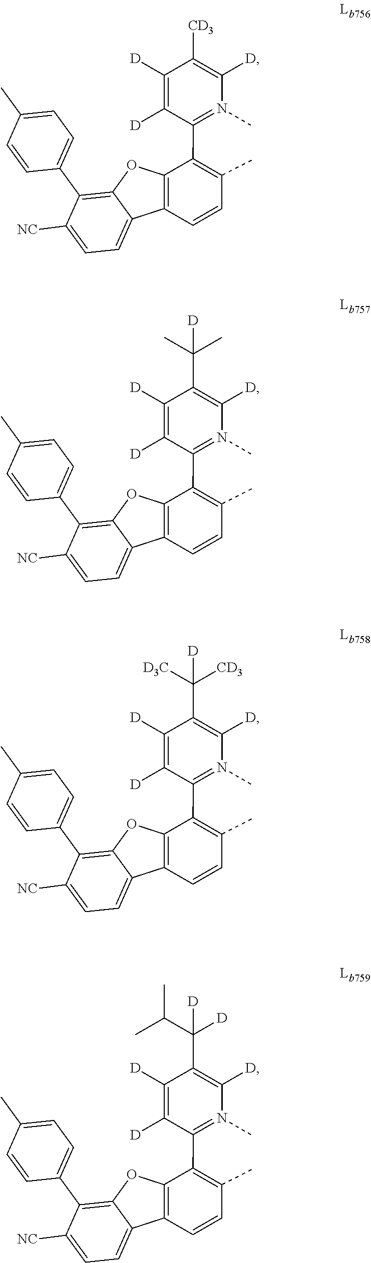

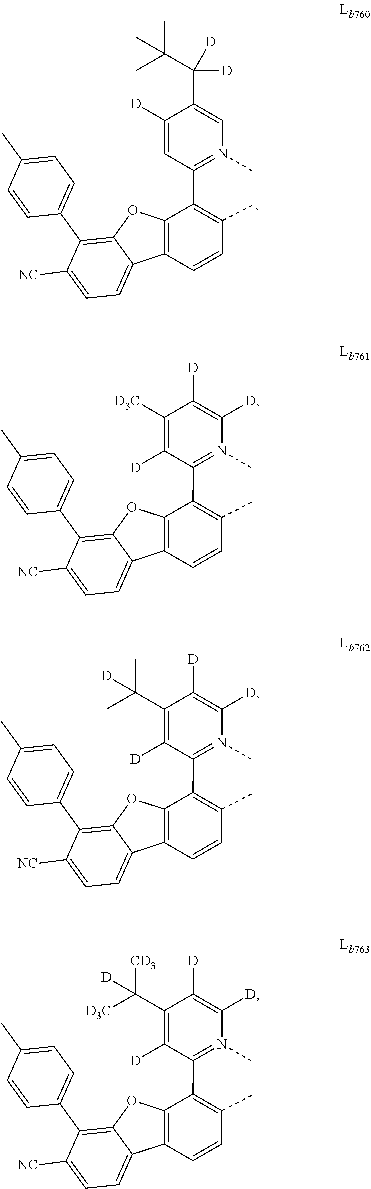

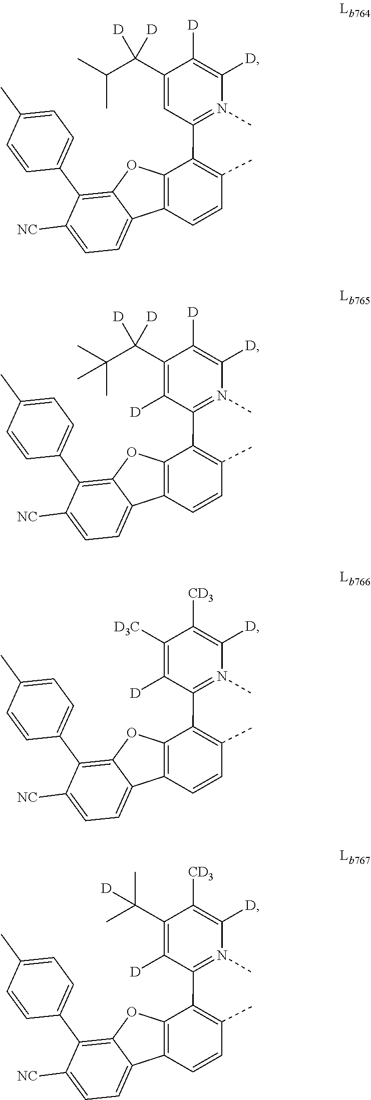

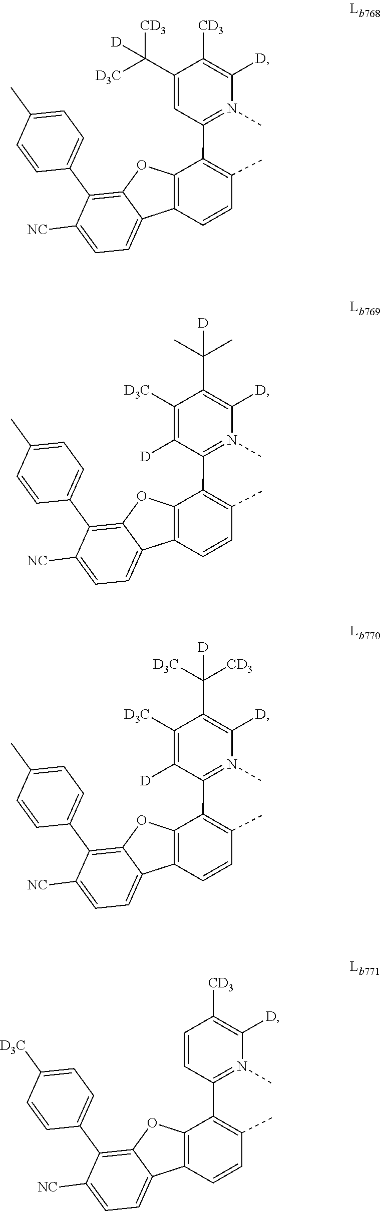

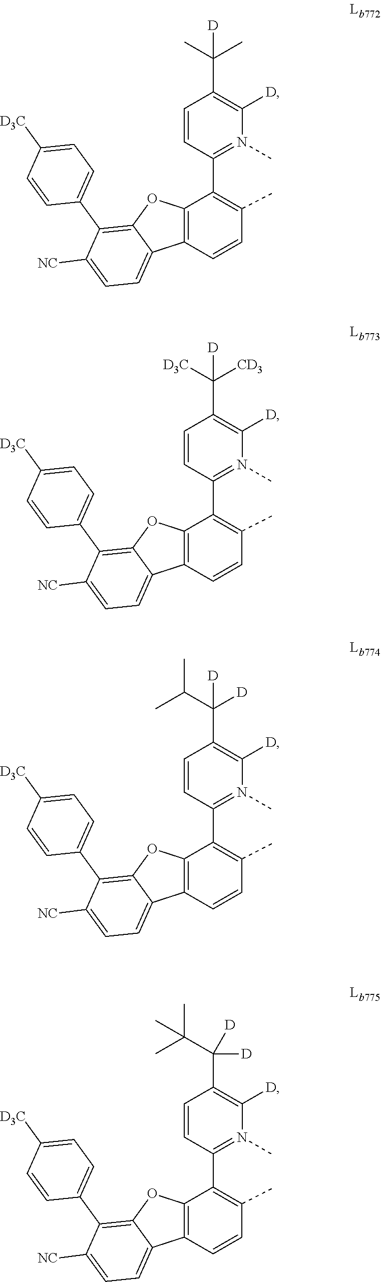

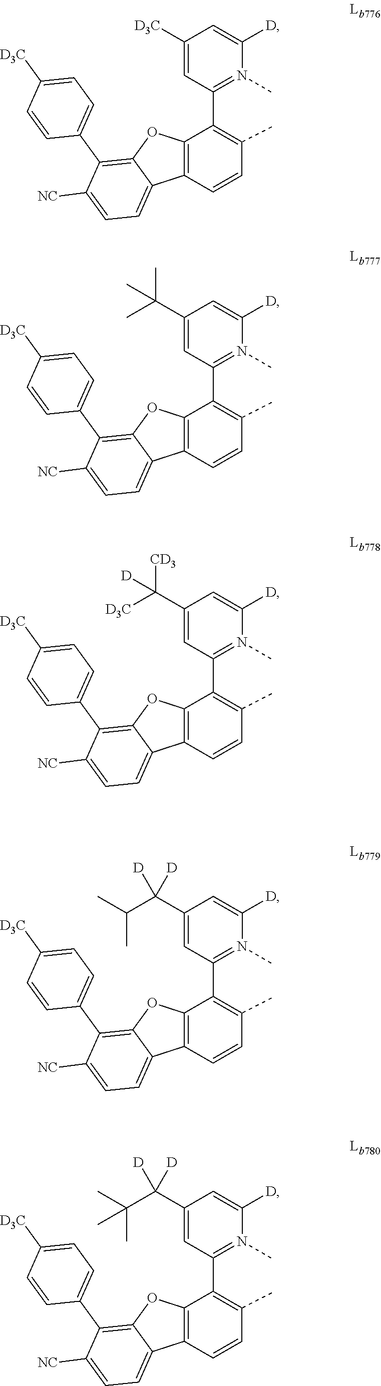

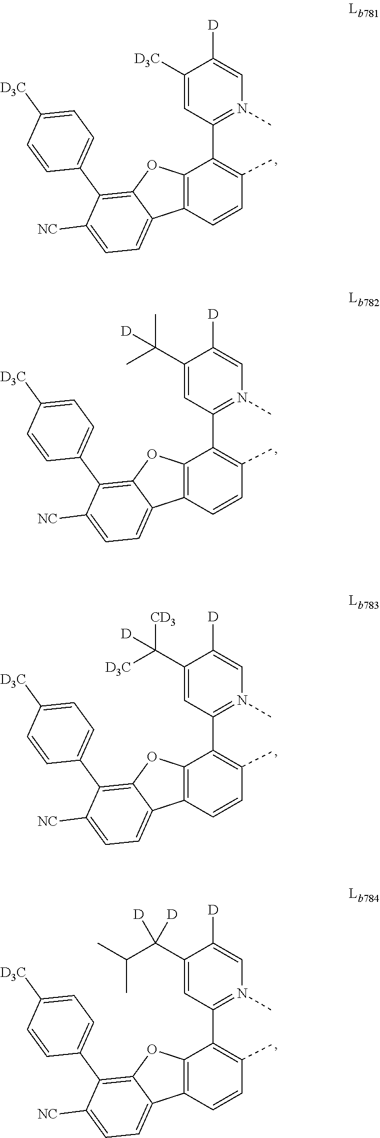

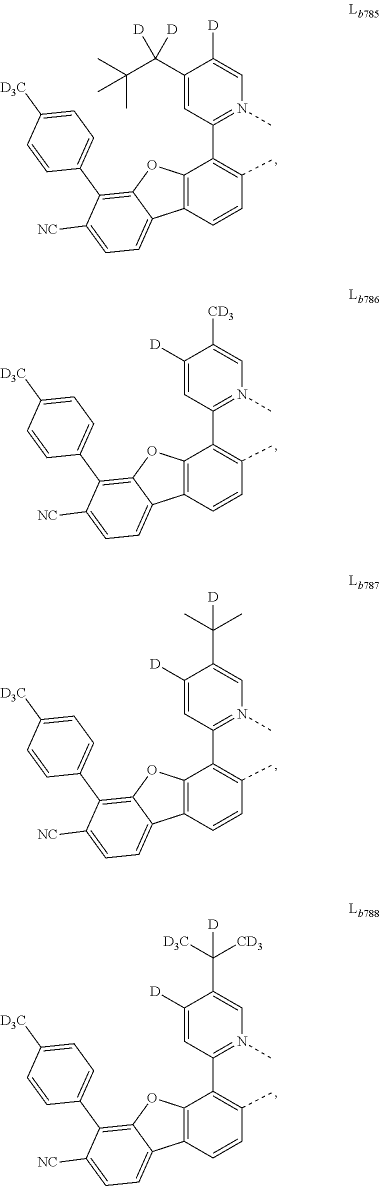

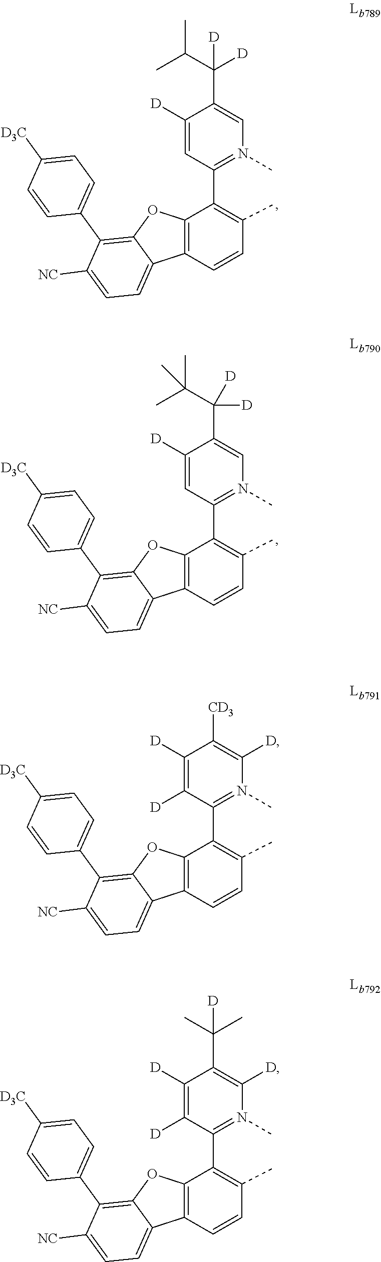

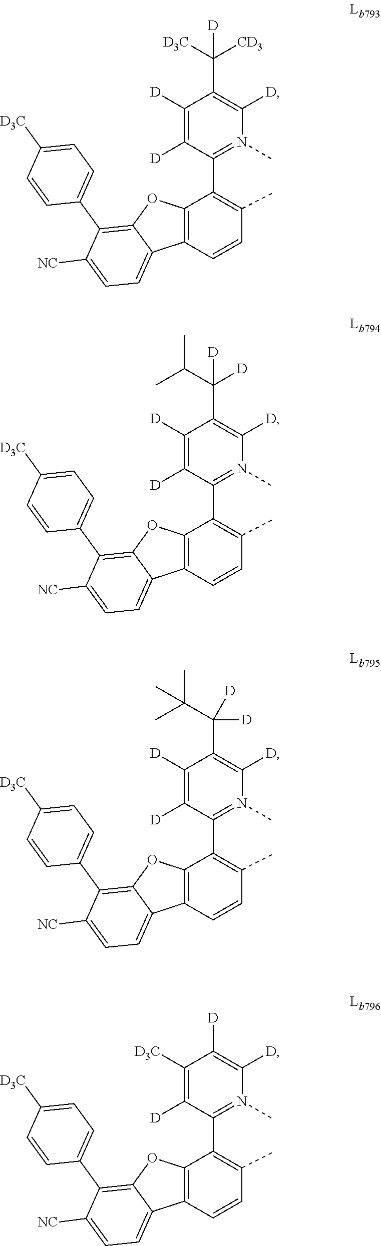

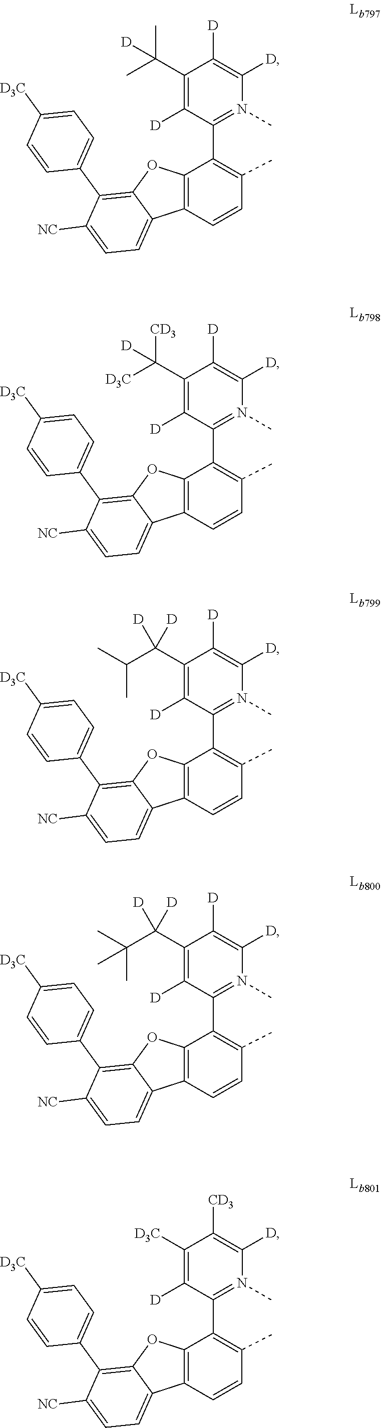

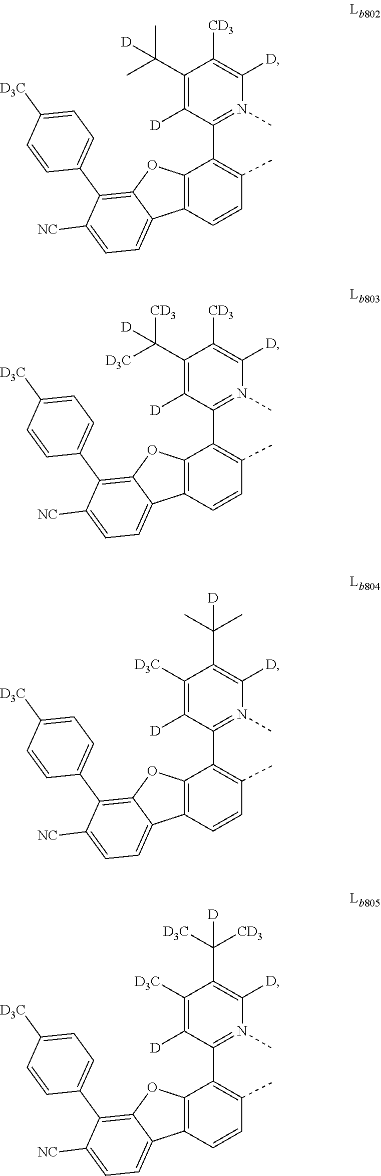

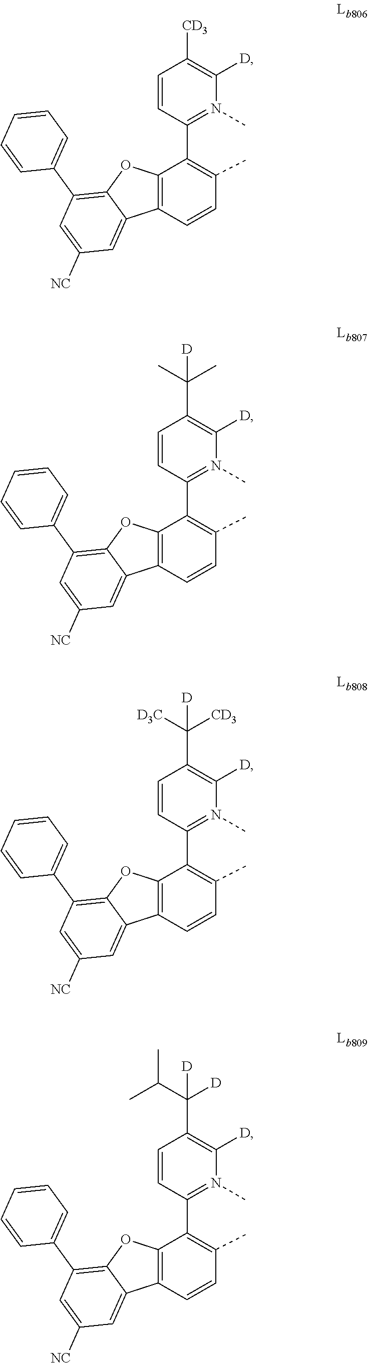

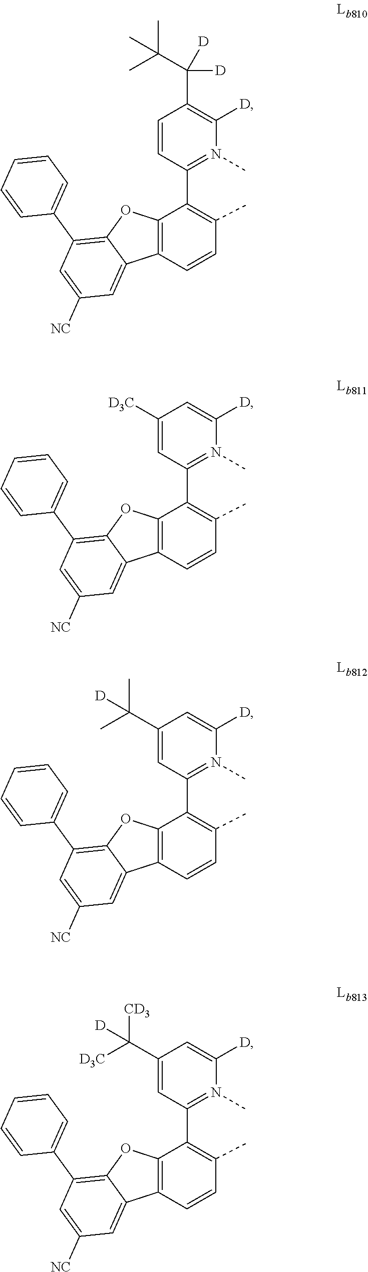

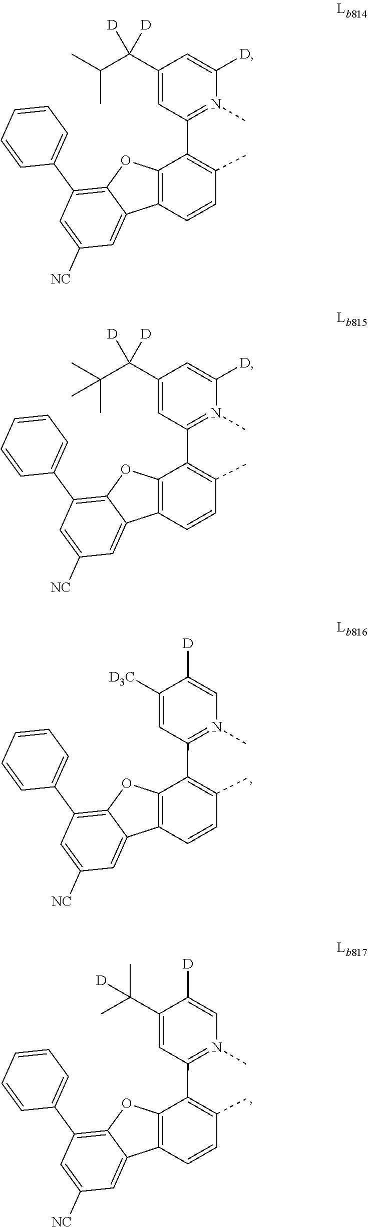

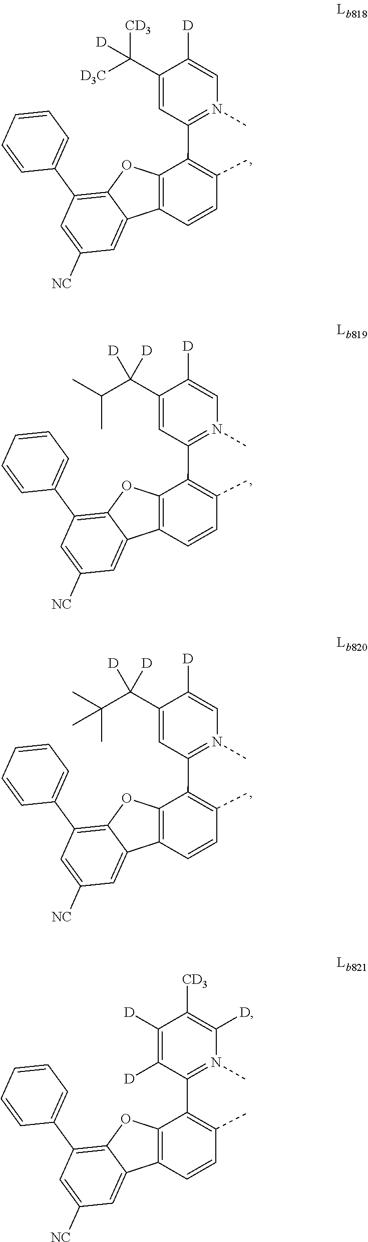

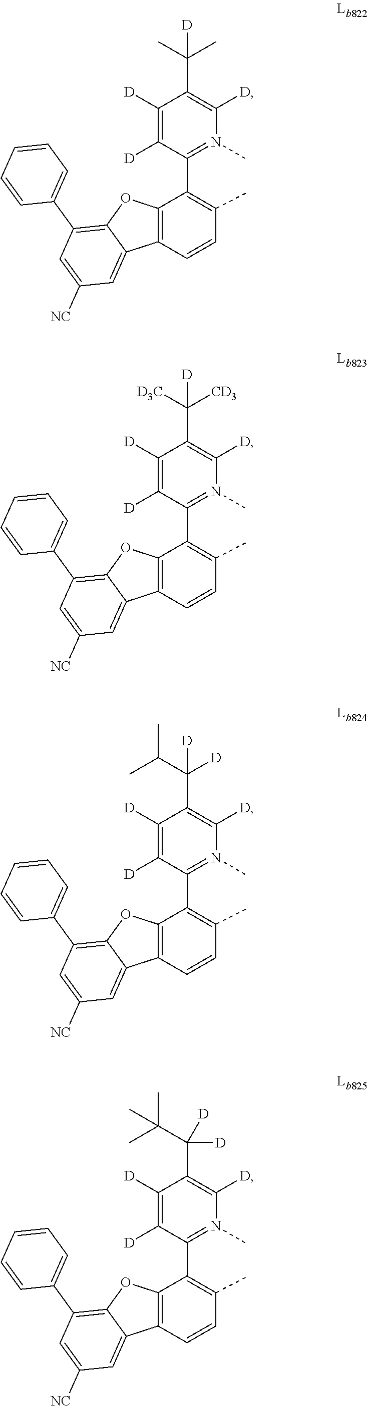

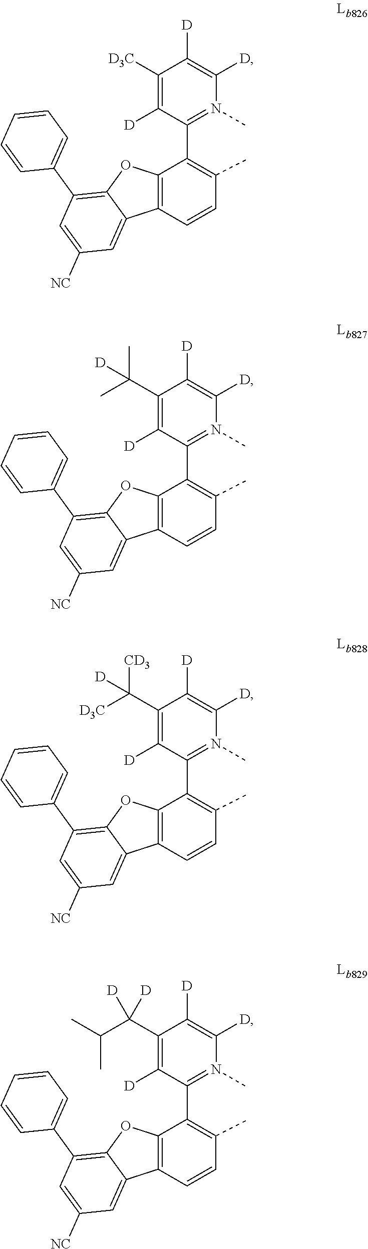

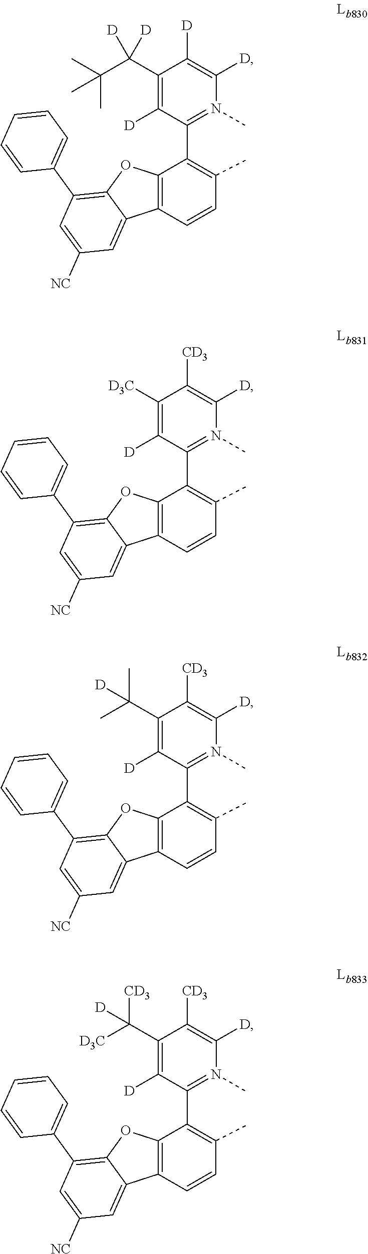

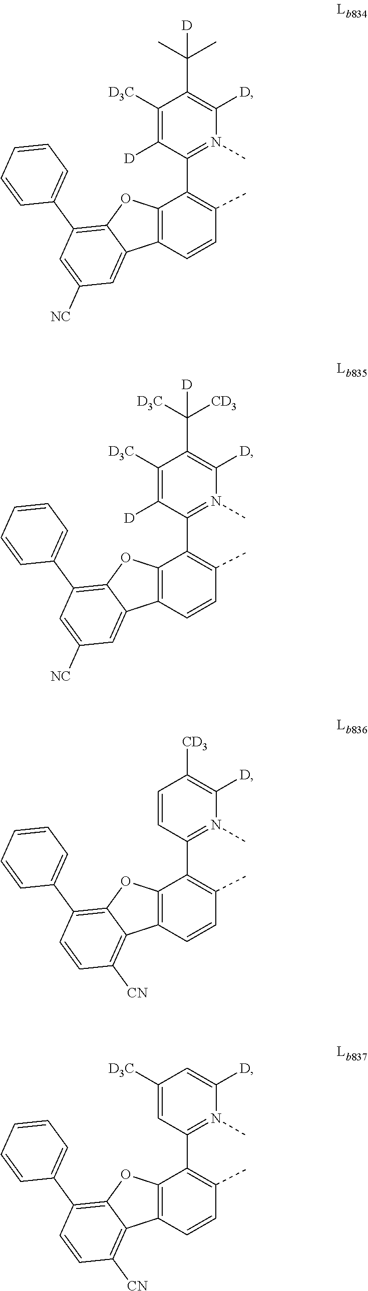

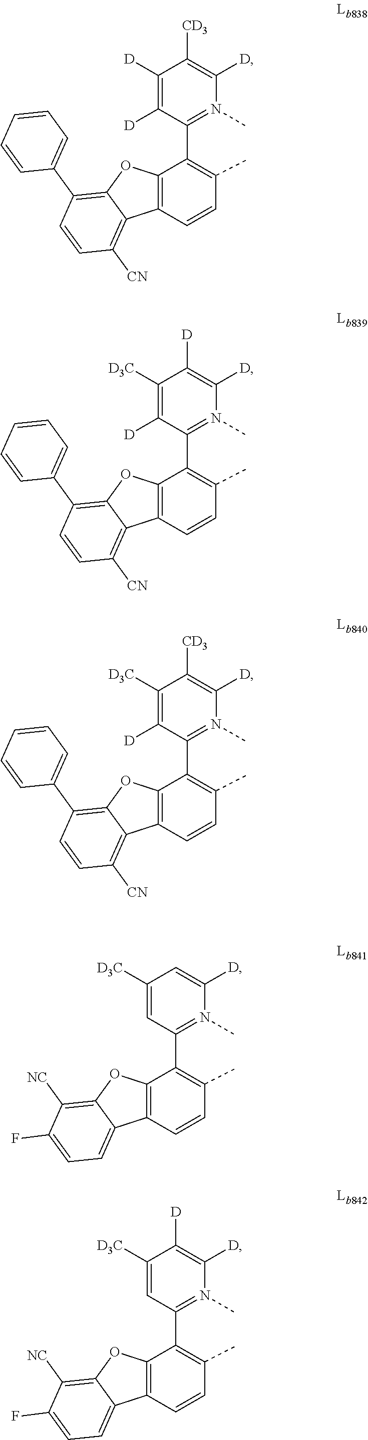

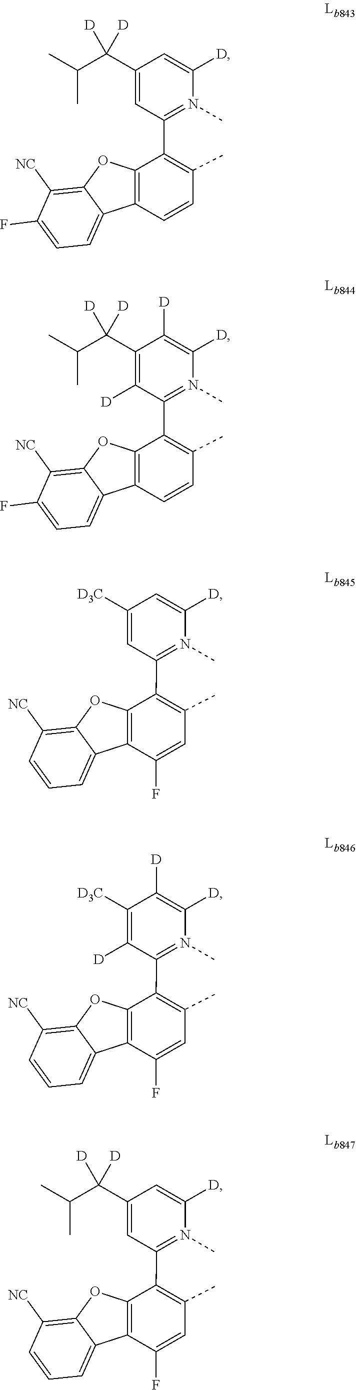

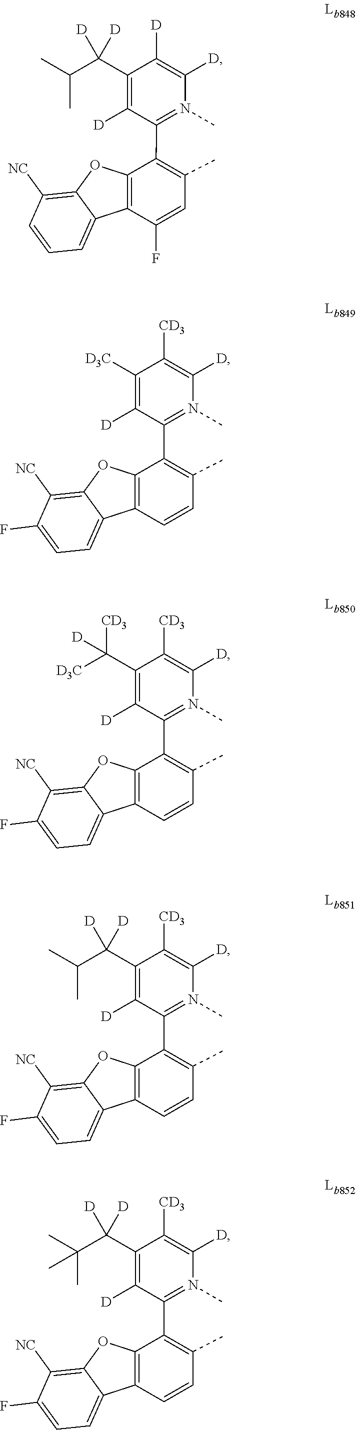

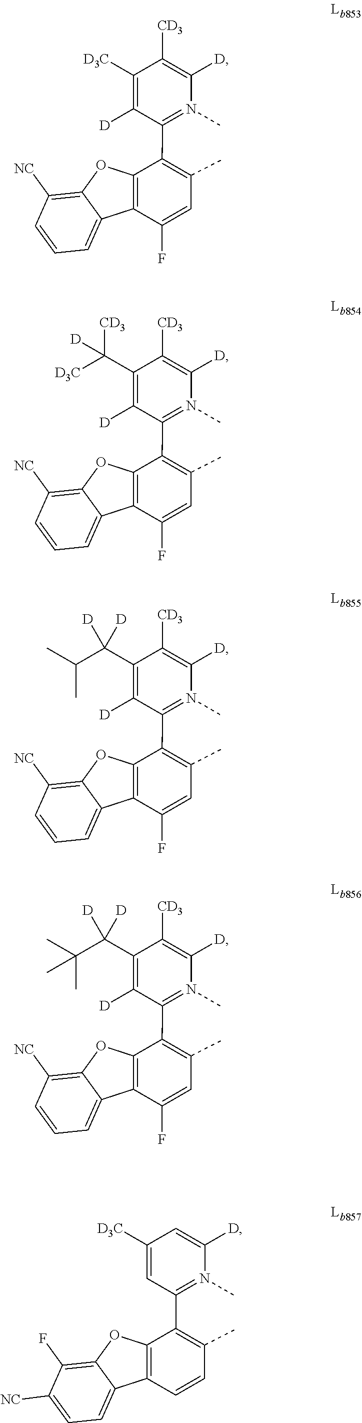

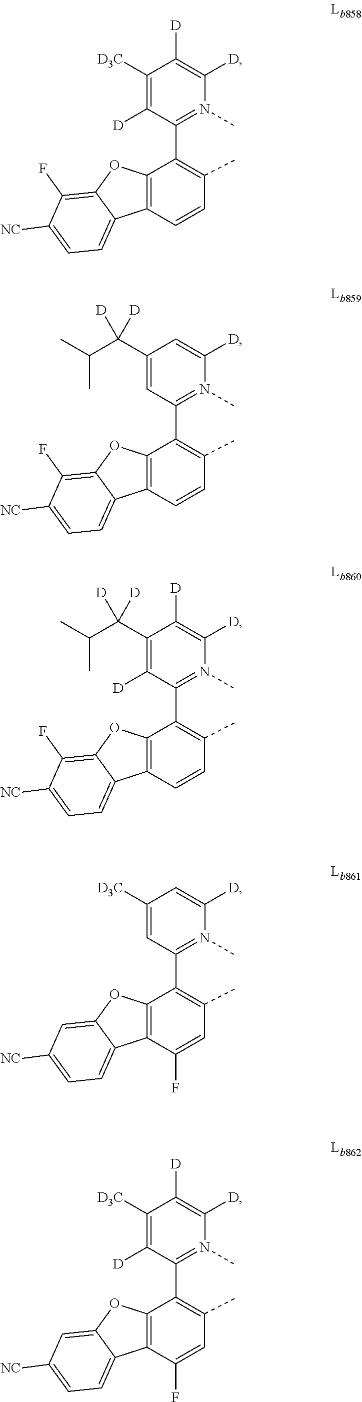

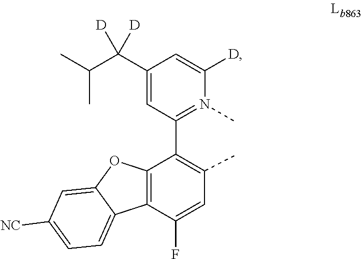

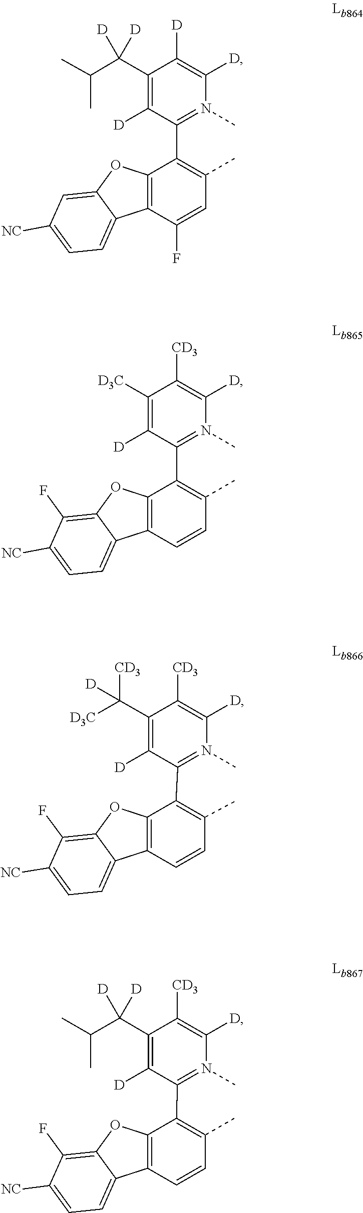

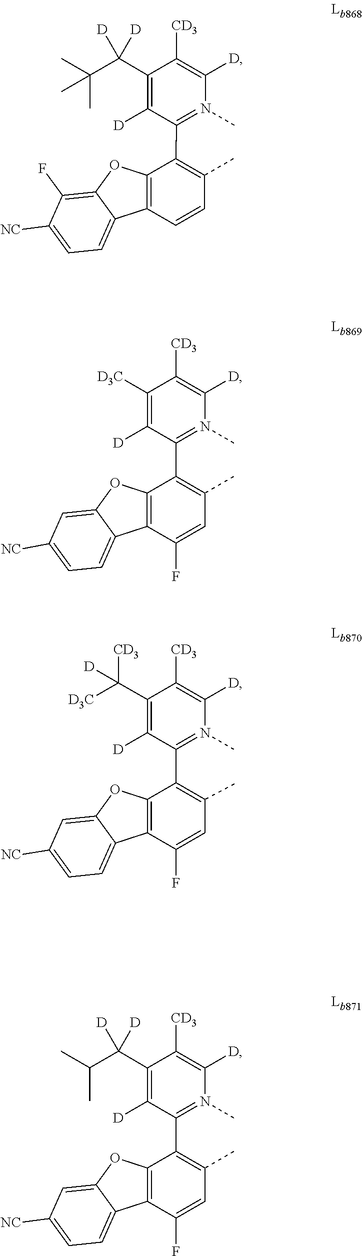

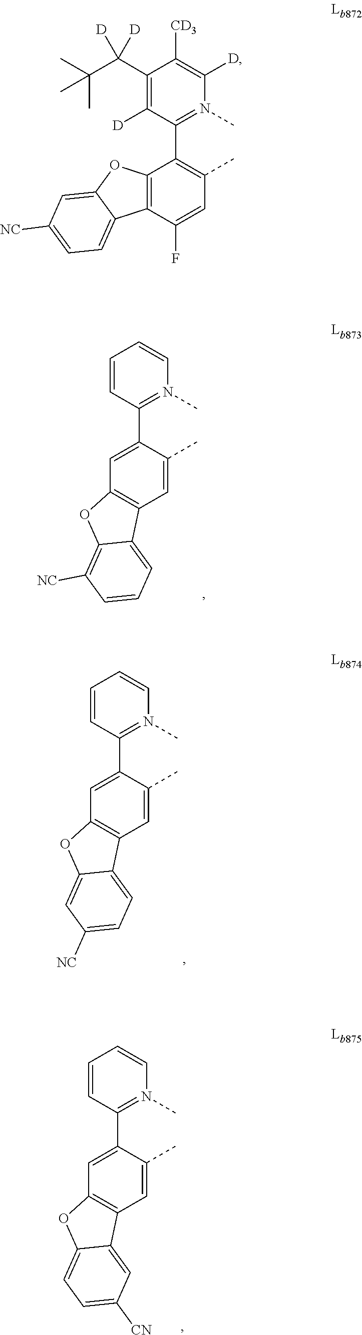

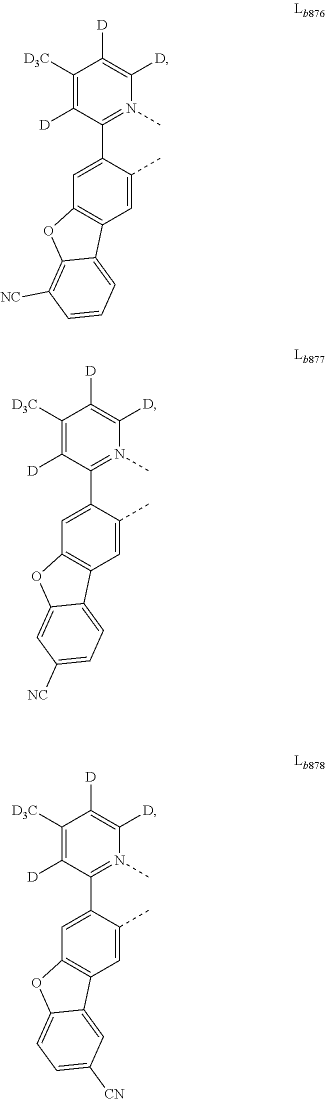

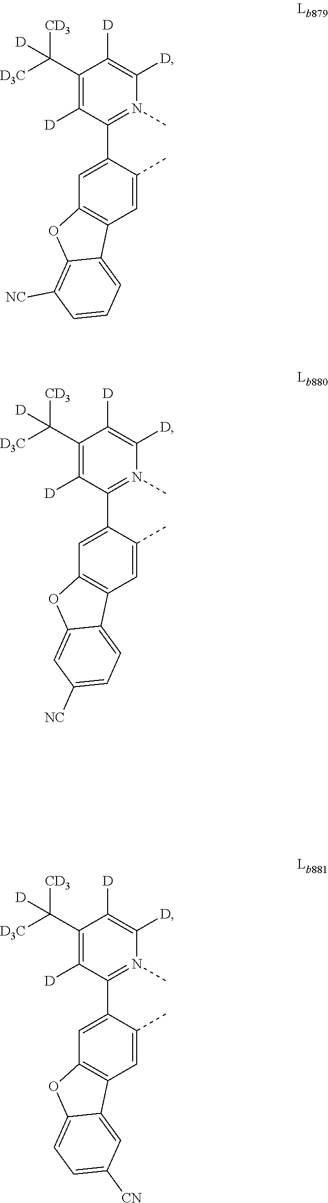

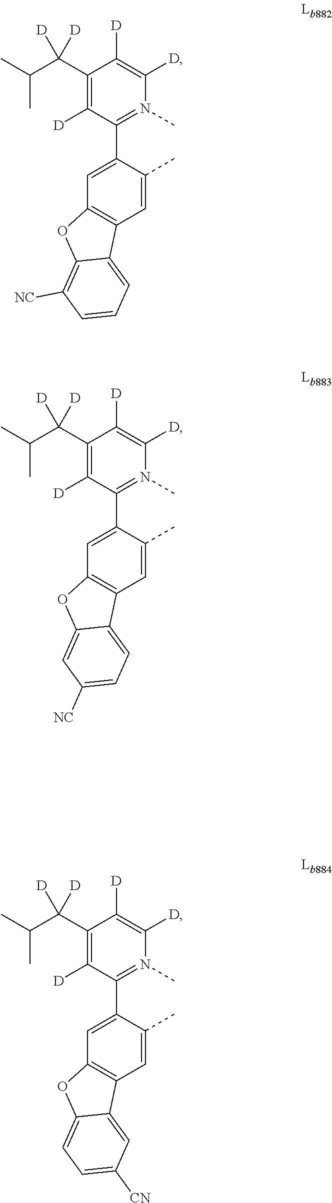

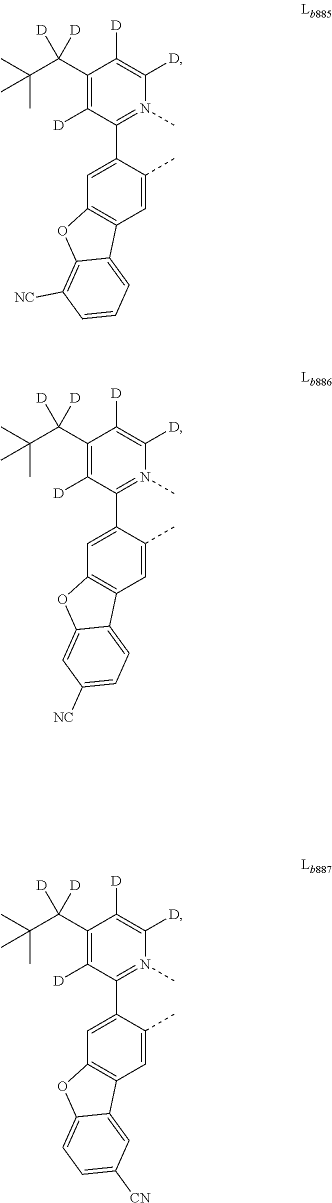

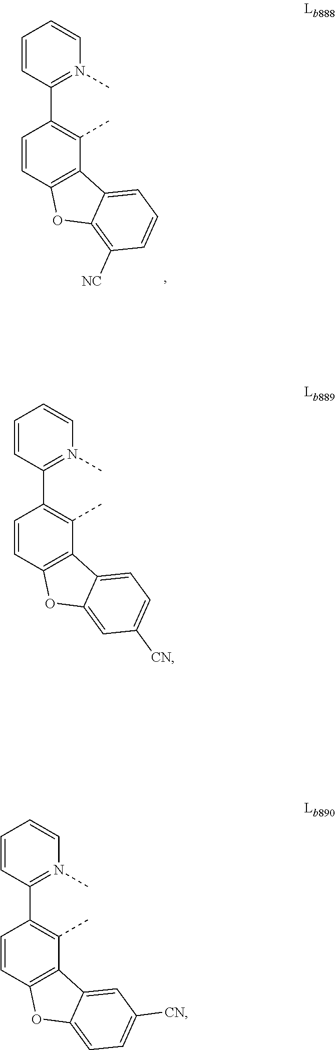

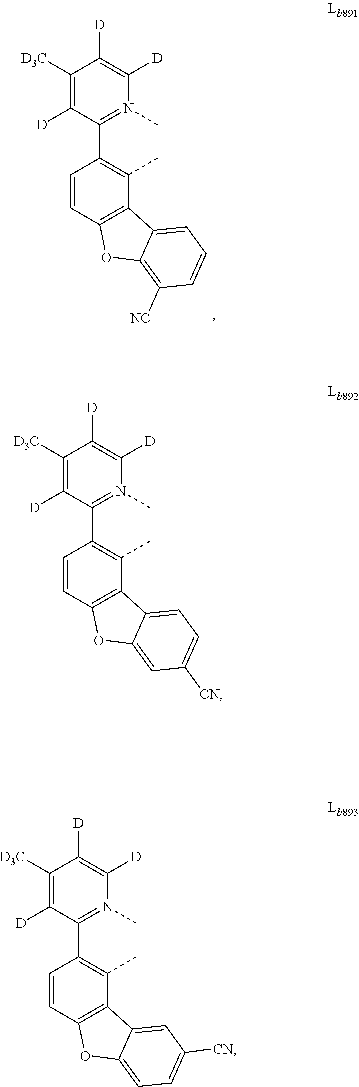

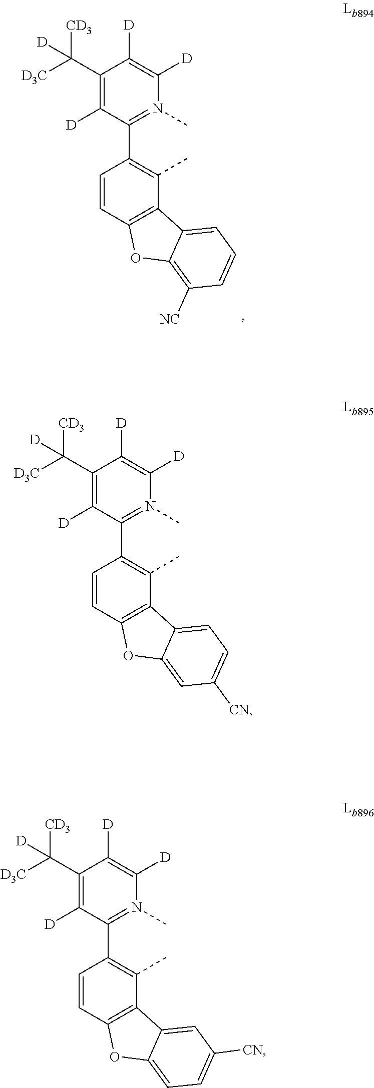

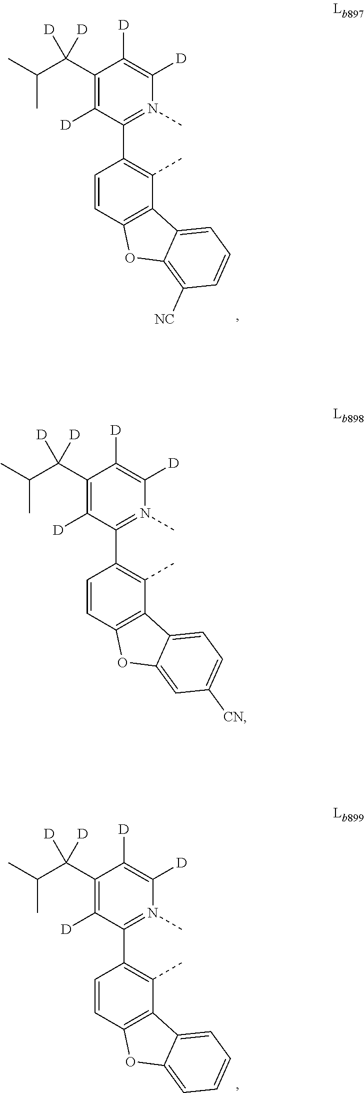

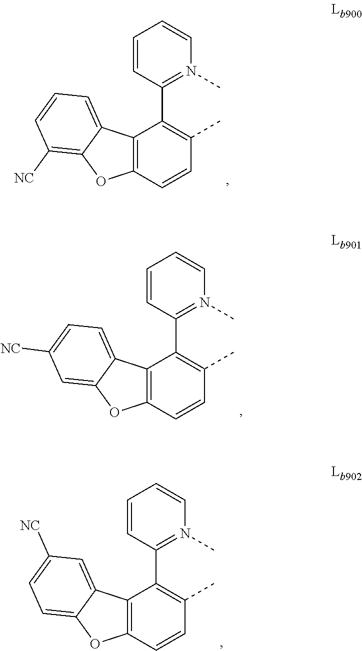

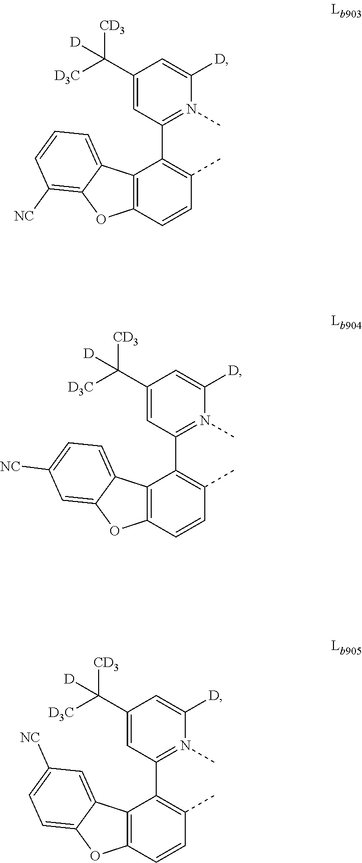

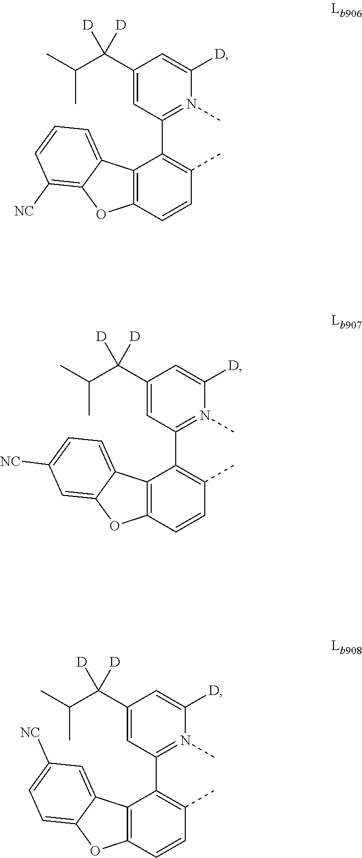

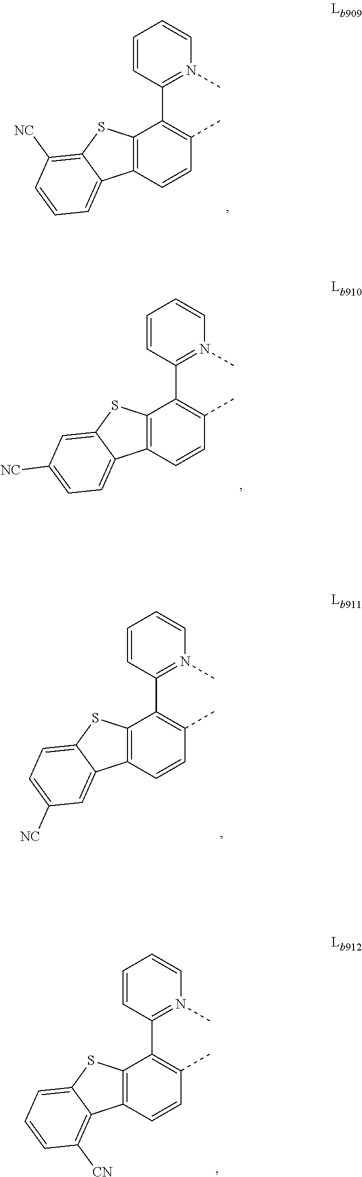

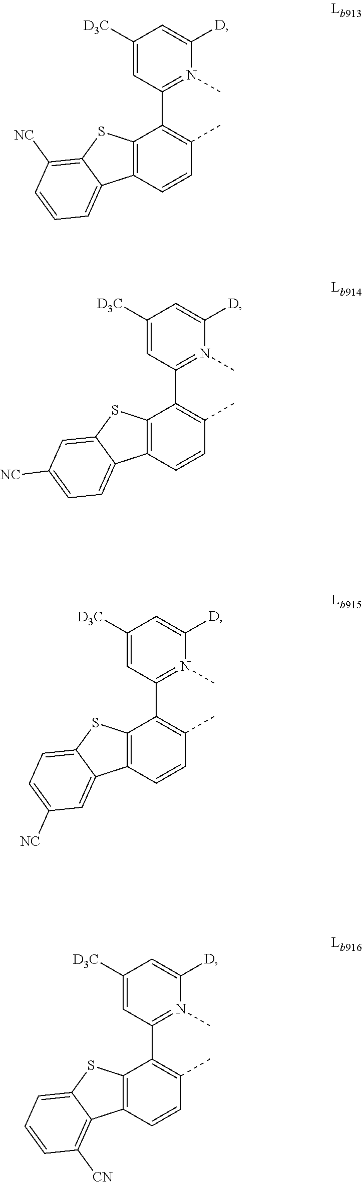

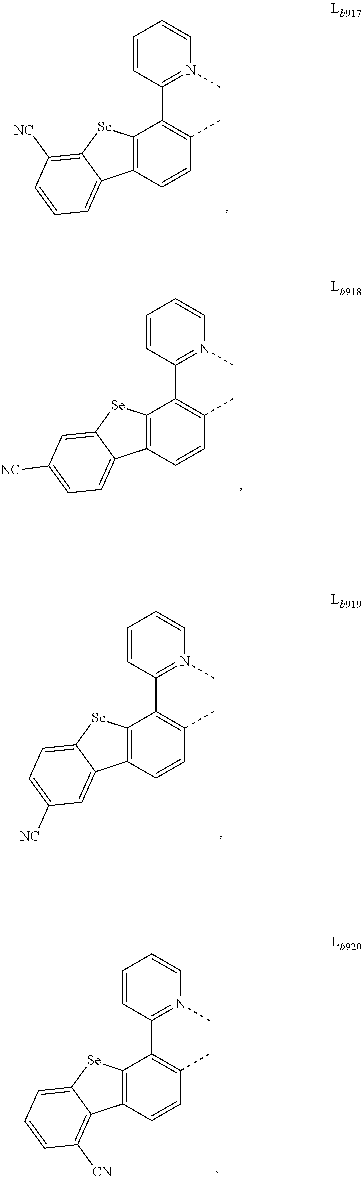

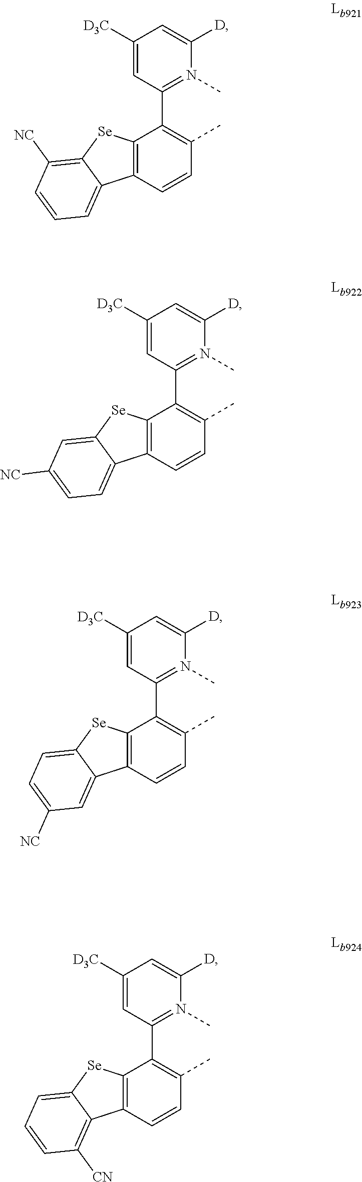

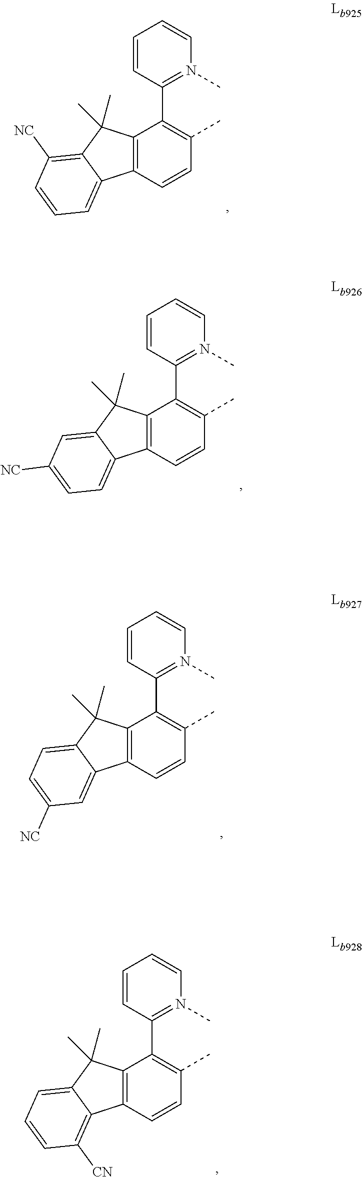

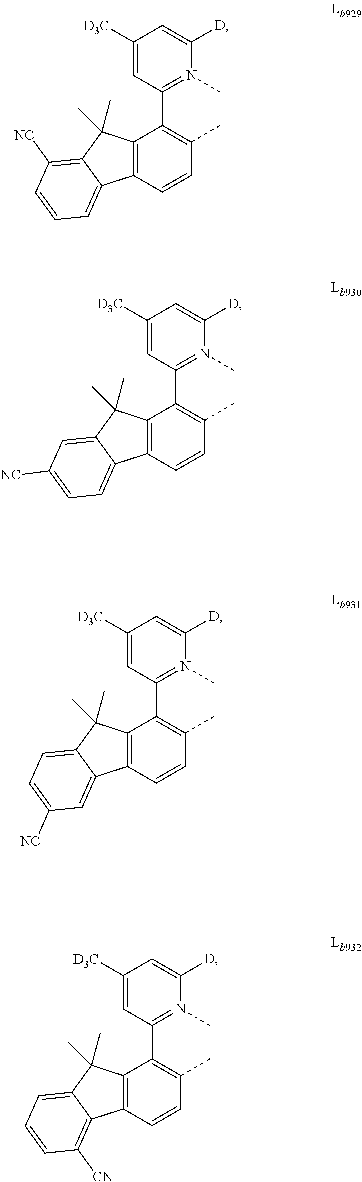

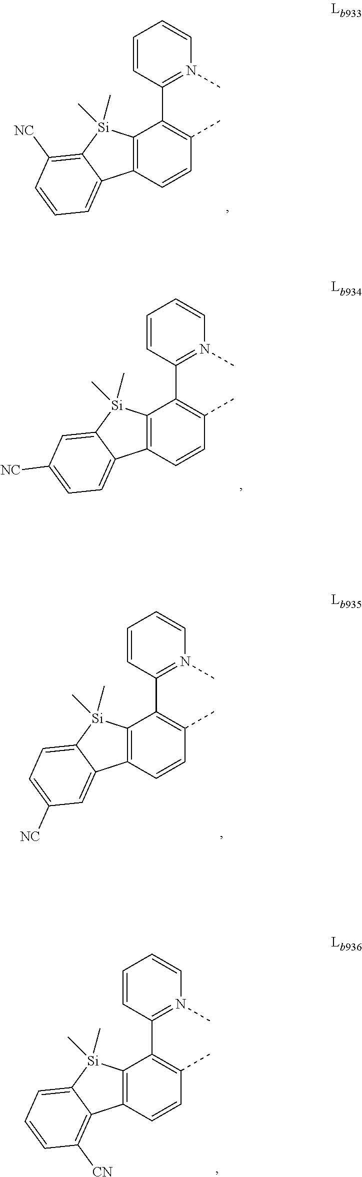

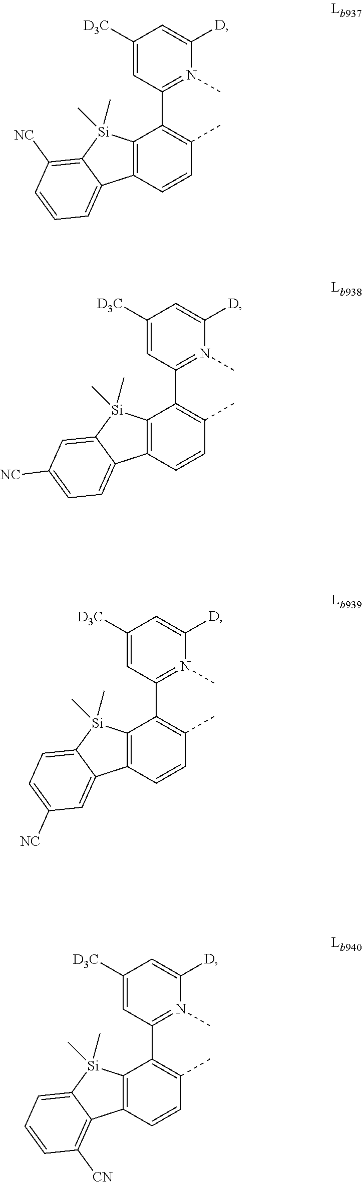

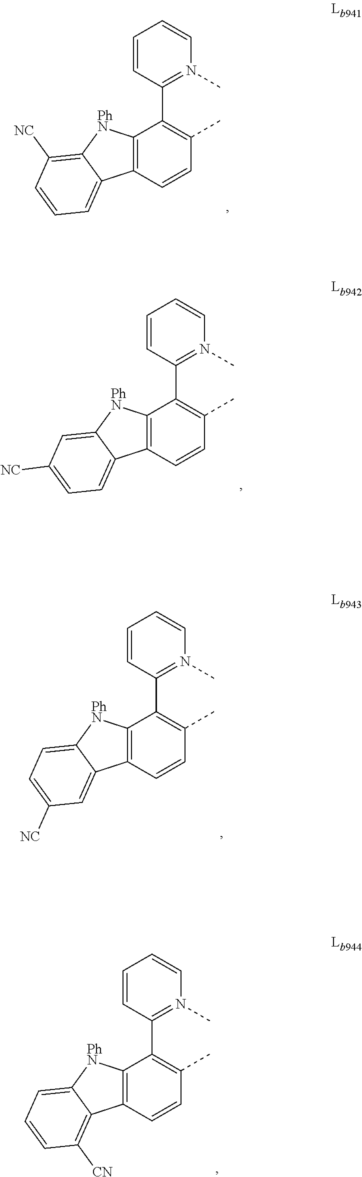

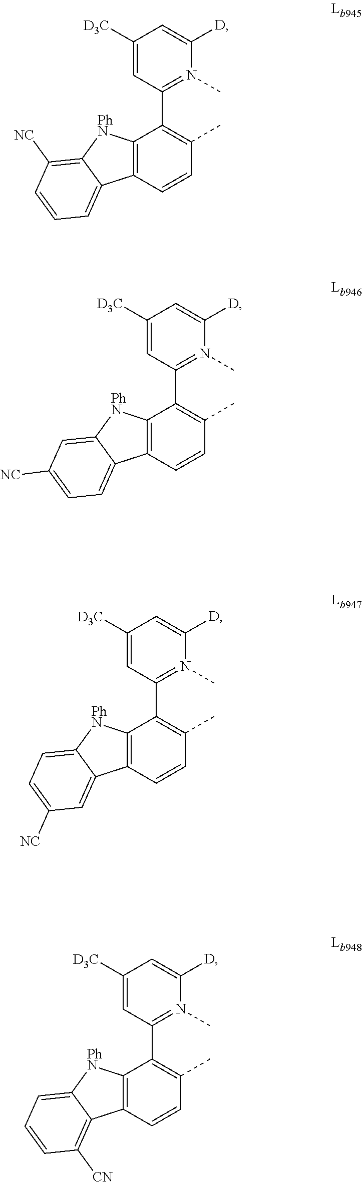

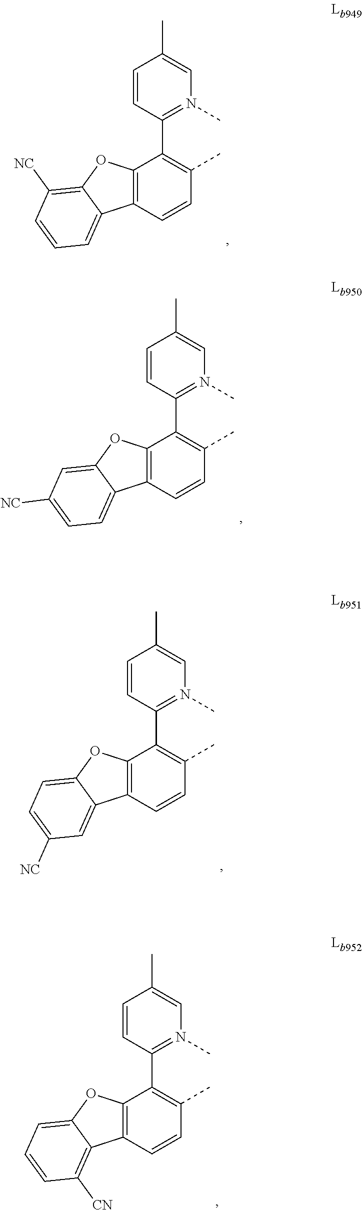

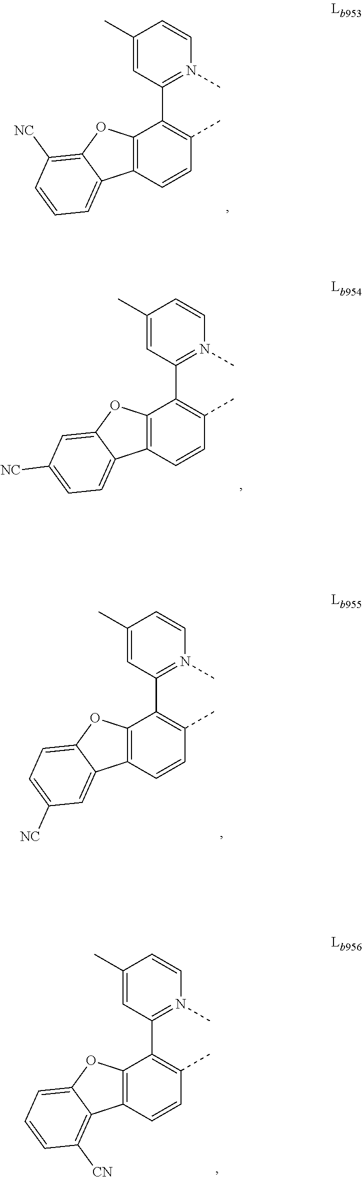

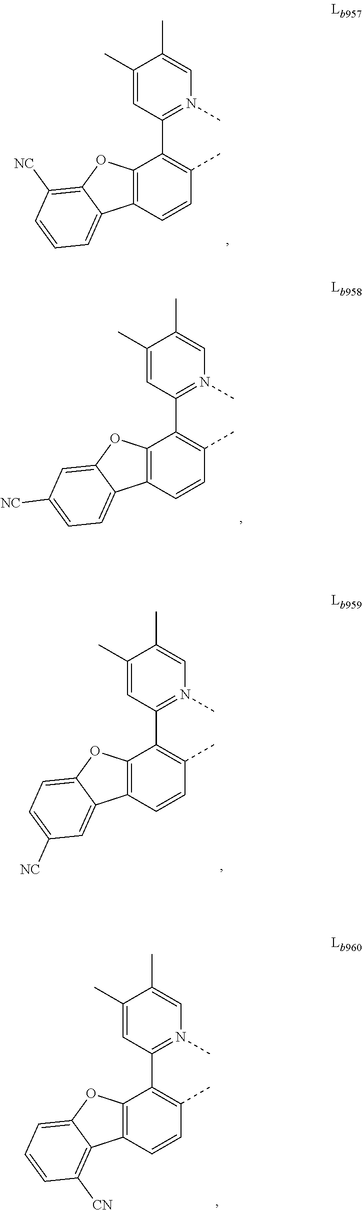

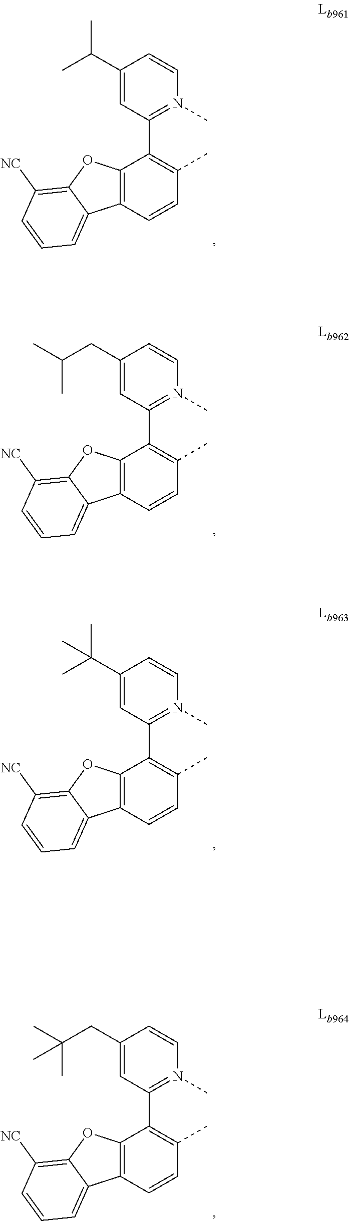

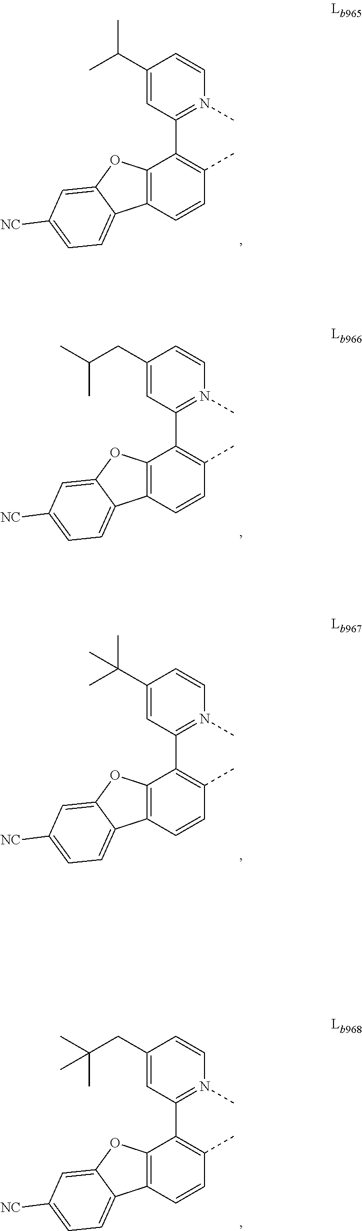

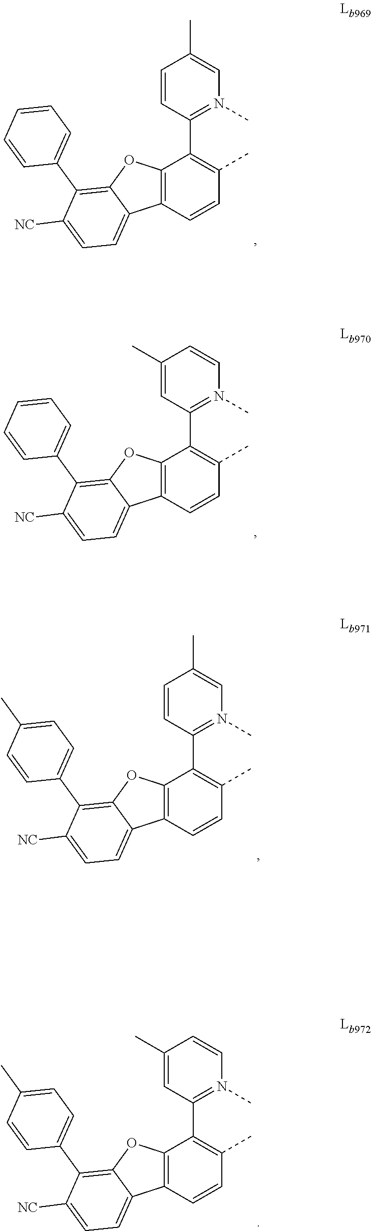

19. The metal complex of claim 1, wherein the metal complex has a structure of Ir(Lₐ)₂L_{b}, wherein the two Lₐ are identical, Lₐ is selected from the group consisting of Lₐ₁ to Lₐ₂₀₆, and L_{b} is selected from the group consisting of L_{b1} to L_{b972};
preferably, the metal complex is selected from the group consisting of Metal Complex 1 to Metal Complex 448, wherein Metal Complex 1 to Metal Complex 448 have the structure of Ir(Lₐ)₂L_{b}, wherein the two Lₐ are identical and Lₐ and L_{b} correspond to structures shown in the following table, respectively:

| Metal | | | Metal | | |
|---|---|---|---|---|---|
| Complex | Lₐ | L_{b} | Complex | Lₐ | L_{b} |
| 1 | Lₐ₁ | L_{b1} | 2 | Lₐ₁ | L_{b2} |
| 3 | Lₐ₁ | L_{b3} | 4 | Lₐ₁ | L_{b4} |
| 5 | Lₐ₁ | L_{b221} | 6 | Lₐ₁ | L_{b227} |
| 7 | Lₐ₁ | L_{b477} | 8 | Lₐ₁ | L_{b481} |
| 9 | Lₐ₁ | L_{b485} | 10 | Lₐ₁ | L_{b497} |
| 11 | Lₐ₁ | L_{b501} | 12 | Lₐ₁ | L_{b597} |
| 13 | Lₐ₁ | L_{b623} | 14 | Lₐ₁ | L_{b624} |
| 15 | Lₐ₁ | L_{b625} | 16 | Lₐ₁ | L_{b632} |
| 17 | Lₐ₁ | L_{b680} | 18 | Lₐ₁ | L_{b687} |
| 19 | Lₐ₁ | L_{b688} | 20 | Lₐ₁ | L_{b689} |
| 21 | Lₐ₁ | L_{b691} | 22 | Lₐ₁ | L_{b736} |
| 23 | Lₐ₁ | L_{b737} | 24 | Lₐ₁ | L_{b743} |
| 25 | Lₐ₁ | L_{b745} | 26 | Lₐ₁ | L_{b750} |
| 27 | Lₐ₁ | L_{b753} | 28 | Lₐ₁ | L_{b763} |
| 29 | Lₐ₁ | L_{b764} | 30 | Lₐ₁ | L_{b766} |
| 31 | Lₐ₄ | L_{b1} | 32 | Lₐ₄ | L_{b2} |
| 33 | Lₐ₄ | L_{b3} | 34 | Lₐ₄ | L_{b4} |
| 35 | Lₐ₄ | L_{b221} | 36 | Lₐ₄ | L_{b227} |
| 37 | Lₐ₄ | L_{b477} | 38 | Lₐ₄ | L_{b481} |
| 39 | Lₐ₄ | L_{b485} | 40 | Lₐ₄ | L_{b497} |
| 41 | Lₐ₄ | L_{b501} | 42 | Lₐ₄ | L_{b597} |
| 43 | Lₐ₄ | L_{b623} | 44 | Lₐ₄ | L_{b624} |
| 45 | Lₐ₄ | L_{b625} | 46 | Lₐ₄ | L_{b632} |
| 47 | Lₐ₄ | L_{b680} | 48 | Lₐ₄ | L_{b687} |
| 49 | Lₐ₄ | L_{b688} | 50 | Lₐ₄ | L_{b689} |
| 51 | Lₐ₄ | L_{b691} | 52 | Lₐ₄ | L_{b736} |
| 53 | Lₐ₄ | L_{b737} | 54 | Lₐ₄ | L_{b743} |
| 55 | Lₐ₄ | L_{b745} | 56 | Lₐ₄ | L_{b750} |
| 57 | Lₐ₄ | L_{b753} | 58 | Lₐ₄ | L_{b763} |
| 59 | Lₐ₄ | L_{b764} | 60 | Lₐ₄ | L_{b766} |
| 61 | Lₐ₅ | L_{b1} | 62 | Lₐ₅ | L_{b2} |
| 63 | Lₐ₅ | L_{b3} | 64 | Lₐ₅ | L_{b4} |
| 65 | Lₐ₅ | L_{b221} | 66 | Lₐ₅ | L_{b227} |
| 67 | Lₐ₅ | L_{b477} | 68 | Lₐ₅ | L_{b481} |
| 69 | Lₐ₅ | L_{b485} | 70 | Lₐ₅ | L_{b497} |
| 71 | Lₐ₅ | L_{b501} | 72 | Lₐ₅ | L_{b597} |
| 73 | Lₐ₅ | L_{b623} | 74 | Lₐ₅ | L_{b624} |
| 75 | Lₐ₅ | L_{b625} | 76 | Lₐ₅ | L_{b632} |
| 77 | Lₐ₅ | L_{b680} | 78 | Lₐ₅ | L_{b687} |
| 79 | Lₐ₅ | L_{b688} | 80 | Lₐ₅ | L_{b689} |
| 81 | Lₐ₅ | L_{b691} | 82 | Lₐ₅ | L_{b736} |
| 83 | Lₐ₅ | L_{b737} | 84 | Lₐ₅ | L_{b743} |
| 85 | Lₐ₅ | L_{b745} | 86 | Lₐ₅ | L_{b750} |
| 87 | Lₐ₅ | L_{b753} | 88 | Lₐ₅ | L_{b763} |
| 89 | Lₐ₅ | L_{b764} | 90 | Lₐ₅ | L_{b766} |
| 91 | Lₐ₁₉ | L_{b1} | 92 | Lₐ₁₉ | L_{b2} |
| 93 | Lₐ₁₉ | L_{b3} | 94 | Lₐ₁₉ | L_{b4} |
| 95 | Lₐ₁₉ | L_{b221} | 96 | Lₐ₁₉ | L_{b227} |
| 97 | Lₐ₁₉ | L_{b477} | 98 | Lₐ₁₉ | L_{b481} |
| 99 | Lₐ₁₉ | L_{b485} | 100 | Lₐ₁₉ | L_{b497} |
| 101 | Lₐ₁₉ | L_{b501} | 102 | Lₐ₁₉ | L_{b597} |
| 103 | Lₐ₁₉ | L_{b623} | 104 | Lₐ₁₉ | L_{b624} |
| 105 | Lₐ₁₉ | L_{b625} | 106 | Lₐ₁₉ | L_{b632} |
| 107 | Lₐ₁₉ | L_{b680} | 108 | Lₐ₁₉ | L_{b687} |
| 109 | Lₐ₁₉ | L_{b688} | 110 | Lₐ₁₉ | L_{b689} |
| 111 | Lₐ₁₉ | L_{b691} | 112 | Lₐ₁₉ | L_{b736} |
| 113 | Lₐ₁₉ | L_{b737} | 114 | Lₐ₁₉ | L_{b743} |
| 115 | Lₐ₁₉ | L_{b745} | 116 | Lₐ₁₉ | L_{b750} |
| 117 | Lₐ₁₉ | L_{b753} | 118 | Lₐ₁₉ | L_{b763} |
| 119 | Lₐ₁₉ | L_{b764} | 120 | Lₐ₁₉ | L_{b766} |
| 121 | Lₐ₂₂ | L_{b1} | 122 | Lₐ₂₂ | L_{b2} |
| 123 | Lₐ₂₂ | L_{b3} | 124 | Lₐ₂₂ | L_{b4} |
| 125 | Lₐ₂₂ | L_{b221} | 126 | Lₐ₂₂ | L_{b227} |
| 127 | Lₐ₂₂ | L_{b477} | 128 | Lₐ₂₂ | L_{b481} |
| 129 | Lₐ₂₂ | L_{b485} | 130 | Lₐ₂₂ | L_{b497} |
| 131 | Lₐ₂₂ | L_{b501} | 132 | Lₐ₂₂ | L_{b597} |
| 133 | Lₐ₂₂ | L_{b623} | 134 | Lₐ₂₂ | L_{b624} |
| 135 | Lₐ₂₂ | L_{b625} | 136 | Lₐ₂₂ | L_{b632} |
| 137 | Lₐ₂₂ | L_{b680} | 138 | Lₐ₂₂ | L_{b687} |
| 139 | Lₐ₂₂ | L_{b688} | 140 | Lₐ₂₂ | L_{b689} |
| 141 | Lₐ₂₂ | L_{b691} | 142 | Lₐ₂₂ | L_{b736} |
| 143 | Lₐ₂₂ | L_{b737} | 144 | Lₐ₂₂ | L_{b743} |
| 145 | Lₐ₂₂ | L_{b745} | 146 | Lₐ₂₂ | L_{b750} |
| 147 | Lₐ₂₂ | L_{b753} | 148 | Lₐ₂₂ | L_{b763} |
| 149 | Lₐ₂₂ | L_{b764} | 150 | Lₐ₂₂ | L_{b766} |
| 151 | Lₐ₃₂ | L_{b1} | 152 | Lₐ₃₂ | L_{b2} |
| 153 | Lₐ₃₂ | L_{b3} | 154 | Lₐ₃₂ | L_{b4} |
| 155 | Lₐ₃₂ | L_{b221} | 156 | Lₐ₃₂ | L_{b227} |
| 157 | Lₐ₃₂ | L_{b477} | 158 | Lₐ₃₂ | L_{b481} |
| 159 | Lₐ₃₂ | L_{b485} | 160 | Lₐ₃₂ | L_{b497} |
| 161 | Lₐ₃₂ | L_{b501} | 162 | Lₐ₃₂ | L_{b597} |
| 163 | Lₐ₃₂ | L_{b623} | 164 | Lₐ₃₂ | L_{b624} |
| 165 | Lₐ₃₂ | L_{b625} | 166 | Lₐ₃₂ | L_{b632} |
| 167 | Lₐ₃₂ | L_{b680} | 168 | Lₐ₃₂ | L_{b687} |
| 169 | Lₐ₃₂ | L_{b688} | 170 | Lₐ₃₂ | L_{b689} |
| 171 | Lₐ₃₂ | L_{b691} | 172 | Lₐ₃₂ | L_{b736} |
| 173 | Lₐ₃₂ | L_{b737} | 174 | Lₐ₃₂ | L_{b743} |
| 175 | Lₐ₃₂ | L_{b745} | 176 | Lₐ₃₂ | L_{b750} |
| 177 | Lₐ₃₂ | L_{b753} | 178 | Lₐ₃₂ | L_{b763} |
| 179 | Lₐ₃₂ | L_{b764} | 180 | Lₐ₃₂ | L_{b766} |
| 181 | Lₐ₇₁ | L_{b1} | 182 | Lₐ₇₁ | L_{b2} |
| 183 | Lₐ₇₁ | L_{b3} | 184 | Lₐ₇₁ | L_{b4} |
| 185 | Lₐ₇₁ | L_{b221} | 186 | Lₐ₇₁ | L_{b227} |
| 187 | Lₐ₇₁ | L_{b477} | 188 | Lₐ₇₁ | L_{b481} |
| 189 | Lₐ₇₁ | L_{b485} | 190 | Lₐ₇₁ | L_{b497} |
| 191 | Lₐ₇₁ | L_{b501} | 192 | Lₐ₇₁ | L_{b597} |
| 193 | Lₐ₇₁ | L_{b623} | 194 | Lₐ₇₁ | L_{b624} |
| 195 | Lₐ₇₁ | L_{b625} | 196 | Lₐ₇₁ | L_{b632} |
| 197 | Lₐ₇₁ | L_{b680} | 198 | Lₐ₇₁ | L_{b687} |
| 199 | Lₐ₇₁ | L_{b688} | 200 | Lₐ₇₁ | L_{b689} |
| 201 | Lₐ₇₁ | L_{b691} | 202 | Lₐ₇₁ | L_{b736} |
| 203 | Lₐ₇₁ | L_{b737} | 204 | Lₐ₇₁ | L_{b743} |
| 205 | Lₐ₇₁ | L_{b745} | 206 | Lₐ₇₁ | L_{b750} |
| 207 | Lₐ₇₁ | L_{b753} | 208 | Lₐ₇₁ | L_{b763} |
| 209 | Lₐ₇₁ | L_{b764} | 210 | Lₐ₇₁ | L_{b766} |
| 211 | Lₐ₈₄ | L_{b1} | 212 | Lₐ₈₄ | L_{b2} |
| 213 | Lₐ₈₄ | L_{b3} | 214 | Lₐ₈₄ | L_{b4} |
| 215 | Lₐ₈₄ | L_{b221} | 216 | Lₐ₈₄ | L_{b227} |
| 217 | Lₐ₈₄ | L_{b477} | 218 | Lₐ₈₄ | L_{b481} |
| 219 | Lₐ₈₄ | L_{b485} | 220 | Lₐ₈₄ | L_{b497} |
| 221 | Lₐ₈₄ | L_{b501} | 222 | Lₐ₈₄ | L_{b597} |
| 223 | Lₐ₈₄ | L_{b623} | 224 | Lₐ₈₄ | L_{b624} |
| 225 | Lₐ₈₄ | L_{b625} | 226 | Lₐ₈₄ | L_{b632} |
| 227 | Lₐ₈₄ | L_{b680} | 228 | Lₐ₈₄ | L_{b687} |
| 229 | Lₐ₈₄ | L_{b688} | 230 | Lₐ₈₄ | L_{b689} |
| 231 | Lₐ₈₄ | L_{b691} | 232 | Lₐ₈₄ | L_{b736} |
| 233 | Lₐ₈₄ | L_{b737} | 234 | Lₐ₈₄ | L_{b743} |
| 235 | Lₐ₈₄ | L_{b745} | 236 | Lₐ₈₄ | L_{b750} |
| 237 | Lₐ₈₄ | L_{b753} | 238 | Lₐ₈₄ | L_{b763} |
| 239 | Lₐ₈₄ | L_{b764} | 240 | Lₐ₈₄ | L_{b766} |
| 241 | Lₐ₈₇ | L_{b1} | 242 | Lₐ₈₇ | L_{b2} |
| 243 | Lₐ₈₇ | L_{b3} | 244 | Lₐ₈₇ | L_{b4} |
| 245 | Lₐ₈₇ | L_{b221} | 246 | Lₐ₈₇ | L_{b227} |
| 247 | Lₐ₈₇ | L_{b477} | 248 | Lₐ₈₇ | L_{b481} |
| 249 | Lₐ₈₇ | L_{b485} | 250 | Lₐ₈₇ | L_{b497} |
| 251 | Lₐ₈₇ | L_{b501} | 252 | Lₐ₈₇ | L_{b597} |
| 253 | Lₐ₈₇ | L_{b623} | 254 | Lₐ₈₇ | L_{b624} |
| 255 | Lₐ₈₇ | L_{b625} | 256 | Lₐ₈₇ | L_{b632} |
| 257 | Lₐ₈₇ | L_{b680} | 258 | Lₐ₈₇ | L_{b687} |
| 259 | Lₐ₈₇ | L_{b688} | 260 | Lₐ₈₇ | L_{b689} |
| 261 | Lₐ₈₇ | L_{b691} | 262 | Lₐ₈₇ | L_{b736} |
| 263 | Lₐ₈₇ | L_{b737} | 264 | Lₐ₈₇ | L_{b743} |
| 265 | Lₐ₈₇ | L_{b745} | 266 | Lₐ₈₇ | L_{b750} |
| 267 | Lₐ₈₇ | L_{b753} | 268 | Lₐ₈₇ | L_{b763} |
| 269 | Lₐ₈₇ | L_{b764} | 270 | Lₐ₈₇ | L_{b766} |
| 271 | Lₐ₁₂₉ | L_{b1} | 272 | Lₐ₁₂₉ | L_{b2} |
| 273 | Lₐ₁₂₉ | L_{b3} | 274 | Lₐ₁₂₉ | L_{b4} |
| 275 | Lₐ₁₂₉ | L_{b221} | 276 | Lₐ₁₂₉ | L_{b227} |
| 277 | Lₐ₁₂₉ | L_{b477} | 278 | Lₐ₁₂₉ | L_{b481} |
| 279 | Lₐ₁₂₉ | L_{b485} | 280 | Lₐ₁₂₉ | L_{b497} |
| 281 | Lₐ₁₂₉ | L_{b501} | 282 | Lₐ₁₂₉ | L_{b597} |
| 283 | Lₐ₁₂₉ | L_{b623} | 284 | Lₐ₁₂₉ | L_{b624} |
| 285 | Lₐ₁₂₉ | L_{b625} | 286 | Lₐ₁₂₉ | L_{b632} |
| 287 | Lₐ₁₂₉ | L_{b680} | 288 | Lₐ₁₂₉ | L_{b687} |
| 289 | Lₐ₁₂₉ | L_{b688} | 290 | Lₐ₁₂₉ | L_{b689} |
| 291 | Lₐ₁₂₉ | L_{b691} | 292 | Lₐ₁₂₉ | L_{b736} |
| 293 | Lₐ₁₂₉ | L_{b737} | 294 | Lₐ₁₂₉ | L_{b743} |
| 295 | Lₐ₁₂₉ | L_{b745} | 296 | Lₐ₁₂₉ | L_{b750} |
| 297 | Lₐ₁₂₉ | L_{b753} | 298 | Lₐ₁₂₉ | L_{b763} |
| 299 | Lₐ₁₂₉ | L_{b764} | 300 | Lₐ₁₂₉ | L_{b766} |
| 301 | Lₐ₁₃₄ | L_{b1} | 302 | Lₐ₁₃₄ | L_{b2} |
| 303 | Lₐ₁₃₄ | L_{b3} | 304 | Lₐ₁₃₄ | L_{b4} |
| 305 | Lₐ₁₃₄ | L_{b221} | 306 | Lₐ₁₃₄ | L_{b227} |
| 307 | Lₐ₁₃₄ | L_{b477} | 308 | Lₐ₁₃₄ | L_{b481} |
| 309 | Lₐ₁₃₄ | L_{b485} | 310 | Lₐ₁₃₄ | L_{b497} |
| 311 | Lₐ₁₃₄ | L_{b501} | 312 | Lₐ₁₃₄ | L_{b597} |
| 313 | Lₐ₁₃₄ | L_{b623} | 314 | Lₐ₁₃₄ | L_{b624} |
| 315 | Lₐ₁₃₄ | L_{b625} | 316 | Lₐ₁₃₄ | L_{b632} |
| 317 | Lₐ₁₃₄ | L_{b680} | 318 | Lₐ₁₃₄ | L_{b687} |
| 319 | Lₐ₁₃₄ | L_{b688} | 320 | Lₐ₁₃₄ | L_{b689} |
| 321 | Lₐ₁₃₄ | L_{b691} | 322 | Lₐ₁₃₄ | L_{b736} |
| 323 | Lₐ₁₃₄ | L_{b737} | 324 | Lₐ₁₃₄ | L_{b743} |
| 325 | Lₐ₁₃₄ | L_{b745} | 326 | Lₐ₁₃₄ | L_{b750} |
| 327 | Lₐ₁₃₄ | L_{b753} | 328 | Lₐ₁₃₄ | L_{b763} |
| 329 | Lₐ₁₃₄ | L_{b764} | 330 | Lₐ₁₃₄ | L_{b766} |
| 331 | Lₐ₁₃₅ | L_{b1} | 332 | Lₐ₁₃₅ | L_{b2} |
| 333 | Lₐ₁₃₅ | L_{b3} | 334 | Lₐ₁₃₅ | L_{b4} |
| 335 | Lₐ₁₃₅ | L_{b221} | 336 | Lₐ₁₃₅ | L_{b227} |
| 337 | Lₐ₁₃₅ | L_{b477} | 338 | Lₐ₁₃₅ | L_{b481} |
| 339 | Lₐ₁₃₅ | L_{b485} | 340 | Lₐ₁₃₅ | L_{b497} |
| 341 | Lₐ₁₃₅ | L_{b501} | 342 | Lₐ₁₃₅ | L_{b597} |
| 343 | Lₐ₁₃₅ | L_{b623} | 344 | Lₐ₁₃₅ | L_{b624} |
| 345 | Lₐ₁₃₅ | L_{b625} | 346 | Lₐ₁₃₅ | L_{b632} |
| 347 | Lₐ₁₃₅ | L_{b680} | 348 | Lₐ₁₃₅ | L_{b687} |
| 349 | Lₐ₁₃₅ | L_{b688} | 350 | Lₐ₁₃₅ | L_{b689} |
| 351 | Lₐ₁₃₅ | L_{b691} | 352 | Lₐ₁₃₅ | L_{b736} |
| 353 | Lₐ₁₃₅ | L_{b737} | 354 | Lₐ₁₃₅ | L_{b743} |
| 355 | Lₐ₁₃₅ | L_{b745} | 356 | Lₐ₁₃₅ | L_{b750} |
| 357 | Lₐ₁₃₅ | L_{b753} | 358 | Lₐ₁₃₅ | L_{b763} |
| 359 | Lₐ₁₃₅ | L_{b764} | 360 | Lₐ₁₃₅ | L_{b766} |
| 361 | Lₐ₁₃₈ | L_{b1} | 362 | Lₐ₁₃₈ | L_{b2} |
| 363 | Lₐ₁₃₈ | L_{b3} | 364 | Lₐ₁₃₈ | L_{b4} |
| 365 | Lₐ₁₃₈ | L_{b221} | 366 | Lₐ₁₃₈ | L_{b227} |
| 367 | Lₐ₁₃₈ | L_{b477} | 368 | Lₐ₁₃₈ | L_{b481} |
| 369 | Lₐ₁₃₈ | L_{b485} | 370 | Lₐ₁₃₈ | L_{b497} |
| 371 | Lₐ₁₃₈ | L_{b501} | 372 | Lₐ₁₃₈ | L_{b597} |
| 373 | Lₐ₁₃₈ | L_{b623} | 374 | Lₐ₁₃₈ | L_{b624} |
| 375 | Lₐ₁₃₈ | L_{b625} | 376 | Lₐ₁₃₈ | L_{b632} |
| 377 | Lₐ₁₃₈ | L_{b680} | 378 | Lₐ₁₃₈ | L_{b687} |
| 379 | Lₐ₁₃₈ | L_{b688} | 380 | Lₐ₁₃₈ | L_{b689} |
| 381 | Lₐ₁₃₈ | L_{b691} | 382 | Lₐ₁₃₈ | L_{b736} |
| 383 | Lₐ₁₃₈ | L_{b737} | 384 | Lₐ₁₃₈ | L_{b743} |
| 385 | Lₐ₁₃₈ | L_{b745} | 386 | Lₐ₁₃₈ | L_{b750} |
| 387 | Lₐ₁₃₈ | L_{b753} | 388 | Lₐ₁₃₈ | L_{b763} |
| 389 | Lₐ₁₃₈ | L_{b764} | 390 | Lₐ₁₃₈ | L_{b766} |
| 391 | Lₐ₁₆₁ | L_{b1} | 392 | Lₐ₁₆₁ | L_{b2} |
| 393 | Lₐ₁₆₁ | L_{b3} | 394 | Lₐ₁₆₁ | L_{b4} |
| 395 | Lₐ₁₆₁ | L_{b221} | 396 | Lₐ₁₆₁ | L_{b227} |
| 397 | Lₐ₁₆₁ | L_{b477} | 398 | Lₐ₁₆₁ | L_{b481} |
| 399 | Lₐ₁₆₁ | L_{b485} | 400 | Lₐ₁₆₁ | L_{b497} |
| 401 | Lₐ₁₆₁ | L_{b501} | 402 | Lₐ₁₆₁ | L_{b597} |
| 403 | Lₐ₁₆₁ | L_{b623} | 404 | Lₐ₁₆₁ | L_{b624} |
| 405 | Lₐ₁₆₁ | L_{b625} | 406 | Lₐ₁₆₁ | L_{b632} |
| 407 | Lₐ₁₆₁ | L_{b680} | 408 | Lₐ₁₆₁ | L_{b687} |
| 409 | Lₐ₁₆₁ | L_{b688} | 410 | Lₐ₁₆₁ | L_{b689} |
| 411 | Lₐ₁₆₁ | L_{b691} | 412 | Lₐ₁₆₁ | L_{b736} |
| 413 | Lₐ₁₆₁ | L_{b737} | 414 | Lₐ₁₆₁ | L_{b743} |
| 415 | Lₐ₁₆₁ | L_{b745} | 416 | Lₐ₁₆₁ | L_{b750} |
| 417 | Lₐ₁₆₁ | L_{b753} | 418 | Lₐ₁₆₁ | L_{b763} |
| 419 | Lₐ₁₆₁ | L_{b764} | 420 | Lₐ₁₆₁ | L_{b766} |
| 421 | Lₐ₁₆₈ | L_{b1} | 422 | Lₐ₁₆₈ | L_{b2} |
| 423 | Lₐ₁₆₈ | L_{b3} | 424 | Lₐ₁₆₈ | L_{b4} |
| 425 | Lₐ₁₆₈ | L_{b221} | 426 | Lₐ₁₆₈ | L_{b227} |
| 427 | Lₐ₁₆₈ | L_{b477} | 428 | Lₐ₁₆₈ | L_{b481} |
| 429 | Lₐ₁₆₈ | L_{b485} | 430 | Lₐ₁₆₈ | L_{b497} |
| 431 | Lₐ₁₆₈ | L_{b501} | 432 | Lₐ₁₆₈ | L_{b597} |
| 433 | Lₐ₁₆₈ | L_{b623} | 434 | Lₐ₁₆₈ | L_{b624} |
| 435 | Lₐ₁₆₈ | L_{b625} | 436 | Lₐ₁₆₈ | L_{b632} |
| 437 | Lₐ₁₆₈ | L_{b680} | 438 | Lₐ₁₆₈ | L_{b687} |
| 439 | Lₐ₁₆₈ | L_{b688} | 440 | Lₐ₁₆₈ | L_{b689} |
| 441 | Lₐ₁₆₈ | L_{b691} | 442 | Lₐ₁₆₈ | L_{b736} |
| 443 | Lₐ₁₆₈ | L_{b737} | 444 | Lₐ₁₆₈ | L_{b743} |
| 445 | Lₐ₁₆₈ | L_{b745} | 446 | Lₐ₁₆₈ | L_{b750} |
| 447 | Lₐ₁₆₈ | L_{b753} | 448 | Lₐ₁₆₈ | L_{b763} |

20. An electroluminescent device, comprising:
an anode,
a cathode, and
an organic layer disposed between the anode and the cathode, wherein at least one layer of the organic layer contains the metal complex of claim 1.

21. The electroluminescent device of claim 20, wherein the organic layer containing the metal complex is a light-emitting layer.

22. The electroluminescent device of claim 21, wherein the light-emitting layer emits green light.

23. The electroluminescent device of claim 21, wherein the light-emitting layer further contains at least one first host compound;
preferably, the light-emitting layer further contains at least two host compounds;
more preferably, at least one of the host compounds comprises at least one chemical group selected from the group consisting of: benzene, pyridine, pyrimidine, triazine, carbazole, azacarbazole, indolocarbazole, dibenzothiophene, aza-dibenzothiophene, dibenzofuran, azadibenzofuran, dibenzoselenophene, triphenylene, azatriphenylene, fluorene, silafluorene, naphthalene, quinoline, isoquinoline, quinazoline, quinoxaline, phenanthrene, azaphenanthrene and combinations thereof.

24. The electroluminescent device of claim 23, wherein the first host compound has a structure represented by Formula 3: 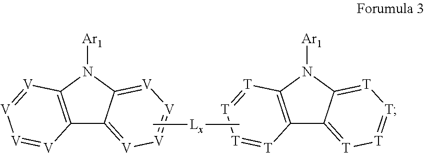
wherein
Lₓ is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or a combination thereof;
V is, at each occurrence identically or differently, selected from C, CRᵥ or N, and at least one of V is C and joined to Lₓ;
T is, at each occurrence identically or differently, selected from C, CRₜ or N, and at least one of T is C and joined to Lₓ;
Rᵥ and Rₜ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
Ar₁ is, at each occurrence identically or differently, selected from substituted or unsubstituted aryl having 6 to 30 carbon atoms, substituted or unsubstituted heteroaryl having 3 to 30 carbon atoms or a combination thereof;
adjacent substituents Rᵥ and Rₜ can be optionally joined to form a ring;
preferably, the first host compound has a structure represented by one of Formulas 3-a to 3-j:

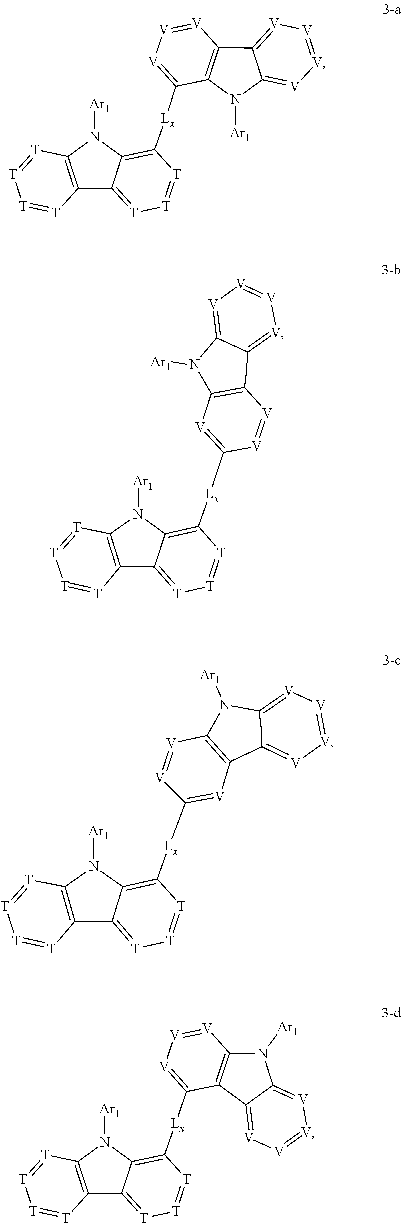

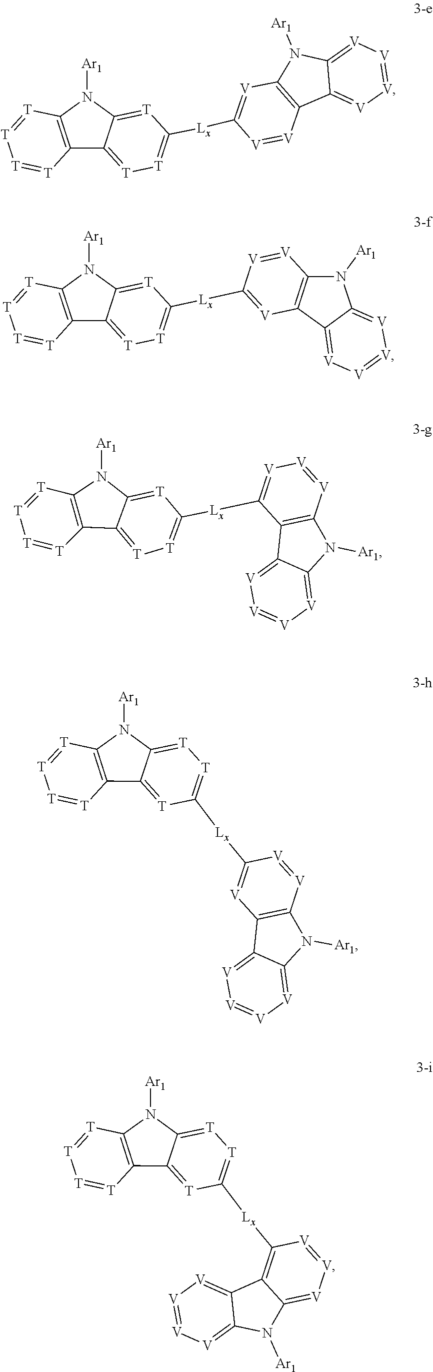

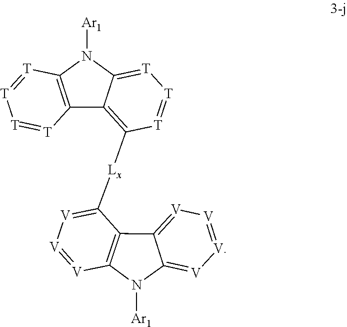

25. The electroluminescent device according to claim 23, wherein the metal complex is doped in the first host compound and the second host compound, and the weight of the metal complex accounts for 1% to 30% of the total weight of the light-emitting layer;
preferably, the weight of the metal complex accounts for 3% to 13% of the total weight of the light-emitting layer.

26. A compound composition containing the metal complex of claim 1.
